(19) United States
(12) Patent Application Publication
(10) Pub. No.: US 2026/0347866 A1
(43) Pub. Date: Nov. 5, 2026

(54) EDITING OF SEQUENCES REGULATING ALLERGEN AND FATTY ACID GENES IN PEANUT

(71) Applicant: BETTERSEEDS LTD, Givat Chen (IL)

(72) Inventors: Ido MARGALIT, Gan Yavne (IL); Tal SHERMAN, Rechovot (IL)

(73) Assignee: BETTERSEEDS LTD, Givat Chen (IL)

(21) Appl. No.: 19/099,663

(22) PCT Filed: Aug. 13, 2023
(86) PCT No.: PCT/IL2023/050844

§ 371 Date: Jan. 29, 2025

Related U.S. Application Data
(60) Provisional application No. 63/371,431, filed on Aug. 15, 2022.

(51) Int. Cl. C12N 15/82; C07K 14/415; C12N 9/02; C12N 9/22

(52) U.S. Cl. CPC C12N 15/8247; C07K 14/415; C12N 9/0071; C12N 9/22; C12N 15/8213; C12N 2310/20; C12Y 114/19006

(57) ABSTRACT

The present invention discloses a method for producing a modified peanut plant with at least one improved phenotypic trait. The method comprises steps of genetically modifying at least one regulatory gene involved in allergen synthesis and/or at least one regulatory gene involved in fatty acid metabolism, the improved phenotypic trait is relative to a corresponding peanut plant lacking the genetic modification.

### FIELD OF THE INVENTION

The present disclosure relates to conferring desirable traits in Peanut plants. More particularly, the current invention pertains to producing Peanut plants containing less allergen content or with different expression of allergen-related genes and/or with improved fatty acid content by manipulating nucleotide sequences that regulate the expression of regulatory genes controlling allergen synthesis and fatty acid metabolism in peanuts.

### BACKGROUND OF THE INVENTION

Peanut (Arachis hypogaea L.), also known as groundnut, is a plant from the family Fabaceae. Peanuts are of great economic importance, particularly in the food industry, being the second most harvested legume in the world behind soybeans.

Peanut plant is an oilseed crop, and its oil content is around 50%. The peanuts kernel contains between 45% and 55% oil by weight, which is rich in monounsaturated (MUFA) and polyunsaturated fatty acids (PUFA). Peanut oil is mostly composed of triglycerides of eight fatty acids. Around 80% of these fatty acids are either oleic acid (monounsaturated, C18:1) or linoleic acid (polyunsaturated, C18:2). Generally, these two fatty acids vary inversely.

However, harvested Peanuts suffer a relatively short shelf-life storage due to their fatty acid content which are sensitive for rapid oxygenation.

In addition, peanut allergy is one of the most common allergies in the general population and the most common in children under the age of 18.

An allergic individual exposed to peanuts can experience a severe, life threating reaction. Exposure can occur by digestion or skin contact and even small amounts can trigger the allergic reaction.

The symptoms range from oral allergy syndrome, mostly related to pollen-associated peanut allergy, to severe dyspnea, anaphylaxis, and sometimes death.

The allergy is mostly likely due the presence of some proteins in the peanut. These proteins include glycoproteins, arachin, conarachin, peanut agglutinin and peanut phospholipase.

Thus, it is highly desirable to develop allergen free peanuts or peanuts conferring reduced allergic symptoms, to minimize the risk for the endangered parts of the population, and improve the fatty acid content to extend the shelf life of harvested peanuts.

### SUMMARY OF THE INVENTION

It is one object of the present invention to disclose a method for producing a modified peanut plant with at least one improved phenotypic trait, wherein the method comprises steps of genetically modifying at least one regulatory gene involved in allergen synthesis and/or at least one regulatory gene involved in fatty acid metabolism, the improved phenotypic trait is relative to a corresponding peanut plant lacking the genetic modification.

It is another object of the present invention to disclose the method as defined above, wherein the genetic modification is introduced through targeted genome editing.

It is another object of the present invention to disclose the method as defined in any of the above, wherein the at least one regulatory gene involved in allergen synthesis is selected from the group consisting of Ara h (Arachis hypogaea) regulatory genes and the at least one regulatory gene involved in fatty acid metabolism is selected from the group consisting of FAD (Fatty Acid Desaturase) regulatory genes.

It is another object of the present invention to disclose the method as defined in any of the above, wherein the at least one regulatory gene involved in allergen synthesis is selected from the group consisting of Ara h1 having a genomic nucleotide sequence as set forth in SEQ ID NO: 1 or a functional variant thereof, Ara h1.1 having a genomic nucleotide sequence as set forth in SEQ ID NO:341 or a functional variant thereof, Ara h2 having a genomic nucleotide sequence as set forth in SEQ ID NO:686 or a functional variant thereof, Ara h3 having a genomic nucleotide sequence as set forth in SEQ ID NO:908 or a functional variant thereof, Ara h5 having a genomic nucleotide sequence as set forth in SEQ ID NO:1161 or a functional variant thereof, Ara h6 having a genomic nucleotide sequence as set forth in SEQ ID NO:1348 or a functional variant thereof, Ara h7 having a genomic nucleotide sequence as set forth in SEQ ID NO:1514 or a functional variant thereof, Ara h8 having a genomic nucleotide sequence as set forth in SEQ ID NO:1690 or a functional variant thereof, Ara h9 having a genomic nucleotide sequence as set forth in SEQ ID NO:1960 or a functional variant thereof, Ara h10 having a genomic nucleotide sequence as set forth in SEQ ID NO:2208 or a functional variant thereof, Ara h11 having a genomic nucleotide sequence as set forth in SEQ ID NO:2476 or a functional variant thereof, Ara h14 having a genomic nucleotide sequence as set forth in SEQ ID NO: 2665 or a functional variant thereof, Ara h15 having a genomic nucleotide sequence as set forth in SEQ ID NO:2854 or a functional variant thereof, or any combination thereof.

It is another object of the present invention to disclose the method as defined in any of the above, wherein the at least one regulatory gene involved in allergen synthesis encodes a polypeptide sequence selected from the group consisting of Ara h1 having a polypeptide sequence as set forth in SEQ ID NO:2 or a functional variant thereof, Ara h1.1 having a polypeptide sequence as set forth in SEQ ID NO:342 or a functional variant thereof, Ara h2 having a polypeptide sequence as set forth in SEQ ID NO:687 or a functional variant thereof, Ara h3 having a polypeptide sequence as set forth in SEQ ID NO:909 or a functional variant thereof, Ara h5 having a polypeptide sequence as set forth in SEQ ID NO:1162 or a functional variant thereof, Ara h6 having a polypeptide sequence as set forth in SEQ ID NO:1349 or a functional variant thereof, Ara h7 having a polypeptide sequence as set forth in SEQ ID NO: 1515 or a functional variant thereof, Ara h8 having a polypeptide sequence as set forth in SEQ ID NO:1691 or a functional variant thereof, Ara h9 having a polypeptide sequence as set forth in SEQ ID NO:1961 or a functional variant thereof, Ara h10 having a polypeptide sequence as set forth in SEQ ID NO:2209 or a functional variant thereof, Ara h11 having a polypeptide sequence as set forth in SEQ ID NO:2477 or a functional variant thereof, Ara h14 having a polypeptide sequence as set forth in SEQ ID NO:2666 or a functional variant thereof, Ara h15 having a polypeptide sequence as set forth in SEQ ID NO:2855 or a functional variant thereof, or any combination thereof.

It is another object of the present invention to disclose the method as defined in any of the above, wherein the functional variant has at least 75% sequence identity to the Ara h nucleotide sequences selected from SEQ ID NO:1, SEQ ID NO:341, SEQ ID NO:686, SEQ ID NO:908, SEQ ID NO:1161, SEQ ID NO: 1348, SEQ ID NO: 1514, SEQ ID NO: 1690, SEQ ID NO:1960, SEQ ID NO:2208, SEQ ID NO:2476, SEQ ID NO:2665, SEQ ID NO:2854, and/or at least 75% sequence identity to the Ara h polypeptide sequences selected from SEQ ID NO:342, SEQ ID NO: 687, SEQ ID NO:909, SEQ ID NO:1162, SEQ ID NO:1349, SEQ ID NO:1515, SEQ ID NO: 1691, SEQ ID NO: 1961, SEQ ID NO:2209, SEQ ID NO:2477, SEQ ID NO:2666, SEQ ID NO: 2855 or any combination thereof.

It is another object of the present invention to disclose the method as defined in any of the above, wherein the at least one regulatory gene involved in fatty acid metabolism is selected from the group consisting of into FAD2-1 having a genomic nucleotide sequence as set forth in SEQ ID NO:3065 or a functional variant thereof, FAD2-2 having a genomic nucleotide sequence as set forth in SEQ ID NO:3410 or a functional variant thereof, FAD2-3 having a genomic nucleotide sequence as set forth in SEQ ID NO:3809 or a functional variant thereof, FAD2-4 having a genomic nucleotide sequence as set forth in SEQ ID NO:4105 or a functional variant thereof, or any combination thereof.

It is another object of the present invention to disclose the method as defined in any of the above, wherein the at least one regulatory gene involved in fatty acid metabolism encodes a polypeptide selected from the group consisting of into FAD2-1 having a polypeptide sequence as set forth in SEQ ID NO:3066 or a functional variant thereof, FAD2-2 having a polypeptide sequence as set forth in SEQ ID NO:3411 or a functional variant thereof, FAD2-3 having a polypeptide sequence as set forth in SEQ ID NO:3810 or a functional variant thereof, FAD2-4 having a polypeptide sequence as set forth in SEQ ID NO:4106 or a functional variant thereof, or any combination thereof.

It is another object of the present invention to disclose the method as defined in any of the above, wherein the functional variant has at least 75% sequence identity to the FAD nucleotide sequences selected from SEQ ID NO:3065, SEQ ID NO:3410, SEQ ID NO:3809, SEQ ID NO: 4105, and/or at least 75% sequence identity to the FAD polypeptide sequences selected from SEQ ID NO:3066, SEQ ID NO: 3411, SEQ ID NO:3810, SEQ ID NO:4106 or any combination thereof.

It is another object of the present invention to disclose the method as defined in any of the above, wherein the genetic modification in the Ara h (Arachis hypogaea) regulatory genes confers altered levels of allergens, different expression of allergens, expression of modified or mutated allergen molecules and/or lower, decreased, reduced and/or eliminated allergic response.

It is another object of the present invention to disclose the method as defined in any of the above, wherein the altered levels of allergens are lower, decreased, reduced and/or eliminated.

It is another object of the present invention to disclose the method as defined in any of the above, wherein the altered levels of allergens, different expression of allergens, expression of modified or mutated allergen molecules confer lower, decreased, reduced and/or eliminated allergic response.

It is another object of the present invention to disclose the method as defined in any of the above, wherein the genetic modification in FAD (Fatty Acid Desaturase) regulatory genes confers altered levels or ratios of fatty acid content.

It is another object of the present invention to disclose the method as defined in any of the above, wherein the altered levels or ratios of fatty acid content are characterized by an altered fatty acid transport and/or an altered fatty acid conversion.

It is another object of the present invention to disclose the method as defined in any of the above, wherein the altered levels or ratios of fatty acid content are selected from oleic:linoleic acid ratio of high oleic to linoleic acid ratio.

It is another object of the present invention to disclose the method as defined in any of the above, wherein the at least one phenotypic trait is selected from the group consisting of lower allergic response, decreased allergic response, reduced allergic response, eliminated allergic response, longer shelf-life, high oleic acid to linoleic acid ratio, healthier fatty acid properties, and any combination thereof.

It is another object of the present invention to disclose the method as defined in any of the above, wherein the peanut plant is an Ara h/FAD double mutant.

It is another object of the present invention to disclose the method as defined in any of the above, wherein the steps of genetically modifying are introduced using mutagenesis, small interfering RNA (siRNA), microRNA (miRNA), artificial miRNA (amiRNA), DNA introgression, endonucleases or any combination thereof.

It is another object of the present invention to disclose the method as defined in any of the above, wherein the targeted genome editing is selected from meganuclease, Clustered regularly interspaced short palindromic repeats (CRISPR) and CRISPR-associated (Cas) gene (CRISPR/Cas), Transcription activator-like effector nucleases (TALEN) or Zinc-finger nucleases (ZFNs) or any combination thereof.

It is another object of the present invention to disclose the method as defined in any of the above, wherein the Cas protein is selected from a group comprising but not limited to Cpf1, Cas9, Cas12, Cas13, Cas14, CasX or CasY.

It is another object of the present invention to disclose the method as defined in any of the above, wherein the mutated regulatory gene is a CRISPR/Cas9-induced heritable mutated allele.

It is another object of the present invention to disclose the method as defined in any of the above, wherein the at least one regulatory gene involved in allergen synthesis and/or fatty acid metabolism is edited through deleting, silencing, downregulating, de-regulating, deactivating, knockout, loss of function, reducing expression, at least partially deleting, at least partially silencing, at least partially deactivating, removing, partially removing, duplicating, inverting, missense mutation, nonsense mutation, insertion, deletion, indel, substitution or duplication or any combination thereof of the regulatory region or coding region of the gene.

It is another object of the present invention to disclose the method as defined in any of the above, wherein the at least one genetic modification in the regulatory gene involved in allergen synthesis is generated in planta via introduction of a construct comprising (a) Cas DNA and guide RNA (gRNA) sequence selected from the group consisting of SEQ ID NO:3-340, SEQ ID NO: 343-685, SEQ ID NO:688-907, SEQ ID NO:910-1160, SEQ ID NO:1163-1347, SEQ ID NO: 1350-1513, SEQ ID NO: 1516-1689, SEQ ID NO: 1692-1959, SEQ ID NO: 1962-2207, SEQ ID NO:2210-2475, SEQ ID NO:2478-2664, SEQ ID NO:2667-2853, and SEQ ID NO: 2856-3064 for the Ara h 1, Ara h1.1, Ara h2, Ara h3, Ara h5, Ara h6, Ara h7, Ara h8, Ara h9, Ara h10, Ara h11, Ara h14, Ara h15, respectively or any combination thereof, or (b) a ribonucleoprotein (RNP) complex comprising Cas protein and gRNA sequence selected from the group consisting of SEQ ID NO:3-340, SEQ ID NO:343-685, SEQ ID NO:688-907, SEQ ID NO: 910-1160, SEQ ID NO:1163-1347, SEQ ID NO:1350-1513, SEQ ID NO: 1516-1689, SEQ ID NO: 1692-1959, SEQ ID NO:1962-2207, SEQ ID NO:2210-2475, SEQ ID NO:2478-2664, SEQ ID NO:2667-2853, and SEQ ID NO:2856-3064 for the Ara h 1, Ara h1.1, Ara h2, Ara h3, Ara h5, Ara h6, Ara h7, Ara h8, Ara h9, Ara h10, Ara h11, Ara h14, Ara h15, respectively, or any combination thereof.

It is another object of the present invention to disclose the method as defined in any of the above, wherein the at least one genetic modification in the regulatory gene involved in fatty acid metabolism is generated in planta via introduction of a construct comprising (a) Cas DNA and guide RNA (gRNA) sequence selected from the group consisting of SEQ ID NO:3067-3409, SEQ ID NO:3412-3808, SEQ ID NO:3811-4104, SEQ ID NO:4107-4400 for the FAD2-1-FAD2-4, respectively, or any combination thereof, or (b) a ribonucleoprotein (RNP) complex comprising Cas protein and gRNA sequence selected from the group consisting of SEQ ID NO:3067-3409, SEQ ID NO:3412-3808, SEQ ID NO:3811-4104, SEQ ID NO:4107-4400 for the FAD2-1-FAD2-4, respectively, or any combination thereof.

It is another object of the present invention to disclose the method as defined in any of the above, wherein the gRNA sequence comprises a 3′ NGG Protospacer Adjacent Motif (PAM).

It is another object of the present invention to disclose the method as defined in any of the above, wherein the method comprises the steps of: (a) selecting, from a peanut plant genome, at least one regulatory gene and/or a promotor of a regulatory gene involved in allergen synthesis and/or fatty acid metabolism of the peanuts plant; (b) synthesizing or designing at least one guide RNA (gRNA) comprising a nucleotide sequence complementary to the at least one selected peanut gene; (c) transforming peanut plant cells, cell nucleus, tissues, seeds, or progeny cell of said peanut plant with a construct comprising (a) Cas nucleotide sequence operably linked to the at least one gRNA, or (b) a ribonucleoprotein (RNP) complex comprising Cas protein and the at least one gRNA; (d) culturing the peanut plant cells, cell nucleus, tissues, seeds, or progeny cell thereof; (e) selecting the peanut cells, cell nucleus, tissues, seeds, or progeny cell thereof which express desired mutations/genomic modifications in the at least one selected peanut regulatory gene and/or a promotor of a regulatory gene, (f) regenerating a peanut plant from the transformed plant cell, cell nucleus, or tissue, seed or plant progeny cell thereof carrying the desired mutations/genomic modifications; and (g) screening the regenerated peanut plants for a peanut plant with at least one improved phenotypic trait related to at least one regulatory gene and/or a promotor of a regulatory gene involved in allergen synthesis and/or at least one regulatory gene and/or a promotor of a regulatory gene involved in fatty acid metabolism.

It is another object of the present invention to disclose the method as defined in any of the above, wherein the method comprises administering a nucleic acid composition that comprises:
a) a first nucleotide sequence encoding the gRNA molecule and b) a second nucleotide sequence encoding the Cas molecule.

It is another object of the present invention to disclose the method as defined in any of the above, wherein the CRISPR/Cas system is delivered to the cell by Agrobacterium infiltration, virus-based plasmids for delivery of the genome editing molecules and mechanical insertion of DNA (PEG mediated DNA transformation, biolistics, etc.).

It is another object of the present invention to disclose the method as defined in any of the above, wherein the Cas protein is selected from a group comprising but not limited to Cpf1, Cas9, Cas12, Cas13, Cas14, CasX or CasY.

It is another object of the present invention to disclose a modified peanut plant, plant part, plant cell, plant progeny, plant tissue, plant seed, plant pod or a tissue culture, produced by the method as defined in any of the above.

It is another object of the present invention to disclose a plant part, plant cell, plant progeny, plant tissue, plant pod, plant seed or a tissue culture of a modified peanut plant produced by the method as defined in any of the above.

It is another object of the present invention to disclose a tissue culture of regenerable cells, protoplasts or callus obtained from the modified peanut plant produced by the method as defined in any of the above.

It is another object of the present invention to disclose a modified peanut plant exhibiting at least one improved phenotypic trait, wherein the modified plant comprises a targeted genome editing mutation in at least one regulatory gene involved in allergen synthesis and/or in at least one regulatory gene involved in fatty acid metabolism, the improved phenotypic trait is relative to a corresponding peanut plant lacking the mutation.

It is another object of the present invention to disclose the modified peanut plant as defined in any of the above, wherein the genetic modification is introduced through targeted genome editing.

It is another object of the present invention to disclose the modified peanut plant as defined in any of the above, wherein the at least one regulatory gene involved in allergen synthesis is selected from the group consisting of Ara h (Arachis hypogaea) regulatory genes and the at least one regulatory gene involved in fatty acid metabolism is selected from the group consisting of FAD (Fatty Acid Desaturase) regulatory genes.

It is another object of the present invention to disclose the modified peanut plant as defined in any of the above, wherein the at least one regulatory gene involved in allergen synthesis is selected from the group consisting of Ara h1 having a genomic nucleotide sequence as set forth in SEQ ID NO:1 or a functional variant thereof, Ara h1.1 having a genomic nucleotide sequence as set forth in SEQ ID NO:341 or a functional variant thereof, Ara h2 having a genomic nucleotide sequence as set forth in SEQ ID NO:686 or a functional variant thereof, Ara h3 having a genomic nucleotide sequence as set forth in SEQ ID NO:908 or a functional variant thereof, Ara h5 having a genomic nucleotide sequence as set forth in SEQ ID NO:1161 or a functional variant thereof, Ara h6 having a genomic nucleotide sequence as set forth in SEQ ID NO:1348 or a functional variant thereof, Ara h7 having a genomic nucleotide sequence as set forth in SEQ ID NO:1514 or a functional variant thereof, Ara h8 having a genomic nucleotide sequence as set forth in SEQ ID NO:1690 or a functional variant thereof, Ara h9 having a genomic nucleotide sequence as set forth in SEQ ID NO:1960 or a functional variant thereof, Ara h10 having a genomic nucleotide sequence as set forth in SEQ ID NO:2208 or a functional variant thereof, Ara h11 having a genomic nucleotide sequence as set forth in SEQ ID NO: 2476 or a functional variant thereof, Ara h14 having a genomic nucleotide sequence as set forth in SEQ ID NO:2665 or a functional variant thereof, Ara h15 having a genomic nucleotide sequence as set forth in SEQ ID NO:2854 or a functional variant thereof, or any combination thereof.

It is another object of the present invention to disclose the modified peanut plant as defined in any of the above, wherein the at least one regulatory gene involved in allergen synthesis encodes a polypeptide sequence selected from the group consisting of Ara h1 having a polypeptide sequence as set forth in SEQ ID NO:2 or a functional variant thereof, Ara h1.1 having a polypeptide sequence as set forth in SEQ ID NO:342 or a functional variant thereof, Ara h2 having a polypeptide sequence as set forth in SEQ ID NO:687 or a functional variant thereof, Ara h3 having a polypeptide sequence as set forth in SEQ ID NO:909 or a functional variant thereof, Ara h5 having a polypeptide sequence as set forth in SEQ ID NO:1162 or a functional variant thereof, Ara h6 having a polypeptide sequence as set forth in SEQ ID NO: 1349 or a functional variant thereof, Ara h7 having a polypeptide sequence as set forth in SEQ ID NO:1515 or a functional variant thereof, Ara h8 having a polypeptide sequence as set forth in SEQ ID NO:1691 or a functional variant thereof, Ara h9 having a polypeptide sequence as set forth in SEQ ID NO:1961 or a functional variant thereof, Ara h10 having a polypeptide sequence as set forth in SEQ ID NO:2209 or a functional variant thereof, Ara h11 having a polypeptide sequence as set forth in SEQ ID NO:2477 or a functional variant thereof, Ara h14 having a polypeptide sequence as set forth in SEQ ID NO:2666 or a functional variant thereof, Ara h15 having a polypeptide sequence as set forth in SEQ ID NO:2855 or a functional variant thereof, or any combination thereof.

It is another object of the present invention to disclose the modified peanut plant as defined in any of the above, wherein the functional variant has at least 75% sequence identity to the Ara h nucleotide sequences selected from SEQ ID NO:1, SEQ ID NO:341, SEQ ID NO:686, SEQ ID NO: 908, SEQ ID NO:1161, SEQ ID NO: 1348, SEQ ID NO: 1514, SEQ ID NO:1690, SEQ ID NO: 1960, SEQ ID NO:2208, SEQ ID NO:2476, SEQ ID NO:2665, SEQ ID NO:2854, and/or at least 75% sequence identity to the Ara h polypeptide sequences selected from SEQ ID NO: 342, SEQ ID NO: 687, SEQ ID NO:909, SEQ ID NO: 1162, SEQ ID NO: 1349, SEQ ID NO: 1515, SEQ ID NO: 1691, SEQ ID NO: 1961, SEQ ID NO:2209, SEQ ID NO:2477, SEQ ID NO: 2666, SEQ ID NO:2855 or any combination thereof.

It is another object of the present invention to disclose the modified peanut plant as defined in any of the above, wherein the at least one regulatory gene involved in fatty acid metabolism is selected from the group consisting of into FAD2-1 having a genomic nucleotide sequence as set forth in SEQ ID NO:3065 or a functional variant thereof, FAD2-2 having a genomic nucleotide sequence as set forth in SEQ ID NO:3410 or a functional variant thereof, FAD2-3 having a genomic nucleotide sequence as set forth in SEQ ID NO:3809 or a functional variant thereof, FAD2-4 having a genomic nucleotide sequence as set forth in SEQ ID NO:4105 or a functional variant thereof, or any combination thereof.

It is another object of the present invention to disclose the modified peanut plant as defined in any of the above, wherein the at least one regulatory gene involved in fatty acid metabolism encodes a polypeptide selected from the group consisting of into FAD2-1 having a polypeptide sequence as set forth in SEQ ID NO:3066 or a functional variant thereof, FAD2-2 having a polypeptide sequence as set forth in SEQ ID NO:3411 or a functional variant thereof, FAD2-3 having a polypeptide sequence as set forth in SEQ ID NO:3810 or a functional variant thereof, FAD2-4 having a polypeptide sequence as set forth in SEQ ID NO:4106 or a functional variant thereof, or any combination thereof.

It is another object of the present invention to disclose the modified peanut plant as defined in any of the above, wherein the functional variant has at least 75% sequence identity to the FAD nucleotide sequences selected from SEQ ID NO:3065, SEQ ID NO:3410, SEQ ID NO:3809, SEQ ID NO:4105, and/or at least 75% sequence identity to the FAD polypeptide sequences selected from SEQ ID NO:3066, SEQ ID NO: 3411, SEQ ID NO:3810, SEQ ID NO:4106 or any combination thereof.

It is another object of the present invention to disclose the modified peanut plant as defined in any of the above, wherein the genetic modification in the Ara h (Arachis hypogaea) regulatory genes confers altered levels of allergens, different expression of allergens, expression of modified or mutated allergen molecules and/or lower, decreased, reduced and/or eliminated allergic response.

It is another object of the present invention to disclose the modified peanut plant as defined in any of the above, wherein the altered levels of allergens are lower, decreased, reduced and/or eliminated.

It is another object of the present invention to disclose the modified peanut plant as defined in any of the above, wherein the altered levels of allergens, different expression of allergens, expression of modified or mutated allergen molecules confer lower, decreased, reduced and/or eliminated allergic response.

It is another object of the present invention to disclose the modified peanut plant as defined in any of the above, wherein the genetic modification in FAD (Fatty Acid Desaturase) regulatory genes confers altered levels or ratios of fatty acid content.

It is another object of the present invention to disclose the modified peanut plant as defined in any of the above, wherein the altered levels or ratios of fatty acid content are characterized by an altered fatty acid transport and/or an altered fatty acid conversion.

It is another object of the present invention to disclose the modified peanut plant as defined in any of the above, wherein the altered levels or ratios of fatty acid content are selected from oleic:linoleic acid ratio of high oleic to linoleic acid ratio.

It is another object of the present invention to disclose the modified peanut plant as defined in any of the above, wherein the at least one phenotypic trait is selected from the group consisting of lower allergic response, decreased allergic response, reduced allergic response, eliminated allergic response, longer shelf-life, high oleic acid to linoleic acid ratio, healthier fatty acid properties, and any combination thereof.

It is another object of the present invention to disclose the modified peanut plant as defined in any of the above, wherein the peanut plant is an Ara h/FAD double mutant.

It is another object of the present invention to disclose the modified peanut plant as defined in any of the above, wherein the steps of genetically modifying are introduced using mutagenesis, small interfering RNA (siRNA), microRNA (miRNA), artificial miRNA (amiRNA), DNA introgression, endonucleases or any combination thereof.

It is another object of the present invention to disclose the modified peanut plant as defined in any of the above, wherein the targeted genome editing is selected from meganuclease, Clustered regularly interspaced short palindromic repeats (CRISPR) and CRISPR-associated (Cas) gene (CRISPR/Cas), Transcription activator-like effector nucleases (TALEN) or Zinc-finger nucleases (ZFNs) or any combination thereof.

It is another object of the present invention to disclose the modified peanut plant as defined in any of the above, wherein the Cas protein is selected from a group comprising but not limited to Cpf1, Cas9, Cas12, Cas13, Cas14, CasX or CasY.

It is another object of the present invention to disclose the modified peanut plant as defined in any of the above, wherein the mutated regulatory gene is a CRISPR/Cas9-induced heritable mutated allele.

It is another object of the present invention to disclose the modified peanut plant as defined in any of the above, wherein the at least one regulatory gene involved in allergen synthesis and/or fatty acid metabolism is edited through deleting, silencing, downregulating, de-regulating, deactivating, knockout, loss of function, reducing expression, at least partially deleting, at least partially silencing, at least partially deactivating, removing, partially removing, duplicating, inverting, missense mutation, nonsense mutation, insertion, deletion, indel, substitution or duplication or any combination thereof of the regulatory region or coding region of the gene.

It is another object of the present invention to disclose the modified peanut plant as defined in any of the above, wherein the at least one genetic modification in the regulatory gene involved in allergen synthesis is generated in planta via introduction of a construct comprising (a) Cas DNA and guide RNA (gRNA) sequence selected from the group consisting of SEQ ID NO:3-340, SEQ ID NO:343-685, SEQ ID NO:688-907, SEQ ID NO:910-1160, SEQ ID NO:1163-1347, SEQ ID NO:1350-1513, SEQ ID NO: 1516-1689, SEQ ID NO:1692-1959, SEQ ID NO: 1962-2207, SEQ ID NO:2210-2475, SEQ ID NO:2478-2664, SEQ ID NO:2667-2853, and SEQ ID NO:2856-3064 for the Ara h 1, Ara h1.1, Ara h2, Ara h3, Ara h5, Ara h6, Ara h7, Ara h8, Ara h9, Ara h10, Ara h11, Ara h14, Ara h15, respectively or any combination thereof, or (b) a ribonucleoprotein (RNP) complex comprising Cas protein and gRNA sequence selected from the group consisting of SEQ ID NO:3-340, SEQ ID NO:343-685, SEQ ID NO:688-907, SEQ ID NO:910-1160, SEQ ID NO:1163-1347, SEQ ID NO:1350-1513, SEQ ID NO:1516-1689, SEQ ID NO:1692-1959, SEQ ID NO:1962-2207, SEQ ID NO:2210-2475, SEQ ID NO: 2478-2664, SEQ ID NO:2667-2853, and SEQ ID NO:2856-3064 for the Ara h 1, Ara h1.1, Ara h2, Ara h3, Ara h5, Ara h6, Ara h7, Ara h8, Ara h9, Ara h10, Ara h11, Ara h14, Ara h15, respectively, or any combination thereof.

It is another object of the present invention to disclose the modified peanut plant as defined in any of the above, wherein the at least one genetic modification in the regulatory gene involved in fatty acid metabolism is generated in planta via introduction of a construct comprising (a) Cas DNA and guide RNA (gRNA) sequence selected from the group consisting of SEQ ID NO: 3067 to SEQ ID NO:3409, SEQ ID NO:3412 to SEQ ID NO:3808, SEQ ID NO:3811 to SEQ ID NO:4104, SEQ ID NO:4107 to SEQ ID NO:4400 for the FAD2-1-FAD2-4, respectively, or any combination thereof, or (b) a ribonucleoprotein (RNP) complex comprising Cas protein and gRNA sequence selected from the group consisting of SEQ ID NO:3067 to SEQ ID NO:3409, SEQ ID NO:3412 to SEQ ID NO:3808, SEQ ID NO:3811 to SEQ ID NO: 4104, SEQ ID NO:4107 to SEQ ID NO:4400 for the FAD2-1-FAD2-4, respectively, or any combination thereof.

It is another object of the present invention to disclose the modified peanut plant as defined in any of the above, wherein the gRNA sequence comprises a 3′ NGG Protospacer Adjacent Motif (PAM).

It is another object of the present invention to disclose the modified peanut plant as defined in any of the above, wherein the peanut is homozygous for the modified gene locus.

It is another object of the present invention to disclose a plant part, plant cell, plant progeny, plant tissue, plant pod, plant seed or a tissue culture of a modified peanut plant as defined in any of the above.

It is another object of the present invention to disclose a tissue culture of regenerable cells, protoplasts or callus obtained from the modified peanut plant as defined in any of the above.

It is another object of the present invention to disclose harvestable parts of a modified peanut plant as defined in any of the above, wherein said harvestable parts are preferably shoot biomass pods and/or seeds.

It is another object of the present invention to disclose products derived from a modified peanut plant as defined in any of the above and/or from harvestable parts of a modified peanut plant as defined in any of the above.

It is another object of the present invention to disclose a method for producing a modified peanut plant characterized as conferring reduced allergic response and/or having altered fatty acid content, comprising steps of genetically modifying using targeted genome editing at least one regulatory gene involved in allergen synthesis selected from Ara h (Arachis hypogaea) regulatory genes for conferring the reduced allergic response, and at least one regulatory gene involved in fatty acid metabolism selected from FAD (Fatty Acid Desaturase) regulatory genes for conferring the altered fatty acid content.

It is another object of the present invention to disclose the method as defined in any of the above, wherein the at least one regulatory gene involved in allergen synthesis is selected from the group consisting of Ara h1 having a genomic nucleotide sequence as set forth in SEQ ID NO: 1 or a functional variant thereof, Ara h1.1 having a genomic nucleotide sequence as set forth in SEQ ID NO:341 or a functional variant thereof, Ara h2 having a genomic nucleotide sequence as set forth in SEQ ID NO:686 or a functional variant thereof, Ara h3 having a genomic nucleotide sequence as set forth in SEQ ID NO:908 or a functional variant thereof, Ara h5 having a genomic nucleotide sequence as set forth in SEQ ID NO:1161 or a functional variant thereof, Ara h6 having a genomic nucleotide sequence as set forth in SEQ ID NO:1348 or a functional variant thereof, Ara h7 having a genomic nucleotide sequence as set forth in SEQ ID NO:1514 or a functional variant thereof, Ara h8 having a genomic nucleotide sequence as set forth in SEQ ID NO:1690 or a functional variant thereof, Ara h9 having a genomic nucleotide sequence as set forth in SEQ ID NO:1960 or a functional variant thereof, Ara h10 having a genomic nucleotide sequence as set forth in SEQ ID NO:2208 or a functional variant thereof, Ara h11 having a genomic nucleotide sequence as set forth in SEQ ID NO:2476 or a functional variant thereof, Ara h14 having a genomic nucleotide sequence as set forth in SEQ ID NO: 2665 or a functional variant thereof, Ara h15 having a genomic nucleotide sequence as set forth in SEQ ID NO:2854 or a functional variant thereof, or any combination thereof.

It is another object of the present invention to disclose the method as defined in any of the above, wherein the at least one regulatory gene involved in allergen synthesis encodes a polypeptide sequence selected from the group consisting of Ara h1 having a polypeptide sequence as set forth in SEQ ID NO:2 or a functional variant thereof, Ara h1.1 having a polypeptide sequence as set forth in SEQ ID NO:342 or a functional variant thereof, Ara h2 having a polypeptide sequence as set forth in SEQ ID NO:687 or a functional variant thereof, Ara h3 having a polypeptide sequence as set forth in SEQ ID NO:909 or a functional variant thereof, Ara h5 having a polypeptide sequence as set forth in SEQ ID NO:1162 or a functional variant thereof, Ara h6 having a polypeptide sequence as set forth in SEQ ID NO:1349 or a functional variant thereof, Ara h7 having a polypeptide sequence as set forth in SEQ ID NO: 1515 or a functional variant thereof, Ara h8 having a polypeptide sequence as set forth in SEQ ID NO:1691 or a functional variant thereof, Ara h9 having a polypeptide sequence as set forth in SEQ ID NO:1961 or a functional variant thereof, Ara h10 having a polypeptide sequence as set forth in SEQ ID NO:2209 or a functional variant thereof, Ara h11 having a polypeptide sequence as set forth in SEQ ID NO:2477 or a functional variant thereof, Ara h14 having a polypeptide sequence as set forth in SEQ ID NO:2666 or a functional variant thereof, Ara h15 having a polypeptide sequence as set forth in SEQ ID NO:2855 or a functional variant thereof, or any combination thereof.

It is another object of the present invention to disclose the method as defined in any of the above, wherein the functional variant has at least 75% sequence identity to the Ara h nucleotide sequences selected from SEQ ID NO:1, SEQ ID NO:341, SEQ ID NO:686, SEQ ID NO:908, SEQ ID NO:1161, SEQ ID NO: 1348, SEQ ID NO: 1514, SEQ ID NO: 1690, SEQ ID NO:1960, SEQ ID NO:2208, SEQ ID NO:2476, SEQ ID NO:2665, SEQ ID NO:2854, and/or at least 75% sequence identity to the Ara h polypeptide sequences selected from SEQ ID NO:342, SEQ ID NO: 687, SEQ ID NO:909, SEQ ID NO:1162, SEQ ID NO: 1349, SEQ ID NO: 1515, SEQ ID NO: 1691, SEQ ID NO: 1961, SEQ ID NO:2209, SEQ ID NO:2477, SEQ ID NO:2666, SEQ ID NO: 2855 or any combination thereof.

It is another object of the present invention to disclose the method as defined in any of the above, wherein the at least one regulatory gene involved in fatty acid metabolism is selected from the group consisting of into FAD2-1 having a genomic nucleotide sequence as set forth in SEQ ID NO:3065 or a functional variant thereof, FAD2-2 having a genomic nucleotide sequence as set forth in SEQ ID NO:3410 or a functional variant thereof, FAD2-3 having a genomic nucleotide sequence as set forth in SEQ ID NO:3809 or a functional variant thereof, FAD2-4 having a genomic nucleotide sequence as set forth in SEQ ID NO:4105 or a functional variant thereof, or any combination thereof.

It is another object of the present invention to disclose the method as defined in any of the above, wherein the at least one regulatory gene involved in fatty acid metabolism encodes a polypeptide selected from the group consisting of into FAD2-1 having a polypeptide sequence as set forth in SEQ ID NO:3066 or a functional variant thereof, FAD2-2 having a polypeptide sequence as set forth in SEQ ID NO:3411 or a functional variant thereof, FAD2-3 having a polypeptide sequence as set forth in SEQ ID NO:3810 or a functional variant thereof, FAD2-4 having a polypeptide sequence as set forth in SEQ ID NO:4106 or a functional variant thereof, or any combination thereof.

It is another object of the present invention to disclose the method as defined in any of the above, wherein the functional variant has at least 75% sequence identity to the FAD nucleotide sequences selected from SEQ ID NO:3065, SEQ ID NO:3410, SEQ ID NO:3809, SEQ ID NO: 4105, and/or at least 75% sequence identity to the FAD polypeptide sequences selected from SEQ ID NO:3066, SEQ ID NO: 3411, SEQ ID NO:3810, SEQ ID NO:4106 or any combination thereof.

It is another object of the present invention to disclose the method as defined in any of the above, wherein the genetic modification in the Ara h (Arachis hypogaea) regulatory genes confers altered levels of allergens, different expression of allergens, expression of modified or mutated allergen molecules and/or lower, decreased, reduced and/or eliminated allergic response.

It is another object of the present invention to disclose the method as defined in any of the above, wherein the altered levels of allergens are lower, decreased, reduced and/or eliminated.

It is another object of the present invention to disclose the method as defined in any of the above, wherein the altered levels of allergens, different expression of allergens, expression of modified or mutated allergen molecules confer lower, decreased, reduced and/or eliminated allergic response.

It is another object of the present invention to disclose the method as defined in any of the above, wherein the genetic modification in FAD (Fatty Acid Desaturase) regulatory genes confers altered levels or ratios of fatty acid content.

It is another object of the present invention to disclose the method as defined in any of the above, wherein the altered levels or ratios of fatty acid content is characterized by an altered fatty acid transport and/or an altered fatty acid conversion.

It is another object of the present invention to disclose the method as defined in any of the above, wherein the altered levels or ratios of fatty acid content is selected from oleic:linoleic acid ratio of high oleic to linoleic acid ratio.

It is another object of the present invention to disclose the method as defined in any of the above, wherein the at least one phenotypic trait is selected from the group consisting of lower allergic response, decreased allergic response, reduced allergic response, eliminated allergic response, longer shelf-life, high oleic acid to linoleic acid ratio, healthier fatty acid properties, and any combination thereof.

It is another object of the present invention to disclose use of (a) a nucleotide sequence as set forth in SEQ ID NO:1, SEQ ID NO:341, SEQ ID NO:686, SEQ ID NO:908, SEQ ID NO:1161, SEQ ID NO: 1348, SEQ ID NO: 1514, SEQ ID NO: 1690, SEQ ID NO:1960, SEQ ID NO:2208, SEQ ID NO:2476, SEQ ID NO:2665 and SEQ ID NO:2854 and/or (b) a nucleotide sequence as set forth in at least one of SEQ ID NO:3-340, SEQ ID NO:3-340, SEQ ID NO:343-685, SEQ ID NO:688-907, SEQ ID NO:910-1160, SEQ ID NO:1163-1347, SEQ ID NO:1350-1513, SEQ ID NO:1516-1689, SEQ ID NO:1692-1959, SEQ ID NO:1962-2207, SEQ ID NO:2210-2475, SEQ ID NO:2478-2664, SEQ ID NO:2667-2853, SEQ ID NO:2856-3064 and any combination thereof for targeted genome modification of peanut, for generating and/or producing a modified peanut plant with at least one genetically modified regulatory gene involved in allergen synthesis.

It is another object of the present invention to disclose use of (a) a nucleotide sequence as set forth in SEQ ID NO:3065, SEQ ID NO:3410, SEQ ID NO:3809 and SEQ ID NO:4105 and/or (b) a nucleotide sequence as set forth in at least one of SEQ ID NO:3067-3409, SEQ ID NO: 3412-3808, SEQ ID NO:3811-4104, SEQ ID NO:4107-4400 and any combination thereof for targeted genome modification of peanut, for generating and/or producing a modified peanut plant with at least one genetically modified regulatory gene involved in fatty acid metabolism.

### DETAILED DESCRIPTION OF THE INVENTION

In the following detailed description, specific details are set forth to facilitate understanding of the invention; however, it should be understood by those skilled in the art that the present invention may be practiced without these specific details. Unless otherwise defined herein, technical terms used shall have the meanings commonly understood by those of ordinary skill in the art. Further, unless otherwise required by context, singular terms shall include pluralities and plural terms shall include the singular. Furthermore, well-known methods, procedures, and components have been omitted to highlight the invention.

The present invention provides a method for producing a modified peanut plant with at least one improved phenotypic trait, wherein the method comprises steps of genetically modifying at least one regulatory gene involved in allergen synthesis and/or at least one regulatory gene involved in fatty acid metabolism, the improved phenotypic trait is relative to a corresponding peanut plant lacking the genetic modification.

The present invention further provides modified peanut plant, tissue, cells, cell nucleus, seed, pod or a progeny cell thereof produced by the method disclosed within the present invention.

According to one embodiment, the method of the present invention enables producing peanut plants with modified allergen content conferring reduced allergic response and/or peanuts with altered fatty acid content, and specifically, through gene editing techniques. Through this method, the present invention made it possible to produce peanut plants, tissue, cells, cell nucleus, seeds, pods or a progeny cell thereof conferring reduced allergic response (peanuts with reduced, modified or free of allergens) and/or with altered levels of fatty acids (more oleic acid than linoleic acid).

According to a specific embodiment, the peanut plants of the present invention have both reduced or altered allergen content and altered fatty acid content, generated by gene editing techniques.

It is herein noted that an allergic individual exposed to peanuts can experience a severe, life threating reaction. Exposure can occur by digestion, skin contact or inhalation, and even small amounts can trigger an allergic reaction. Contact with the slightest amount of peanut protein can be life threatening to particularly sensitive individuals.

It is within the scope of the present invention that The World Health Organization and International Union of Immunological Societies (WHO/IUIS) Allergen Nomenclature Subcommittee has published and named 16 kinds of peanut allergen, named Ara h1 to Ara h17 (Ara h4 was renamed to Ara h 3.02). The major peanut allergens are Ara h1, Ara h2, Ara h3, and Ara h6, but Ara h4, Ara h5, Ara h7, Ara h8, Ara h9, Ara h10, Ara h11 Ara h12, Ara h13, Ara h14, Ara h15, Ara h16, and Ara h17 are also described.

Without wishing to be bound by theory, Ara h1 and Ara h3 are seed storage proteins. Both allergens possess two beta-barrel cupin domains and are members of the cupin superfamily and the cupin 1 protein family. Cupin seed storage proteins serve as source of amino acids and energy during seed germination. In many plant species, cupins are involved in the defense against fungi and insects. It is herein acknowledged that Cupin storage proteins play an important role as allergens for primary peanut allergy. Ara h2, Ara h6 and Ara h7 are part of the prolamin superfamily comprising the family 2S albumins and the nonspecific lipid transfer proteins (nsLTPs), two families of low molecular weight proteins. These proteins are heavily disulfide-bonded, resulting in the high stability of the allergenic peanut 2S albumins Ara h2, Ara h6 and Ara h7. 2S albumins in seeds store amino acids, but also possess antifungal and antibacterial properties. Ara h 9, Ara h 16 and Ara h 17 are part of the nonspecific lipid transfer proteins (nsLTPs) family. They are found in seeds where they play important roles in resistance to biotic and abiotic stress, plant growth, and development.

In addition, Ara h10, Ara h11, Ara h14 and Ara h15 have been identified as oleosins, to which all patients with severe allergic reactions have IgE antibodies. Ara h12 and Ara h13 are peanut defensins (peptides involved in the protection of seeds and reproductive tissues), to which not all patients with severe allergic reactions have IgE antibodies.

It is further acknowledged herein that Ara h5 is a peanut profilin (highly conserved in higher plants), involved in the turnover and restructuring of the actin cytoskeleton. Ara h8 is a peanut Bet v 1 homolog, associated with lipids via its hydrophobic cavity, thereby protecting the protein from degradation. Ara h8 is linked to pollen-associated peanut allergy with mostly mild or moderate symptoms, thus may also be responsible for severe allergic reactions.

It is interestingly pointed out that, while peanut allergens are important to seed storage, development and resistance to biotic and abiotic stress, they are associated (e.g. due to their high stability) with severe allergic symptoms. This problem is approached and solved by the present invention of producing modified peanut plants with targeted genetically modification in at least one regulatory gene involved in allergen synthesis and/or at least one regulatory gene involved in fatty acid metabolism.

With respect to the fatty acid metabolism, it is herein acknowledged that the biological switch of oleic acid to linoleic acid is facilitated by fatty acid desaturase 2 enzyme that is further classified into FAD2-1, FAD2-2, FAD2-3, and FAD2-4. It is noted that the second double bond in a linoleic acid molecule renders it far more susceptible to oxidation. Thus, harvested peanuts suffer a relatively short shelf-life storage due to their fatty acid content which is sensitive for rapid oxygenation. Therefore, this ‘oxidative rancidity’ generates off-flavors in peanut products and hence limits the shelf life of products in retail outlets.

The current invention solves this problem by producing modified (genome edited) peanut plants with higher ratio of oleic acid, which has many healthy-related advantages, to linoleic acid, therefore conferring enhanced shelf life (due to high oleic acid content and low linoleic acid level).

According to aspects of the present invention, the oxidation stability of oleic acid is 10 times higher than that of linoleic acid; therefore, peanuts with high oleic acid to linoleic acid ratio, provided by the current invention, and their products, have a longer shelf life. In addition, compared with wild type peanuts, high-oleic-acid peanuts of the present invention, and their products, are beneficial to the health of the cardiovascular system.

By knock-out of FAD2 genes using targeted genome editing, the present invention provides peanuts with high levels of oleic acid (above 30%, preferably in the range of 30%-80%, more preferably, above 45%). these modified peanuts have lower level of linoleic acid (lower than 40%, preferably in the range of 40%-5%, more preferably, below 30%)

As used herein the term “about” denotes a measurable value such as an amount, a temporal duration, and the like, and is meant to encompass variations of ±20%, ±10%, ±5%, ±1%, or ±0.1% from the specified value, as such variations are appropriate to perform the disclosed methods.

The term “essentially” as used herein means in essence, namely basically or fundamentally, it is generally used to identify or stress the basic or essential character or nature of a thing or to say that a description is basically true or accurate. In other aspects, it means relating to the most important characteristics of something. In the current disclosure, it may further mean mostly, or basically or fundamentally. It may refer to a description which is true or accurate in at least 50%, preferably at least 60%, at least 70%, at least 80%, at least 90% or at least 95% of the cases.

As used herein the term “average” refers to the mean value as obtained by measuring a predetermined parameter in each plant of a certain plant population and calculating the mean value according to the number of plants in said population.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same peanut species or strain or variety or to sibling plant, or one or more individuals having one or both parents in common.

The term “plant” refers, without limitation, to a whole plant, grafted plant, ancestors and progeny of the plants, or any parts or derivatives thereof. According to the present invention, a non-liming list of plant part includes plant cells, plant tissue, plant protoplasts, plant organ, anthers, flowers, flower parts, scion, fruit, leaves, cuticle, indumentum, stomata, stomatal cells, epidermal cells, canopy, cotyledons, pistil, stem, anther, pods, seeds, seed coat, cutting, seed coat, roots, root tips, rootstock, shoot, bud, meristem, suspension cultures, plant cell or tissue culture from which plants can be regenerated, plant callus or calli, meristematic regions, meristematic cells, gametophytes, sporophyte, microspores, embryos, immature embryos, pollen, ovules, egg cells, zygotes, and the like. According to the present invention, the plant is a peanut plant, tissue, cell, or progeny cell thereof.

The term “plant cell” as used herein refers, without limitation, to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein refers, without limitation, to cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” as used herein refers, without limitation, to leaves, stems, pods, roots, root tips, flowers or flower parts, fruits, pollen, egg cells, zygotes, seeds, seed coat, cuttings, cell or tissue cultures, or any other part or product of a plant or a combination thereof.

The term “plant organ” as used herein refers, without limitation, to a distinct and visibly structured and differentiated part of a plant such as a root, stem, leaf, flower, flower bud, or embryo.

The term “plant tissue” refers, without limitation, to a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

The term “in planta” means in the context of the present invention, within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term “phenotype” refers, without limitation, to distinguishable characteristics from a genetically controlled trait, which can be a physical feature. It is within the scope that the term phenotype refers to the characteristics of a plant resulting from the expression of genes. As used herein, the term “phenotype” is interchangeable with “characteristic” or “phenotypic feature” or trait. Therefore, this term encompasses any characteristic or phenotypic feature or trait of the plant. A non-limiting list of phenotypes including features (e.g. physical features or characteristics) is selected from conferring lower allergic response, decreased allergic response, reduced allergic response, eliminated allergic response, longer shelf-life, linoleic acid:oleic acid ratio of higher linoleic acid, healthier fatty acid properties. In the context of the present invention, the improved phenotypic trait is relative to a corresponding peanut plant lacking the genetic modification.

According to further aspects of the invention, the modified plant exhibits at least one improved phenotypic trait, wherein the modified plant comprises a targeted genome editing mutation in at least one regulatory gene involved in allergen synthesis and/or in at least one regulatory gene involved in fatty acid metabolism, the improved phenotypic trait is relative to a corresponding Peanut plant lacking the mutation.

According to further aspects of the invention, the modified plant, plant part, plant cell, plant progeny, plant tissue, or plant seed or pod is from peanut and it is produced by the method disclosed herein, wherein the modified plant does not comprise a transgene. According to further aspects of the invention, the tissue culture of regenerable cells, protoplasts or callus is obtained from the modified peanut plant produced by the method disclosed herein.

According to further aspects of the invention, the method disclosed herein comprises a) selecting, from a eanut plant genome, at least one regulatory gene and/or a promotor of a regulatory gene involved in allergen synthesis and/or fatty acid metabolism of the Peanuts plants; b) synthesizing or designing at least one guide RNA (gRNA) comprising a nucleotide sequence complementary to the at least one selected Peanut gene; c) transforming Peanut plant cells, cell nucleus, tissues, seeds, or progeny cell of said Peanut plant with a construct comprising (a) Cas nucleotide sequence operably linked to the at least one gRNA, or (b) a ribonucleoprotein (RNP) complex comprising Cas protein and the at least one gRNA; d) culturing the Peanut plant cells, cell nucleus, tissues, seeds, or progeny cell thereof; e) selecting the Peanut cells, cell nucleus, tissues, seeds, or progeny cell thereof which express desired mutations/genomic modifications in the editing target region, and f) regenerating a Peanut plant from the transformed plant cell, cell nucleus, or tissue, seed or plant progeny cell thereof carrying the desired mutations/genomic modifications; and g) screening the regenerated Peanut plants for a peanut plant with at least one improved phenotypic trait related to at least one regulatory gene involved in allergen synthesis and/or at least one regulatory gene involved in fatty acid metabolism.

The term “peanut” refers, without limitation, to a plant member of the family Fabaceae, genus Arachis, and that is widely distributed as food, and more specifically, Arachis hypogaea, peanuts are legumes, a plant family that includes soybeans, green beans, and lentils. It is further within the scope of the present invention that the peanut (Arachis hypogaea), also known as the groundnut, goober, pindar or monkey nut, is a legume crop grown mainly for its edible seeds. According to some aspects of the invention, the peanut is an Ara h and/or FAD mutant.

The term “food allergen” refers, without limitation, to food proteins/glycoproteins that trigger allergic reactions via IgE response in an individual. The most common allergens are found in peanuts, tree nuts, fish, and shellfish. These food proteins are taken up by specialized epithelial cells (M cells), transferred to antigen-presenting cells such as dendritic cells, and processed into peptide fragments presented on the cell surface by class II MHC molecules. Peptides are then presented to naive T helper (Th) cells via MHC/T cell receptor interaction, resulting in Th cell priming and activation. This event initiates humoral and cellular events associated with, in the context of the present invention, peanut allergy. The allergic reaction caused by allergens is usually comprised of systemic symptoms, such as airway obstruction, hives, low blood pressure, and arrhythmia; and local symptoms, such as itching, swelling, nausea, vomiting, cramping, and diarrhea.

The term “allergen” refers, without limitation, to any protein or protein fragment thereof, molecule or any mixture thereof that are known to induce, cause, elicit or trigger an allergic response, e.g., an IgE-mediated immune response, in an individual, e.g., a human. As such, allergens are typically referred to as antigens. An allergen is typically a protein or a polypeptide.

According to the various aspects of the invention, the food allergens are selected from the group consisting of peanut Ara h allergens. According to further aspects of the invention, the peanut Ara h allergens are selected from the group consisting of Ara h1, Ara h1.1, Ara h2, Ara h3, Ara h5, Ara h6, Ara h7, Ara h8, Ara h9, Ara h10, Ara h11, Ara h14, Ara h15, or any combination thereof.

The term “fatty acid” refers, without limitation, to a carboxylic acid (or organic acid), often with a long aliphatic tail, either saturated or unsaturated. Typically, fatty acids have a carbon-carbon bonded chain of at least 8 carbon atoms in length, more often at least 12 carbons in length. Most naturally occurring fatty acids have an even number of carbon atoms because their biosynthesis involves acetate which has two carbon atoms. The fatty acids may be in a free state (non-esterified) or in an esterified form such as part of a triglyceride, diacylglyceride, monoacylglyceride, acyl-CoA (thio-ester) bound or other bound form. The fatty acid may be esterified as a phospholipid such as phosphatidylcholine, phosphatidylethanolamine, phosphatidylserine, phosphatidylglycerol, phosphatidylinositol or diphosphatidylglycerol forms. Peanut oil is mostly composed of triglycerides of eight fatty acids. In an embodiment, the fatty acid is oleic acid or linoleic acid or palmitic acid. In an embodiment, oleic acid, linoleic acid and palmitic acid account for 90% of the fatty acids in the peanut.

The term “oleic acid” refers, without limitation, to a fatty acid found in animals and plants. Oleic acid is also known as a monounsaturated omega-9 fatty acid. In one embodiment, oleic acid is derived from peanut plant. In one embodiment, oleic acid is derived from peanut seeds.

It is characterized by the formula CH3-(CH2)7-CH—CH—(CH2)7-COOH).

The term “linoleic acid” refers, without limitation, to a polyunsaturated omega-6 fatty acid. In one embodiment, linoleic acid is derived from peanut plants. In one embodiment, linoleic acid is derived from peanut seeds. The biological switch of oleic acid into linoleic acid is facilitated by fatty acid desaturase 2 enzyme (FAD2). FAD2 is located in the endoplasmic reticulum, with cytochrome b5 as the electron donor. Six members of the FAD2 family were cloned from peanut and named FAD2-1 to FAD2-6. FAD2-1, FAD2-2, FAD2-3, and FAD2-4 encode the endoplasmic reticulum omega-3 fatty acid dehydrogenase, while FAD2-5 and FAD2-6 encode the chloroplast omega-6 fatty acid dehydrogenase. It is characterized by the formula

HOOC(CH2)7CH—CHCH2CH—CH(CH2)4CH3

According to the various aspects of the invention, the fatty acid metabolism is related to FAD enzymes. According to further aspects of the invention, the FAD enzymes are selected from the group consisting of FAD2-1, FAD2-2, FAD2-3, FAD2-4, or any combination thereof.

According to some embodiments of the invention, the modified peanut plant has altered levels or ratios of fatty acid content as compared to wild type peanut or to peanut not mutated in one or more FAD2 genes, wherein the oleic:linoleic acid ratio is high oleic to linoleic acid ratio.

According to some embodiments of the invention, the oleic acid is in a range of 30% to 80%, such as above 40%, preferably from 50% to 90%, while the linoleic acid is in a range of 5% to 40%, such as lower than 25%, preferably between 2% to 30%.

The term “gene” refers, without limitation, to the basic unit of inheritance. Genes are passed from parents to offspring and contain the information needed to specify physical and biological traits. Most genes code for specific proteins, or segments of proteins, which have differing functions within the body. It is within the scope that the term “gene” as used herein encompasses sequence of a regulatory region of the gene, such as the promoter region, and/or sequence of the coding sequence (CDS) or region of the gene. Thus, the present invention provides a method for targeted genome editing of a regulatory sequence of a gene (e.g. promoter), as well as targeting the coding sequence of a gene.

According to some aspects of the invention, the gene is a regulatory gene involved in allergen synthesis and/or a regulatory gene involved in fatty acid metabolism. According to further aspects of the invention, the regulatory gene involved in allergen synthesis is selected from Ara h regulatory genes, and the regulatory gene involved in fatty acid metabolism is selected from FAD regulatory genes.

The term “gene encoding” sequence refers, without limitation, to the information encoded in a gene that is used to either make RNA molecules that code for proteins or to make non-coding RNA molecules that serve other functions.

The term “genetic modification” refers, without limitation, to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified peanut plants with improved traits are generated using targeted genome editing mechanism. This technique enables to achieve in planta modification of specific genes that relate to and/or control the fatty acid conversion and allergen-related proteins in the Peanut plant.

According to some aspects of the invention, the genetic modification is selected from the group consisting of altered levels of allergens, different expression of allergens, altered levels fatty acid content and any combination thereof. According to further aspects of the invention, the altered levels of allergens are lower, decreased, reduced and/or eliminated. According to further aspects of the invention, the different or modified expression of allergens results in lower, decreased, reduced and/or eliminated allergic response. According to further aspects of the invention, the altered levels of fatty acid content are characterized by an altered fatty acid transport and/or an altered fatty acid conversion.

The mutations within the scope of the present invention, i.e. generated by genome editing in Ara h/FAD sequences, include, but are not limited to, deletion, silencing, downregulating, de-regulating, deactivating, knockout, loss of function, reducing expression, at least partially deleting, at least partially silencing, at least partially deactivating, removing, partially removing, duplicating, inverting, missense mutation, nonsense mutation, insertion, indel, substitution or duplication or any combination thereof of the regulatory region or coding region of the gene.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and/or gene knockout mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonym of the term included within the scope of the present invention is null mutation.

The term “gene editing” refers, without limitation, to the addition, removal, or alteration of a genetic material at a particular desired location in the genome. Gene editing can be used to correct, introduce or delete almost any DNA sequence in many different types of cells and organisms, such as animals, plants and bacteria. Gene editing is performed using enzymes, particularly nucleases, which have been engineered to target a specific DNA sequence, where they introduce cuts into the DNA strands, enabling the removal of existing DNA and the insertion of replacement DNA. A non-limiting list of techniques for genome editing are restriction enzymes, zinc finger nucleases, prime editing, and Programmable Addition via Site-specific Targeting Elements (PASTE), zinc-finger nucleases (ZFNs), transcription activator-like effector nucleases (TALENs), and meganucleases, and clustered regularly interspaced short palindromic repeats (CRISPR/Cas9). CRISPR/Cas9 is an RNA-based system, and it works by cutting a DNA sequence at a specific genetic location and deleting or inserting DNA sequences, which can change a single base pair of DNA, large pieces of chromosomes, or regulation of gene expression levels. Unlike previous genetic engineering techniques that randomly insert genetic material into a host genome, genome editing targets the insertions to site specific locations. As used herein, the term “gene editing” is interchangeable with “genome modification”, “gene modification”, “genetic modification”, “genome editing”, or “genome engineering”.

According to some aspects of the invention, the genetic modification is introduced using mutagenesis, small interfering RNA (siRNA), microRNA (miRNA), artificial miRNA (amiRNA), DNA introgression, endonucleases or any combination thereof.

According to some aspects of the invention, genome editing techniques are selected from meganuclease, Clustered regularly interspaced short palindromic repeats (CRISPR) and CRISPR-associated (Cas) gene (CRISPR/Cas), Transcription activator-like effector nucleases (TALEN) or Zinc-finger nucleases (ZFNs) or any combination thereof. According to further aspects of the invention, wherein the Cas protein is selected from a group comprising but not limited to Cpf1, Cas9, Cas12, Cas13, Cas14, CasX or CasY.

According to further aspects of the invention, the CRISPR/Cas system is delivered to the cell by Agrobacterium infiltration, virus based plasmids for delivery of the genome editing molecules and mechanical insertion of DNA (PEG mediated DNA transformation, biolistics, etc.).

According to further aspects of the invention, mutated regulatory gene is a CRISPR/Cas9-induced heritable mutated allele.

According to further aspects of the invention, the regulatory gene involved in allergen synthesis and/or fatty acid metabolism is edited through deleting, silencing, downregulating, de-regulating, deactivating, knockout, loss of function, reducing expression, at least partially deleting, at least partially silencing, at least partially deactivating, removing, partially removing, duplicating, inverting, missense mutation, nonsense mutation, insertion, deletion, indel, substitution or duplication or any combination thereof of the regulatory gene involved in allergen synthesis and/or in fatty acid metabolism.

The term gene editing also includes and encompasses applying base editing technique as known to a person skilled in the relevant art.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:

Genome Editing Glossary

Cas = CRISPR-associated genes
Cas9, Csn1 = a CRISPR-associated protein containing two nuclease
domains, that is programmed by small RNAs to cleave DNA
crRNA = CRISPR RNA
dCAS9 = nuclease-deficient Cas9
DSB = Double-Stranded Break
gRNA = guide RNA
HDR = Homology-Directed Repair
HNH = an endonuclease domain named for characteristic histidine
and asparagine residues
Indel = insertion and/or deletion
NHEJ = Non-Homologous End Joining
PAM = Protospacer-Adjacent Motif
RuvC = an endonuclease domain named for an E. coli protein
involved in DNA repair
sgRNA = single guide RNA
tracrRNA, trRNA = trans-activating crRNA
TALEN = Transcription-Activator Like Effector Nuclease
ZFN = Zinc-Finger Nuclease

According to specific aspects of the present invention, the CRISPR (Clustered Regularly Interspaced Short Palindromic Repeats) and CRISPR-associated (Cas) genes are used for the first time for generating genome modification in targeted genes in the Peanut plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9's function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA.

The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3′- of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9).

Reference is now made to CRISPR/Cas9 mechanism of action as depicted by Xie, Kabin, and Yinong Yang. “RNA-guided genome editing in plants using a CRISPR-Cas system.” Molecular plant 6.6 (2013): 1975-1983, schematically presenting an example of CRISPR/Cas9 mechanism. As shown in this publication, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and a motif called protospacer-adjacent motif or PAM that follows the base-pairing region in the complementary strand of the targeted DNA.

The commonly-used Cas9 from Streptococcus pyogenes (SpCas9) recognizes the PAM sequence 5′-NGG-3′ (where “N” can be any nucleotide base).

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

According to some aspects of the invention, the gRNA sequence comprises a 3′ NGG Protospacer Adjacent Motif (PAM), represented herein for each regulatory gene Ara h 1, Ara h1.1, Ara h2, Ara h3, Ara h5, Ara h6, Ara h7, Ara h8, Ara h9, Ara h10, Ara h11, Ara h14, Ara h15 and FAD2-1-FAD2-4 in Tables 2-14 and 16-19.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “orthologue” as used herein refers hereinafter to one of two or more homologous gene sequences found in different species.

The term “functional variant” or “functional variant of a nucleic acid or amino acid sequence” as used herein refers, without limitation, to a variant of a sequence or part of a sequence which retains the biological function of the full non-variant allele (e.g. Ara h1 or FAD2 allele) and hence has the activity of Ara h1 or FAD2 expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein refers, without limitation, to any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mats are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the identified Ara h 1, Ara h1.1, Ara h2, Ara h3, Ara h5, Ara h6, Ara h7, Ara h8, Ara h9, Ara h10, Ara h11, Ara h14, Ara h15 and FAD2-1-FAD2-4 regulatory genes in Peanut, namely Ara h 1, Ara h1.1, Ara h2, Ara h3, Ara h5, Ara h6, Ara h7, Ara h8, Ara h9, Ara h10, Ara h11, Ara h14, Ara h15 and FAD2-1-FAD2-4 having the genomic nucleotide sequence as set forth in SEQ ID NOs: 1, SEQ ID NO:341, SEQ ID NO:686, SEQ ID NO:908, SEQ ID NO:1161, SEQ ID NO:1348, SEQ ID NO: 1514, SEQ ID NO: 1690, SEQ ID NO: 1960, SEQ ID NO:2208, SEQ ID NO:2476, SEQ ID NO: 2665, SEQ ID NO:2854, SEQ ID NO:3065, SEQ ID NO:3410, SEQ ID NO:3809 and SEQ ID NO:4105, respectively.

As used herein, the term “locus” (loci plural) means a specific place or places or region or a site on a chromosome where, for example, a gene or genetic marker element or factor is found.

In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

In specific embodiments, the Peanut plants of the present invention comprise homozygous configuration of at least one of the mutated Ara h genes (i.e. Ara h1, Ara h1.1, Ara h2, Ara h3, Ara h5, Ara h6, Ara h7, Ara h8, Ara h9, Ara h10, Ara h11, Ara h14, Ara h15) and/or the mutated FAD2 genes (i.e. FAD2-1, FAD2-2, FAD2-3, FAD2-4).

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) plant. Specifically, the endogenous nucleic acid sequences of each of the Ara h or FAD2 homologs in peanut (nucleic acid sequences Ara h 1, Ara h1.1, Ara h2, Ara h3, Ara h5, Ara h6, Ara h7, Ara h8, Ara h9, Ara h10, Ara h11, Ara h14, Ara h15 and/or FAD2-1-FAD2-4) have been altered compared to wild type sequences using mutagenesis and/or genome editing methods as described herein. This causes inactivation of the endogenous Ara h and/or FAD2 genes and thus disables Ara h 1, Ara h1.1, Ara h2, Ara h3, Ara h5, Ara h6, Ara h7, Ara h8, Ara h9, Ara h10, Ara h11, Ara h14, Ara h15 and/or FAD2-1-FAD2-4 function. Such plants have an altered phenotype and show improved traits such as lower concentrations of allergens and/or altered levels of fatty acid (namely more linoleic acid than oleic acid) compared to wild type plants. Therefore, the improved phenotype is conferred by the presence of at least one mutated endogenous Ara h 1, Ara h1.1, Ara h2, Ara h3, Ara h5, Ara h6, Ara h7, Ara h8, Ara h9, Ara h10, Ara h11, Ara h14, Ara h15 and/or FAD2-FAD2-4 gene in the Peanut plant genome which has been specifically targeted using genome editing technique.

It is further within the scope of the current invention that Ara h 1, Ara h1.1, Ara h2, Ara h3, Ara h5, Ara h6, Ara h7, Ara h8, Ara h9, Ara h10, Ara h11, Ara h14, Ara h15 and/or FAD2-1-FAD2-4 mutations that down-regulate or disrupt functional expression of the wild-type Ara h 1, Ara h1.1, Ara h2, Ara h3, Ara h5, Ara h6, Ara h7, Ara h8, Ara h9, Ara h10, Ara h11, Ara h14, Ara h15 and/or FAD2-1-FAD2-4 sequences respectively, may be recessive, such that they are complemented by expression of a wild-type sequence.

It is further noted that a wild-type Peanut plant is a plant that does not have any mutant Ara h 1, Ara h1.1, Ara h2, Ara h3, Ara h5, Ara h6, Ara h7, Ara h8, Ara h9, Ara h10, Ara h11, Ara h14, Ara h15 and/or FAD2-1-FAD2-4 alleles.

Main aspects of the invention involve targeted mutagenesis methods, specifically genome editing, and exclude embodiments that are solely based on generating plants by traditional breeding methods. In a further embodiment of the current invention, as explained herein, the improved at least one trait is not due to the presence of a transgene. Therefore, according to some aspects of the invention, the Peanut plant does not comprise a transgene.

The inventors have generated mutant Peanut lines with mutations inactivating at least one Ara h regulatory gene among Ara h 1, Ara h1.1, Ara h2, Ara h3, Ara h5, Ara h6, Ara h7, Ara h8, Ara h9, Ara h10, Ara h11, Ara h14, Ara h15 and/or at least one FAD regulatory gene among FAD2-1-FAD2-4 which confer heritable improved trait(s). In this way, no functional Ara h and/or FAD2 protein is made. Thus, the invention relates to these mutant Peanut lines and related methods.

It is further within the scope of the present invention that breeding Peanut cultivars with mutated FAD allele extends the shelf-life of the generated plants, results in a Peanut with healthier fatty acid properties, and with a higher content of linoleic acid in comparison to oleic acid. According to a further aspect of the present invention, loss of Ara h function results in Peanut plants completely free of allergens, or with different expression of allergens, thus reducing the allergic response in a subject.

To edit multiple genes simultaneously and stack the resulting allelic variants, one option is that several gRNAs can be assembled to edit several genes into one construct, by using the Csy4 multi-gRNA system. The construct is then transformed via an appropriate vector into several wild-Peanuts accessions.

It is further within the scope of the current invention that Peanut Ara h genes, namely Ara h1 having genomic nucleotide sequence as set forth in SEQ. ID. NO: 1, were targeted using guide RNAs as set forth in SEQ ID NO:3 to SEQ ID NO:340; Ara h1.1 having genomic nucleotide sequence as set forth in SEQ. ID. NO: 341, were targeted using guide RNAs as set forth in SEQ ID NO: 343 to SEQ ID NO:685; Ara h2 having genomic nucleotide sequence as set forth in SEQ. ID. NO: 686, were targeted using guide RNAs as set forth in SEQ ID NO:688 to SEQ ID NO: 907; Ara h3 having genomic nucleotide sequence as set forth in SEQ. ID. NO: 908, were targeted using guide RNAs as set forth in SEQ ID NO:910 to SEQ ID NO:1160; Ara h5 having genomic nucleotide sequence as set forth in SEQ. ID. NO: 1161, were targeted using guide RNAs as set forth in SEQ ID NO:1163 to SEQ ID NO: 1347; Ara h6 having genomic nucleotide sequence as set forth in SEQ. ID. NO: 1348, were targeted using guide RNAs as set forth in SEQ ID NO:1350 to SEQ ID NO:1513; Ara h7 having genomic nucleotide sequence as set forth in SEQ. ID. NO: 1514, were targeted using guide RNAs as set forth in SEQ ID NO:1516 to SEQ ID NO:1689; Ara h8 having genomic nucleotide sequence as set forth in SEQ. ID. NO: 1690, were targeted using guide RNAs as set forth in SEQ ID NO:1692 to SEQ ID NO: 1959; Ara h9 having genomic nucleotide sequence as set forth in SEQ. ID. NO: 1960, were targeted using guide RNAs as set forth in SEQ ID NO:1962 to SEQ ID NO:2207; Ara h10 having genomic nucleotide sequence as set forth in SEQ. ID NO: 2208, were targeted using guide RNAs as set forth in SEQ ID NO:2210 to SEQ ID NO:2475; Ara h11 having genomic nucleotide sequence as set forth in SEQ. ID. NO: 2476, were targeted using guide RNAs as set forth in SEQ ID NO:2478 to SEQ ID NO:2664; Ara h14 having genomic nucleotide sequence as set forth in SEQ. ID. NO: 2665, were targeted using guide RNAs as set forth in SEQ ID NO: 2667 to SEQ ID NO:2853; and/or Ara h15 having genomic nucleotide sequence as set forth in SEQ. ID. NO: 2854, were targeted using guide RNAs as set forth in SEQ ID NO:2856 to SEQ ID NO:3064.

It is further withing the scope of the current invention that the functional variant has at least 75% sequence identity to the Ara h nucleotide sequences SEQ ID NO:1, SEQ ID NO:341, SEQ ID NO: 686, SEQ ID NO:908, SEQ ID NO:1161, SEQ ID NO:1348, SEQ ID NO:1514, SEQ ID NO: 1690, SEQ ID NO: 1960, SEQ ID NO:2208, SEQ ID NO:2476, SEQ ID NO:2665, SEQ ID NO: 2854, or any combination thereof.

It is further within the scope of the current invention that Peanut FAD genes, namely FAD2-1 having genomic nucleotide sequence as set forth in SEQ. ID NO: 3065, were targeted using guide RNAs as set forth in SEQ ID NO:3067 to SEQ ID NO:3409;

FAD2-2 having genomic nucleotide sequence as set forth in SEQ. ID. NO: 3410, were targeted using guide RNAs as set forth in SEQ ID NO:3412 to SEQ ID NO:3808;

FAD2-3 having genomic nucleotide sequence as set forth in SEQ. ID. NO: 3809, were targeted using guide RNAs as set forth in SEQ ID NO:3811 to SEQ ID NO:4104; and/or

FAD2-4 having genomic nucleotide sequence as set forth in SEQ. ID. NO: 4105, were targeted using guide RNAs as set forth in SEQ ID NO:4107 to SEQ ID NO:4400.

It is further withing the scope of the current invention that the functional variant has at least 75% sequence identity to the FAD nucleotide sequences SEQ ID NO:3065, SEQ ID NO:3410, SEQ ID NO:3809, SEQ ID NO:4105, or any combination thereof.

It is further withing the scope of the current invention that the regulatory gene involved in allergen synthesis is generated in planta via introduction of a construct comprising (a) Cas DNA and guide RNA (gRNA) sequence selected from the group consisting of SEQ ID NO:3-340, SEQ ID NO:343-685, SEQ ID NO:688-907, SEQ ID NO:910-1160, SEQ ID NO:1163-1347, SEQ ID NO:1350-1513, SEQ ID NO:1516-1689, SEQ ID NO:1692-1959, SEQ ID NO: 1962-2207, SEQ ID NO:2210-2475, SEQ ID NO:2478-2664, SEQ ID NO:2667-2853, and SEQ ID NO:2856-3064 for the regulator gene, or any combination thereof, or (b) a ribonucleoprotein (RNP) complex comprising Cas protein and gRNA sequence selected from the group consisting of SEQ ID NO:3-340, SEQ ID NO:343-685, SEQ ID NO:688-907, SEQ ID NO: 910-1160, SEQ ID NO:1163-1347, SEQ ID NO:1350-1513, SEQ ID NO:1516-1689, SEQ ID NO:1692-1959, SEQ ID NO:1962-2207, SEQ ID NO:2210-2475, SEQ ID NO:2478-2664, SEQ ID NO:2667-2853, and SEQ ID NO:2856-3064 for the regulatory gene, or any combination thereof.

It is further withing the scope of the current invention that the regulatory gene involved in fatty acid metabolism is generated in planta via introduction of a construct comprising (a) Cas DNA and guide RNA (gRNA) sequence selected from the group consisting of SEQ ID NO:3067 to SEQ ID NO:3409, SEQ ID NO:3412 to SEQ ID NO:3808, SEQ ID NO:3811 to SEQ ID NO: 4104, SEQ ID NO:4107 to SEQ ID NO:4400 for the regulatory gene, or any combination thereof, or (b) a ribonucleoprotein (RNP) complex comprising Cas protein and gRNA sequence selected from the group consisting of SEQ ID NO:3067 to SEQ ID NO:3409, SEQ ID NO:3412 to SEQ ID NO:3808, SEQ ID NO:3811 to SEQ ID NO:4104, SEQ ID NO:4107 to SEQ ID NO: 4400 for the regulatory gene, or any combination thereof.

### Example 1

Production of Peanut Plants with Improved Phenotypic Traits by Targeted Genome Editing.

Production of peanut lines with mutated Ara h, Ara h 1, Ara h1.1, Ara h2, Ara h3, Ara h5, Ara h6, Ara h7, Ara h8, Ara h9, Ara h10, Ara h11, Ara h14, Ara h15 and/or mutated FAD2-1, FAD2-2, FAD2-3, FAD2-4 genes may be achieved by at least one of the following breeding/cultivation schemes:

### Scheme 1:

line stabilization by self-pollination.
Generation of F6 parental lines.
Genome editing of parental lines.
Crossing edited parental lines to generate an F1 hybrid plant

### Scheme 2:

Identifying genes/alleles of interest.
Designing gRNA
Transformation of plants with Cas9+gRNA constructs.
Screening and identifying editing events
Genome editing of parental lines
   It is noted that line stabilization may be performed by the following:
Induction of male flowering on female plants.
Self pollination

According to some embodiments of the present invention, line stabilization requires about 6 self-crossing (6 generations) and done through a single seed descent (SSD) approach.

F1 hybrid seed production: Novel hybrids are produced by crosses between different peanut strains.

According to a further aspect of the current invention, shortening line stabilization is performed by Doubled Haploids (DH). More specifically, the CRISPR-Cas9 system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for peanuts are developed and provided by the current invention:
Genotyping markers—germplasm used in the current invention is genotyped using molecular markers, in order to allow a more efficient breeding process and identification of the Ara h and/or FAD2 editing event.

It is further within the scope of the current invention that allele and genetic variation is analyzed for the peanut strains used.

Reference is now made to optional stages that have been used for the production of mutated Ara h and/or FAD2 peanut plants by genome editing:
Stage 1: Identifying peanut Ara h and FAD2 orthologues.

13 Ara h orthologues and four FAD2 orthologues have herein been identified in peanut plants, namely Ara h 1, Ara h1.1, Ara h2, Ara h3, Ara h5, Ara h6, Ara h7, Ara h8, Ara h9, Ara h10, Ara h11, Ara h14, Ara h15; and FAD2-1, FAD2-2, FAD2-3, FAD2-4. These homologous genes have been sequenced and mapped.

Peanut Ara h as set forth in SEQ ID NO:1, SEQ ID NO:341, SEQ ID NO:686, SEQ ID NO:908, SEQ ID NO:1161, SEQ ID NO: 1348, SEQ ID NO: 1514, SEQ ID NO: 1690, SEQ ID NO:1960, SEQ ID NO:2208, SEQ ID NO:2476, SEQ ID NO:2665, SEQ ID NO:2854 for Ara h 1, Ara h1.1, Ara h2, Ara h3, Ara h5, Ara h6, Ara h7, Ara h8, Ara h9, Ara h10, Ara h11, Ara h14, Ara h15, respectively; and these genes encode amino acid sequences as set forth in SEQ ID NO:342, SEQ ID NO:687, SEQ ID NO:909, SEQ ID NO:1162, SEQ ID NO:1349, SEQ ID NO: 1515, SEQ ID NO: 1691, SEQ ID NO:1961, SEQ ID NO:2209, SEQ ID NO:2477, SEQ ID NO:2666, SEQ ID NO:2855, respectively.

Peanut FAD2 genes as set forth in SEQ ID NO:3065, SEQ ID NO:3410, SEQ ID NO:3809, SEQ ID NO:4105 for FAD2-1, FAD2-2, FAD2-3, FAD2-4, respectively; and these genes encode amino acid sequences as set forth in SEQ ID NO:3066, SEQ ID NO: 3411, SEQ ID NO: 3810, SEQ ID NO:4106, respectively.
Stage 2: Designing and synthesizing gRNA molecules corresponding to the sequence targeted for editing, i.e. sequences of each of the genes Ara h 1, Ara h1.1, Ara h2, Ara h3, Ara h5, Ara h6, Ara h7, Ara h8, Ara h9, Ara h10, Ara h11, Ara h14, Ara h15; and FAD2-1, FAD2-2, FAD2-3, FAD2-4. It is noted that the editing event is preferably targeted to a unique restriction site sequence to allow easier screening for plants carrying an editing event within their genome.

According to some aspects of the invention, the nucleotide sequence of the gRNAs should be completely compatible with the genomic sequence of the target gene. Therefore, for example, suitable gRNA molecules should be constructed for different Ara h and/or FAD2 homologues of different peanut strains.

Reference is now made to Table 1, listing the genomic sequences, the amino acid sequences and the gRNA's targeting the gene sequences or regulatory sequences upstream of first ATG of each of Ara h gene Ara h 1, Ara h1.1, Ara h2, Ara h3, Ara h5, Ara h6, Ara h7, Ara h8, Ara h9, Ara h10, Ara h11, Ara h14, Ara h15.

TABLE 1
Genomic sequences, the amino acid sequences and the
gRNA's targeting sequences of each Ara h gene.

| | Genomic | AA | gRNA |
|---|---|---|---|
| Gene name | SEQ ID NO: | SEQ ID NO: | SEQ ID NO: |
| Arah1 | 1 | 2 | 3-340 |
| Arah1.1 | 341 | 342 | 343-685 |
| Arah2 | 686 | 687 | 688-907 |
| Arah3 | 908 | 909 | 910-1160 |
| Arah5 | 1161 | 1162 | 1163-1347 |
| Arah6 | 1348 | 1349 | 1350-1513 |
| Arah7 | 1514 | 1515 | 1516-1689 |
| Arah8 | 1690 | 1691 | 1692-1959 |
| Arah9 | 1960 | 1961 | 1962-2207 |
| Arah10 | 2208 | 2209 | 2210-2475 |
| Arah11 | 2476 | 2477 | 2478-2664 |
| Arah14 | 2665 | 2666 | 2667-2853 |
| Arah15 | 2854 | 2855 | 2856-3064 |

Reference is now made to Tables 2-14 presenting sequences of gRNA molecules targeted for Ara h 1, Ara h1.1, Ara h2, Ara h3, Ara h5, Ara h6, Ara h7, Ara h8, Ara h9, Ara h10, Ara h11, Ara h14 and Ara h15. The term ‘PAM’ refers hereinafter to Protospacer Adjacent Motif, which is a 2-6 base pair DNA sequence immediately following the DNA sequence targeted by the Cas9 nuclease in the CRISPR bacterial adaptive immune system.

The gRNAs are targeting coding sequences of Ara h genes and regulatory sequences upstream of Ara h genes first ATG.

TABLE 2
gRNA's targeting sequences of Ara h1.

| Position | | | | |
|---|---|---|---|---|
| on SEQ ID | | | | SEQ ID |
| NO: 1 | Strand | Sequence | PAM | NO: |
| 28 | 1 | CAAAAGCAGTAAACTTCAAA | TGG | 3 |
| 53 | 1 | GTTTCTCATTAGTTCACCGT | TGG | 4 |
| 58 | 1 | TCATTAGTTCACCGTTGGAT | CGG | 5 |
| 58 | −1 | CAAATTTCAAGCCGATCCAA | CGG | 6 |
| 105 | 1 | TCTCATCTTGTTCTTCATTC | TGG | 7 |
| 109 | 1 | ATCTTGTTCTTCATTCTGGA | CGG | 8 |
| 112 | 1 | TTGTTCTTCATTCTGGACGG | TGG | 9 |
| 148 | 1 | AAGTCTGCAGTGAGAGAAAT | TGG | 10 |
| 169 | 1 | GGCTTCGCAAGAGAAAAATT | AGG | 11 |
| 185 | −1 | ATTGCTACTCTGTGTAAAAA | TGG | 12 |
| 206 | 1 | ACAGAGTAGCAATTTTTGAA | CGG | 13 |
| 231 | 1 | GTTTCTCATTTTTTCACCGT | TGG | 14 |
| 236 | −1 | CAAATTTCACGTCGATCCAA | CGG | 15 |
| 248 | 1 | CGTTGGATCGACGTGAAATT | TGG | 16 |
| 298 | −1 | GAAGCACTCACAACACAATG | TGG | 17 |
| 320 | −1 | TATCCAATACAACAATAGTA | TGG | 18 |
| 328 | 1 | CTTCCATACTATTGTTGTAT | TGG | 19 |
| 374 | 1 | AGACTCTTTCAATAATGTCT | AGG | 20 |
| 443 | 1 | TTAAACGAAAAGATAAATGT | TGG | 21 |
| 473 | −1 | TTATTCTATTGGGAAAAGAT | TGG | 22 |
| 483 | −1 | GGGGCATAATTTATTCTATT | GGG | 23 |
| 484 | −1 | TGGGGCATAATTTATTCTAT | TGG | 24 |
| 502 | −1 | ACATATTGATAAAATAATTG | GGG | 25 |
| 503 | −1 | AACATATTGATAAAATAATT | GGG | 26 |
| 504 | −1 | TAACATATTGATAAAATAAT | TGG | 27 |
| 521 | 1 | ATTTTATCAATATGTTAATT | AGG | 28 |
| 535 | 1 | TTAATTAGGAATGAACATTT | TGG | 29 |
| 589 | 1 | GATTACTTTGTTTCCATAGT | TGG | 30 |
| 591 | −1 | TACAAAAGATAATCCAACTA | TGG | 31 |
| 613 | 1 | TTATCTTTTGTAGAATAACG | AGG | 32 |
| 614 | 1 | TATCTTTTGTAGAATAACGA | GGG | 33 |
| 640 | 1 | AATCTCTAATATACATTTGT | CGG | 34 |
| 647 | 1 | AATATACATTTGTCGGCCTT | AGG | 35 |
| 652 | −1 | AGCATTAATAACACAACCTA | AGG | 36 |
| 673 | 1 | TGTTATTAATGCTTCTCATA | CGG | 37 |
| 687 | −1 | AAGCTCTAGCTACAGAGTTG | TGG | 38 |
| 744 | 1 | GATGATATCAAAAAGAAAGA | AGG | 39 |
| 775 | 1 | AAACGTATGTGATGTTTAAA | TGG | 40 |
| 789 | 1 | TTTAAATGGATATGAAATTT | TGG | 41 |
| 840 | 1 | TGATACGCAGATACGCTAAA | AGG | 42 |
| 856 | −1 | GCACCCCTACCTTACGGTAT | TGG | 43 |
| 858 | 1 | AAAGGCAAGCCAATACCGTA | AGG | 44 |
| 862 | 1 | GCAAGCCAATACCGTAAGGT | AGG | 45 |
| 862 | −1 | ACATATGCACCCCTACCTTA | CGG | 46 |
| 863 | 1 | CAAGCCAATACCGTAAGGTA | GGG | 47 |
| 864 | 1 | AAGCCAATACCGTAAGGTAG | GGG | 48 |
| 878 | 1 | AGGTAGGGGTGCATATGTGC | CGG | 49 |
| 879 | 1 | GGTAGGGGTGCATATGTGCC | GGG | 50 |
| 886 | −1 | ACATCAAACCCGACTTCACC | CGG | 51 |
| 888 | 1 | GCATATGTGCCGGGTGAAGT | CGG | 52 |
| 889 | 1 | CATATGTGCCGGGTGAAGTC | GGG | 53 |
| 911 | −1 | GTATATTTCGGATCGGGTCT | GGG | 54 |
| 912 | −1 | TGTATATTTCGGATCGGGTC | TGG | 55 |
| 917 | −1 | CCCGGTGTATATTTCGGATC | GGG | 56 |
| 918 | −1 | ACCCGGTGTATATTTCGGAT | CGG | 57 |
| 923 | −1 | AATAAACCCGGTGTATATTT | CGG | 58 |
| 927 | 1 | ACCCGATCCGAAATATACAC | CGG | 59 |
| 928 | 1 | CCCGATCCGAAATATACACC | GGG | 60 |
| 935 | −1 | TCGGGTCTAATAAATAAACC | CGG | 61 |
| 953 | −1 | TCGGGTCTAGGGTCGGGTTC | GGG | 62 |
| 954 | −1 | ATCGGGTCTAGGGTCGGGTT | CGG | 63 |
| 959 | −1 | GTTTCATCGGGTCTAGGGTC | GGG | 64 |
| 960 | −1 | GGTTTCATCGGGTCTAGGGT | CGG | 65 |
| 964 | −1 | TATTGGTTTCATCGGGTCTA | GGG | 66 |
| 965 | −1 | GTATTGGTTTCATCGGGTCT | AGG | 67 |
| 971 | −1 | GAAAGTGTATTGGTTTCATC | GGG | 68 |
| 972 | −1 | CGAAAGTGTATTGGTTTCAT | CGG | 69 |
| 981 | −1 | ATTGTGGCCCGAAAGTGTAT | TGG | 70 |
| 984 | 1 | CGATGAAACCAATACACTTT | CGG | 71 |
| 985 | 1 | GATGAAACCAATACACTTTC | GGG | 72 |
| 997 | −1 | GTTTTTACCCGGTATAATTG | TGG | 73 |
| 1000 | 1 | CTTTCGGGCCACAATTATAC | CGG | 74 |
| 1001 | 1 | TTTCGGGCCACAATTATACC | GGG | 75 |
| 1008 | −1 | GTTTTCACCCGGTTTTTACC | CGG | 76 |
| 1011 | 1 | CAATTATACCGGGTAAAAAC | CGG | 77 |
| 1012 | 1 | AATTATACCGGGTAAAAACC | GGG | 78 |
| 1019 | −1 | TTAATGGCCCGGTTTTCACC | CGG | 79 |
| 1022 | 1 | GGTAAAAACCGGGTGAAAAC | CGG | 80 |
| 1023 | 1 | GTAAAAACCGGGTGAAAACC | GGG | 81 |
| 1030 | −1 | GTAATGTAATGTTAATGGCC | CGG | 82 |
| 1035 | −1 | TCAACGTAATGTAATGTTAA | TGG | 83 |
| 1060 | −1 | TTACATGCTAGCTTACAAGA | AGG | 84 |
| 1089 | −1 | TGGTTGGAGTCTTGAAAATT | TGG | 85 |
| 1105 | −1 | TTACCATGTTAAATAATGGT | TGG | 86 |
| 1109 | −1 | AATTTTACCATGTTAAATAA | TGG | 87 |
| 1113 | 1 | ACTCCAACCATTATTTAACA | TGG | 88 |
| 1155 | −1 | CTTTAATTTTAAAGAAGGGT | TGG | 89 |
| 1159 | −1 | TATGCTTTAATTTTAAAGAA | GGG | 90 |
| 1160 | −1 | TTATGCTTTAATTTTAAAGA | AGG | 91 |
| 1183 | −1 | TTTTGCTTTAGTATGGATTG | TGG | 92 |
| 1190 | −1 | ATTGTGGTTTTGCTTTAGTA | TGG | 93 |
| 1206 | −1 | GACAATATTAGTATTGATTG | TGG | 94 |
| 1237 | −1 | TTTGTATTGATTTAAATATT | TGG | 95 |
| 1374 | −1 | GGATCATGACTATTCTTATG | TGG | 96 |
| 1395 | −1 | ATATTTTGTGTTATTAGTGA | TGG | 97 |
| 1426 | 1 | TATTAATTGTGTATGATGAC | CGG | 98 |
| 1427 | 1 | ATTAATTGTGTATGATGACC | GGG | 99 |
| 1434 | −1 | CCGAAGTCGGCCTGGTGGCC | CGG | 100 |
| 1435 | 1 | TGTATGATGACCGGGCCACC | AGG | 101 |
| 1439 | −1 | GTCACCCGAAGTCGGCCTGG | TGG | 102 |
| 1442 | −1 | CGGGTCACCCGAAGTCGGCC | TGG | 103 |
| 1445 | 1 | CCGGGCCACCAGGCCGACTT | CGG | 104 |
| 1446 | 1 | CGGGCCACCAGGCCGACTTC | GGG | 105 |
| 1447 | −1 | TAGCTCGGGTCACCCGAAGT | CGG | 106 |
| 1461 | 1 | ACTTCGGGTGACCCGAGCTA | TGG | 107 |
| 1461 | −1 | TCGGGTCCGGGCCATAGCTC | GGG | 108 |
| 1462 | −1 | GTCGGGTCCGGGCCATAGCT | CGG | 109 |
| 1466 | 1 | GGGTGACCCGAGCTATGGCC | CGG | 110 |
| 1473 | −1 | CATTATTTCGGGTCGGGTCC | GGG | 111 |
| 1474 | −1 | TCATTATTTCGGGTCGGGTC | CGG | 112 |
| 1479 | −1 | CCCGGTCATTATTTCGGGTC | GGG | 113 |
| 1480 | −1 | ACCCGGTCATTATTTCGGGT | CGG | 114 |
| 1484 | −1 | ATAGACCCGGTCATTATTTC | GGG | 115 |
| 1485 | −1 | AATAGACCCGGTCATTATTT | CGG | 116 |
| 1489 | 1 | ACCCGACCCGAAATAATGAC | CGG | 117 |
| 1490 | 1 | CCCGACCCGAAATAATGACC | GGG | 118 |
| 1497 | −1 | AAGGGTCTCAAAAATAGACC | CGG | 119 |
| 1515 | −1 | TCGGGTTTAGGGTCGGGTAA | GGG | 120 |
| 1516 | −1 | ATCGGGTTTAGGGTCGGGTA | AGG | 121 |
| 1521 | −1 | ATTTCATCGGGTTTAGGGTC | GGG | 122 |
| 1522 | −1 | GATTTCATCGGGTTTAGGGT | CGG | 123 |
| 1526 | −1 | GTATGATTTCATCGGGTTTA | GGG | 124 |
| 1527 | −1 | GGTATGATTTCATCGGGTTT | AGG | 125 |
| 1533 | −1 | TAATTTGGTATGATTTCATC | GGG | 126 |
| 1534 | −1 | CTAATTTGGTATGATTTCAT | CGG | 127 |
| 1548 | −1 | GAACATTTTAGGGGCTAATT | TGG | 128 |
| 1557 | −1 | CCCGGTCCCGAACATTTTAG | GGG | 129 |
| 1558 | −1 | GCCCGGTCCCGAACATTTTA | GGG | 130 |
| 1559 | −1 | GGCCCGGTCCCGAACATTTT | AGG | 131 |
| 1561 | 1 | AAATTAGCCCCTAAAATGTT | CGG | 132 |
| 1562 | 1 | AATTAGCCCCTAAAATGTTC | GGG | 133 |
| 1567 | 1 | GCCCCTAAAATGTTCGGGAC | CGG | 134 |
| 1568 | 1 | CCCCTAAAATGTTCGGGACC | GGG | 135 |
| 1572 | 1 | TAAAATGTTCGGGACCGGGC | CGG | 136 |
| 1573 | 1 | AAAATGTTCGGGACCGGGCC | GGG | 137 |
| 1575 | −1 | CCCGGCCCGAAGGCCCGGCC | CGG | 138 |
| 1580 | 1 | TCGGGACCGGGCCGGGCCTT | CGG | 139 |
| 1580 | −1 | CCCGGCCCGGCCCGAAGGCC | CGG | 140 |
| 1581 | 1 | CGGGACCGGGCCGGGCCTTC | GGG | 141 |
| 1585 | 1 | ACCGGGCCGGGCCTTCGGGC | CGG | 142 |
| 1585 | −1 | ACAGACCCGGCCCGGCCCGA | AGG | 143 |
| 1586 | 1 | CCGGGCCGGGCCTTCGGGCC | GGG | 144 |
| 1590 | 1 | GCCGGGCCTTCGGGCCGGGC | CGG | 145 |
| 1591 | 1 | CCGGGCCTTCGGGCCGGGCC | GGG | 146 |
| 1593 | −1 | AGGGGTGCACAGACCCGGCC | CGG | 147 |
| 1598 | −1 | ACGGTAGGGGTGCACAGACC | CGG | 148 |
| 1611 | −1 | TTCAGCTATCCTTACGGTAG | GGG | 149 |
| 1612 | −1 | ATTCAGCTATCCTTACGGTA | GGG | 150 |
| 1613 | 1 | GTCTGTGCACCCCTACCGTA | AGG | 151 |
| 1613 | −1 | CATTCAGCTATCCTTACGGT | AGG | 152 |
| 1617 | −1 | CACCCATTCAGCTATCCTTA | CGG | 153 |
| 1625 | 1 | CTACCGTAAGGATAGCTGAA | TGG | 154 |
| 1626 | 1 | TACCGTAAGGATAGCTGAAT | GGG | 155 |
| 1654 | 1 | ATATTTTATCAAAAGACAAA | AGG | 156 |
| 1669 | −1 | GTTCCACTGCACATACAGGA | TGG | 157 |
| 1673 | −1 | AAAGGTTCCACTGCACATAC | AGG | 158 |
| 1677 | 1 | AATCCATCCTGTATGTGCAG | TGG | 159 |
| 1691 | −1 | TCACTCCATTGTCTGGGAAA | AGG | 160 |
| 1697 | 1 | TGGAACCTTTTCCCAGACAA | TGG | 161 |
| 1697 | −1 | TGTTCATCACTCCATTGTCT | GGG | 162 |
| 1698 | −1 | ATGTTCATCACTCCATTGTC | TGG | 163 |
| 1732 | −1 | GATATGTATGGTGATGGACG | CGG | 164 |
| 1738 | −1 | GCTTTGGATATGTATGGTGA | TGG | 165 |
| 1744 | −1 | TTTTTTGCTTTGGATATGTA | TGG | 166 |
| 1754 | −1 | ATTTGTTAAATTTTTTGCTT | TGG | 167 |
| 1783 | −1 | GTTAATATGCATGGCAATTG | GGG | 168 |
| 1784 | −1 | TGTTAATATGCATGGCAATT | GGG | 169 |
| 1785 | −1 | GTGTTAATATGCATGGCAAT | TGG | 170 |
| 1792 | −1 | AGTGTACGTGTTAATATGCA | TGG | 171 |
| 1831 | −1 | GGATGGATTGTGGTGCATGG | TGG | 172 |
| 1834 | −1 | GATGGATGGATTGTGGTGCA | TGG | 173 |
| 1841 | −1 | AACAAGAGATGGATGGATTG | TGG | 174 |
| 1848 | −1 | AGAGATAAACAAGAGATGGA | TGG | 175 |
| 1852 | −1 | GCTTAGAGATAAACAAGAGA | TGG | 176 |
| 1874 | −1 | GTGGCAACAACACTAGGTCT | TGG | 177 |
| 1880 | −1 | TGTGAGGTGGCAACAACACT | AGG | 178 |
| 1893 | −1 | TGAAATTGAGAAATGTGAGG | TGG | 179 |
| 1896 | −1 | TGTTGAAATTGAGAAATGTG | AGG | 180 |
| 1927 | −1 | TGGGCATTGGAGTGGCATGC | AGG | 181 |
| 1935 | −1 | TGTACACTTGGGCATTGGAG | TGG | 182 |
| 1940 | −1 | ATGGATGTACACTTGGGCAT | TGG | 183 |
| 1946 | −1 | GATGATATGGATGTACACTT | GGG | 184 |
| 1947 | −1 | GGATGATATGGATGTACACT | TGG | 185 |
| 1959 | −1 | ATAGTTGCGTTAGGATGATA | TGG | 186 |
| 1968 | −1 | GGTATTTATATAGTTGCGTT | AGG | 187 |
| 1989 | −1 | AAATGATGGAGGGAGGTGAG | TGG | 188 |
| 1996 | −1 | GATGGTGAAATGATGGAGGG | AGG | 189 |
| 1999 | −1 | GTGGATGGTGAAATGATGGA | GGG | 190 |
| 2000 | −1 | TGTGGATGGTGAAATGATGG | AGG | 191 |
| 2003 | −1 | GAGTGTGGATGGTGAAATGA | TGG | 192 |
| 2014 | −1 | ATGATTATTATGAGTGTGGA | TGG | 193 |
| 2018 | −1 | ATATATGATTATTATGAGTG | TGG | 194 |
| 2061 | 1 | TCATCTATATAAGTAGTAGC | AGG | 195 |
| 2073 | 1 | GTAGTAGCAGGAGCAATGAG | AGG | 196 |
| 2074 | 1 | TAGTAGCAGGAGCAATGAGA | GGG | 197 |
| 2077 | 1 | TAGCAGGAGCAATGAGAGGG | AGG | 198 |
| 2078 | 1 | AGCAGGAGCAATGAGAGGGA | GGG | 199 |
| 2095 | −1 | ATCCCTAGCAACAGCATCAG | TGG | 200 |
| 2103 | 1 | TCTCCACTGATGCTGTTGCT | AGG | 201 |
| 2104 | 1 | CTCCACTGATGCTGTTGCTA | GGG | 202 |
| 2117 | 1 | GTTGCTAGGGATCCTTGTCC | TGG | 203 |
| 2118 | −1 | CAGAAACTGAAGCCAGGACA | AGG | 204 |
| 2124 | −1 | GCGTTGCAGAAACTGAAGCC | AGG | 205 |
| 2150 | −1 | CTTCTGGTAAGGTGATGACT | TGG | 206 |
| 2161 | −1 | TTCTCTGTTTTCTTCTGGTA | AGG | 207 |
| 2166 | −1 | AGGGGTTCTCTGTTTTCTTC | TGG | 208 |
| 2184 | −1 | GGAGGCACCTCTGGGCGCAG | GGG | 209 |
| 2185 | −1 | TGGAGGCACCTCTGGGCGCA | GGG | 210 |
| 2186 | −1 | CTGGAGGCACCTCTGGGCGC | AGG | 211 |
| 2188 | 1 | CAGAGAACCCCTGCGCCCAG | AGG | 212 |
| 2192 | −1 | ACAACTCTGGAGGCACCTCT | GGG | 213 |
| 2193 | −1 | GACAACTCTGGAGGCACCTC | TGG | 214 |
| 2202 | −1 | GTTCCTGTTGACAACTCTGG | AGG | 215 |
| 2205 | −1 | CCGGTTCCTGTTGACAACTC | TGG | 216 |
| 2210 | 1 | GTGCCTCCAGAGTTGTCAAC | AGG | 217 |
| 2216 | 1 | CCAGAGTTGTCAACAGGAAC | CGG | 218 |
| 2224 | −1 | GCCTTTTGCTTCAAGTCATC | CGG | 219 |
| 2234 | 1 | ACCGGATGACTTGAAGCAAA | AGG | 220 |
| 2264 | −1 | ACGAGGATCATACTCGAGCT | TGG | 221 |
| 2281 | −1 | CGAGGATCATAGACACAACG | AGG | 222 |
| 2295 | 1 | CGTTGTGTCTATGATCCTCG | AGG | 223 |
| 2299 | −1 | GTGGTGCCAGTGTGTCCTCG | AGG | 224 |
| 2304 | 1 | TATGATCCTCGAGGACACAC | TGG | 225 |
| 2318 | −1 | TGGAGGGGAACGTTGGTTGG | TGG | 226 |
| 2321 | −1 | CCCTGGAGGGGAACGTTGGT | TGG | 227 |
| 2325 | −1 | GCTCCCCTGGAGGGGAACGT | TGG | 228 |
| 2331 | 1 | ACCAACCAACGTTCCCCTCC | AGG | 229 |
| 2332 | 1 | CCAACCAACGTTCCCCTCCA | GGG | 230 |
| 2333 | 1 | CAACCAACGTTCCCCTCCAG | GGG | 231 |
| 2333 | −1 | ACGTGTCCGCTCCCCTGGAG | GGG | 232 |
| 2334 | −1 | CACGTGTCCGCTCCCCTGGA | GGG | 233 |
| 2335 | −1 | CCACGTGTCCGCTCCCCTGG | AGG | 234 |
| 2338 | 1 | AACGTTCCCCTCCAGGGGAG | CGG | 235 |
| 2338 | −1 | CGGCCACGTGTCCGCTCCCC | TGG | 236 |
| 2346 | 1 | CCTCCAGGGGAGCGGACACG | TGG | 237 |
| 2358 | 1 | CGGACACGTGGCCGCCAACC | CGG | 238 |
| 2358 | −1 | CATCGTAGTCTCCGGGTTGG | CGG | 239 |
| 2361 | −1 | CATCATCGTAGTCTCCGGGT | TGG | 240 |
| 2365 | −1 | CGGTCATCATCGTAGTCTCC | GGG | 241 |
| 2366 | −1 | GCGGTCATCATCGTAGTCTC | CGG | 242 |
| 2385 | −1 | CCTCTCTTCGGGGTTGACGG | CGG | 243 |
| 2388 | −1 | CTTCCTCTCTTCGGGGTTGA | CGG | 244 |
| 2395 | −1 | CGGCCTCCTTCCTCTCTTCG | GGG | 245 |
| 2396 | 1 | CCGCCGTCAACCCCGAAGAG | AGG | 246 |
| 2396 | −1 | TCGGCCTCCTTCCTCTCTTC | GGG | 247 |
| 2397 | −1 | ATCGGCCTCCTTCCTCTCTT | CGG | 248 |
| 2400 | 1 | CGTCAACCCCGAAGAGAGGA | AGG | 249 |
| 2403 | 1 | CAACCCCGAAGAGAGGAAGG | AGG | 250 |
| 2410 | 1 | GAAGAGAGGAAGGAGGCCGA | TGG | 251 |
| 2411 | 1 | AAGAGAGGAAGGAGGCCGAT | GGG | 252 |
| 2412 | 1 | AGAGAGGAAGGAGGCCGATG | GGG | 253 |
| 2415 | 1 | TCGGTCCAGCTGGTCCCCAT | CGG | 254 |
| 2421 | 1 | GGAGGCCGATGGGGACCAGC | TGG | 255 |
| 2425 | −1 | TCACGCTCCCTCGGTCCAGC | TGG | 256 |
| 2428 | 1 | GATGGGGACCAGCTGGACCG | AGG | 257 |
| 2429 | 1 | ATGGGGACCAGCTGGACCGA | GGG | 258 |
| 2434 | −1 | TCTTCTCTTTCACGCTCCCT | CGG | 259 |
| 2452 | 1 | AGCGTGAAAGAGAAGAAGAC | TGG | 260 |
| 2470 | 1 | GGTCGCCTCCAATCTTCTCT | TGG | 261 |
| 2473 | 1 | GGAGACAACCAAGAGAAGAT | TGG | 262 |
| 2476 | 1 | GACAACCAAGAGAAGATTGG | AGG | 263 |
| 2491 | −1 | TTCCGTGGCTGCTGATGACT | TGG | 264 |
| 2500 | 1 | GACCAAGTCATCAGCAGCCA | CGG | 265 |
| 2506 | −1 | CCTTCGGGCCTTATTTTCCG | TGG | 266 |
| 2509 | 1 | ATCAGCAGCCACGGAAAATA | AGG | 267 |
| 2517 | 1 | CCACGGAAAATAAGGCCCGA | AGG | 268 |
| 2521 | −1 | TGTTCTCCTTCTCTTCCTTC | GGG | 269 |
| 2522 | −1 | TTGTTCTCCTTCTCTTCCTT | CGG | 270 |
| 2526 | 1 | ATAAGGCCCGAAGGAAGAGA | AGG | 271 |
| 2539 | 1 | GAAGAGAAGGAGAACAAGAG | TGG | 272 |
| 2540 | 1 | AAGAGAAGGAGAACAAGAGT | GGG | 273 |
| 2541 | 1 | AGAGAAGGAGAACAAGAGTG | GGG | 274 |
| 2550 | 1 | GAACAAGAGTGGGGAACACC | AGG | 275 |
| 2557 | −1 | TCTTCCCTCACATGGCTACC | TGG | 276 |
| 2563 | 1 | GAACACCAGGTAGCCATGTG | AGG | 277 |
| 2564 | 1 | AACACCAGGTAGCCATGTGA | GGG | 278 |
| 2565 | −1 | GAGATGTTTCTTCCCTCACA | TGG | 279 |
| 2578 | 1 | ATGTGAGGGAAGAAACATCT | CGG | 280 |
| 2595 | −1 | GCCTTGACGGGAAGTAGAAA | GGG | 281 |
| 2596 | 1 | CGCCTTGACGGGAAGTAGAA | AGG | 282 |
| 2605 | 1 | ACCCTTTCTACTTCCCGTCA | AGG | 283 |
| 2607 | −1 | GGGTGCTAAACCGCCTTGAC | GGG | 284 |
| 2608 | 1 | CTTTCTACTTCCCGTCAAGG | CGG | 285 |
| 2608 | −1 | CGGGTGCTAAACCGCCTTGA | CGG | 286 |
| 2625 | 1 | AGGCGGTTTAGCACCCGCTA | CGG | 287 |
| 2626 | 1 | GGCGGTTTAGCACCCGCTAC | GGG | 288 |
| 2627 | −1 | ACCGTTTTGGTTCCCGTAGC | GGG | 289 |
| 2628 | −1 | TACCGTTTTGGTTCCCGTAG | CGG | 290 |
| 2637 | 1 | ACCCGCTACGGGAACCAAAA | CGG | 291 |
| 2640 | −1 | GGACCCGGATCCTACCGTTT | TGG | 292 |
| 2641 | 1 | GCTACGGGAACCAAAACGGT | AGG | 293 |
| 2647 | 1 | GGAACCAAAACGGTAGGATC | CGG | 294 |
| 2648 | 1 | GAACCAAAACGGTAGGATCC | GGG | 295 |
| 2655 | −1 | GGTCAAACCTCTGCAGGACC | CGG | 296 |
| 2659 | 1 | GTAGGATCCGGGTCCTGCAG | AGG | 297 |
| 2661 | −1 | ACCTTTGGTCAAACCTCTGC | AGG | 298 |
| 2671 | 1 | TCCTGCAGAGGTTTGACCAA | AGG | 299 |
| 2676 | −1 | TCTGAAACTGCCTTGACCTT | TGG | 300 |
| 2677 | 1 | AGAGGTTTGACCAAAGGTCA | AGG | 301 |
| 2703 | −1 | TCTGCACAATACGGTGATTC | TGG | 302 |
| 2712 | −1 | TGGCCTCGATCTGCACAATA | CGG | 303 |
| 2720 | 1 | TCACCGTATTGTGCAGATCG | AGG | 304 |
| 2732 | −1 | AAGAACAAGAGTGTTAGGTT | TGG | 305 |
| 2737 | −1 | TTGGGAAGAACAAGAGTGTT | AGG | 306 |
| 2755 | −1 | TTATCAGCATCAGCGTGCTT | GGG | 307 |
| 2756 | −1 | GTTATCAGCATCAGCGTGCT | TGG | 308 |
| 2781 | −1 | TGATACCTTGCTGGATAACA | AGG | 309 |
| 2787 | 1 | AACATCCTTGTTATCCAGCA | AGG | 310 |
| 2790 | −1 | AATTAGATTTGATACCTTGC | TGG | 311 |
| 2850 | 1 | GCTTGATACATATGATTCAT | TGG | 312 |
| 2858 | 1 | CATATGATTCATTGGATTGC | AGG | 313 |
| 2859 | 1 | ATATGATTCATTGGATTGCA | GGG | 314 |
| 2875 | −1 | GCCATTTGCTACGGTCACGG | TGG | 315 |
| 2878 | −1 | ATTGCCATTTGCTACGGTCA | CGG | 316 |
| 2884 | −1 | TCTGTTATTGCCATTTGCTA | CGG | 317 |
| 2885 | 1 | GCCACCGTGACCGTAGCAAA | TGG | 318 |
| 2917 | 1 | AAAGAGCTTTAATCTTGACG | AGG | 319 |
| 2918 | 1 | AAGAGCTTTAATCTTGACGA | GGG | 320 |
| 2930 | −1 | CGGATGGGATTCTGAGTGCA | TGG | 321 |
| 2942 | 1 | CATGCACTCAGAATCCCATC | CGG | 322 |
| 2945 | −1 | TGTAGGAAATGAAACCGGAT | GGG | 323 |
| 2946 | −1 | ATGTAGGAAATGAAACCGGA | TGG | 324 |
| 2950 | −1 | CAAGATGTAGGAAATGAAAC | CGG | 325 |
| 2962 | −1 | GTCATGGCGGTTCAAGATGT | AGG | 326 |
| 2975 | −1 | TGAGGTTCTGGTTGTCATGG | CGG | 327 |
| 2978 | −1 | CTCTGAGGTTCTGGTTGTCA | TGG | 328 |
| 2987 | −1 | TTTTAGCTACTCTGAGGTTC | TGG | 329 |
| 2993 | −1 | TGGAGATTTTAGCTACTCTG | AGG | 330 |
| 3013 | −1 | GCCGGGTGTGTTAACGGGCA | TGG | 331 |
| 3018 | −1 | AACTGGCCGGGTGTGTTAAC | GGG | 332 |
| 3019 | −1 | AAACTGGCCGGGTGTGTTAA | CGG | 333 |
| 3023 | 1 | TCCATGCCCGTTAACACACC | CGG | 334 |
| 3030 | −1 | AGAGGTACCTCAAACTGGCC | GGG | 335 |
| 3031 | −1 | AAGAGGTACCTCAAACTGGC | CGG | 336 |
| 3034 | 1 | TAACACACCCGGCCAGTTTG | AGG | 337 |
| 3035 | −1 | AAGAAAGAGGTACCTCAAAC | TGG | 338 |
| 3048 | −1 | ATATATATGTGAGAAGAAAG | AGG | 339 |
| 3067 | 1 | TTCTCACATATATATTCAAG | AGG | 340 |

TABLE 3
gRNA's targeting sequences of Ara h1.1.

| Position | | | | |
|---|---|---|---|---|
| on SEQ ID | | | | SEQ ID |
| NO: 341 | Strand | Sequence | PAM | NO: |
| 35 | 1 | ACAGGAGAAGTACATAAAGA | AGG | 343 |
| 46 | 1 | ACATAAAGAAGGTGTTTCAA | AGG | 344 |
| 51 | 1 | AAGAAGGTGTTTCAAAGGTT | TGG | 345 |
| 74 | −1 | AAAAGAACTAGCAACACATT | TGG | 346 |
| 100 | −1 | TTGGTGCTCAACTTAAAATG | AGG | 347 |
| 119 | −1 | ATCAGTGGTTGGACACTGCT | TGG | 348 |
| 130 | −1 | TGTTTCTCTTCATCAGTGGT | TGG | 349 |
| 134 | −1 | TGCTTGTTTCTCTTCATCAG | TGG | 350 |
| 149 | 1 | TGATGAAGAGAAACAAGCAA | TGG | 351 |
| 176 | −1 | CATCAAGCTTCCAACAGCTA | AGG | 352 |
| 177 | 1 | ATTTCTTATGCCTTAGCTGT | TGG | 353 |
| 197 | 1 | TGGAAGCTTGATGTATGTTA | TGG | 354 |
| 220 | −1 | CCAACCGCATGAGCAATGTC | TGG | 355 |
| 227 | 1 | TAGACCAGACATTGCTCATG | CGG | 356 |
| 231 | 1 | CCAGACATTGCTCATGCGGT | TGG | 357 |
| 261 | 1 | AGTCATTTTCTCTCTAATCC | AGG | 358 |
| 262 | 1 | GTCATTTTCTCTCTAATCCA | GGG | 359 |
| 268 | −1 | ACATTCCAATGTTCTTTCCC | TGG | 360 |
| 274 | 1 | CTAATCCAGGGAAAGAACAT | TGG | 361 |
| 289 | 1 | AACATTGGAATGTTATTAAA | TGG | 362 |
| 309 | 1 | TGGATTATGAGATATCTCAA | AGG | 363 |
| 336 | 1 | ACTAACTTGAGTTTGAGTTT | TGG | 364 |
| 339 | 1 | AACTTGAGTTTGAGTTTTGG | TGG | 365 |
| 358 | −1 | TCAGTAAAGCCAACTAGTAA | AGG | 366 |
| 360 | 1 | GGTGAGAAACCTTTACTAGT | TGG | 367 |
| 384 | 1 | TTTACTGATGCAGACATGAC | AGG | 368 |
| 422 | 1 | AAAGTTTACTTCAGATTATT | TGG | 369 |
| 435 | 1 | GATTATTTGGTCAAGTTTGC | AGG | 370 |
| 436 | 1 | ATTATTTGGTCAAGTTTGCA | GGG | 371 |
| 437 | 1 | TTATTTGGTCAAGTTTGCAG | GGG | 372 |
| 451 | 1 | TTGCAGGGGAAGCTATTTCA | TGG | 373 |
| 460 | 1 | AAGCTATTTCATGGCAGTCA | AGG | 374 |
| 487 | −1 | TTTACCTCTGTGGTAGAAAG | TGG | 375 |
| 494 | 1 | TGTTCCACTTTCTACCACAG | AGG | 376 |
| 497 | −1 | TGCAATAAACTTTACCTCTG | TGG | 377 |
| 536 | 1 | TGAAGCATGTAAAGAGTTGT | TGG | 378 |
| 537 | 1 | GAAGCATGTAAAGAGTTGTT | GGG | 379 |
| 563 | 1 | AAGAAGTTTCTTGCAGCACT | TGG | 380 |
| 611 | −1 | TGGCAAGATGAATAGCACTT | TGG | 381 |
| 631 | −1 | TGCATGAAAAGTATAATTCT | TGG | 382 |
| 666 | 1 | GATCTAAACATATTGATGTT | AGG | 383 |
| 675 | 1 | ATATTGATGTTAGGTATCAC | TGG | 384 |
| 681 | 1 | ATGTTAGGTATCACTGGATA | CGG | 385 |
| 682 | 1 | TGTTAGGTATCACTGGATAC | GGG | 386 |
| 683 | 1 | GTTAGGTATCACTGGATACG | GGG | 387 |
| 706 | 1 | TGTGTTAGATTCCAAATTGT | TGG | 388 |
| 706 | −1 | TTTCTCAAGTTCCAACAATT | TGG | 389 |
| 737 | 1 | AAAGTTCACACTGATGACAA | TGG | 390 |
| 800 | 1 | GAAACATGTTGTTTGATCGC | CGG | 391 |
| 805 | 1 | ATGTTGTTTGATCGCCGGAA | TGG | 392 |
| 808 | −1 | GGTGGAGGCCATCGCCATTC | CGG | 393 |
| 811 | 1 | TTTGATCGCCGGAATGGCGA | TGG | 394 |
| 823 | −1 | CCCCCTTCTCGACTAGGTGG | AGG | 395 |
| 826 | −1 | TCTCCCCCTTCTCGACTAGG | TGG | 396 |
| 829 | −1 | AAATCTCCCCCTTCTCGACT | AGG | 397 |
| 831 | 1 | TGGCCTCCACCTAGTCGAGA | AGG | 398 |
| 832 | 1 | GGCCTCCACCTAGTCGAGAA | GGG | 399 |
| 833 | 1 | GCCTCCACCTAGTCGAGAAG | GGG | 400 |
| 834 | 1 | CCTCCACCTAGTCGAGAAGG | GGG | 401 |
| 845 | 1 | TCGAGAAGGGGGAGATTTGT | TGG | 402 |
| 846 | 1 | CGAGAAGGGGGAGATTTGTT | GGG | 403 |
| 879 | −1 | ACCCTCAAAATGTTGGGCTT | GGG | 404 |
| 880 | −1 | CACCCTCAAAATGTTGGGCT | TGG | 405 |
| 885 | −1 | AGTGACACCCTCAAAATGTT | GGG | 406 |
| 886 | −1 | AAGTGACACCCTCAAAATGT | TGG | 407 |
| 888 | 1 | GACCCAAGCCCAACATTTTG | AGG | 408 |
| 889 | 1 | ACCCAAGCCCAACATTTTGA | GGG | 409 |
| 928 | −1 | TGACCCCAAAAATCTGATTA | GGG | 410 |
| 929 | −1 | TTGACCCCAAAAATCTGATT | AGG | 411 |
| 934 | 1 | CACAACCCTAATCAGATTTT | TGG | 412 |
| 935 | 1 | ACAACCCTAATCAGATTTTT | GGG | 413 |
| 936 | 1 | CAACCCTAATCAGATTTTTG | GGG | 414 |
| 971 | −1 | TCGCTTCTTTCTCTCTTTGG | TGG | 415 |
| 974 | −1 | TTCTCGCTTCTTTCTCTCTT | TGG | 416 |
| 1028 | 1 | CAAAAGCAGTAAACTTCAAA | TGG | 417 |
| 1053 | 1 | GTTTCTCATTAGTTCACCGT | TGG | 418 |
| 1058 | 1 | TCATTAGTTCACCGTTGGAT | CGG | 419 |
| 1058 | −1 | CAAATTTCAAGCCGATCCAA | CGG | 420 |
| 1105 | 1 | TCTCATCTTGTTCTTCATTC | TGG | 421 |
| 1109 | 1 | ATCTTGTTCTTCATTCTGGA | CGG | 422 |
| 1112 | 1 | TTGTTCTTCATTCTGGACGG | TGG | 423 |
| 1148 | 1 | AAGTCTGCAGTGAGAGAAAT | TGG | 424 |
| 1169 | 1 | GGCTTCGCAAGAGAAAAATT | AGG | 425 |
| 1185 | −1 | ATTGCTACTCTGTGTAAAAA | TGG | 426 |
| 1206 | 1 | ACAGAGTAGCAATTTTTGAA | CGG | 427 |
| 1231 | 1 | GTTTCTCATTTTTTCACCGT | TGG | 428 |
| 1236 | −1 | CAAATTTCACGTCGATCCAA | CGG | 429 |
| 1248 | 1 | CGTTGGATCGACGTGAAATT | TGG | 430 |
| 1298 | −1 | GAAGCACTCACAACACAATG | TGG | 431 |
| 1320 | −1 | TATCCAATACAACAATAGTA | TGG | 432 |
| 1328 | 1 | CTTCCATACTATTGTTGTAT | TGG | 433 |
| 1374 | 1 | AGACTCTTTCAATAATGTCT | AGG | 434 |
| 1443 | 1 | TTAAACGAAAAGATAAATGT | TGG | 435 |
| 1473 | −1 | TTATTCTATTGGGAAAAGAT | TGG | 436 |
| 1483 | −1 | GGGGCATAATTTATTCTATT | GGG | 437 |
| 1484 | −1 | TGGGGCATAATTTATTCTAT | TGG | 438 |
| 1502 | −1 | ACATATTGATAAAATAATTG | GGG | 439 |
| 1503 | −1 | AACATATTGATAAAATAATT | GGG | 440 |
| 1504 | −1 | TAACATATTGATAAAATAAT | TGG | 441 |
| 1521 | 1 | ATTTTATCAATATGTTAATT | AGG | 442 |
| 1535 | 1 | TTAATTAGGAATGAACATTT | TGG | 443 |
| 1589 | 1 | GATTACTTTGTTTCCATAGT | TGG | 444 |
| 1591 | −1 | TACAAAAGATAATCCAACTA | TGG | 445 |
| 1613 | 1 | TTATCTTTTGTAGAATAACG | AGG | 446 |
| 1614 | 1 | TATCTTTTGTAGAATAACGA | GGG | 447 |
| 1640 | 1 | AATCTCTAATATACATTTGT | CGG | 448 |
| 1647 | 1 | AATATACATTTGTCGGCCTT | AGG | 449 |
| 1652 | −1 | AGCATTAATAACACAACCTA | AGG | 450 |
| 1673 | 1 | TGTTATTAATGCTTCTCATA | CGG | 451 |
| 1687 | −1 | AAGCTCTAGCTACAGAGTTG | TGG | 452 |
| 1744 | 1 | GATGATATCAAAAAGAAAGA | AGG | 453 |
| 1775 | 1 | AAACGTATGTGATGTTTAAA | TGG | 454 |
| 1789 | 1 | TTTAAATGGATATGAAATTT | TGG | 455 |
| 1840 | 1 | TGATACGCAGATACGCTAAA | AGG | 456 |
| 1856 | −1 | GCACCCCTACCTTACGGTAT | TGG | 457 |
| 1858 | 1 | AAAGGCAAGCCAATACCGTA | AGG | 458 |
| 1862 | 1 | GCAAGCCAATACCGTAAGGT | AGG | 459 |
| 1862 | −1 | ACATATGCACCCCTACCTTA | CGG | 460 |
| 1863 | 1 | CAAGCCAATACCGTAAGGTA | GGG | 461 |
| 1864 | 1 | AAGCCAATACCGTAAGGTAG | GGG | 462 |
| 1878 | 1 | AGGTAGGGGTGCATATGTGC | CGG | 463 |
| 1879 | 1 | GGTAGGGGTGCATATGTGCC | GGG | 464 |
| 1886 | −1 | ACATCAAACCCGACTTCACC | CGG | 465 |
| 1888 | 1 | GCATATGTGCCGGGTGAAGT | CGG | 466 |
| 1889 | 1 | CATATGTGCCGGGTGAAGTC | GGG | 467 |
| 1911 | −1 | GTATATTTCGGATCGGGTCT | GGG | 468 |
| 1912 | −1 | TGTATATTTCGGATCGGGTC | TGG | 469 |
| 1917 | −1 | CCCGGTGTATATTTCGGATC | GGG | 470 |
| 1918 | −1 | ACCCGGTGTATATTTCGGAT | CGG | 471 |
| 1923 | −1 | AATAAACCCGGTGTATATTT | CGG | 472 |
| 1927 | 1 | ACCCGATCCGAAATATACAC | CGG | 473 |
| 1928 | 1 | CCCGATCCGAAATATACACC | GGG | 474 |
| 1935 | −1 | TCGGGTCTAATAAATAAACC | CGG | 475 |
| 1953 | −1 | TCGGGTCTAGGGTCGGGTTC | GGG | 476 |
| 1954 | −1 | ATCGGGTCTAGGGTCGGGTT | CGG | 477 |
| 1959 | −1 | GTTTCATCGGGTCTAGGGTC | GGG | 478 |
| 1960 | −1 | GGTTTCATCGGGTCTAGGGT | CGG | 479 |
| 1964 | −1 | TATTGGTTTCATCGGGTCTA | GGG | 480 |
| 1965 | −1 | GTATTGGTTTCATCGGGTCT | AGG | 481 |
| 1971 | −1 | GAAAGTGTATTGGTTTCATC | GGG | 482 |
| 1972 | −1 | CGAAAGTGTATTGGTTTCAT | CGG | 483 |
| 1981 | −1 | ATTGTGGCCCGAAAGTGTAT | TGG | 484 |
| 1984 | 1 | CGATGAAACCAATACACTTT | CGG | 485 |
| 1985 | 1 | GATGAAACCAATACACTTTC | GGG | 486 |
| 1997 | −1 | GTTTTTACCCGGTATAATTG | TGG | 487 |
| 2000 | 1 | CTTTCGGGCCACAATTATAC | CGG | 488 |
| 2001 | 1 | TTTCGGGCCACAATTATACC | GGG | 489 |
| 2008 | −1 | GTTTTCACCCGGTTTTTACC | CGG | 490 |
| 2011 | 1 | CAATTATACCGGGTAAAAAC | CGG | 491 |
| 2012 | 1 | AATTATACCGGGTAAAAACC | GGG | 492 |
| 2019 | −1 | TTAATGGCCCGGTTTTCACC | CGG | 493 |
| 2022 | 1 | GGTAAAAACCGGGTGAAAAC | CGG | 494 |
| 2023 | 1 | GTAAAAACCGGGTGAAAACC | GGG | 495 |
| 2030 | −1 | GTAATGTAATGTTAATGGCC | CGG | 496 |
| 2035 | −1 | TCAACGTAATGTAATGTTAA | TGG | 497 |
| 2060 | −1 | TTACATGCTAGCTTACAAGA | AGG | 498 |
| 2089 | −1 | TGGTTGGAGTCTTGAAAATT | TGG | 499 |
| 2105 | −1 | TTACCATGTTAAATAATGGT | TGG | 500 |
| 2109 | −1 | AATTTTACCATGTTAAATAA | TGG | 501 |
| 2113 | 1 | ACTCCAACCATTATTTAACA | TGG | 502 |
| 2155 | −1 | CTTTAATTTTAAAGAAGGGT | TGG | 503 |
| 2159 | −1 | TATGCTTTAATTTTAAAGAA | GGG | 504 |
| 2160 | −1 | TTATGCTTTAATTTTAAAGA | AGG | 505 |
| 2183 | −1 | TTTTGCTTTAGTATGGATTG | TGG | 506 |
| 2190 | −1 | ATTGTGGTTTTGCTTTAGTA | TGG | 507 |
| 2206 | −1 | GACAATATTAGTATTGATTG | TGG | 508 |
| 2237 | −1 | TTTGTATTGATTTAAATATT | TGG | 509 |
| 2374 | −1 | GGATCATGACTATTCTTATG | TGG | 510 |
| 2395 | −1 | ATATTTTGTGTTATTAGTGA | TGG | 511 |
| 2426 | 1 | TATTAATTGTGTATGATGAC | CGG | 512 |
| 2427 | 1 | ATTAATTGTGTATGATGACC | GGG | 513 |
| 2434 | −1 | CCGAAGTCGGCCTGGTGGCC | CGG | 514 |
| 2435 | 1 | TGTATGATGACCGGGCCACC | AGG | 515 |
| 2439 | −1 | GTCACCCGAAGTCGGCCTGG | TGG | 516 |
| 2442 | −1 | CGGGTCACCCGAAGTCGGCC | TGG | 517 |
| 2445 | 1 | CCGGGCCACCAGGCCGACTT | CGG | 518 |
| 2446 | 1 | CGGGCCACCAGGCCGACTTC | GGG | 519 |
| 2447 | −1 | TAGCTCGGGTCACCCGAAGT | CGG | 520 |
| 2461 | 1 | ACTTCGGGTGACCCGAGCTA | TGG | 521 |
| 2461 | −1 | TCGGGTCCGGGCCATAGCTC | GGG | 522 |
| 2462 | −1 | GTCGGGTCCGGGCCATAGCT | CGG | 523 |
| 2466 | 1 | GGGTGACCCGAGCTATGGCC | CGG | 524 |
| 2473 | −1 | CATTATTTCGGGTCGGGTCC | GGG | 525 |
| 2474 | −1 | TCATTATTTCGGGTCGGGTC | CGG | 526 |
| 2479 | −1 | CCCGGTCATTATTTCGGGTC | GGG | 527 |
| 2480 | −1 | ACCCGGTCATTATTTCGGGT | CGG | 528 |
| 2484 | −1 | ATAGACCCGGTCATTATTTC | GGG | 529 |
| 2485 | −1 | AATAGACCCGGTCATTATTT | CGG | 530 |
| 2489 | 1 | ACCCGACCCGAAATAATGAC | CGG | 531 |
| 2490 | 1 | CCCGACCCGAAATAATGACC | GGG | 532 |
| 2497 | −1 | AAGGGTCTCAAAAATAGACC | CGG | 533 |
| 2515 | −1 | TCGGGTTTAGGGTCGGGTAA | GGG | 534 |
| 2516 | −1 | ATCGGGTTTAGGGTCGGGTA | AGG | 535 |
| 2521 | −1 | ATTTCATCGGGTTTAGGGTC | GGG | 536 |
| 2522 | −1 | GATTTCATCGGGTTTAGGGT | CGG | 537 |
| 2526 | −1 | GTATGATTTCATCGGGTTTA | GGG | 538 |
| 2527 | −1 | GGTATGATTTCATCGGGTTT | AGG | 539 |
| 2533 | −1 | TAATTTGGTATGATTTCATC | GGG | 540 |
| 2534 | −1 | CTAATTTGGTATGATTTCAT | CGG | 541 |
| 2548 | −1 | GAACATTTTAGGGGCTAATT | TGG | 542 |
| 2557 | −1 | CCCGGTCCCGAACATTTTAG | GGG | 543 |
| 2558 | −1 | GCCCGGTCCCGAACATTTTA | GGG | 544 |
| 2559 | −1 | GGCCCGGTCCCGAACATTTT | AGG | 545 |
| 2561 | 1 | AAATTAGCCCCTAAAATGTT | CGG | 546 |
| 2562 | 1 | AATTAGCCCCTAAAATGTTC | GGG | 547 |
| 2567 | 1 | GCCCCTAAAATGTTCGGGAC | CGG | 548 |
| 2568 | 1 | CCCCTAAAATGTTCGGGACC | GGG | 549 |
| 2572 | 1 | TAAAATGTTCGGGACCGGGC | CGG | 550 |
| 2573 | 1 | AAAATGTTCGGGACCGGGCC | GGG | 551 |
| 2575 | −1 | CCCGGCCCGAAGGCCCGGCC | CGG | 552 |
| 2580 | 1 | TCGGGACCGGGCCGGGCCTT | CGG | 553 |
| 2580 | −1 | CCCGGCCCGGCCCGAAGGCC | CGG | 554 |
| 2581 | 1 | CGGGACCGGGCCGGGCCTTC | GGG | 555 |
| 2585 | 1 | ACCGGGCCGGGCCTTCGGGC | CGG | 556 |
| 2585 | −1 | ACAGACCCGGCCCGGCCCGA | AGG | 557 |
| 2586 | 1 | CCGGGCCGGGCCTTCGGGCC | GGG | 558 |
| 2590 | 1 | GCCGGGCCTTCGGGCCGGGC | CGG | 559 |
| 2591 | 1 | CCGGGCCTTCGGGCCGGGCC | GGG | 560 |
| 2593 | −1 | AGGGGTGCACAGACCCGGCC | CGG | 561 |
| 2598 | −1 | ACGGTAGGGGTGCACAGACC | CGG | 562 |
| 2611 | −1 | TTCAGCTATCCTTACGGTAG | GGG | 563 |
| 2612 | −1 | ATTCAGCTATCCTTACGGTA | GGG | 564 |
| 2613 | 1 | GTCTGTGCACCCCTACCGTA | AGG | 565 |
| 2613 | −1 | CATTCAGCTATCCTTACGGT | AGG | 566 |
| 2617 | −1 | CACCCATTCAGCTATCCTTA | CGG | 567 |
| 2625 | 1 | CTACCGTAAGGATAGCTGAA | TGG | 568 |
| 2626 | 1 | TACCGTAAGGATAGCTGAAT | GGG | 569 |
| 2654 | 1 | ATATTTTATCAAAAGACAAA | AGG | 570 |
| 2669 | −1 | GTTCCACTGCACATACAGGA | TGG | 571 |
| 2673 | −1 | AAAGGTTCCACTGCACATAC | AGG | 572 |
| 2677 | 1 | AATCCATCCTGTATGTGCAG | TGG | 573 |
| 2691 | −1 | TCACTCCATTGTCTGGGAAA | AGG | 574 |
| 2697 | 1 | TGGAACCTTTTCCCAGACAA | TGG | 575 |
| 2697 | −1 | TGTTCATCACTCCATTGTCT | GGG | 576 |
| 2698 | −1 | ATGTTCATCACTCCATTGTC | TGG | 577 |
| 2732 | −1 | GATATGTATGGTGATGGACG | CGG | 578 |
| 2738 | −1 | GCTTTGGATATGTATGGTGA | TGG | 579 |
| 2744 | −1 | TTTTTTGCTTTGGATATGTA | TGG | 580 |
| 2754 | −1 | ATTTGTTAAATTTTTTGCTT | TGG | 581 |
| 2783 | −1 | GTTAATATGCATGGCAATTG | GGG | 582 |
| 2784 | −1 | TGTTAATATGCATGGCAATT | GGG | 583 |
| 2785 | −1 | GTGTTAATATGCATGGCAAT | TGG | 584 |
| 2792 | −1 | AGTGTACGTGTTAATATGCA | TGG | 585 |
| 2831 | −1 | GGATGGATTGTGGTGCATGG | TGG | 586 |
| 2834 | −1 | GATGGATGGATTGTGGTGCA | TGG | 587 |
| 2841 | −1 | AACAAGAGATGGATGGATTG | TGG | 588 |
| 2848 | −1 | AGAGATAAACAAGAGATGGA | TGG | 589 |
| 2852 | −1 | GCTTAGAGATAAACAAGAGA | TGG | 590 |
| 2874 | −1 | GTGGCAACAACACTAGGTCT | TGG | 591 |
| 2880 | −1 | TGTGAGGTGGCAACAACACT | AGG | 592 |
| 2893 | −1 | TGAAATTGAGAAATGTGAGG | TGG | 593 |
| 2896 | −1 | TGTTGAAATTGAGAAATGTG | AGG | 594 |
| 2927 | −1 | TGGGCATTGGAGTGGCATGC | AGG | 595 |
| 2935 | −1 | TGTACACTTGGGCATTGGAG | TGG | 596 |
| 2940 | −1 | ATGGATGTACACTTGGGCAT | TGG | 597 |
| 2946 | −1 | GATGATATGGATGTACACTT | GGG | 598 |
| 2947 | −1 | GGATGATATGGATGTACACT | TGG | 599 |
| 2959 | −1 | ATAGTTGCGTTAGGATGATA | TGG | 600 |
| 2968 | −1 | GGTATTTATATAGTTGCGTT | AGG | 601 |
| 2989 | −1 | AAATGATGGAGGGAGGTGAG | TGG | 602 |
| 2996 | −1 | GATGGTGAAATGATGGAGGG | AGG | 603 |
| 2999 | −1 | GTGGATGGTGAAATGATGGA | GGG | 604 |
| 3000 | −1 | TGTGGATGGTGAAATGATGG | AGG | 605 |
| 3003 | −1 | GAGTGTGGATGGTGAAATGA | TGG | 606 |
| 3014 | −1 | ATGATTATTATGAGTGTGGA | TGG | 607 |
| 3018 | −1 | ATATATGATTATTATGAGTG | TGG | 608 |
| 3061 | 1 | TCATCTATATAAGTAGTAGC | AGG | 609 |
| 3073 | 1 | GTAGTAGCAGGAGCAATGAG | AGG | 610 |
| 3074 | 1 | TAGTAGCAGGAGCAATGAGA | GGG | 611 |
| 3077 | 1 | TAGCAGGAGCAATGAGAGGG | AGG | 612 |
| 3078 | 1 | AGCAGGAGCAATGAGAGGGA | GGG | 613 |
| 3095 | −1 | ATCCCTAGCAACAGCATCAG | TGG | 614 |
| 3103 | 1 | TCTCCACTGATGCTGTTGCT | AGG | 615 |
| 3104 | 1 | CTCCACTGATGCTGTTGCTA | GGG | 616 |
| 3117 | 1 | GTTGCTAGGGATCCTTGTCC | TGG | 617 |
| 3118 | −1 | CAGAAACTGAAGCCAGGACA | AGG | 618 |
| 3124 | −1 | GCGTTGCAGAAACTGAAGCC | AGG | 619 |
| 3150 | −1 | CTTCTGGTAAGGTGATGACT | TGG | 620 |
| 3161 | −1 | TTCTCTGTTTTCTTCTGGTA | AGG | 621 |
| 3166 | −1 | AGGGGTTCTCTGTTTTCTTC | TGG | 622 |
| 3184 | −1 | GGAGGCACCTCTGGGCGCAG | GGG | 623 |
| 3185 | −1 | TGGAGGCACCTCTGGGCGCA | GGG | 624 |
| 3186 | −1 | CTGGAGGCACCTCTGGGCGC | AGG | 625 |
| 3188 | 1 | CAGAGAACCCCTGCGCCCAG | AGG | 626 |
| 3192 | −1 | ACAACTCTGGAGGCACCTCT | GGG | 627 |
| 3193 | −1 | GACAACTCTGGAGGCACCTC | TGG | 628 |
| 3202 | −1 | GTTCCTGTTGACAACTCTGG | AGG | 629 |
| 3205 | −1 | CCGGTTCCTGTTGACAACTC | TGG | 630 |
| 3210 | 1 | GTGCCTCCAGAGTTGTCAAC | AGG | 631 |
| 3216 | 1 | CCAGAGTIGTCAACAGGAAC | CGG | 632 |
| 3224 | −1 | GCCTTTTGCTTCAAGTCATC | CGG | 633 |
| 3234 | 1 | ACCGGATGACTTGAAGCAAA | AGG | 634 |
| 3264 | −1 | ACGAGGATCATACTCGAGCT | TGG | 635 |
| 3281 | −1 | CGAGGATCATAGACACAACG | AGG | 636 |
| 3295 | 1 | CGTTGTGTCTATGATCCTCG | AGG | 637 |
| 3299 | −1 | GTGGTGCCAGTGTGTCCTCG | AGG | 638 |
| 3304 | 1 | TATGATCCTCGAGGACACAC | TGG | 639 |
| 3318 | − 1 | TGGAGGGGAACGTTGGTTGG | TGG | 640 |
| 3321 | −1 | CCCTGGAGGGGAACGTTGGT | TGG | 641 |
| 3325 | −1 | GCTCCCCTGGAGGGGAACGT | TGG | 642 |
| 3331 | 1 | ACCAACCAACGTTCCCCTCC | AGG | 643 |
| 3332 | 1 | CCAACCAACGTTCCCCTCCA | GGG | 644 |
| 3333 | 1 | CAACCAACGTTCCCCTCCAG | GGG | 645 |
| 3333 | −1 | ACGTGTCCGCTCCCCTGGAG | GGG | 646 |
| 3334 | −1 | CACGTGTCCGCTCCCCTGGA | GGG | 647 |
| 3335 | −1 | CCACGTGTCCGCTCCCCTGG | AGG | 648 |
| 3338 | 1 | AACGTTCCCCTCCAGGGGAG | CGG | 649 |
| 3338 | 1 | CGGCCACGTGTCCGCTCCCC | TGG | 650 |
| 3346 | 1 | CCTCCAGGGGAGCGGACACG | TGG | 651 |
| 3358 | 1 | CGGACACGTGGCCGCCAACC | CGG | 652 |
| 3358 | −1 | CATCGTAGTCTCCGGGTTGG | CGG | 653 |
| 3361 | −1 | CATCATCGTAGTCTCCGGGT | TGG | 654 |
| 3365 | −1 | CGGTCATCATCGTAGTCTCC | GGG | 655 |
| 3366 | −1 | GCGGTCATCATCGTAGTCTC | CGG | 656 |
| 3385 | −1 | CCTCTCTTCGGGGTTGACGG | CGG | 657 |
| 3388 | −1 | CTTCCTCTCTTCGGGGTTGA | CGG | 658 |
| 3395 | −1 | CGGCCTCCTTCCTCTCTTCG | GGG | 659 |
| 3396 | 1 | CCGCCGTCAACCCCGAAGAG | AGG | 660 |
| 3396 | −1 | TCGGCCTCCTTCCTCTCTTC | GGG | 661 |
| 3397 | −1 | ATCGGCCTCCTTCCTCTCTT | CGG | 662 |
| 3400 | 1 | CGTCAACCCCGAAGAGAGGA | AGG | 663 |
| 3403 | 1 | CAACCCCGAAGAGAGGAAGG | AGG | 664 |
| 3410 | 1 | GAAGAGAGGAAGGAGGCCGA | TGG | 665 |
| 3411 | 1 | AAGAGAGGAAGGAGGCCGAT | GGG | 666 |
| 3412 | 1 | AGAGAGGAAGGAGGCCGATG | GGG | 667 |
| 3415 | −1 | TCGGTCCAGCTGGTCCCCAT | CGG | 668 |
| 3421 | 1 | GGAGGCCGATGGGGACCAGC | TGG | 669 |
| 3425 | −1 | TCACGCTCCCTCGGTCCAGC | TGG | 670 |
| 3428 | 1 | GATGGGGACCAGCTGGACCG | AGG | 671 |
| 3429 | 1 | ATGGGGACCAGCTGGACCGA | GGG | 672 |
| 3434 | −1 | TCTTCTCTTTCACGCTCCCT | CGG | 673 |
| 3452 | 1 | AGCGTGAAAGAGAAGAAGAC | TGG | 674 |
| 3470 | −1 | GGTCGCCTCCAATCTTCTCT | TGG | 675 |
| 3473 | 1 | GGAGACAACCAAGAGAAGAT | TGG | 676 |
| 3476 | 1 | GACAACCAAGAGAAGATTGG | AGG | 677 |
| 3491 | −1 | TTCCGTGGCTGCTGATGACT | TGG | 678 |
| 3500 | 1 | GACCAAGTCATCAGCAGCCA | CGG | 679 |
| 3506 | −1 | CCTTCGGGCCTTATTTTCCG | TGG | 680 |
| 3509 | 1 | ATCAGCAGCCACGGAAAATA | AGG | 681 |
| 3517 | 1 | CCACGGAAAATAAGGCCCGA | AGG | 682 |
| 3521 | −1 | TGTTCTCCTTCTCTTCCTTC | GGG | 683 |
| 3522 | −1 | TTGTTCTCCTTCTCTTCCTT | CGG | 684 |
| 3526 | 1 | ATAAGGCCCGAAGGAAGAGA | AGG | 685 |

TABLE 4
gRNA's targeting sequences of Ara h2.

| Position | | | | |
|---|---|---|---|---|
| on SEQ ID | | | | SEQ |
| NO: 686 | Strand | Sequence | PAM | ID NO: |
| 20 | 1 | TTTTGTTTTAATTTATATAT | TGG | 688 |
| 166 | −1 | CTATCCTTTTGTGAGACAGA | TGG | 689 |
| 173 | 1 | ATATCCATCTGTCTCACAAA | AGG | 690 |
| 180 | 1 | TCTGTCTCACAAAAGGATAG | AGG | 691 |
| 181 | 1 | CTGTCTCACAAAAGGATAGA | GGG | 692 |
| 192 | 1 | AAGGATAGAGGGAAAACGTA | AGG | 693 |
| 220 | 1 | TTCTAAATAAAAACAGAGAA | CGG | 694 |
| 227 | 1 | TAAAAACAGAGAACGGAAAA | AGG | 695 |
| 243 | −1 | TGGCAATGATATTCATATGG | TGG | 696 |
| 246 | −1 | AAATGGCAATGATATTCATA | TGG | 697 |
| 263 | −1 | TCCTCTATTGTCATTAGAAA | TGG | 698 |
| 273 | 1 | GCCATTTCTAATGACAATAG | AGG | 699 |
| 284 | 1 | TGACAATAGAGGAGTTTGAT | TGG | 700 |
| 285 | 1 | GACAATAGAGGAGTTTGATT | GGG | 701 |
| 305 | 1 | GGGTAATTGATGTTGAAAAA | TGG | 702 |
| 328 | −1 | GTCAATTTTTTTTAAATTTA | CGG | 703 |
| 352 | 1 | AAAAATTGACTAATTTTCTA | CGG | 704 |
| 434 | 1 | CATACTTTATGATTTTTATT | AGG | 705 |
| 446 | 1 | TTTTTATTAGGAAAAGTATA | AGG | 706 |
| 536 | −1 | ATCTTTATAAATATATTTTC | TGG | 707 |
| 576 | −1 | TAATAAAAATGTCTTTTTTA | TGG | 708 |
| 638 | 1 | ACACAATTATTAATAAGAGT | TGG | 709 |
| 674 | 1 | AAATACTGTTGATAATGTAG | CGG | 710 |
| 697 | 1 | AATTGTTTTTATTATTATTT | AGG | 711 |
| 713 | −1 | TTTCTTGGTCTTAAGCGTTA | AGG | 712 |
| 728 | −1 | AAGGTTTTTTTTTTTTTTCT | TGG | 713 |
| 742 | 1 | AGAAAAAAAAAAAAAACCTT | AGG | 714 |
| 747 | −1 | TTGAGAGATTTGTGCACCTA | AGG | 715 |
| 771 | 1 | AATCTCTCAAATAAGAATTT | CGG | 716 |
| 772 | 1 | ATCTCTCAAATAAGAATTTC | GGG | 717 |
| 775 | 1 | TCTCAAATAAGAATTTCGGG | TGG | 718 |
| 783 | 1 | AAGAATTTCGGGTGGCAAGA | TGG | 719 |
| 802 | 1 | ATGGAAAAGCACATCGATGA | AGG | 720 |
| 808 | 1 | AAGCACATCGATGAAGGCGT | TGG | 721 |
| 830 | 1 | GAAGACTCATCGTTTTCTCG | TGG | 722 |
| 861 | 1 | GCGAAGTCTTCATTCTTGTA | TGG | 723 |
| 867 | 1 | TCTTCATTCTTGTATGGTTG | CGG | 724 |
| 870 | 1 | TCATTCTTGTATGGTTGCGG | AGG | 725 |
| 890 | 1 | AGGCGCAAGAAGCTAAGATT | TGG | 726 |
| 901 | 1 | GCTAAGATTTGGAAGAATAG | TGG | 727 |
| 904 | 1 | AAGATTTGGAAGAATAGTGG | CGG | 728 |
| 953 | −1 | TTTTGTCTTCATACAACATG | TGG | 729 |
| 980 | −1 | CTGGATAACCTGCTTTTGTG | GGG | 730 |
| 981 | −1 | GCTGGATAACCTGCTTTTGT | GGG | 731 |
| 982 | −1 | AGCTGGATAACCTGCTTTTG | TGG | 732 |
| 983 | 1 | AAAATCAACCCCACAAAAGC | AGG | 733 |
| 999 | −1 | TATAGATAGACAAAGAGAGC | TGG | 734 |
| 1051 | −1 | AAAAACAAACTAAACTGTGC | TGG | 735 |
| 1078 | −1 | AGGAAGATAGAGTAAAATAA | AGG | 736 |
| 1098 | −1 | CACTAAGAGCCTTGGAAATG | AGG | 737 |
| 1100 | 1 | CTCTATCTTCCTCATTTCCA | AGG | 738 |
| 1106 | −1 | GAATGACACACTAAGAGCCT | TGG | 739 |
| 1159 | −1 | AATAGGACAATAGTGTGACG | AGG | 740 |
| 1176 | −1 | TAGTGCTTCAATAATTAAAT | AGG | 741 |
| 1202 | −1 | TATATATGTTTTTGGTAATG | AGG | 742 |
| 1210 | −1 | CCTAATCCTATATATGTTTT | TGG | 743 |
| 1215 | 1 | TCATTACCAAAAACATATAT | AGG | 744 |
| 1221 | 1 | CCAAAAACATATATAGGATT | AGG | 745 |
| 1281 | 1 | TTTACATTATGTATTTAATA | TGG | 746 |
| 1331 | −1 | AAACGAGTTTAGCTAGCTAA | AGG | 747 |
| 1362 | −1 | TGATTTGTCAAATTATCGAT | GGG | 748 |
| 1363 | −1 | TTGATTTGTCAAATTATCGA | TGG | 749 |
| 1507 | −1 | TATTTATGTTAAACTTGTTT | TGG | 750 |
| 1531 | 1 | AACATAAATATTACAACCCA | AGG | 751 |
| 1536 | −1 | TTCTTTTAGTTTGTTTCCTT | GGG | 752 |
| 1537 | −1 | TTTCTTTTAGTTTGTTTCCT | TGG | 753 |
| 1643 | 1 | CACTCATATGTATACGATAT | AGG | 754 |
| 1680 | −1 | TTTTTTAAGTATATTTAAAT | GGG | 755 |
| 1681 | −1 | ATTTTTTAAGTATATTTAAA | TGG | 756 |
| 1708 | −1 | AAGATGTTTTTTTTTATTTT | TGG | 757 |
| 1870 | 1 | TATGTTTAATAGCTATATGT | TGG | 758 |
| 1949 | −1 | TAAAATTAAAGAAAGAAATT | TGG | 759 |
| 1975 | −1 | TACCTCTTCTTTCTTACATT | TGG | 760 |
| 1984 | 1 | TTCCAAATGTAAGAAAGAAG | AGG | 761 |
| 2036 | 1 | TATATAATTTATAATATCAC | TGG | 762 |
| 2052 | −1 | ATAACATTTTTTTTATTTTG | TGG | 763 |
| 2120 | 1 | TGAAATGCAGAAATAGAAAG | TGG | 764 |
| 2121 | 1 | GAAATGCAGAAATAGAAAGT | GGG | 765 |
| 2168 | −1 | TTAGTCATGTGAATATTTGC | AGG | 766 |
| 2265 | 1 | ACACACAATATTTTCTATGA | AGG | 767 |
| 2266 | 1 | CACACAATATTTTCTATGAA | GGG | 768 |
| 2311 | 1 | AAATAAAATAAATGCAGAGA | AGG | 769 |
| 2323 | 1 | TGCAGAGAAGGCAGAATAAG | AGG | 770 |
| 2333 | 1 | GCAGAATAAGAGGACAAATA | TGG | 771 |
| 2382 | 1 | AAGTAAAATCTCAACACGAG | AGG | 772 |
| 2388 | 1 | AATCTCAACACGAGAGGAGA | AGG | 773 |
| 2389 | 1 | ATCTCAACACGAGAGGAGAA | GGG | 774 |
| 2398 | 1 | CGAGAGGAGAAGGGATGATT | TGG | 775 |
| 2409 | 1 | GGGATGATTTGGTTTCTCTC | CGG | 776 |
| 2417 | −1 | AATAATAAAGCTGGACATAC | CGG | 777 |
| 2426 | −1 | AATTACCCTAATAATAAAGC | TGG | 778 |
| 2431 | 1 | GTATGTCCAGCTTTATTATT | AGG | 779 |
| 2432 | 1 | TATGTCCAGCTTTATTATTA | GGG | 780 |
| 2507 | 1 | TATCTAAATTATTAGATACT | AGG | 781 |
| 2538 | 1 | ACTTTTTATTTAAGAGAAGA | TGG | 782 |
| 2539 | 1 | CTTTTTATTTAAGAGAAGAT | GGG | 783 |
| 2544 | 1 | TATTTAAGAGAAGATGGGAG | TGG | 784 |
| 2562 | −1 | CGAAAATATGTATAAATTTA | AGG | 785 |
| 2574 | 1 | CTTAAATTTATACATATTTT | CGG | 786 |
| 2652 | 1 | TATTTTTTCTTATTCTGAAA | TGG | 787 |
| 2663 | 1 | ATTCTGAAATGGAAAATGCT | AGG | 788 |
| 2706 | 1 | TTAGTCTTGATTCAAAACTA | AGG | 789 |
| 2722 | 1 | ACTAAGGAAAAAAACATTTA | TGG | 790 |
| 2774 | 1 | AGAGTTAAACATCAAACAGA | AGG | 791 |
| 2818 | −1 | AAGCATGCCATGTTGTTATG | TGG | 792 |
| 2822 | 1 | GATTATACCACATAACAACA | TGG | 793 |
| 2832 | 1 | CATAACAACATGGCATGCTT | AGG | 794 |
| 2846 | −1 | GTCCAAGATGCCTTTTTGTT | TGG | 795 |
| 2847 | 1 | TGCTTAGGTACCAAACAAAA | AGG | 796 |
| 2855 | 1 | TACCAAACAAAAAGGCATCT | TGG | 797 |
| 2883 | 1 | ATTTGCATGAAAATGTAACG | TGG | 798 |
| 2927 | −1 | AAGGGGGAAGGAGGGTGGGG | TGG | 799 |
| 2930 | −1 | GGAAAGGGGGAAGGAGGGTG | GGG | 800 |
| 2931 | −1 | GGGAAAGGGGGAAGGAGGGT | GGG | 801 |
| 2932 | −1 | AGGGAAAGGGGGAAGGAGGG | TGG | 802 |
| 2935 | −1 | AAAAGGGAAAGGGGGAAGGA | GGG | 803 |
| 2936 | −1 | GAAAAGGGAAAGGGGGAAGG | AGG | 804 |
| 2939 | −1 | GTGGAAAAGGGAAAGGGGGA | AGG | 805 |
| 2943 | −1 | GTACGTGGAAAAGGGAAAGG | GGG | 806 |
| 2944 | −1 | GGTACGTGGAAAAGGGAAAG | GGG | 807 |
| 2945 | −1 | AGGTACGTGGAAAAGGGAAA | GGG | 808 |
| 2946 | −1 | GAGGTACGTGGAAAAGGGAA | AGG | 809 |
| 2951 | −1 | CATGTGAGGTACGTGGAAAA | GGG | 810 |
| 2952 | −1 | GCATGTGAGGTACGTGGAAA | AGG | 811 |
| 2958 | −1 | GATTTTGCATGTGAGGTACG | TGG | 812 |
| 2965 | −1 | AAGGAGGGATTTTGCATGTG | AGG | 813 |
| 2980 | −1 | GGTATTTATAGGTGAAAGGA | GGG | 814 |
| 2981 | −1 | TGGTATTTATAGGTGAAAGG | AGG | 815 |
| 2984 | −1 | TGATGGTATTTATAGGTGAA | AGG | 816 |
| 2991 | −1 | ATGGTGGTGATGGTATTTAT | AGG | 817 |
| 3001 | −1 | TTGAGTGTGGATGGTGGTGA | TGG | 818 |
| 3007 | −1 | TGTTTTTTGAGTGTGGATGG | TGG | 819 |
| 3010 | −1 | TGGTGTTTTTTGAGTGTGGA | TGG | 820 |
| 3014 | −1 | TGTGTGGTGTTTTTTGAGTG | TGG | 821 |
| 3030 | −1 | AATGTGTATTGAAGAGTGTG | TGG | 822 |
| 3053 | −1 | CATTGTTGTTGTGGTGGTTA | TGG | 823 |
| 3059 | −1 | CTTGGCCATTGTTGTTGTGG | TGG | 824 |
| 3062 | −1 | GAGCTTGGCCATTGTTGTTG | TGG | 825 |
| 3065 | 1 | CATAACCACCACAACAACAA | TGG | 826 |
| 3077 | −1 | GGCTACTAGTATGGTGAGCT | TGG | 827 |
| 3086 | −1 | AAGGGCGAGGGCTACTAGTA | TGG | 828 |
| 3098 | −1 | AGCGAGGAGGAAAAGGGCGA | GGG | 829 |
| 3099 | −1 | CAGCGAGGAGGAAAAGGGCG | AGG | 830 |
| 3104 | −1 | GTGGGCAGCGAGGAGGAAAA | GGG | 831 |
| 3105 | −1 | CGTGGGCAGCGAGGAGGAAA | AGG | 832 |
| 3111 | −1 | CAGATGCGTGGGCAGCGAGG | AGG | 833 |
| 3114 | −1 | TCGCAGATGCGTGGGCAGCG | AGG | 834 |
| 3122 | −1 | CTGCTGCCTCGCAGATGCGT | GGG | 835 |
| 3123 | −1 | ACTGCTGCCTCGCAGATGCG | TGG | 836 |
| 3127 | 1 | TCGCTGCCCACGCATCTGCG | AGG | 837 |
| 3136 | 1 | ACGCATCTGCGAGGCAGCAG | TGG | 838 |
| 3137 | 1 | CGCATCTGCGAGGCAGCAGT | GGG | 839 |
| 3147 | 1 | AGGCAGCAGTGGGAACTACA | AGG | 840 |
| 3171 | 1 | TCGCCCTCTCGAGCTGGCTC | TGG | 841 |
| 3177 | −1 | TAAGGTTCGCCCTCTCGAGC | TGG | 842 |
| 3178 | 1 | GATGCCAGAGCCAGCTCGAG | AGG | 843 |
| 3179 | 1 | ATGCCAGAGCCAGCTCGAGA | GGG | 844 |
| 3190 | 1 | AGCTCGAGAGGGCGAACCTT | AGG | 845 |
| 3195 | −1 | GATGTTGCTCGCAGGGCCTA | AGG | 846 |
| 3202 | −1 | TGCATGAGATGTTGCTCGCA | GGG | 847 |
| 3203 | −1 | CTGCATGAGATGTTGCTCGC | AGG | 848 |
| 3231 | −1 | CATATGAATCCTCGTCACGT | TGG | 849 |
| 3233 | 1 | GCAGAAAATCCAACGTGACG | AGG | 850 |
| 3243 | 1 | CAACGTGACGAGGATTCATA | TGG | 851 |
| 3247 | 1 | GTGACGAGGATTCATATGGA | CGG | 852 |
| 3248 | 1 | TGACGAGGATTCATATGGAC | GGG | 853 |
| 3261 | −1 | GATCCTGACTAGGGCTGTAC | GGG | 854 |
| 3262 | −1 | GGATCCTGACTAGGGCTGTA | CGG | 855 |
| 3269 | 1 | GGACCCGTACAGCCCTAGTC | AGG | 856 |
| 3270 | −1 | GGCTGTACGGATCCTGACTA | GGG | 857 |
| 3271 | −1 | GGGCTGTACGGATCCTGACT | AGG | 858 |
| 3283 | −1 | GGGTCCTGACTAGGGCTGTA | CGG | 859 |
| 3290 | 1 | GGATCCGTACAGCCCTAGTC | AGG | 860 |
| 3291 | −1 | GTCTGTCCGGGTCCTGACTA | GGG | 861 |
| 3292 | −1 | CGTCTGTCCGGGTCCTGACT | AGG | 862 |
| 3296 | 1 | GTACAGCCCTAGTCAGGACC | CGG | 863 |
| 3303 | −1 | TGTACGGATCACGTCTGTCC | GGG | 864 |
| 3304 | −1 | CTGTACGGATCACGTCTGTC | CGG | 865 |
| 3319 | −1 | TCATATGGACTAGGGCTGTA | CGG | 866 |
| 3327 | −1 | CTCTCCGATCATATGGACTA | GGG | 867 |
| 3328 | −1 | CCTCTCCGATCATATGGACT | AGG | 868 |
| 3334 | 1 | ACAGCCCTAGTCCATATGAT | CGG | 869 |
| 3334 | −1 | CCAGCGCCTCTCCGATCATA | TGG | 870 |
| 3339 | 1 | CCTAGTCCATATGATCGGAG | AGG | 871 |
| 3345 | 1 | CCATATGATCGGAGAGGCGC | TGG | 872 |
| 3359 | −1 | ACACCTCTCTTGGTGCTGAG | AGG | 873 |
| 3367 | 1 | GATCCTCTCAGCACCAAGAG | AGG | 874 |
| 3369 | −1 | GCTCATTGCAACACCTCTCT | TGG | 875 |
| 3406 | 1 | ACGAGTTTGAGAACAACCAA | AGG | 876 |
| 3411 | −1 | ATGCCTCGCACATGCACCTT | TGG | 877 |
| 3419 | 1 | CAACCAAAGGTGCATGTGCG | AGG | 878 |
| 3437 | 1 | CGAGGCATTGCAACAGATAA | TGG | 879 |
| 3453 | −1 | TCCCCTGCAACCTATCGCTC | TGG | 880 |
| 3454 | 1 | TAATGGAGAACCAGAGCGAT | AGG | 881 |
| 3461 | 1 | GAACCAGAGCGATAGGTTGC | AGG | 882 |
| 3462 | 1 | AACCAGAGCGATAGGTTGCA | GGG | 883 |
| 3463 | 1 | ACCAGAGCGATAGGTTGCAG | GGG | 884 |
| 3466 | 1 | AGAGCGATAGGTTGCAGGGG | AGG | 885 |
| 3473 | 1 | TAGGTTGCAGGGGAGGCAAC | AGG | 886 |
| 3490 | 1 | AACAGGAGCAACAGTTCAAG | AGG | 887 |
| 3491 | 1 | ACAGGAGCAACAGTTCAAGA | GGG | 888 |
| 3499 | 1 | AACAGTTCAAGAGGGAGCTC | AGG | 889 |
| 3517 | −1 | GCCCTGAGGCCGCACTGTTG | AGG | 890 |
| 3519 | 1 | AGGAACTTGCCTCAACAGTG | CGG | 891 |
| 3526 | 1 | TGCCTCAACAGTGCGGCCTC | AGG | 892 |
| 3527 | 1 | GCCTCAACAGTGCGGCCTCA | GGG | 893 |
| 3531 | −1 | CGCAACGCTGTGGTGCCCTG | AGG | 894 |
| 3541 | −1 | ACTTCCAAGTCGCAACGCTG | TGG | 895 |
| 3548 | 1 | GGCACCACAGCGTTGCGACT | TGG | 896 |
| 3561 | 1 | TGCGACTTGGAAGTCGAAAG | TGG | 897 |
| 3564 | 1 | GACTTGGAAGTCGAAAGTGG | CGG | 898 |
| 3595 | −1 | TTCTTTTCTTTTTTTGAGAT | AGG | 899 |
| 3646 | 1 | TATATATAAGCTATTATCTA | TGG | 900 |
| 3661 | 1 | ATCTATGGTAATGTTAGTTT | TGG | 901 |
| 3711 | 1 | TGTGTTGATCGTGTTAACTA | AGG | 902 |
| 3721 | 1 | GTGTTAACTAAGGCAAGCTT | AGG | 903 |
| 3744 | −1 | ACGCCATAAAAGCACTCTAA | AGG | 904 |
| 3752 | 1 | GCACCTTTAGAGTGCTTTTA | TGG | 905 |
| 3797 | −1 | CTTTTTATATTATTTCAAGA | TGG | 906 |
| 3825 | 1 | AAAAAGATCATGTTTTGTTT | TGG | 907 |

TABLE 5
gRNA's targeting sequences of Ara h3.

| Position | | | | |
|---|---|---|---|---|
| on SEQ ID | | | | SEQ |
| NO: 908 | Strand | Sequence | PAM | ID NO: |
| 40 | −1 | CTGATTTTAGTGGTTGATTT | TGG | 910 |
| 50 | −1 | ATACTCGTGACTGATTTTAG | TGG | 911 |
| 131 | −1 | AATTAACCCAAAATCAAATT | AGG | 912 |
| 135 | 1 | TGTTGAGCCTAATTTGATTT | TGG | 913 |
| 136 | 1 | GTTGAGCCTAATTTGATTTT | GGG | 914 |
| 165 | 1 | TATGATGACAAACATTCAAT | TGG | 915 |
| 166 | 1 | ATGATGACAAACATTCAATT | GGG | 916 |
| 185 | −1 | AACACACATTAAATAATATT | GGG | 917 |
| 186 | −1 | AAACACACATTAAATAATAT | TGG | 918 |
| 205 | 1 | ATTTAATGTGTGTTTTATTG | TGG | 919 |
| 209 | 1 | AATGTGTGTTTTATTGTGGC | AGG | 920 |
| 221 | −1 | TTGGCTGTGAGAGAATTCAT | GGG | 921 |
| 222 | −1 | TTTGGCTGTGAGAGAATTCA | TGG | 922 |
| 240 | −1 | AATGAACATGTAGCATTGTT | TGG | 923 |
| 292 | −1 | GTGAAACAAGAGTGAGGTAT | TGG | 924 |
| 298 | −1 | TGAACGGTGAAACAAGAGTG | AGG | 925 |
| 314 | −1 | AGCCTTTCATTCTTGCTGAA | CGG | 926 |
| 323 | 1 | CACCGTTCAGCAAGAATGAA | AGG | 927 |
| 335 | 1 | AGAATGAAAGGCTATACGTT | TGG | 928 |
| 377 | 1 | ATCTATATCATGAGCAAAAT | AGG | 929 |
| 385 | 1 | CATGAGCAAAATAGGACAAC | TGG | 930 |
| 395 | 1 | ATAGGACAACTGGAAAACAA | AGG | 931 |
| 419 | 1 | AGCAACAACACAACAAGAAC | AGG | 932 |
| 424 | 1 | CAACACAACAAGAACAGGTG | TGG | 933 |
| 444 | 1 | TGGCTCTGTAAAAGCAAGAA | AGG | 934 |
| 452 | 1 | TAAAAGCAAGAAAGGACACT | AGG | 935 |
| 468 | −1 | AGCTGATGTCCTTGGATTAG | TGG | 936 |
| 470 | 1 | CTAGGATAACCACTAATCCA | AGG | 937 |
| 476 | −1 | TATTGCTCAGCTGATGTCCT | TGG | 938 |
| 492 | 1 | GACATCAGCTGAGCAATACT | AGG | 939 |
| 553 | 1 | GAGCAGCAGCAGCAAGAAAA | TGG | 940 |
| 583 | 1 | TGCAAAAGAAGAGCATGCCA | AGG | 941 |
| 589 | −1 | CTTTGCTTGACCTTCTTCCT | TGG | 942 |
| 590 | 1 | GAAGAGCATGCCAAGGAAGA | AGG | 943 |
| 601 | 1 | CAAGGAAGAAGGTCAAGCAA | AGG | 944 |
| 611 | 1 | GGTCAAGCAAAGGACAGCAG | AGG | 945 |
| 636 | −1 | TCCACTACCTCTGCTGCTTG | AGG | 946 |
| 640 | 1 | TCGAGCTCCTCAAGCAGCAG | AGG | 947 |
| 646 | 1 | TCCTCAAGCAGCAGAGGTAG | TGG | 948 |
| 653 | 1 | GCAGCAGAGGTAGTGGAGCA | TGG | 949 |
| 662 | 1 | GTAGTGGAGCATGGTGAAAA | AGG | 950 |
| 681 | −1 | CTCGTTGGAGCTGTCATAGC | TGG | 951 |
| 696 | −1 | TCCTTGTACCAGCTACTCGT | TGG | 952 |
| 699 | 1 | TGACAGCTCCAACGAGTAGC | TGG | 953 |
| 706 | 1 | TCCAACGAGTAGCTGGTACA | AGG | 954 |
| 718 | 1 | CTGGTACAAGGAAATGAAAG | AGG | 955 |
| 741 | −1 | CTTCTTATATATCTTCAACT | TGG | 956 |
| 771 | −1 | CTTTTTTGTTTTTGAAATGG | TGG | 957 |
| 774 | −1 | TCTCTTTTTTGTTTTTGAAA | TGG | 958 |
| 819 | 1 | TTCCAGCTCTGTGTTAGAGA | TGG | 959 |
| 828 | 1 | CACCATCTCTAACACAGAGC | TGG | 960 |
| 874 | 1 | TAATTCTGATTTTTATGTTA | TGG | 961 |
| 907 | 1 | TTGTTTTCTGTTTTGAGAAA | AGG | 962 |
| 920 | 1 | TGAGAAAAGGCAATTATGCG | AGG | 963 |
| 943 | 1 | TTCAAAAGTCAAGAGAGAAA | AGG | 964 |
| 974 | −1 | CAAAATACATCAGAAATTAG | TGG | 965 |
| 986 | 1 | CACTAATTTCTGATGTATTT | TGG | 966 |
| 987 | 1 | ACTAATTTCTGATGTATTTT | GGG | 967 |
| 1000 | 1 | GTATTTTGGGTTTATGAATT | AGG | 968 |
| 1020 | −1 | GTGCTAACCCAACTTGGTAA | GGG | 969 |
| 1021 | −1 | AGTGCTAACCCAACTTGGTA | AGG | 970 |
| 1023 | 1 | ATTAATTTCCCTTACCAAGT | TGG | 971 |
| 1024 | 1 | TTAATTTCCCTTACCAAGTT | GGG | 972 |
| 1026 | −1 | TCCCAAGTGCTAACCCAACT | TGG | 973 |
| 1035 | 1 | TACCAAGTTGGGTTAGCACT | TGG | 974 |
| 1036 | 1 | ACCAAGTTGGGTTAGCACTT | GGG | 975 |
| 1050 | 1 | GCACTTGGGAAAATTGAATC | TGG | 976 |
| 1068 | −1 | GTGCTGACCCAACTAGTAGT | GGG | 977 |
| 1069 | −1 | AGTGCTGACCCAACTAGTAG | TGG | 978 |
| 1071 | 1 | GGTTGATTCCCACTACTAGT | TGG | 979 |
| 1072 | 1 | GTTGATTCCCACTACTAGTT | GGG | 980 |
| 1084 | 1 | TACTAGTTGGGTCAGCACTA | TGG | 981 |
| 1085 | 1 | ACTAGTTGGGTCAGCACTAT | GGG | 982 |
| 1100 | 1 | ACTATGGGAAGCTAAACTTT | TGG | 983 |
| 1115 | 1 | ACTTTTGGTGATGAAAGCTT | AGG | 984 |
| 1116 | 1 | CTTTTGGTGATGAAAGCTTA | GGG | 985 |
| 1117 | 1 | TTTTGGTGATGAAAGCTTAG | GGG | 986 |
| 1118 | 1 | TTTGGTGATGAAAGCTTAGG | GGG | 987 |
| 1138 | 1 | GGGTAGATACTAGTTGAATT | AGG | 988 |
| 1158 | 1 | AGGAAAAAATTCAATACTAT | TGG | 989 |
| 1202 | −1 | GTCTCCACCACAAACTATGG | TGG | 990 |
| 1205 | −1 | CCAGTCTCCACCACAAACTA | TGG | 991 |
| 1206 | 1 | GAAAATTCCACCATAGTTTG | TGG | 992 |
| 1209 | 1 | AATTCCACCATAGTTTGTGG | TGG | 993 |
| 1216 | 1 | CCATAGTTTGTGGTGGAGAC | TGG | 994 |
| 1223 | 1 | TTGTGGTGGAGACTGGATGT | AGG | 995 |
| 1231 | 1 | GAGACTGGATGTAGGATTCA | TGG | 996 |
| 1253 | 1 | GACCAGCATGTATCTTGGTT | CGG | 997 |
| 1258 | −1 | ATGAAGACCAGCATGTATCT | TGG | 998 |
| 1262 | 1 | ATCCGAACCAAGATACATGC | TGG | 999 |
| 1289 | −1 | ACGCAGAACATAAAAGAGCA | GGG | 1000 |
| 1290 | −1 | TACGCAGAACATAAAAGAGC | AGG | 1001 |
| 1334 | −1 | ATCTGTTGCTTTCAAGTTGC | AGG | 1002 |
| 1386 | −1 | AGAAGGGGGTTGAATCAAGT | TGG | 1003 |
| 1400 | −1 | TCTAGTTGAACTTGAGAAGG | GGG | 1004 |
| 1401 | −1 | TTCTAGTTGAACTTGAGAAG | GGG | 1005 |
| 1402 | −1 | GTTCTAGTTGAACTTGAGAA | GGG | 1006 |
| 1403 | −1 | GGTTCTAGTTGAACTTGAGA | AGG | 1007 |
| 1424 | −1 | GTATTTATGTATAAATATGA | TGG | 1008 |
| 1443 | 1 | TTTATACATAAATACATTAG | TGG | 1009 |
| 1585 | 1 | TATATTGATATAAGATACTT | AGG | 1010 |
| 1586 | 1 | ATATTGATATAAGATACTTA | GGG | 1011 |
| 1590 | 1 | TGATATAAGATACTTAGGGT | CGG | 1012 |
| 1641 | −1 | TAAAAAAAATCGCTAGTTAC | GGG | 1013 |
| 1642 | −1 | CTAAAAAAAATCGCTAGTTA | CGG | 1014 |
| 1714 | −1 | TTAGCGTTTATATCTTTTAT | GGG | 1015 |
| 1715 | −1 | ATTAGCGTTTATATCTTTTA | TGG | 1016 |
| 1768 | 1 | TAAAACGACGATTATAATTA | TGG | 1017 |
| 1775 | 1 | ACGATTATAATTATGGAAGA | TGG | 1018 |
| 1835 | −1 | CCTCTGTATGTCAAAAATAT | CGG | 1019 |
| 1846 | 1 | CCGATATTTTTGACATACAG | AGG | 1020 |
| 1858 | −1 | ACAATTGTAACCACGAAAGA | TGG | 1021 |
| 1859 | 1 | CATACAGAGGCCATCTTTCG | TGG | 1022 |
| 1909 | −1 | ACACATGAAAAATATAGACG | CGG | 1023 |
| 2007 | −1 | AAGTACATAGGCATCAGGAA | GGG | 1024 |
| 2008 | −1 | AAAGTACATAGGCATCAGGA | AGG | 1025 |
| 2012 | −1 | ATAAAAAGTACATAGGCATC | AGG | 1026 |
| 2019 | −1 | ATTAAAAATAAAAAGTACAT | AGG | 1027 |
| 2035 | 1 | GTACTTTTTATTTTTAATAT | TGG | 1028 |
| 2046 | 1 | TTTTAATATTGGAGTAGTAG | TGG | 1029 |
| 2065 | 1 | GTGGTGCGTTTTTTAAAATG | TGG | 1030 |
| 2070 | 1 | GCGTTTTTTAAAATGTGGAT | TGG | 1031 |
| 2075 | 1 | TTTTAAAATGTGGATTGGTT | AGG | 1032 |
| 2107 | 1 | CAAATGTAGAATAGTTTAAC | TGG | 1033 |
| 2122 | 1 | TTAACTGGAGAAGTAGAAAT | CGG | 1034 |
| 2126 | 1 | CTGGAGAAGTAGAAATCGGA | CGG | 1035 |
| 2139 | −1 | TTTCTGTATCTCTAATAAAT | TGG | 1036 |
| 2167 | −1 | GTGTACTTCTAAAAATCGGA | CGG | 1037 |
| 2171 | −1 | ATTTGTGTACTTCTAAAAAT | CGG | 1038 |
| 2185 | 1 | ATTTTTAGAAGTACACAAAT | CGG | 1039 |
| 2205 | 1 | CGGACTGTTTGATTTATGAG | AGG | 1040 |
| 2231 | −1 | AAATTTTTTTTAACACAAAT | CGG | 1041 |
| 2267 | 1 | AAAATTTGAAATAGAGAAAT | CGG | 1042 |
| 2280 | −1 | GTGAAAATAAACAAATCGGA | GGG | 1043 |
| 2281 | −1 | TGTGAAAATAAACAAATCGG | AGG | 1044 |
| 2284 | −1 | AACTGTGAAAATAAACAAAT | CGG | 1045 |
| 2357 | −1 | TCGGTTAATTTTTTGTGTTA | TGG | 1046 |
| 2376 | −1 | TAATGTTAGGTAATAAACAT | CGG | 1047 |
| 2389 | −1 | TTTTTATTATTCTTAATGTT | AGG | 1048 |
| 2560 | −1 | TTTTCATCTTTTAAGTGTGA | TGG | 1049 |
| 2598 | −1 | TCAGATATCAATTTTATGTG | AGG | 1050 |
| 2647 | 1 | AATTTAGTAAAATCATCTAA | CGG | 1051 |
| 2648 | 1 | ATTTAGTAAAATCATCTAAC | GGG | 1052 |
| 2671 | 1 | CTCTCAAATATCAATTTTAT | AGG | 1053 |
| 2695 | −1 | GTTTAATGTTTATGAGTGGG | CGG | 1054 |
| 2698 | −1 | ATTGTTTAATGTTTATGAGT | GGG | 1055 |
| 2699 | −1 | CATTGTTTAATGTTTATGAG | TGG | 1056 |
| 2721 | 1 | CATTAAACAATGTATCTAAC | CGG | 1057 |
| 2729 | −1 | CTTGCTCATGCCTAATTCTC | CGG | 1058 |
| 2730 | 1 | ATGTATCTAACCGGAGAATT | AGG | 1059 |
| 2770 | −1 | CAACGGGGACCACGCATGCA | CGG | 1060 |
| 2772 | 1 | GAACTGAAGCCGTGCATGCG | TGG | 1061 |
| 2785 | −1 | TCTGCGATTTCTTCACAACG | GGG | 1062 |
| 2786 | −1 | ATCTGCGATTTCTTCACAAC | GGG | 1063 |
| 2787 | −1 | TATCTGCGATTTCTTCACAA | CGG | 1064 |
| 2817 | 1 | GATAAATGCTTAGCTGTGCA | TGG | 1065 |
| 2825 | 1 | CTTAGCTGTGCATGGAGACA | AGG | 1066 |
| 2871 | 1 | TGTTATAGAGATGCTTTGCG | AGG | 1067 |
| 2882 | 1 | TGCTTTGCGAGGTGTAACAA | AGG | 1068 |
| 2886 | 1 | TTGCGAGGTGTAACAAAGGA | AGG | 1069 |
| 2887 | 1 | TGCGAGGTGTAACAAAGGAA | GGG | 1070 |
| 2901 | −1 | CTCTTCAGCATGCATGGCTA | TGG | 1071 |
| 2907 | −1 | GGGACACTCTTCAGCATGCA | TGG | 1072 |
| 2927 | −1 | AGTTGGTAGACTGAGCAATT | GGG | 1073 |
| 2928 | −1 | GAGTTGGTAGACTGAGCAAT | TGG | 1074 |
| 2944 | −1 | ACACGTCGGAGAAAGTGAGT | TGG | 1075 |
| 2958 | −1 | GGGTGAAGGAAGGGACACGT | CGG | 1076 |
| 2967 | −1 | GCGAGAGGAGGGTGAAGGAA | GGG | 1077 |
| 2968 | −1 | GGCGAGAGGAGGGTGAAGGA | AGG | 1078 |
| 2972 | −1 | AGGGGGCGAGAGGAGGGTGA | AGG | 1079 |
| 2978 | −1 | ATTTATAGGGGGCGAGAGGA | GGG | 1080 |
| 2979 | −1 | GATTTATAGGGGGCGAGAGG | AGG | 1081 |
| 2982 | −1 | CGTGATTTATAGGGGGCGAG | AGG | 1082 |
| 2989 | −1 | GAAGCGACGTGATTTATAGG | GGG | 1083 |
| 2990 | −1 | AGAAGCGACGTGATTTATAG | GGG | 1084 |
| 2991 | −1 | GAGAAGCGACGTGATTTATA | GGG | 1085 |
| 2992 | −1 | TGAGAAGCGACGTGATTTAT | AGG | 1086 |
| 3019 | −1 | TTGGTGTGGTTGTGATGAAG | AGG | 1087 |
| 3033 | −1 | TGCTATTATTGTTATTGGTG | TGG | 1088 |
| 3038 | −1 | ATGGCTGCTATTATTGTTAT | TGG | 1089 |
| 3051 | 1 | AATAACAATAATAGCAGCCA | TGG | 1090 |
| 3057 | −1 | AAGCGCAAGAAGCTTAGCCA | TGG | 1091 |
| 3096 | 1 | TTGCTTTTGCTTTCTAGTTC | TGG | 1092 |
| 3097 | 1 | TGCTTTTGCTTTCTAGTTCT | GGG | 1093 |
| 3119 | 1 | GAGCTAGCAGCATCTCCTTC | AGG | 1094 |
| 3123 | −1 | CTCCTCCGGCTGCTGCCTGA | AGG | 1095 |
| 3129 | 1 | CATCTCCTTCAGGCAGCAGC | CGG | 1096 |
| 3132 | 1 | CTCCTTCAGGCAGCAGCCGG | AGG | 1097 |
| 3137 | −1 | AACTGGCACGCATTCTCCTC | CGG | 1098 |
| 3154 | −1 | GCGCATTGAGGCGCTGGAAC | TGG | 1099 |
| 3160 | −1 | GTCTCTGCGCATTGAGGCGC | TGG | 1100 |
| 3166 | −1 | TGTCAGGTCTCTGCGCATTG | AGG | 1101 |
| 3182 | −1 | TCTGATTCAATGCGATTGTC | AGG | 1102 |
| 3195 | 1 | TGACAATCGCATTGAATCAG | AGG | 1103 |
| 3196 | 1 | GACAATCGCATTGAATCAGA | GGG | 1104 |
| 3199 | 1 | AATCGCATTGAATCAGAGGG | CGG | 1105 |
| 3215 | 1 | AGGGCGGTTACATTGAGACT | TGG | 1106 |
| 3229 | −1 | ATTCGAACTCCTGGTTGTTG | GGG | 1107 |
| 3230 | −1 | CATTCGAACTCCTGGTTGTT | GGG | 1108 |
| 3231 | 1 | GACTTGGAACCCCAACAACC | AGG | 1109 |
| 3231 | −1 | GCATTCGAACTCCTGGTTGT | TGG | 1110 |
| 3238 | −1 | CGCCGGCGCATTCGAACTCC | TGG | 1111 |
| 3247 | 1 | AACCAGGAGTTCGAATGCGC | CGG | 1112 |
| 3255 | −1 | TAAGCGAGAGAGGGCGACGC | CGG | 1113 |
| 3264 | −1 | GCGGAGGACTAAGCGAGAGA | GGG | 1114 |
| 3265 | −1 | GGCGGAGGACTAAGCGAGAG | AGG | 1115 |
| 3280 | −1 | TACGAAGGGCGTTGCGGCGG | AGG | 1116 |
| 3283 | −1 | GCCTACGAAGGGCGTTGCGG | CGG | 1117 |
| 3286 | −1 | AAGGCCTACGAAGGGCGTTG | CGG | 1118 |
| 3293 | 1 | TCCGCCGCAACGCCCTTCGT | AGG | 1119 |
| 3294 | −1 | GGAGTAGAAAGGCCTACGAA | GGG | 1120 |
| 3295 | −1 | TGGAGTAGAAAGGCCTACGA | AGG | 1121 |
| 3305 | −1 | TGGGGAGCATTGGAGTAGAA | AGG | 1122 |
| 3315 | −1 | GAAGATCTCCTGGGGAGCAT | TGG | 1123 |
| 3318 | 1 | TTTCTACTCCAATGCTCCCC | AGG | 1124 |
| 3323 | −1 | TGCTGGATGAAGATCTCCTG | GGG | 1125 |
| 3324 | −1 | TTGCTGGATGAAGATCTCCT | GGG | 1126 |
| 3325 | −1 | CTTGCTGGATGAAGATCTCC | TGG | 1127 |
| 3337 | 1 | CAGGAGATCTTCATCCAGCA | AGG | 1128 |
| 3340 | −1 | GTAATAAGTAATAACCTTGC | TGG | 1129 |
| 3399 | −1 | AAATTAACAAACAAAGATCA | TGG | 1130 |
| 3433 | 1 | CATTTTGAATGAACACATAC | AGG | 1131 |
| 3437 | 1 | TTGAATGAACACATACAGGA | AGG | 1132 |
| 3438 | 1 | TGAATGAACACATACAGGAA | GGG | 1133 |
| 3439 | 1 | GAATGAACACATACAGGAAG | GGG | 1134 |
| 3448 | 1 | CATACAGGAAGGGGATACTT | TGG | 1135 |
| 3449 | 1 | ATACAGGAAGGGGATACTTT | GGG | 1136 |
| 3463 | 1 | TACTTTGGGTTGATATTCCC | TGG | 1137 |
| 3469 | −1 | CATATGTGCTAGGACAACCA | GGG | 1138 |
| 3470 | −1 | TCATATGTGCTAGGACAACC | AGG | 1139 |
| 3479 | −1 | GCAGGCTCTTCATATGTGCT | AGG | 1140 |
| 3497 | −1 | GATCGACGTCCTTGTTGTGC | AGG | 1141 |
| 3499 | 1 | TATGAAGAGCCTGCACAACA | AGG | 1142 |
| 3525 | −1 | AAGACGTCTTGGTGGTCTTT | GGG | 1143 |
| 3526 | −1 | GAAGACGTCTTGGTGGTCTT | TGG | 1144 |
| 3533 | −1 | TCTCCTTGAAGACGTCTTGG | TGG | 1145 |
| 3536 | −1 | TCTTCTCCTTGAAGACGTCT | TGG | 1146 |
| 3541 | 1 | AGACCACCAAGACGTCTTCA | AGG | 1147 |
| 3559 | −1 | TATCTTGTTGCTGTTGGCTT | TGG | 1148 |
| 3565 | −1 | GGTGACTATCTTGTTGCTGT | TGG | 1149 |
| 3582 | 1 | GCAACAAGATAGTCACCAGA | AGG | 1150 |
| 3586 | −1 | CATCGAAACGGTGCACCTTC | TGG | 1151 |
| 3598 | −1 | TGAGATCACCCTCATCGAAA | CGG | 1152 |
| 3600 | 1 | GAAGGTGCACCGTTTCGATG | AGG | 1153 |
| 3601 | 1 | AAGGTGCACCGTTTCGATGA | GGG | 1154 |
| 3625 | 1 | GATCTCATTGCAGTTCCCAC | CGG | 1155 |
| 3629 | −1 | AGCCAGAAAGCAACACCGGT | GGG | 1156 |
| 3630 | −1 | GAGCCAGAAAGCAACACCGG | TGG | 1157 |
| 3633 | −1 | GTAGAGCCAGAAAGCAACAC | CGG | 1158 |
| 3638 | 1 | TTCCCACCGGTGTTGCTTTC | TGG | 1159 |
| 3661 | −1 | CAGCAACAACATCAGTGTCG | TGG | 1160 |

TABLE 6
gRNA's targeting sequences of Ara h5.

| Position | | | | |
|---|---|---|---|---|
| on SEQ ID | | | | SEQ |
| NO: 1161 | Strand | Sequence | PAM | ID NO: |
| 36 | 1 | TCTTCTCTCTTGAAAATGAT | AGG | 1163 |
| 58 | 1 | GAAAAAGACAGAGTTGCAGA | TGG | 1164 |
| 93 | 1 | TTACTATTTTAGTAATAACT | AGG | 1165 |
| 144 | 1 | TATATTAAAAGTGCATCTAG | TGG | 1166 |
| 151 | 1 | AAAGTGCATCTAGTGGTAAG | TGG | 1167 |
| 214 | 1 | ATAGACTATAGTGCATAATA | TGG | 1168 |
| 241 | −1 | GAAACTTAACTCTGAAATAT | TGG | 1169 |
| 301 | −1 | AGATTAAATACAAAGTGTTC | AGG | 1170 |
| 338 | −1 | TAATCACGGCAAATTAATAA | AGG | 1171 |
| 352 | −1 | CCCAAAAAAGTCAGTAATCA | CGG | 1172 |
| 362 | 1 | GCCGTGATTACTGACTTTTT | TGG | 1173 |
| 363 | 1 | CCGTGATTACTGACTTTTTT | GGG | 1174 |
| 367 | 1 | GATTACTGACTTTTTTGGGT | AGG | 1175 |
| 368 | 1 | ATTACTGACTTTTTTGGGTA | GGG | 1176 |
| 374 | 1 | GACTTTTTTGGGTAGGGATC | TGG | 1177 |
| 377 | 1 | TTTTTTGGGTAGGGATCTGG | AGG | 1178 |
| 394 | −1 | AGCTTGCCCGGTTTTCTTTA | TGG | 1179 |
| 398 | 1 | GGCATCACCATAAAGAAAAC | CGG | 1180 |
| 399 | 1 | GCATCACCATAAAGAAAACC | GGG | 1181 |
| 406 | −1 | GCCGAAAACTAGAGCTTGCC | CGG | 1182 |
| 416 | 1 | ACCGGGCAAGCTCTAGTTTT | CGG | 1183 |
| 427 | 1 | TCTAGTTTTCGGCGTCTATG | AGG | 1184 |
| 441 | −1 | TTGCATTGTCCAGGAGTAAC | TGG | 1185 |
| 443 | 1 | TATGAGGAACCAGTTACTCC | TGG | 1186 |
| 450 | −1 | ACAACCATGTTGCATTGTCC | AGG | 1187 |
| 457 | 1 | TACTCCTGGACAATGCAACA | TGG | 1188 |
| 468 | 1 | AATGCAACATGGTTGTTGAG | AGG | 1189 |
| 472 | 1 | CAACATGGTTGTTGAGAGGT | TGG | 1190 |
| 473 | 1 | AACATGGTTGTTGAGAGGTT | GGG | 1191 |
| 491 | −1 | TCTACAGACCCTGATCAACG | AGG | 1192 |
| 493 | 1 | GGGAGATTACCTCGTTGATC | AGG | 1193 |
| 494 | 1 | GGAGATTACCTCGTTGATCA | GGG | 1194 |
| 511 | 1 | TCAGGGTCTGTAGATGAACC | TGG | 1195 |
| 518 | −1 | GTTCAAATCTTCAAAGATCC | AGG | 1196 |
| 542 | 1 | AGATTTGAACAATTTTTGTT | TGG | 1197 |
| 543 | 1 | GATTTGAACAATTTTTGTTT | GGG | 1198 |
| 575 | 1 | CTTTTTATTATATTCAGTTT | TGG | 1199 |
| 606 | 1 | TCTACATTTCAACCCTAAAT | TGG | 1200 |
| 607 | 1 | CTACATTTCAACCCTAAATT | GGG | 1201 |
| 607 | −1 | CTGCGACAACACCCAATTTA | GGG | 1202 |
| 608 | −1 | TCTGCGACAACACCCAATTT | AGG | 1203 |
| 635 | 1 | TCGCAGACAATAACCATTTG | AGG | 1204 |
| 637 | −1 | TAAACTGTTTAATCCTCAAA | TGG | 1205 |
| 659 | 1 | TTAAACAGTTTATTTCTTGT | TGG | 1206 |
| 709 | −1 | GCTCATAACAGTACAGGATA | TGG | 1207 |
| 715 | −1 | TGTAGTGCTCATAACAGTAC | AGG | 1208 |
| 733 | 1 | TGTTATGAGCACTACATGAG | AGG | 1209 |
| 752 | −1 | TCATGATTATCATACATTAT | TGG | 1210 |
| 780 | 1 | TCATGATTTGCGTCCTAAGC | AGG | 1211 |
| 782 | −1 | TGCAAAATTAAGTCCTGCTT | AGG | 1212 |
| 808 | 1 | ATTTTGCAACATCCTGAAGT | TGG | 1213 |
| 809 | −1 | AGTTCAATCTTTCCAACTTC | AGG | 1214 |
| 839 | −1 | GTAATCACTGCATTAATATG | CGG | 1215 |
| 881 | 1 | TTTTGTCTGAATTACTGAAT | TGG | 1216 |
| 882 | 1 | TTTGTCTGAATTACTGAATT | GGG | 1217 |
| 916 | −1 | ACATTTCTAATTCAAAATAA | TGG | 1218 |
| 929 | 1 | ATTATTTTGAATTAGAAATG | TGG | 1219 |
| 947 | 1 | TGTGGTATTTCGAGCAAAAA | AGG | 1220 |
| 966 | −1 | TTCTTCGGTGGAAAATTAAC | AGG | 1221 |
| 978 | −1 | TGTACATTCTAATTCTTCGG | TGG | 1222 |
| 981 | −1 | GTTTGTACATTCTAATTCTT | CGG | 1223 |
| 1003 | −1 | TTTCATTCCACGCTTCTAAA | AGG | 1224 |
| 1007 | 1 | GTACAAACCTTTTAGAAGCG | TGG | 1225 |
| 1049 | 1 | AAATGCATAATCAATAATTA | AGG | 1226 |
| 1112 | 1 | AACAAGACAATATAGTGTTT | TGG | 1227 |
| 1157 | −1 | ATTGAGGAAGTGCAAGTTTA | GGG | 1228 |
| 1158 | −1 | GATTGAGGAAGTGCAAGTTT | AGG | 1229 |
| 1173 | −1 | CCCTCTGCCCTCTAGGATTG | AGG | 1230 |
| 1176 | 1 | TTGCACTTCCTCAATCCTAG | AGG | 1231 |
| 1177 | 1 | TGCACTTCCTCAATCCTAGA | GGG | 1232 |
| 1180 | −1 | AAGTGCACCCTCTGCCCTCT | AGG | 1233 |
| 1183 | 1 | TCCTCAATCCTAGAGGGCAG | AGG | 1234 |
| 1184 | 1 | CCTCAATCCTAGAGGGCAGA | GGG | 1235 |
| 1217 | −1 | ACAAAAATACATAAAAATAA | CGG | 1236 |
| 1252 | 1 | AATTTTTAACAAATTGATAG | TGG | 1237 |
| 1271 | 1 | GTGGTTCGATTCTAATGATT | TGG | 1238 |
| 1272 | 1 | TGGTTCGATTCTAATGATTT | GGG | 1239 |
| 1291 | −1 | GATACATACAAAAGAGGAGT | AGG | 1240 |
| 1297 | −1 | AAAATTGATACATACAAAAG | AGG | 1241 |
| 1342 | 1 | TCAAAGTGTCTTCGATTAGA | CGG | 1242 |
| 1343 | 1 | CAAAGTGTCTTCGATTAGAC | GGG | 1243 |
| 1440 | −1 | AAATTTTGACGCAATCTTTT | CGG | 1244 |
| 1480 | 1 | AGCTATAAAATATCCTCGAC | TGG | 1245 |
| 1482 | −1 | AAACCCCTTTGATCCAGTCG | AGG | 1246 |
| 1488 | 1 | AATATCCTCGACTGGATCAA | AGG | 1247 |
| 1489 | 1 | ATATCCTCGACTGGATCAAA | GGG | 1248 |
| 1490 | 1 | TATCCTCGACTGGATCAAAG | GGG | 1249 |
| 1511 | 1 | GGTTTTGACATAAAACTTAA | AGG | 1250 |
| 1528 | 1 | TAAAGGTGTGAAATATAAAT | TGG | 1251 |
| 1559 | −1 | TTCAAGTAAATTTATCTTTC | AGG | 1252 |
| 1603 | 1 | TACGTAAAGATAAACTGAGA | AGG | 1253 |
| 1614 | 1 | AAACTGAGAAGGTAAAATTA | TGG | 1254 |
| 1632 | −1 | GATCGAGTCTCTTTTCTGCA | GGG | 1255 |
| 1633 | −1 | CGATCGAGTCTCTTTTCTGC | AGG | 1256 |
| 1765 | −1 | AGTATTGTGTAATTAATGAT | TGG | 1257 |
| 1798 | 1 | TGTCTTGTGCACGCACTTTC | TGG | 1258 |
| 1813 | −1 | TATTGGCAAATGAGTGAAAA | TGG | 1259 |
| 1830 | −1 | ATTAATGGGTAAAATTTTAT | TGG | 1260 |
| 1844 | −1 | GTACCATGTAAAGAATTAAT | GGG | 1261 |
| 1845 | −1 | TGTACCATGTAAAGAATTAA | TGG | 1262 |
| 1852 | 1 | TTACCCATTAATTCTTTACA | TGG | 1263 |
| 1870 | −1 | TACATAAGTGGAGGAGTTGA | GGG | 1264 |
| 1871 | −1 | TTACATAAGTGGAGGAGTTG | AGG | 1265 |
| 1879 | −1 | TCCAACGATTACATAAGTGG | AGG | 1266 |
| 1882 | −1 | GAATCCAACGATTACATAAG | TGG | 1267 |
| 1889 | 1 | TCCTCCACTTATGTAATCGT | TGG | 1268 |
| 1944 | −1 | ATTGAGTGGGTTTAATAGAA | TGG | 1269 |
| 1957 | −1 | GACCCGTTATTAAATTGAGT | GGG | 1270 |
| 1958 | −1 | GGACCCGTTATTAAATTGAG | TGG | 1271 |
| 1965 | 1 | AAACCCACTCAATTTAATAA | CGG | 1272 |
| 1966 | 1 | AACCCACTCAATTTAATAAC | GGG | 1273 |
| 1979 | −1 | ATTCAATACAAGTTCGATTT | CGG | 1274 |
| 2017 | −1 | GAAATATTTGAAAGTGCACA | TGG | 1275 |
| 2053 | −1 | TGGTCGAAAAATATTTAGAT | TGG | 1276 |
| 2069 | 1 | CTAAATATTTTTCGACCAAC | AGG | 1277 |
| 2073 | −1 | AAGGGAAATGATACTCCTGT | TGG | 1278 |
| 2091 | −1 | TCCATTTTTTTGCATAGAAA | GGG | 1279 |
| 2092 | −1 | GTCCATTTTTTTGCATAGAA | AGG | 1280 |
| 2101 | 1 | TCCCTTTCTATGCAAAAAAA | TGG | 1281 |
| 2134 | 1 | CACTATTTTTTAATTATGAA | TGG | 1282 |
| 2166 | 1 | TCAATTTACATAAAAAAGAG | AGG | 1283 |
| 2178 | −1 | TTCAATGGAAATTCGTGTAG | TGG | 1284 |
| 2193 | −1 | ACTTATATGTCATATTTCAA | TGG | 1285 |
| 2210 | 1 | AAATATGACATATAAGTAAA | AGG | 1286 |
| 2211 | 1 | AATATGACATATAAGTAAAA | GGG | 1287 |
| 2223 | 1 | AAGTAAAAGGGTAAAAAAGC | CGG | 1288 |
| 2231 | −1 | AATCATTTCATCAGGTCATC | CGG | 1289 |
| 2239 | −1 | CTAGATTTAATCATTTCATC | AGG | 1290 |
| 2299 | −1 | TCATACGTATTTATCTCTGA | TGG | 1291 |
| 2351 | −1 | ATTTAATCATATAAATCAAC | TGG | 1292 |
| 2512 | 1 | TAAAATCACAAAACACACAA | TGG | 1293 |
| 2767 | 1 | TTAAAAATAAAGTAAAAATT | CGG | 1294 |
| 2801 | 1 | ATAAGAAAAGAAAATTGAAA | AGG | 1295 |
| 2857 | 1 | AAACGACACTTTTGTTTGAA | AGG | 1296 |
| 2871 | 1 | TTTGAAAGGAGTCAAGTCTA | AGG | 1297 |
| 2888 | 1 | CTAAGGAGTAGCGAATATTT | AGG | 1298 |
| 2889 | 1 | TAAGGAGTAGCGAATATTTA | GGG | 1299 |
| 2890 | 1 | AAGGAGTAGCGAATATTTAG | GGG | 1300 |
| 2891 | 1 | AGGAGTAGCGAATATTTAGG | GGG | 1301 |
| 2897 | 1 | AGCGAATATTTAGGGGGAAT | TGG | 1302 |
| 2909 | 1 | GGGGGAATTGGTAATGAAAA | TGG | 1303 |
| 2922 | 1 | ATGAAAATGGCAAAAAGTAC | TGG | 1304 |
| 2933 | 1 | AAAAAGTACTGGTAATGACA | AGG | 1305 |
| 2934 | 1 | AAAAGTACTGGTAATGACAA | GGG | 1306 |
| 2957 | 1 | CATTCTAGTAATTCAACGTG | AGG | 1307 |
| 2974 | 1 | GTGAGGACAATATCAACGTA | TGG | 1308 |
| 2995 | 1 | GGATTTTTTTTATAGCTCCG | TGG | 1309 |
| 3001 | −1 | ATGCTGTAGCGTTGAAACCA | CGG | 1310 |
| 3074 | 1 | ATTAGTACTCAAATTTTCTC | AGG | 1311 |
| 3103 | −1 | TTTCTCTTTCTCTGGAAGTT | GGG | 1312 |
| 3104 | −1 | TTTTCTCTTTCTCTGGAAGT | TGG | 1313 |
| 3111 | −1 | CATCTTGTTTTCTCTTTCTC | TGG | 1314 |
| 3128 | 1 | AAAGAGAAAACAAGATGTCG | TGG | 1315 |
| 3144 | −1 | GAGAAGGTGGTCATCGACGT | AGG | 1316 |
| 3157 | −1 | CTTCAATTTCGCAGAGAAGG | TGG | 1317 |
| 3160 | −1 | TGCCTTCAATTTCGCAGAGA | AGG | 1318 |
| 3169 | 1 | CACCTTCTCTGCGAAATTGA | AGG | 1319 |
| 3184 | −1 | GGATTGCGGCGGAGGAGAGG | TGG | 1320 |
| 3187 | −1 | CGAGGATTGCGGCGGAGGAG | AGG | 1321 |
| 3192 | −1 | TTGGCCGAGGATTGCGGCGG | AGG | 1322 |
| 3195 | −1 | GTCTTGGCCGAGGATTGCGG | CGG | 1323 |
| 3198 | −1 | GCCGTCTTGGCCGAGGATTG | CGG | 1324 |
| 3199 | 1 | CTCTCCTCCGCCGCAATCCT | CGG | 1325 |
| 3205 | −1 | AAACACTGCCGTCTTGGCCG | AGG | 1326 |
| 3208 | 1 | GCCGCAATCCTCGGCCAAGA | CGG | 1327 |
| 3211 | −1 | GAGCCCAAACACTGCCGTCT | TGG | 1328 |
| 3218 | 1 | TCGGCCAAGACGGCAGTGTT | TGG | 1329 |
| 3219 | 1 | CGGCCAAGACGGCAGTGTTT | GGG | 1330 |
| 3243 | 1 | TCAGAGCTCTAATTTCCCTC | AGG | 1331 |
| 3247 | −1 | AAGTATTAAAGATAACCTGA | GGG | 1332 |
| 3248 | −1 | GAAGTATTAAAGATAACCTG | AGG | 1333 |
| 3284 | −1 | AGAAAGAAGAAGGAGAGTTT | TGG | 1334 |
| 3294 | −1 | CAAAGAACTAAGAAAGAAGA | AGG | 1335 |
| 3321 | −1 | AAAAAAGAACGAGAAGAATC | TGG | 1336 |
| 3348 | −1 | AAATCGGAGGAAAATTAACG | GGG | 1337 |
| 3349 | −1 | TAAATCGGAGGAAAATTAAC | GGG | 1338 |
| 3350 | −1 | ATAAATCGGAGGAAAATTAA | CGG | 1339 |
| 3361 | −1 | AATCAAATCTGATAAATCGG | AGG | 1340 |
| 3364 | −1 | GCAAATCAAATCTGATAAAT | CGG | 1341 |
| 3393 | 1 | TTTGCATGAGACATATCCTA | TGG | 1342 |
| 3398 | −1 | CAAATTAACACCAAAACCAT | AGG | 1343 |
| 3399 | 1 | TGAGACATATCCTATGGTTT | TGG | 1344 |
| 3438 | −1 | TTGATAAAACTAAACAGCTA | TGG | 1345 |
| 3453 | 1 | AGCTGTTTAGTTTTATCAAC | TGG | 1346 |
| 3528 | 1 | TTATGCTGTAAATTAAAGCT | TGG | 1347 |

TABLE 7
gRNA's targeting sequences of Ara h6.

| Position | | | | |
|---|---|---|---|---|
| on SEQ ID | | | | SEQ |
| NO: 1348 | Strand | Sequence | PAM | ID NO: |
| 104 | −1 | TGAAATAATTAATAAATATG | TGG | 1350 |
| 205 | −1 | AAAATGATTAATAAATATGT | GGG | 1351 |
| 206 | −1 | GAAAATGATTAATAAATATG | TGG | 1352 |
| 265 | 1 | AAAAATTCAAAAGACATAAA | AGG | 1353 |
| 367 | 1 | AAAAAAAAGCAATTAAACTC | AGG | 1354 |
| 368 | 1 | AAAAAAAGCAATTAAACTCA | GGG | 1355 |
| 395 | 1 | TGAGAACAAGAAAAAAAAAG | AGG | 1356 |
| 396 | 1 | GAGAACAAGAAAAAAAAAGA | GGG | 1357 |
| 432 | −1 | CCCTTAAAATTCGTCAAATT | TGG | 1358 |
| 442 | 1 | ACCAAATTTGACGAATTTTA | AGG | 1359 |
| 443 | 1 | CCAAATTTGACGAATTTTAA | GGG | 1360 |
| 815 | −1 | ATTTAAGTTTTAACTGACTA | TGG | 1361 |
| 837 | 1 | TAAAACTTAAATTAAATAAC | AGG | 1362 |
| 883 | −1 | ATTACATGTCACAATATTAG | TGG | 1363 |
| 1025 | −1 | ATTCAAAAATTAGTGAATTT | TGG | 1364 |
| 1150 | −1 | ATTGAATAACAGTTCTGCTA | AGG | 1365 |
| 1174 | −1 | TTGGTAAAGTATTAAAATAA | CGG | 1366 |
| 1189 | 1 | TATTTTAATACTTTACCAAT | TGG | 1367 |
| 1193 | −1 | GCGAAATTTAAAATACCAAT | TGG | 1368 |
| 1219 | 1 | AATTTCGCTTGACTGTATTA | AGG | 1369 |
| 1255 | 1 | AGTAAATTTAGAATTTTTAC | CGG | 1370 |
| 1263 | −1 | ATTGAAAGGACTATTTTATC | CGG | 1371 |
| 1277 | −1 | ACCCTTTTTTTTCAATTGAA | AGG | 1372 |
| 1286 | 1 | GTCCTTTCAATTGAAAAAAA | AGG | 1373 |
| 1287 | 1 | TCCTTTCAATTGAAAAAAAA | GGG | 1374 |
| 1295 | 1 | ATTGAAAAAAAAGGGTACAG | TGG | 1375 |
| 1305 | 1 | AAGGGTACAGTGGTTAATTT | CGG | 1376 |
| 1485 | −1 | TCTTTTAAGTAAATTTCTTT | TGG | 1377 |
| 1525 | 1 | TATAATATAACTATTAATCC | AGG | 1378 |
| 1532 | −1 | GAAATTGCCACTAACAATCC | TGG | 1379 |
| 1536 | 1 | TATTAATCCAGGATTGTTAG | TGG | 1380 |
| 1555 | 1 | GTGGCAATTTCATGCATTTC | TGG | 1381 |
| 1563 | 1 | TTCATGCATTTCTGGCGAGA | AGG | 1382 |
| 1564 | 1 | TCATGCATTTCTGGCGAGAA | GGG | 1383 |
| 1579 | 1 | GAGAAGGGAAACGATTTTTC | CGG | 1384 |
| 1587 | −1 | CACCATCTCCATGCTGCTAC | CGG | 1385 |
| 1590 | 1 | CGATTTTTCCGGTAGCAGCA | TGG | 1386 |
| 1596 | 1 | TTCCGGTAGCAGCATGGAGA | TGG | 1387 |
| 1599 | 1 | CGGTAGCAGCATGGAGATGG | TGG | 1388 |
| 1615 | 1 | ATGGTGGCATCAATATTTGA | TGG | 1389 |
| 1623 | 1 | ATCAATATTTGATGGTGTTA | AGG | 1390 |
| 1633 | 1 | GATGGTGTTAAGGAATATAA | CGG | 1391 |
| 1660 | −1 | TATGAGTTATTATTAATAGA | AGG | 1392 |
| 1693 | −1 | TCATTTGAGTTATAACTCAT | TGG | 1393 |
| 1732 | 1 | TCTTTTCATACTCAATTAAG | AGG | 1394 |
| 1738 | 1 | CATACTCAATTAAGAGGTTA | CGG | 1395 |
| 1739 | 1 | ATACTCAATTAAGAGGTTAC | GGG | 1396 |
| 1825 | 1 | TTTATTAACAAATCAGTAGC | TGG | 1397 |
| 1837 | 1 | TCAGTAGCTGGAGCATGTCA | TGG | 1398 |
| 1867 | 1 | AATTACTCATTTCGTATTGT | TGG | 1399 |
| 1868 | 1 | ATTACTCATTTCGTATTGTT | GGG | 1400 |
| 2059 | 1 | ATCGTTTAAGACATAAAAAT | AGG | 1401 |
| 2094 | 1 | AGTTGAAGATGTCTCTTTAA | TGG | 1402 |
| 2095 | 1 | GTTGAAGATGTCTCTTTAAT | GGG | 1403 |
| 2101 | 1 | GATGTCTCTTTAATGGGCCA | TGG | 1404 |
| 2107 | −1 | TAATTAAAACAAAATGTCCA | TGG | 1405 |
| 2166 | 1 | ACATGTGTGCTTTTAGCGAG | TGG | 1406 |
| 2167 | 1 | CATGTGTGCTTTTAGCGAGT | GGG | 1407 |
| 2212 | −1 | TATTTTATTTTTTTTTAAAT | TGG | 1408 |
| 2359 | −1 | GCAGTTATTGATACATTAAC | AGG | 1409 |
| 2560 | 1 | ACGATTAATATATTGAAGTA | AGG | 1410 |
| 2704 | 1 | TCAGAAGATATCAAATTTTT | AGG | 1411 |
| 2718 | −1 | TTTCTTCCATCGGGATCTTA | TGG | 1412 |
| 2723 | 1 | TAGGCACCATAAGATCCCGA | TGG | 1413 |
| 2727 | −1 | AACAGCCAATTTCTTCCATC | GGG | 1414 |
| 2728 | −1 | CAACAGCCAATTTCTTCCAT | CGG | 1415 |
| 2733 | 1 | AAGATCCCGATGGAAGAAAT | TGG | 1416 |
| 2762 | 1 | TAAATATCTGTGTTTACTGA | AGG | 1417 |
| 2836 | −1 | TTTTCCTTAATCAGCATGTC | AGG | 1418 |
| 2843 | 1 | AAAGCCTGACATGCTGATTA | AGG | 1419 |
| 2855 | 1 | GCTGATTAAGGAAAAACAAA | TGG | 1420 |
| 2856 | 1 | CTGATTAAGGAAAAACAAAT | GGG | 1421 |
| 2864 | 1 | GGAAAAACAAATGGGAATCT | TGG | 1422 |
| 2894 | 1 | GCATGCATGAAAATGTAACG | TGG | 1423 |
| 2938 | −1 | AAAGGGAATGGAGGGTGGTG | TGG | 1424 |
| 2943 | 1 | CGTGTAAAGGGAATGGAGGG | TGC | 1425 |
| 2946 | −1 | GTACGTGTAAAGGGAATGGA | GGG | 1426 |
| 2947 | −1 | GGTACGTGTAAAGGGAATGG | AGG | 1427 |
| 2950 | −1 | TGAGGTACGTGTAAAGGGAA | TGG | 1428 |
| 2955 | −1 | GTATGTGAGGTACGTGTAAA | GGG | 1429 |
| 2956 | −1 | AGTATGTGAGGTACGTGTAA | AGG | 1430 |
| 2968 | −1 | GAAACAGGGCTTAGTATGTG | AGG | 1431 |
| 2982 | −1 | GGGTATTTATAGGGGAAACA | GGG | 1432 |
| 2983 | −1 | GGGGTATTTATAGGGGAAAC | AGG | 1433 |
| 2990 | −1 | GGCAGCAGGGGTATTTATAG | GGG | 1434 |
| 2991 | −1 | TGGCAGCAGGGGTATTTATA | GGG | 1435 |
| 2992 | −1 | GTGGCAGCAGGGGTATTTAT | AGG | 1436 |
| 3002 | −1 | AGTGTTGATGGTGGCAGCAG | GGG | 1437 |
| 3003 | −1 | TAGTGTTGATGGTGGCAGCA | GGG | 1438 |
| 3004 | −1 | GTAGTGTTGATGGTGGCAGC | AGG | 1439 |
| 3011 | −1 | TTTATGAGTAGTGTTGATGG | TGG | 1440 |
| 3014 | −1 | TTTTTTATGAGTAGTGTTGA | TGG | 1441 |
| 3061 | −1 | GTGGAATGTGATGATGCTTG | AGG | 1442 |
| 3080 | −1 | GGACTTGGCCATGGTGGTGG | TGG | 1443 |
| 3083 | 1 | ATCACATTCCACCACCACCA | TGG | 1444 |
| 3083 | −1 | GGTGGACTTGGCCATGGTGG | TGG | 1445 |
| 3086 | −1 | GATGGTGGACTTGGCCATGG | TGG | 1446 |
| 3089 | −1 | CAGGATGGTGGACTTGGCCA | TGG | 1447 |
| 3095 | −1 | AGCTACCAGGATGGTGGACT | TGG | 1448 |
| 3101 | 1 | CATGGCCAAGTCCACCATCC | TGG | 1449 |
| 3101 | −1 | AAGGAGAGCTACCAGGATGG | TGG | 1450 |
| 3104 | −1 | GGCAAGGAGAGCTACCAGGA | TGG | 1451 |
| 3108 | −1 | CGAGGGCAAGGAGAGCTACC | AGG | 1452 |
| 3120 | −1 | GTGCCACCAGGACGAGGGCA | AGG | 1453 |
| 3125 | 1 | AGCTCTCCTTGCCCTCGTCC | TGG | 1454 |
| 3125 | −1 | GGCGTGTGCCACCAGGACGA | GGG | 1455 |
| 3126 | −1 | AGGCGTGTGCCACCAGGACG | AGG | 1456 |
| 3128 | 1 | TCTCCTTGCCCTCGTCCTGG | TGG | 1457 |
| 3132 | −1 | TTGCGGAGGCGTGTGCCACC | AGG | 1458 |
| 3146 | −1 | CCTCTCGCGCCTCATTGCGG | AGG | 1459 |
| 3148 | 1 | TGGCACACGCCTCCGCAATG | AGG | 1460 |
| 3149 | −1 | CCCCCTCTCGCGCCTCATTG | CGG | 1461 |
| 3157 | 1 | CCTCCGCAATGAGGCGCGAG | AGG | 1462 |
| 3158 | 1 | CTCCGCAATGAGGCGCGAGA | GGG | 1463 |
| 3159 | 1 | TCCGCAATGAGGCGCGAGAG | GGG | 1464 |
| 3160 | 1 | CCGCAATGAGGCGCGAGAGG | GGG | 1465 |
| 3167 | 1 | GAGGCGCGAGAGGGGGAGAC | AGG | 1466 |
| 3168 | 1 | AGGCGCGAGAGGGGGAGACA | GGG | 1467 |
| 3169 | 1 | GGCGCGAGAGGGGGAGACAG | GGG | 1468 |
| 3170 | 1 | GCGCGAGAGGGGGAGACAGG | GGG | 1469 |
| 3190 | 1 | GGGACTCATCAAGCTGCGAG | AGG | 1470 |
| 3194 | 1 | CTCATCAAGCTGCGAGAGGC | AGG | 1471 |
| 3202 | 1 | GCTGCGAGAGGCAGGTAGAC | AGG | 1472 |
| 3203 | 1 | CTGCGAGAGGCAGGTAGACA | GGG | 1473 |
| 3219 | −1 | TGTGCTGCTCGCAGGGCTTG | AGG | 1474 |
| 3226 | −1 | TGCATTATGTGCTGCTCGCA | GGG | 1475 |
| 3227 | −1 | CTGCATTATGTGCTGCTCGC | AGG | 1476 |
| 3241 | 1 | GCGAGCAGCACATAATGCAG | AGG | 1477 |
| 3248 | 1 | GCACATAATGCAGAGGATCA | TGG | 1478 |
| 3249 | 1 | CACATAATGCAGAGGATCAT | GGG | 1479 |
| 3281 | −1 | TCGAGTACTCCTAATATCGT | AGG | 1480 |
| 3283 | 1 | AGTACGACTCCTACGATATT | AGG | 1481 |
| 3305 | −1 | GCACCTCTGTTGCTGGTCGG | AGG | 1482 |
| 3308 | −1 | GCAGCACCTCTGTTGCTGGT | CGG | 1483 |
| 3312 | −1 | CATCGCAGCACCTCTGTTGC | TGG | 1484 |
| 3313 | 1 | GATCCTCCGACCAGCAACAG | AGG | 1485 |
| 3338 | 1 | CTGCGATGAGCTGAACGAGA | TGG | 1486 |
| 3365 | 1 | CACACAGAGATGCATGTGCG | AGG | 1487 |
| 3383 | 1 | CGAGGCATTGCAGCAGATAA | TGG | 1488 |
| 3399 | −1 | TGTCCTGCAACCTATCGCAC | TGG | 1489 |
| 3400 | 1 | TAATGGAGAACCAGTGCGAT | AGG | 1490 |
| 3407 | 1 | GAACCAGTGCGATAGGTTGC | AGG | 1491 |
| 3412 | 1 | AGTGCGATAGGTTGCAGGAC | AGG | 1492 |
| 3419 | 1 | TAGGTTGCAGGACAGGCAAA | TGG | 1493 |
| 3463 | −1 | GCCCTAAAGTTACACTGTTG | GGG | 1494 |
| 3464 | −1 | TGCCCTAAAGTTACACTGTT | GGG | 1495 |
| 3465 | −1 | GTGCCCTAAAGTTACACTGT | TGG | 1496 |
| 3472 | 1 | TGCCCCAACAGTGTAACTTT | AGG | 1497 |
| 3473 | 1 | GCCCCAACAGTGTAACTTTA | GGG | 1498 |
| 3487 | −1 | ACGTCCAAATCGCAACGCTG | TGG | 1499 |
| 3494 | 1 | GGCACCACAGCGTTGCGATT | TGG | 1500 |
| 3504 | 1 | CGTTGCGATTTGGACGTGAG | TGG | 1501 |
| 3507 | 1 | TGCGATTTGGACGTGAGTGG | CGG | 1502 |
| 3550 | −1 | TACTTCCTACTAGTTTGTTT | TGG | 1503 |
| 3556 | 1 | CTGTGCCAAAACAAACTAGT | AGG | 1504 |
| 3629 | 1 | ATATCATCACTGTATGAATG | TGG | 1505 |
| 3636 | 1 | CACTGTATGAATGTGGTGAT | AGG | 1506 |
| 3641 | 1 | TATGAATGTGGTGATAGGTA | AGG | 1507 |
| 3659 | 1 | TAAGGTTATATGAGCACCTT | CGG | 1508 |
| 3664 | −1 | AAGCCATAAGAGCACACCGA | AGG | 1509 |
| 3672 | 1 | GCACCTTCGGTGTGCTCTTA | TGG | 1510 |
| 3689 | −1 | ACTTTGCAGTAGCAAAACAT | AGG | 1511 |
| 3717 | −1 | GAAGATCTTTTATTTCATGG | TGG | 1512 |
| 3720 | −1 | CTTGAAGATCTTTTATTTCA | TGG | 1513 |

TABLE 8
gRNA's targeting sequences of Ara h7.

| Position | | | | |
|---|---|---|---|---|
| on SEQ ID | | | | SEQ ID |
| NO: 1514 | Strand | Sequence | PAM | NO: |
| 10 | −1 | ATTAATTTTAAAAAAACGAG | TGG | 1516 |
| 24 | 1 | CTCGTTTTTTTAAAATTAAT | AGG | 1517 |
| 239 | −1 | ACTAAATGAATTAAGAAGGA | CGG | 1518 |
| 243 | −1 | TAATACTAAATGAATTAAGA | AGG | 1519 |
| 579 | −1 | GATTCAGAAATATTATTACG | AGG | 1520 |
| 601 | −1 | ACTAGCTACCACTCATAGAA | TGG | 1521 |
| 604 | 1 | TTCTGAATCCATTCTATGAG | TGG | 1522 |
| 626 | −1 | TTCTAGTAAGGTATGTTTTA | AGG | 1523 |
| 638 | −1 | TAAAAAATAAAATTCTAGTA | AGG | 1524 |
| 698 | 1 | TAAAATAATTCTTTACAAGT | TGG | 1525 |
| 711 | −1 | GTCAAATGTTAGTTAAAATT | TGG | 1526 |
| 738 | 1 | ATTTGACATTTAATTTGATT | TGG | 1527 |
| 763 | 1 | TTAGTATATATGTATAGTAA | AGG | 1528 |
| 782 | 1 | AAGGCTTTAATTCCTAAAGA | TGG | 1529 |
| 783 | 1 | AGGCTTTAATTCCTAAAGAT | GGG | 1530 |
| 783 | −1 | GGAGGCCAAAACCCATCTTT | AGG | 1531 |
| 789 | 1 | TAATTCCTAAAGATGGGTTT | TGG | 1532 |
| 801 | −1 | CACAAAAGAGACTGATGAGG | AGG | 1533 |
| 804 | −1 | CTTCACAAAAGAGACTGATG | AGG | 1534 |
| 823 | 1 | GTCTCTTTTGTGAAGAGAAA | AGG | 1535 |
| 854 | −1 | GAGACAGATCTTGAGGTGAT | TGG | 1536 |
| 861 | −1 | AACGATAGAGACAGATCTTG | AGG | 1537 |
| 1037 | −1 | ATTTTGATGAATTAAACAAT | AGG | 1538 |
| 1071 | 1 | TCAAAAGATTAGACGCATTT | AGG | 1539 |
| 1174 | −1 | TTAATCAATCCAGATTGATT | TGG | 1540 |
| 1176 | 1 | TAACTGAATCCAAATCAATC | TGG | 1541 |
| 1196 | 1 | TGGATTGATTAAACTCGAAT | TGG | 1542 |
| 1214 | 1 | ATTGGATTAGTTCAGATGAT | TGG | 1543 |
| 1215 | 1 | TTGGATTAGTTCAGATGATT | GGG | 1544 |
| 1230 | −1 | TTTTTAATTTTATTTTTAAT | TGG | 1545 |
| 1354 | 1 | TTTAAATAACTATTATTATT | TGG | 1546 |
| 1438 | 1 | TTAATTAATATTAAATTAAT | TGG | 1547 |
| 1439 | 1 | TAATTAATATTAAATTAATT | GGG | 1548 |
| 1472 | −1 | TTGGCTTAATTATTCATTCT | GGG | 1549 |
| 1473 | −1 | ATTGGCTTAATTATTCATTC | TGG | 1550 |
| 1491 | −1 | CAATTAAACTCAATCTTAAT | TGG | 1551 |
| 1531 | −1 | TTGAATTTAAATAAATAATT | AGG | 1552 |
| 1602 | −1 | GAGTCTTTTGTATTATGGTA | CGG | 1553 |
| 1607 | −1 | TGTAGGAGTCTTTTGTATTA | TGG | 1554 |
| 1624 | −1 | TATCAATTAACATAAGATGT | AGG | 1555 |
| 1731 | 1 | TCTAAAATTTATATAATGAT | TGG | 1556 |
| 1769 | 1 | AAACATAATTTATTATATTG | TGG | 1557 |
| 1787 | 1 | TGTGGCAGACACATGTTTAC | AGG | 1558 |
| 1858 | −1 | ATATATATATTTTTTCAATT | TGG | 1559 |
| 2088 | −1 | AAATTAAGAATTATTATTGT | CGG | 1560 |
| 2177 | −1 | ACATTATACATTTTTATTAA | TGG | 1561 |
| 2197 | 1 | AAAATGTATAATGTGACAAA | CGG | 1562 |
| 2198 | 1 | AAATGTATAATGTGACAAAC | GGG | 1563 |
| 2216 | −1 | ATTAGTACAATGAAATATTA | AGG | 1564 |
| 2280 | 1 | ATTTTTGAATTTTTTTGATG | TGG | 1565 |
| 2454 | −1 | TAAACCAGATAAAAATAAAA | AGG | 1566 |
| 2461 | 1 | ATCACCTTTTTATTTTTATC | TGG | 1567 |
| 2494 | −1 | ATTTATCACATTGGATACAG | AGG | 1568 |
| 2503 | −1 | CTCACTTTGATTTATCACAT | TGG | 1569 |
| 2818 | −1 | TTCAATTCGCCGCATTTTGT | TGG | 1570 |
| 2820 | 1 | AATTATTCACCAACAAAATG | CGG | 1571 |
| 2841 | 1 | GGCGAATTGAAGCCTGCTGC | TGG | 1572 |
| 2842 | −1 | TTACTACACATGCCAGCAGC | AGG | 1573 |
| 2868 | −1 | CTCGTTCATGCGTTTTTGTT | TGG | 1574 |
| 2892 | 1 | CATGAACGAGCAGTGAGACG | TGG | 1575 |
| 2936 | −1 | GAAGGGGAAAGAGGGTTGGG | TGG | 1576 |
| 2939 | −1 | GTGGAAGGGGAAAGAGGGTT | GGG | 1577 |
| 2940 | −1 | CGTGGAAGGGGAAAGAGGGT | TGG | 1578 |
| 2944 | −1 | GGTACGTGGAAGGGGAAAGA | GGG | 1579 |
| 2945 | −1 | TGGTACGTGGAAGGGGAAAG | AGG | 1580 |
| 2952 | −1 | GCATGTGTGGTACGTGGAAG | GGG | 1581 |
| 2953 | −1 | TGCATGTGTGGTACGTGGAA | GGG | 1582 |
| 2954 | −1 | TTGCATGTGTGGTACGTGGA | AGG | 1583 |
| 2958 | −1 | GATATTGCATGTGTGGTACG | TGG | 1584 |
| 2965 | −1 | TAAATGGGATATTGCATGTG | TGG | 1585 |
| 2980 | −1 | GTGGTATTTATAGGATAAAT | GGG | 1586 |
| 2981 | −1 | GGTGGTATTTATAGGATAAA | TGG | 1587 |
| 2989 | −1 | AGGTGCATGGTGGTATTTAT | AGG | 1588 |
| 2999 | −1 | TGGGTGTTGAAGGTGCATGG | TGG | 1589 |
| 3002 | −1 | AGTTGGGTGTTGAAGGTGCA | TGG | 1590 |
| 3009 | −1 | GAATGTGAGTTGGGTGTTGA | AGG | 1591 |
| 3018 | −1 | TGTTTTTTCGAATGTGAGTT | GGG | 1592 |
| 3019 | −1 | GTGTTTTTTCGAATGTGAGT | TGG | 1593 |
| 3067 | 1 | ATCATCATATTCTTCAATAA | TGG | 1594 |
| 3092 | −1 | GGGCGCCCAGGAGAGCTACT | AGG | 1595 |
| 3097 | 1 | CAGCATCCTAGTAGCTCTCC | TGG | 1596 |
| 3098 | 1 | AGCATCCTAGTAGCTCTCCT | GGG | 1597 |
| 3104 | −1 | CTACGACAAGAAGGGCGCCC | AGG | 1598 |
| 3112 | −1 | CGCGGAGGCTACGACAAGAA | GGG | 1599 |
| 3113 | −1 | TCGCGGAGGCTACGACAAGA | AGG | 1600 |
| 3127 | −1 | GGGATCCCATCTTGTCGCGG | AGG | 1601 |
| 3130 | −1 | ATCGGGATCCCATCTTGTCG | CGG | 1602 |
| 3132 | 1 | TCGTAGCCTCCGCGACAAGA | TGG | 1603 |
| 3133 | 1 | CGTAGCCTCCGCGACAAGAT | GGG | 1604 |
| 3146 | 1 | ACAAGATGGGATCCCGATCG | AGG | 1605 |
| 3147 | 1 | CAAGATGGGATCCCGATCGA | GGG | 1606 |
| 3147 | −1 | GACCCTCTGGACCCTCGATC | GGG | 1607 |
| 3148 | −1 | CGACCCTCTGGACCCTCGAT | CGG | 1608 |
| 3155 | 1 | GATCCCGATCGAGGGTCCAG | AGG | 1609 |
| 3156 | 1 | ATCCCGATCGAGGGTCCAGA | GGG | 1610 |
| 3160 | −1 | TGCGTCCCATCTCGACCCTC | TGG | 1611 |
| 3165 | 1 | GAGGGTCCAGAGGGTCGAGA | TGG | 1612 |
| 3166 | 1 | AGGGTCCAGAGGGTCGAGAT | GGG | 1613 |
| 3182 | 1 | AGATGGGACGCACCGAGCAG | AGG | 1614 |
| 3183 | 1 | GATGGGACGCACCGAGCAGA | GGG | 1615 |
| 3183 | −1 | CACTGGTCATCCCCTCTGCT | CGG | 1616 |
| 3184 | 1 | ATGGGACGCACCGAGCAGAG | GGG | 1617 |
| 3200 | −1 | TCTGCAACTGCCTCTGGCAC | TGG | 1618 |
| 3201 | 1 | GAGGGGATGACCAGTGCCAG | AGG | 1619 |
| 3206 | −1 | TTGCCCTCTGCAACTGCCTC | TGG | 1620 |
| 3213 | 1 | AGTGCCAGAGGCAGTTGCAG | AGG | 1621 |
| 3214 | 1 | GTGCCAGAGGCAGTTGCAGA | GGG | 1622 |
| 3225 | 1 | AGTTGCAGAGGGCAAACCTG | AGG | 1623 |
| 3230 | −1 | TATGTTCCTCACAGGGCCTC | AGG | 1624 |
| 3235 | 1 | GGCAAACCTGAGGCCCTGTG | AGG | 1625 |
| 3237 | −1 | TGCCTTATATGTTCCTCACA | GGG | 1626 |
| 3238 | −1 | TTGCCTTATATGTTCCTCAC | AGG | 1627 |
| 3246 | 1 | GGCCCTGTGAGGAACATATA | AGG | 1628 |
| 3252 | 1 | GTGAGGAACATATAAGGCAA | AGG | 1629 |
| 3253 | 1 | TGAGGAACATATAAGGCAAA | GGG | 1630 |
| 3256 | 1 | GGAACATATAAGGCAAAGGG | TGG | 1631 |
| 3299 | −1 | TGGATCCCCGTTGGATGTAC | GGG | 1632 |
| 3300 | −1 | CTGGATCCCCGTTGGATGTA | CGG | 1633 |
| 3303 | 1 | ACGAGTACCCGTACATCCAA | CGG | 1634 |
| 3304 | 1 | CGAGTACCCGTACATCCAAC | GGG | 1635 |
| 3305 | 1 | GAGTACCCGTACATCCAACG | GGG | 1636 |
| 3308 | −1 | GTTGTCCTCTGGATCCCCGT | TGG | 1637 |
| 3314 | 1 | TACATCCAACGGGGATCCAG | AGG | 1638 |
| 3319 | −1 | TTCGCCGGGTCGTTGTCCTC | TGG | 1639 |
| 3326 | 1 | GGATCCAGAGGACAACGACC | CGG | 1640 |
| 3333 | −1 | TGGTCTTCGTCAGATTCGCC | GGG | 1641 |
| 3334 | −1 | TTGGTCTTCGTCAGATTCGC | CGG | 1642 |
| 3353 | −1 | CGTTGCAGCACCTTTGCTCT | TGG | 1643 |
| 3354 | 1 | CTGACGAAGACCAAGAGCAA | AGG | 1644 |
| 3375 | 1 | GGTGCTGCAACGAGCTCAAC | CGG | 1645 |
| 3383 | −1 | ACCTTTGGTTATTCTGGAAC | CGG | 1646 |
| 3389 | −1 | ACATGCACCTTTGGTTATTC | TGG | 1647 |
| 3393 | 1 | ACCGGTTCCAGAATAACCAA | AGG | 1648 |
| 3398 | −1 | GTGCCTGGCACATGCACCTT | TGG | 1649 |
| 3406 | 1 | TAACCAAAGGTGCATGTGCC | AGG | 1650 |
| 3413 | −1 | GGAGGATCTGTTGAAGTGCC | TGG | 1651 |
| 3431 | −1 | ACCTAAAGCTCTGGTTCTGG | AGG | 1652 |
| 3434 | −1 | GGAACCTAAAGCTCTGGTTC | TGG | 1653 |
| 3440 | −1 | CCTGCTGGAACCTAAAGCTC | TGG | 1654 |
| 3441 | 1 | TCCTCCAGAACCAGAGCTTT | AGG | 1655 |
| 3451 | 1 | CCAGAGCTTTAGGTTCCAGC | AGG | 1656 |
| 3455 | −1 | GCAACTGGCTCCTGTCCTGC | TGG | 1657 |
| 3456 | 1 | GCTTTAGGTTCCAGCAGGAC | AGG | 1658 |
| 3470 | −1 | CCCTCTCCATCTGATGCAAC | TGG | 1659 |
| 3475 | 1 | CAGGAGCCAGTTGCATCAGA | TGG | 1660 |
| 3480 | 1 | GCCAGTTGCATCAGATGGAG | AGG | 1661 |
| 3481 | 1 | CCAGTTGCATCAGATGGAGA | GGG | 1662 |
| 3489 | 1 | ATCAGATGGAGAGGGAGCTC | AGG | 1663 |
| 3507 | −1 | GACCTGAACCCGCAGTTCTG | GGG | 1664 |
| 3508 | −1 | TGACCTGAACCCGCAGTTCT | GGG | 1665 |
| 3509 | 1 | AGGAACTTGCCCCAGAACTG | CGG | 1666 |
| 3509 | −1 | GTGACCTGAACCCGCAGTTC | TGG | 1667 |
| 3510 | 1 | GGAACTTGCCCCAGAACTGC | GGG | 1668 |
| 3516 | 1 | TGCCCCAGAACTGCGGGTTC | AGG | 1669 |
| 3531 | −1 | CTACTGAGGTCGCAACGGCT | TGG | 1670 |
| 3536 | −1 | TGCGGCTACTGAGGTCGCAA | CGG | 1671 |
| 3545 | −1 | AGTAGGGCGTGCGGCTACTG | AGG | 1672 |
| 3554 | −1 | AGTCTGTTTAGTAGGGCGTG | CGG | 1673 |
| 3561 | −1 | CTATACTAGTCTGTTTAGTA | GGG | 1674 |
| 3562 | −1 | ACTATACTAGTCTGTTTAGT | AGG | 1675 |
| 3597 | −1 | TCATTGTTATTTCTCTTGTT | GGG | 1676 |
| 3598 | −1 | ATCATTGTTATTTCTCTTGT | TGG | 1677 |
| 3651 | 1 | ACAAGCTATTTCTATGTCTA | TGG | 1678 |
| 3659 | 1 | TTTCTATGTCTATGGTGTTT | TGG | 1679 |
| 3689 | −1 | ACGATCACCACTACATAAAA | TGG | 1680 |
| 3693 | 1 | ATCATCACCATTTTATGTAG | TGG | 1681 |
| 3711 | 1 | AGTGGTGATCGTATTGTCTG | TGG | 1682 |
| 3724 | 1 | TTGTCTGTGGCGAAGTTATA | TGG | 1683 |
| 3725 | 1 | TGTCTGTGGCGAAGTTATAT | GGG | 1684 |
| 3726 | 1 | GTCTGTGGCGAAGTTATATG | GGG | 1685 |
| 3749 | 1 | CACTTTGAAATGTGCTTTTA | TGG | 1686 |
| 3766 | −1 | TTTTACATTATCAAAAGCAC | AGG | 1687 |
| 3798 | 1 | AAGAGTTGTAAACATGTCGA | TGG | 1688 |
| 3820 | 1 | GAATAATAATCTTGTTTCTT | TGG | 1689 |

TABLE 9
gRNA's targeting sequences of Ara h8.

| Position | | | | |
|---|---|---|---|---|
| on SEQ ID | | | | SEQ ID |
| NO: 1690 | Strand | Sequence | PAM | NO: |
| 7 | −1 | GGTGCGCTAGGTCACCCTGT | TGG | 1692 |
| 19 | −1 | ATGACGCATGAGGGTGCGCT | AGG | 1693 |
| 28 | −1 | CTGGACGCCATGACGCATGA | GGG | 1694 |
| 29 | −1 | CCTGGACGCCATGACGCATG | AGG | 1695 |
| 32 | 1 | TAGCGCACCCTCATGCGTCA | TGG | 1696 |
| 40 | 1 | CCTCATGCGTCATGGCGTCC | AGG | 1697 |
| 47 | −1 | ATATTTTTCTTCTCTGGACC | TGG | 1698 |
| 53 | −1 | TCCACCATATTTTTCTTCTC | TGG | 1699 |
| 60 | 1 | AGGTCCAGAGAAGAAAAATA | TGG | 1700 |
| 63 | 1 | TCCAGAGAAGAAAAATATGG | TGG | 1701 |
| 88 | −1 | GCTGGCGGTGGTTCTAGCTC | TGG | 1702 |
| 100 | −1 | GGGCCTGCATTGGCTGGCGG | TGG | 1703 |
| 103 | −1 | GCTGGGCCTGCATTGGCTGG | CGG | 1704 |
| 106 | −1 | GCGGCTGGGCCTGCATTGGC | TGG | 1705 |
| 108 | 1 | GAACCACCGCCAGCCAATGC | AGG | 1706 |
| 110 | −1 | TCAAGCGGCTGGGCCTGCAT | TGG | 1707 |
| 120 | −1 | GGGACGAGGTTCAAGCGGCT | GGG | 1708 |
| 121 | −1 | GGGGACGAGGTTCAAGCGGC | TGG | 1709 |
| 125 | −1 | TAGCGGGGACGAGGTTCAAG | CGG | 1710 |
| 134 | −1 | TGAGGATGATAGCGGGGACG | AGG | 1711 |
| 140 | −1 | AACGATTGAGGATGATAGCG | GGG | 1712 |
| 141 | −1 | AAACGATTGAGGATGATAGC | GGG | 1713 |
| 142 | −1 | GAAACGATTGAGGATGATAG | CGG | 1714 |
| 152 | −1 | CGTTGAGCCCGAAACGATTG | AGG | 1715 |
| 155 | 1 | GCTATCATCCTCAATCGTTT | CGG | 1716 |
| 156 | 1 | CTATCATCCTCAATCGTTTC | GGG | 1717 |
| 177 | 1 | GGCTCAACGTCGTCCTCATC | AGG | 1718 |
| 179 | −1 | AGAATTGGGTGATCCTGATG | AGG | 1719 |
| 193 | −1 | GGCGTCGGAGACGGAGAATT | GGG | 1720 |
| 194 | −1 | TGGCGTCGGAGACGGAGAAT | TGG | 1721 |
| 202 | −1 | CCACCGGTTGGCGTCGGAGA | CGG | 1722 |
| 208 | −1 | GGCGTTCCACCGGTTGGCGT | CGG | 1723 |
| 210 | 1 | TCTCCGTCTCCGACGCCAAC | CGG | 1724 |
| 213 | 1 | CCGTCTCCGACGCCAACCGG | TGG | 1725 |
| 214 | −1 | TTACAGGGCGTTCCACCGGT | TGG | 1726 |
| 218 | −1 | CGCTTTACAGGGCGTTCCAC | CGG | 1727 |
| 229 | −1 | GTTCTGGCAAACGCTTTACA | GGG | 1728 |
| 230 | −1 | TGTTCTGGCAAACGCTTTAC | AGG | 1729 |
| 245 | −1 | GGTAGGATCCTTTGATGTTC | TGG | 1730 |
| 248 | 1 | AGCGTTTGCCAGAACATCAA | AGG | 1731 |
| 262 | −1 | GACGGCGTCCGCGACGAGGT | AGG | 1732 |
| 265 | 1 | CAAAGGATCCTACCTCGTCG | CGG | 1733 |
| 266 | −1 | GAATGACGGCGTCCGCGACG | AGG | 1734 |
| 280 | −1 | TTTGCTGTTGATTTGAATGA | CGG | 1735 |
| 297 | 1 | TTCAAATCAACAGCAAATGA | CGG | 1736 |
| 302 | 1 | ATCAACAGCAAATGACGGAG | AGG | 1737 |
| 313 | 1 | ATGACGGAGAGGCGACAAGT | TGG | 1738 |
| 314 | 1 | TGACGGAGAGGCGACAAGTT | GGG | 1739 |
| 321 | 1 | GAGGCGACAAGTTGGGCCGC | TGG | 1740 |
| 326 | −1 | CGTACCAGTTTACGAGCCAG | CGG | 1741 |
| 333 | 1 | TGGGCCGCTGGCTCGTAAAC | TGG | 1742 |
| 338 | 1 | CGCTGGCTCGTAAACTGGTA | CGG | 1743 |
| 341 | 1 | TGGCTCGTAAACTGGTACGG | AGG | 1744 |
| 347 | 1 | GTAAACTGGTACGGAGGAAG | AGG | 1745 |
| 359 | −1 | CTCCAGTTCGATGGCTGCTG | TGG | 1746 |
| 368 | 1 | GGCCACAGCAGCCATCGAAC | TGG | 1747 |
| 368 | −1 | GGCAGCTGGCTCCAGTTCGA | TGG | 1748 |
| 382 | −1 | ACCAATGTAGCCACGGCAGC | TGG | 1749 |
| 383 | 1 | CGAACTGGAGCCAGCTGCCG | TGG | 1750 |
| 389 | −1 | GAATACCACCAATGTAGCCA | CGG | 1751 |
| 392 | 1 | GCCAGCTGCCGTGGCTACAT | TGG | 1752 |
| 395 | 1 | AGCTGCCGTGGCTACATTGG | TGG | 1753 |
| 403 | 1 | TGGCTACATTGGTGGTATTC | CGG | 1754 |
| 411 | −1 | CCAGCTCAGGGGGTTCGAAC | CGG | 1755 |
| 421 | −1 | GATGTGGTATCCAGCTCAGG | GGG | 1756 |
| 422 | 1 | CCGGTTCGAACCCCCTGAGC | TGG | 1757 |
| 422 | −1 | TGATGTGGTATCCAGCTCAG | GGG | 1758 |
| 423 | −1 | TTGATGTGGTATCCAGCTCA | GGG | 1759 |
| 424 | −1 | GTTGATGTGGTATCCAGCTC | AGG | 1760 |
| 437 | −1 | GCTGGCAAAGCTGGTTGATG | TGG | 1761 |
| 446 | −1 | ACCAGAGTTGCTGGCAAAGC | TGG | 1762 |
| 455 | −1 | GGTCCGCACACCAGAGTTGC | TGG | 1763 |
| 456 | 1 | ACCAGCTTTGCCAGCAACTC | TGG | 1764 |
| 463 | 1 | TTGCCAGCAACTCTGGTGTG | CGG | 1765 |
| 471 | 1 | AACTCTGGTGTGCGGACCTC | CGG | 1766 |
| 476 | −1 | TTGTCGGAGGCAGTTTCCGG | AGG | 1767 |
| 479 | −1 | TCTTTGTCGGAGGCAGTTTC | CGG | 1768 |
| 489 | −1 | AAGTCATGTTTCTTTGTCGG | AGG | 1769 |
| 492 | −1 | TCCAAGTCATGTTTCTTTGT | CGG | 1770 |
| 502 | 1 | TCCGACAAAGAAACATGACT | TGG | 1771 |
| 505 | 1 | GACAAAGAAACATGACTTGG | AGG | 1772 |
| 518 | −1 | TCTGACGATTGGTAGTGATG | AGG | 1773 |
| 529 | −1 | AAGTATGATTGTCTGACGAT | TGG | 1774 |
| 554 | −1 | AGTCACGATCCTGCACGATA | CGG | 1775 |
| 556 | 1 | CATACTTCACCGTATCGTGC | AGG | 1776 |
| 567 | 1 | GTATCGTGCAGGATCGTGAC | TGG | 1777 |
| 603 | −1 | TTGGCCTGGAAGGTGCGAAG | CGG | 1778 |
| 610 | 1 | TAATCCGCTTCGCACCTTCC | AGG | 1779 |
| 613 | −1 | CTGATGAAACTTGGCCTGGA | AGG | 1780 |
| 617 | −1 | TGTGCTGATGAAACTTGGCC | TGG | 1781 |
| 622 | −1 | AGAGCTGTGCTGATGAAACT | TGG | 1782 |
| 640 | 1 | TCATCAGCACAGCTCTAACC | AGG | 1783 |
| 647 | −1 | AACGACAAGCTATGATGACC | TGG | 1784 |
| 677 | −1 | TAAGAATCCTCTCTATATTG | TGG | 1785 |
| 681 | 1 | CGATTCACCACAATATAGAG | AGG | 1786 |
| 707 | 1 | CTTATCTGTGAACTTCACTC | CGG | 1787 |
| 715 | −1 | ATCAAGAGGAAAACTCGGTC | CGG | 1788 |
| 720 | −1 | GATCCATCAAGAGGAAAACT | CGG | 1789 |
| 728 | 1 | GGACCGAGTTTTCCTCTTGA | TGG | 1790 |
| 729 | −1 | ACCACAAAGGATCCATCAAG | AGG | 1791 |
| 739 | 1 | TCCTCTTGATGGATCCTTTG | TGG | 1792 |
| 742 | −1 | GCTGTTTTGGTACACCACAA | AGG | 1793 |
| 755 | −1 | GGAGGAGAGTTTTGCTGTTT | TGG | 1794 |
| 773 | −1 | ACGTAACATGGCTAGTGAGG | AGG | 1795 |
| 776 | −1 | AGGACGTAACATGGCTAGTG | AGG | 1796 |
| 785 | −1 | TCTCATTAGAGGACGTAACA | TGG | 1797 |
| 796 | −1 | ACGTGAATTGCTCTCATTAG | AGG | 1798 |
| 846 | −1 | CCAGGTTAGTTCGAATTAGT | AGG | 1799 |
| 857 | 1 | CCTACTAATTCGAACTAACC | TGG | 1800 |
| 864 | −1 | CATGTATGATGGTTCGAACC | AGG | 1801 |
| 875 | −1 | GGTTCGAATTACATGTATGA | TGG | 1802 |
| 893 | 1 | ACATGTAATTCGAACCAAGC | CGG | 1803 |
| 896 | −1 | TGTAGTAATTCGAACCGGCT | TGG | 1804 |
| 901 | −1 | TTGGTTGTAGTAATTCGAAC | CGG | 1805 |
| 920 | −1 | GGTTCGAACCAAAGAGAGGT | TGG | 1806 |
| 923 | 1 | TACTACAACCAACCTCTCTT | TGG | 1807 |
| 924 | −1 | AACTGGTTCGAACCAAAGAG | AGG | 1808 |
| 938 | 1 | CTCTTTGGTTCGAACCAGTT | TGG | 1809 |
| 941 | −1 | TTTCTTGGTTCGAACCAAAC | TGG | 1810 |
| 956 | −1 | GTTCGAATTACATGCTTTCT | TGG | 1811 |
| 975 | 1 | GCATGTAATTCGAACCAGCC | TGG | 1812 |
| 978 | −1 | TGTAGTAATTCGAACCAGGC | TGG | 1813 |
| 982 | −1 | TGGTTGTAGTAATTCGAACC | AGG | 1814 |
| 1002 | −1 | CGAATTATGGGGAGAGAGCT | TGG | 1815 |
| 1013 | −1 | CCTGGTTGGTTCGAATTATG | GGG | 1816 |
| 1014 | −1 | ACCTGGTTGGTTCGAATTAT | GGG | 1817 |
| 1015 | −1 | AACCTGGTTGGTTCGAATTA | TGG | 1818 |
| 1024 | 1 | CCCCATAATTCGAACCAACC | AGG | 1819 |
| 1027 | −1 | GCATGTAATTCGAACCTGGT | TGG | 1820 |
| 1031 | −1 | GAAAGCATGTAATTCGAACC | TGG | 1821 |
| 1046 | 1 | GTTCGAATTACATGCTTTCT | TGG | 1822 |
| 1061 | 1 | TTTCTTGGTTCGAACCAATC | TGG | 1823 |
| 1064 | −1 | CTGAATAATTCGAACCAGAT | TGG | 1824 |
| 1106 | 1 | AGTAGTAGTTCGAACCATGT | TGG | 1825 |
| 1109 | −1 | ACTAGTAATTCGAACCAACA | TGG | 1826 |
| 1143 | 1 | TAATACAGTTCGAACCATGC | TGG | 1827 |
| 1146 | −1 | TTATATAAATCGAACCAGCA | TGG | 1828 |
| 1170 | 1 | ATTTATATAAAAATGCCTCT | TGG | 1829 |
| 1174 | −1 | TGTTTTAGCAATAAGCCAAG | AGG | 1830 |
| 1198 | 1 | TGCTAAAACAAATTTCTGTT | TGG | 1831 |
| 1208 | 1 | AATTTCTGTTTGGCGTATTT | TGG | 1832 |
| 1228 | 1 | TGGTTAAACTTTTCTGCATG | TGG | 1833 |
| 1238 | 1 | TTTCTGCATGTGGCTTAATA | TGG | 1834 |
| 1257 | −1 | TATTTTTTATATATTTTTTA | GGG | 1835 |
| 1258 | −1 | ATATTTTTTATATATTTTTT | AGG | 1836 |
| 1330 | −1 | AAACATACACTTAAGTCTTA | AGG | 1837 |
| 1402 | −1 | AAGCGTGCCTATCACTAGGA | AGG | 1838 |
| 1406 | 1 | TCTTTTTCCTTCCTAGTGAT | AGG | 1839 |
| 1406 | −1 | TATAAAGCGTGCCTATCACT | AGG | 1840 |
| 1491 | 1 | ATATACAAAATATGAAAAAT | TGG | 1841 |
| 1492 | 1 | TATACAAAATATGAAAAATT | GGG | 1842 |
| 1504 | 1 | GAAAAATTGGGAGTTCTAGA | AGG | 1843 |
| 1505 | 1 | AAAAATTGGGAGTTCTAGAA | GGG | 1844 |
| 1534 | −1 | ACATGTAGTGGCATTTAAAG | TGG | 1845 |
| 1546 | −1 | AAATGAATGTAGACATGTAG | TGG | 1846 |
| 1566 | 1 | GTCTACATTCATTTTGTTTC | TGG | 1847 |
| 1749 | 1 | CAAGTTAGATAATTTAGACA | TGG | 1848 |
| 1785 | −1 | ATATTTGATGCAATAAATAA | AGG | 1849 |
| 1798 | 1 | TTTATTTATTGCATCAAATA | TGG | 1850 |
| 1799 | 1 | TTATTTATTGCATCAAATAT | GGG | 1851 |
| 1821 | 1 | GTAAACATTGAATACGACTG | AGG | 1852 |
| 1839 | −1 | TCACTTTCAAGTTTCAAATA | AGG | 1853 |
| 1903 | −1 | AAATTGTCAAATATTGCAAT | TGG | 1854 |
| 1943 | −1 | GGGTGGAAGAATATTATTAG | TGG | 1855 |
| 1960 | −1 | TCGAAGTTAAGATATTCGGG | TGG | 1856 |
| 1963 | −1 | TTTTCGAAGTTAAGATATTC | GGG | 1857 |
| 1964 | −1 | ATTTTCGAAGTTAAGATATT | CGG | 1858 |
| 1977 | 1 | GAATATCTTAACTTCGAAAA | TGG | 1859 |
| 1978 | 1 | AATATCTTAACTTCGAAAAT | GGG | 1860 |
| 2120 | −1 | AATAAAAGAAAATATAAATA | AGG | 1861 |
| 2215 | 1 | ATATATATAAAATTCACAAT | TGG | 1862 |
| 2222 | 1 | TAAAATTCACAATTGGTATA | AGG | 1863 |
| 2243 | −1 | CTAAATACTATTCTACTCAT | CGG | 1864 |
| 2277 | 1 | AATAAAATTAAAGATATGTT | TGG | 1865 |
| 2314 | 1 | TTTTATTTTTAAATTATTTT | AGG | 1866 |
| 2444 | 1 | AACATTAAAAATACCCAACA | TGG | 1867 |
| 2446 | −1 | TTTATTGTGTCTGCCATGTT | GGG | 1868 |
| 2447 | −1 | ATTTATTGTGTCTGCCATGT | TGG | 1869 |
| 2516 | −1 | ACTCCTCTTAATGAAAATAT | TGG | 1870 |
| 2524 | 1 | GAACCAATATTTTCATTAAG | AGG | 1871 |
| 2534 | 1 | TTTCATTAAGAGGAGTGTTA | AGG | 1872 |
| 2535 | 1 | TTCATTAAGAGGAGTGTTAA | GGG | 1873 |
| 2551 | 1 | TTAAGGGTCAGTCATTTTTA | TGG | 1874 |
| 2570 | −1 | TACTATTGATAGTCAATTGA | TGG | 1875 |
| 2611 | 1 | TTATGTGAGATTACATCTAA | TGG | 1876 |
| 2614 | 1 | TGTGAGATTACATCTAATGG | TGG | 1877 |
| 2615 | 1 | GTGAGATTACATCTAATGGT | GGG | 1878 |
| 2638 | 1 | ATATTACTCACTTTTTTTTA | TGG | 1879 |
| 2649 | 1 | TTTTTTTTATGGTTAAGTGT | TGG | 1880 |
| 2683 | −1 | ATTTTAATGAGAAAGTTTAG | GGG | 1881 |
| 2684 | −1 | AATTTTAATGAGAAAGTTTA | GGG | 1882 |
| 2685 | −1 | AAATTTTAATGAGAAAGTTT | AGG | 1883 |
| 2698 | 1 | TAAACTTTCTCATTAAAATT | TGG | 1884 |
| 2721 | −1 | ATTACTCATTTTTCTTTTGT | TGG | 1885 |
| 2771 | 1 | CATCAATAATAGCTAATTGA | TGG | 1886 |
| 2794 | 1 | TTACAAATTATAAAATTTGC | TGG | 1887 |
| 2808 | −1 | ATTTTAATAAGAAGAGTATT | AGG | 1888 |
| 2822 | 1 | AATACTCTTCTTATTAAAAT | CGG | 1889 |
| 2959 | −1 | CAAGGGAATTGCAGATGGAA | TGG | 1890 |
| 2964 | −1 | GGGGTCAAGGGAATTGCAGA | TGG | 1891 |
| 2976 | −1 | TAGGCATATAATGGGGTCAA | GGG | 1892 |
| 2977 | −1 | ATAGGCATATAATGGGGTCA | AGG | 1893 |
| 2983 | −1 | CTATTTATAGGCATATAATG | GGG | 1894 |
| 2984 | −1 | TCTATTTATAGGCATATAAT | GGG | 1895 |
| 2985 | −1 | TTCTATTTATAGGCATATAA | TGG | 1896 |
| 2995 | −1 | GAGGAGTGGCTTCTATTTAT | AGG | 1897 |
| 3009 | −1 | GTGGTTGATGAGCAGAGGAG | TGG | 1898 |
| 3014 | −1 | AGTGTGTGGTTGATGAGCAG | AGG | 1899 |
| 3028 | −1 | GAGGTTAGATTTGTAGTGTG | TGG | 1900 |
| 3047 | −1 | GAAGTAGATAGTAATAAGTG | AGG | 1901 |
| 3075 | −1 | AAGCTAAGGGAAGGAATGAA | AGG | 1902 |
| 3084 | −1 | TGAGTTAAGAAGCTAAGGGA | AGG | 1903 |
| 3088 | −1 | TGACTGAGTTAAGAAGCTAA | GGG | 1904 |
| 3089 | −1 | ATGACTGAGTTAAGAAGCTA | AGG | 1905 |
| 3102 | 1 | TTAGCTTCTTAACTCAGTCA | TGG | 1906 |
| 3103 | 1 | TAGCTTCTTAACTCAGTCAT | GGG | 1907 |
| 3120 | 1 | CATGGGCGTCTTCACTTTTG | AGG | 1908 |
| 3141 | −1 | CTTAGCAGGGGGCAGGGTGG | AGG | 1909 |
| 3144 | −1 | AAGCTTAGCAGGGGGCAGGG | TGG | 1910 |
| 3147 | −1 | GTAAAGCTTAGCAGGGGGCA | GGG | 1911 |
| 3148 | −1 | TGTAAAGCTTAGCAGGGGGC | AGG | 1912 |
| 3152 | −1 | GCGTTGTAAAGCTTAGCAGG | GGG | 1913 |
| 3153 | −1 | AGCGTTGTAAAGCTTAGCAG | GGG | 1914 |
| 3154 | −1 | GAGCGTTGTAAAGCTTAGCA | GGG | 1915 |
| 3155 | −1 | AGAGCGTTGTAAAGCTTAGC | AGG | 1916 |
| 3171 | 1 | TAAGCTTTACAACGCTCTGA | AGG | 1917 |
| 3186 | −1 | AATCTTAGGGGTGATGGTAT | CGG | 1918 |
| 3192 | −1 | ATCAATAATCTTAGGGGTGA | TGG | 1919 |
| 3198 | −1 | GACGTCATCAATAATCTTAG | GGG | 1920 |
| 3199 | −1 | TGACGTCATCAATAATCTTA | GGG | 1921 |
| 3200 | −1 | TTGACGTCATCAATAATCTT | AGG | 1922 |
| 3231 | 1 | CAAGAGTGTTGAAATCGTTG | AGG | 1923 |
| 3232 | 1 | AAGAGTGTTGAAATCGTTGA | GGG | 1924 |
| 3238 | 1 | GTTGAAATCGTTGAGGGAAA | CGG | 1925 |
| 3241 | 1 | GAAATCGTTGAGGGAAACGG | TGG | 1926 |
| 3247 | 1 | GTTGAGGGAAACGGTGGTCC | TGG | 1927 |
| 3254 | −1 | GTGAGTTTCTTGATGGTTCC | AGG | 1928 |
| 3261 | −1 | GACAATGGTGAGTTTCTTGA | TGG | 1929 |
| 3276 | 1 | CAAGAAACTCACCATTGTCG | AGG | 1930 |
| 3276 | −1 | TAGTTACTGACCCTCGACAA | TGG | 1931 |
| 3277 | 1 | AAGAAACTCACCATTGTCGA | GGG | 1932 |
| 3425 | −1 | ATAGACCGAGATCGTTTACA | TGG | 1933 |
| 3431 | 1 | GATCTCCATGTAAACGATCT | CGG | 1934 |
| 3467 | 1 | ATATACATAACAGAGCACAA | TGG | 1935 |
| 3498 | −1 | CTTGTCCCACTAAATAGGGT | CGG | 1936 |
| 3502 | −1 | AAGTCTTGTCCCACTAAATA | GGG | 1937 |
| 3503 | 1 | ATGTAGCCGACCCTATTTAG | TGG | 1938 |
| 3503 | −1 | AAAGTCTTGTCCCACTAAAT | AGG | 1939 |
| 3504 | 1 | TGTAGCCGACCCTATTTAGT | GGG | 1940 |
| 3811 | 1 | GTATGTACATGAAATAAATA | TGG | 1941 |
| 3812 | 1 | TATGTACATGAAATAAATAT | GGG | 1942 |
| 3855 | 1 | AATGATGATATATATGCAGA | TGG | 1943 |
| 3872 | −1 | CACCTTGTGCAAGATAAACT | TGG | 1944 |
| 3881 | 1 | AACCAAGTTTATCTTGCACA | AGG | 1945 |
| 3884 | 1 | CAAGTTTATCTTGCACAAGG | TGG | 1946 |
| 3899 | 1 | CAAGGTGGAGTCAATAGATG | AGG | 1947 |
| 3911 | −1 | GCTGTAGTTGTATGCATAGT | TGG | 1948 |
| 3930 | 1 | GCATACAACTACAGCGTTGT | TGG | 1949 |
| 3933 | 1 | TACAACTACAGCGTTGTTGG | AGG | 1950 |
| 3938 | 1 | CTACAGCGTTGTTGGAGGAG | TGG | 1951 |
| 3953 | 1 | AGGAGTGGCTCTGCCTCCCA | CGG | 1952 |
| 3955 | −1 | GTTATCTTCTCCGCCGTGGG | AGG | 1953 |
| 3956 | 1 | AGTGGCTCTGCCTCCCACGG | CGG | 1954 |
| 3958 | −1 | AATGTTATCTTCTCCGCCGT | GGG | 1955 |
| 3959 | −1 | AAATGTTATCTTCTCCGCCG | TGG | 1956 |
| 3983 | 1 | GATAACATTTGAGACAAAGC | TGG | 1957 |
| 3990 | 1 | TTTGAGACAAAGCTGGTTGA | AGG | 1958 |
| 3999 | 1 | AAGCTGGTTGAAGGACCCAA | CGG | 1959 |

TABLE 10
gRNA's targeting sequences of Ara h9.

| Position | | | | |
|---|---|---|---|---|
| on SEQ ID | | | | SEQ ID |
| NO: 1960 | Strand | Sequence | PAM | NO: |
| 19 | −1 | TAAGTAGAAGACAGTGTTAA | AGG | 1962 |
| 54 | −1 | TATTGAAACTTATATGAATA | TGG | 1963 |
| 129 | 1 | AAAGAAAACACAAACTTGCA | TGG | 1964 |
| 145 | −1 | CTTTGTCTCTTGTCGATGCT | GGG | 1965 |
| 146 | −1 | ACTTTGTCTCTTGTCGATGC | TGG | 1966 |
| 197 | −1 | CTTCCCCAATATTGCTGTGT | AGG | 1967 |
| 203 | 1 | TAGTTCCTACACAGCAATAT | TGG | 1968 |
| 204 | 1 | AGTTCCTACACAGCAATATT | GGG | 1969 |
| 205 | 1 | GTTCCTACACAGCAATATTG | GGG | 1970 |
| 221 | 1 | ATTGGGGAAGAATTAGTGAA | TGG | 1971 |
| 273 | −1 | ATGTTTATCTAATATTCTCT | TGG | 1972 |
| 292 | 1 | ATATTAGATAAACATAAAAA | AGG | 1973 |
| 293 | 1 | TATTAGATAAACATAAAAAA | GGG | 1974 |
| 302 | 1 | AACATAAAAAAGGGCTGCTT | AGG | 1975 |
| 303 | 1 | ACATAAAAAAGGGCTGCTTA | GGG | 1976 |
| 317 | −1 | GAATTAGAAAGAAGAATGGA | AGG | 1977 |
| 321 | −1 | TCCTGAATTAGAAAGAAGAA | TGG | 1978 |
| 331 | 1 | TCCATTCTTCTTTCTAATTC | AGG | 1979 |
| 339 | 1 | TCTTTCTAATTCAGGATTGC | CGG | 1980 |
| 347 | −1 | AGCGAAAATCAAGATTTTGC | CGG | 1981 |
| 365 | 1 | AATCTTGATTTTCGCTATTT | TGG | 1982 |
| 366 | 1 | ATCTTGATTTTCGCTATTTT | GGG | 1983 |
| 377 | 1 | CGCTATTTTGGGACCTTCTT | TGG | 1984 |
| 379 | −1 | CAGCGTTAAGTCACCAAAGA | AGG | 1985 |
| 402 | 1 | ACTTAACGCTGAATTTACCA | AGG | 1986 |
| 408 | −1 | GTTCAGTTTCTCTGTATCCT | TGG | 1987 |
| 439 | 1 | AACAATAGTGTGAACCTGTG | AGG | 1988 |
| 440 | 1 | ACAATAGTGTGAACCTGTGA | GGG | 1989 |
| 442 | −1 | AATTGGAAGAACTCCCTCAC | AGG | 1990 |
| 459 | −1 | AAGCCCACTCACTAGAAAAT | TGG | 1991 |
| 466 | 1 | TCTTCCAATTTTCTAGTGAG | TGG | 1992 |
| 467 | 1 | CTTCCAATTTTCTAGTGAGT | GGG | 1993 |
| 499 | −1 | ACTTTTACAAGTGAAATTTT | GGG | 1994 |
| 500 | −1 | AACTTTTACAAGTGAAATTT | TGG | 1995 |
| 609 | −1 | TATGTACCGTCTTTGAAATA | CGG | 1996 |
| 614 | 1 | TATATACCGTATTTCAAAGA | CGG | 1997 |
| 663 | 1 | AAACTTATGTTAATTTATGT | TGG | 1998 |
| 722 | 1 | AAAATAAAAAAATAAAATTT | AGG | 1999 |
| 776 | −1 | TATAAAATTCATGATAAAAA | AGG | 2000 |
| 885 | −1 | CGAAGATGCATTAATTTTTT | TGG | 2001 |
| 897 | 1 | CAAAAAAATTAATGCATCTT | CGG | 2002 |
| 922 | 1 | ACTCAATGCTGAATTTACTA | AGG | 2003 |
| 928 | 1 | TGCTGAATTTACTAAGGATA | CGG | 2004 |
| 979 | −1 | AAGCCCACTCACTAGAAAAT | CGG | 2005 |
| 986 | 1 | TCTTCCGATTTTCTAGTGAG | TGG | 2006 |
| 987 | 1 | CTTCCGATTTTCTAGTGAGT | GGG | 2007 |
| 998 | 1 | CTAGTGAGTGGGCTTTACTT | TGG | 2008 |
| 1033 | −1 | TACACAAGTGAAATTTTGGG | CGG | 2009 |
| 1036 | −1 | CAATACACAAGTGAAATTTT | GGG | 2010 |
| 1037 | −1 | ACAATACACAAGTGAAATTT | TGG | 2011 |
| 1161 | 1 | TATATATCATAATTCAAAGA | CGG | 2012 |
| 1176 | 1 | AAAGACGGTACATACTAATT | AGG | 2013 |
| 1183 | 1 | GTACATACTAATTAGGATTA | TGG | 2014 |
| 1195 | 1 | TAGGATTATGGATTTTGACT | CGG | 2015 |
| 1231 | −1 | TTTTAATATTTTTTTTTCTC | TGG | 2016 |
| 1278 | −1 | TATCCGACATAAGTTAATGT | AGG | 2017 |
| 1286 | 1 | AAACCTACATTAACTTATGT | CGG | 2018 |
| 1341 | 1 | AAAATAGAAAAATAAAATTT | AGG | 2019 |
| 1478 | −1 | TTGATAAGATATGCATTAGT | TGG | 2020 |
| 1499 | 1 | CATATCTTATCAATTGAAAA | TGG | 2021 |
| 1503 | 1 | TCTTATCAATTGAAAATGGC | TGG | 2022 |
| 1508 | 1 | TCAATTGAAAATGGCTGGAA | AGG | 2023 |
| 1535 | −1 | TAATAAATTTCAATTTTTAA | GGG | 2024 |
| 1536 | −1 | ATAATAAATTTCAATTTTTA | AGG | 2025 |
| 1563 | 1 | TTATTATTAAAGTTTAAATT | TGG | 2026 |
| 1573 | 1 | AGTTTAAATTTGGAAAAAAT | AGG | 2027 |
| 1590 | −1 | ATCCCTTTCAAAAATTTTAT | AGG | 2028 |
| 1598 | 1 | CTGCCTATAAAATTTTTGAA | AGG | 2029 |
| 1599 | 1 | TGCCTATAAAATTTTTGAAA | GGG | 2030 |
| 1603 | 1 | TATAAAATTTTTGAAAGGGA | TGG | 2031 |
| 1607 | 1 | AAATTTTTGAAAGGGATGGA | AGG | 2032 |
| 1669 | 1 | GATTTCGTTTGATTTCGCCA | CGG | 2033 |
| 1675 | −1 | CACAGACCCAATGCTCACCG | TGG | 2034 |
| 1679 | 1 | GATTTCGCCACGGTGAGCAT | TGG | 2035 |
| 1680 | 1 | ATTTCGCCACGGTGAGCATT | GGG | 2036 |
| 1687 | 1 | CACGGTGAGCATTGGGTCTG | TGG | 2037 |
| 1712 | −1 | TGTCTGTGGCCAATTTAAAT | TGG | 2038 |
| 1714 | 1 | TGAGAGAGTCCAATTTAAAT | TGG | 2039 |
| 1726 | −1 | CCCACGTTTTTGTTTGTCTG | TGG | 2040 |
| 1736 | 1 | GCCACAGACAAACAAAAACG | TGG | 2041 |
| 1737 | 1 | CCACAGACAAACAAAAACGT | GGG | 2042 |
| 1768 | −1 | TGCAACGTTTTACTTTTTCG | TGG | 2043 |
| 1818 | −1 | TACTCACGAACAAGAAGTAG | TGG | 2044 |
| 1840 | 1 | TGTTCGTGAGTATTTTTCCG | TGG | 2045 |
| 1846 | −1 | AGAAACACGGTAGTTTACCA | CGG | 2046 |
| 1859 | 1 | GTGGTAAACTACCGTGTTTC | TGG | 2047 |
| 1859 | −1 | AACTTACACTACCAGAAACA | CGG | 2048 |
| 1872 | 1 | GTGTTTCTGGTAGTGTAAGT | TGG | 2049 |
| 1893 | 1 | GGATATTTCTTGTCATCCTT | TGG | 2050 |
| 1898 | −1 | AACCAGTTCTTCAAATCCAA | AGG | 2051 |
| 1907 | 1 | ATCCTTTGGATTTGAAGAAC | TGG | 2052 |
| 1943 | −1 | AGTCCCGAAAAGAACTTGAA | CGG | 2053 |
| 1950 | 1 | GCATCCGTTCAAGTTCTTTT | CGG | 2054 |
| 1951 | 1 | CATCCGTTCAAGTTCTTTTC | GGG | 2055 |
| 1958 | 1 | TCAAGTTCTTTTCGGGACTT | TGG | 2056 |
| 1961 | 1 | AGTTCTTTTCGGGACTTTGG | TGG | 2057 |
| 1967 | 1 | TTTCGGGACTTTGGTGGATA | TGG | 2058 |
| 1976 | 1 | TTTGGTGGATATGGCGAGCA | AGG | 2059 |
| 2006 | −1 | TCCGGAGGCCAAGTTTCATC | GGG | 2060 |
| 2007 | 1 | TTCCGGAGGCCAAGTTTCAT | CGG | 2061 |
| 2009 | 1 | TCTTTAATCCCGATGAAACT | TGG | 2062 |
| 2016 | 1 | TCCCGATGAAACTTGGCCTC | CGG | 2063 |
| 2021 | −1 | AGACAAATCACTTTTTCCGG | AGG | 2064 |
| 2024 | −1 | GCCAGACAAATCACTTTTTC | CGG | 2065 |
| 2034 | 1 | TCCGGAAAAAGTGATTTGTC | TGG | 2066 |
| 2052 | 1 | TCTGGCATTAACTTCAGAAA | AGG | 2067 |
| 2060 | 1 | TAACTTCAGAAAAGGAGCTT | AGG | 2068 |
| 2097 | 1 | ACCATTTAGAGTAGAGGGAA | GGG | 2069 |
| 2098 | −1 | AACCATTTAGAGTAGAGGGA | AGG | 2070 |
| 2102 | −1 | CAAAAACCATTTAGAGTAGA | GGG | 2071 |
| 2103 | −1 | CCAAAAACCATTTAGAGTAG | AGG | 2072 |
| 2107 | 1 | TCCCTTCCCTCTACTCTAAA | TGG | 2073 |
| 2114 | 1 | CCTCTACTCTAAATGGTTTT | TGG | 2074 |
| 2129 | −1 | TTAAAAGTACCAATGGATGG | GGG | 2075 |
| 2130 | −1 | CTTAAAAGTACCAATGGATG | GGG | 2076 |
| 2131 | 1 | TTTTGGAATCCCCCATCCAT | TGG | 2077 |
| 2131 | −1 | TCTTAAAAGTACCAATGGAT | GGG | 2078 |
| 2132 | −1 | ATCTTAAAAGTACCAATGGA | TGG | 2079 |
| 2136 | −1 | ATTAATCTTAAAAGTACCAA | TGG | 2080 |
| 2167 | 1 | AATTGTGATGCTAGTTATTT | TGG | 2081 |
| 2172 | 1 | TGATGCTAGTTATTTTGGTT | CGG | 2082 |
| 2173 | 1 | GATGCTAGTTATTTTGGTTC | GGG | 2083 |
| 2185 | 1 | TTTGGTTCGGGTGATAGTGT | TGG | 2084 |
| 2215 | 1 | TGTGTTATTAGAGATTGTAA | TGG | 2085 |
| 2216 | 1 | GTGTTATTAGAGATTGTAAT | GGG | 2086 |
| 2222 | 1 | TTAGAGATTGTAATGGGAGC | TGG | 2087 |
| 2228 | 1 | ATTGTAATGGGAGCTGGCAA | AGG | 2088 |
| 2229 | 1 | TTGTAATGGGAGCTGGCAAA | GGG | 2089 |
| 2230 | 1 | TGTAATGGGAGCTGGCAAAG | GGG | 2090 |
| 2231 | 1 | GTAATGGGAGCTGGCAAAGG | GGG | 2091 |
| 2238 | 1 | GAGCTGGCAAAGGGGGTGTT | TGG | 2092 |
| 2239 | 1 | AGCTGGCAAAGGGGGTGTTT | GGG | 2093 |
| 2269 | 1 | GAGAGTAATAATATTCTTCA | AGG | 2094 |
| 2288 | 1 | AAGGAGAATTGTTTGCTATT | TGG | 2095 |
| 2293 | 1 | GAATTGTTTGCTATTTGGAG | AGG | 2096 |
| 2309 | 1 | GGAGAGGATATCTCTTAGCT | TGG | 2097 |
| 2310 | 1 | GAGAGGATATCTCTTAGCTT | GGG | 2098 |
| 2316 | 1 | ATATCTCTTAGCTTGGGATG | TGG | 2099 |
| 2317 | 1 | TATCTCTTAGCTTGGGATGT | GGG | 2100 |
| 2343 | 1 | ACGAGATGTTATTTGTGAGA | CGG | 2101 |
| 2352 | 1 | TATTTGTGAGACGGATTGTG | TGG | 2102 |
| 2380 | 1 | TTTAATCTTGTTACTCAAGA | TGG | 2103 |
| 2386 | 1 | CTTGTTACTCAAGATGGTTT | TGG | 2104 |
| 2387 | 1 | TTGTTACTCAAGATGGTTTT | GGG | 2105 |
| 2403 | 1 | TTTTGGGTTTATTGATCCAC | TGG | 2106 |
| 2408 | −1 | TCTCTTATTTTGAGCACCAG | TGG | 2107 |
| 2432 | 1 | AAATAAGAGATATCATGCAT | TGG | 2108 |
| 2438 | 1 | GAGATATCATGCATTGGAAT | TGG | 2109 |
| 2441 | 1 | ATATCATGCATTGGAATTGG | CGG | 2110 |
| 2442 | 1 | TATCATGCATTGGAATTGGC | GGG | 2111 |
| 2478 | 1 | GATTATGAGAGATGCAAACA | CGG | 2112 |
| 2481 | 1 | TATGAGAGATGCAAACACGG | TGG | 2113 |
| 2493 | 1 | AAACACGGTGGCAGATACTA | TGG | 2114 |
| 2502 | 1 | GGCAGATACTATGGCAAAGA | TGG | 2115 |
| 2529 | 1 | GAAGTTAAAACTTTCGCATG | TGG | 2116 |
| 2546 | 1 | ATGTGGAGTTTCTTTCACCT | TGG | 2117 |
| 2547 | 1 | TGTGGAGTTTCTTTCACCTT | GGG | 2118 |
| 2550 | 1 | GGAGTTTCTTTCACCTTGGG | AGG | 2119 |
| 2552 | −1 | CTACTCTTAAACTCCTCCCA | AGG | 2120 |
| 2573 | 1 | AGTTTAAGAGTAGTCTTAAA | CGG | 2121 |
| 2574 | 1 | GTTTAAGAGTAGTCTTAAAC | GGG | 2122 |
| 2591 | −1 | CAAGGAACTGTCTAAATAGA | TGG | 2123 |
| 2609 | −1 | AAAACAAAACAAACAAAACA | AGG | 2124 |
| 2656 | −1 | CAACTCGAATTAATTTTTTT | TGG | 2125 |
| 2668 | 1 | CAAAAAAAATTAATTCGAGT | TGG | 2126 |
| 2728 | −1 | AAACATTACATGCACAAGTT | GGG | 2127 |
| 2729 | −1 | GAAACATTACATGCACAAGT | TGG | 2128 |
| 2791 | −1 | AATCATTGATTAATTTGTTG | TGG | 2129 |
| 2898 | 1 | TTAAATAAGATAATTTTGAA | TGG | 2130 |
| 2922 | −1 | AATAGGAGAAATCAATAATG | GGG | 2131 |
| 2923 | −1 | GAATAGGAGAAATCAATAAT | GGG | 2132 |
| 2924 | −1 | AGAATAGGAGAAATCAATAA | TGG | 2133 |
| 2939 | −1 | GGTTAATTAGTGAAAAGAAT | AGG | 2134 |
| 2960 | −1 | ATGAGATTATGAGAGTGCTG | TGG | 2135 |
| 2986 | −1 | TTCTCTTTATATAGAAGTGG | GGG | 2136 |
| 2987 | −1 | GTTCTCTTTATATAGAAGTG | GGG | 2137 |
| 2988 | −1 | TGTTCTCTTTATATAGAAGT | GGG | 2138 |
| 2989 | −1 | GTGTTCTCTTTATATAGAAG | TGG | 2139 |
| 3011 | −1 | TGGTGACCTGAAGTGGTGCA | AGG | 2140 |
| 3016 | 1 | AGAACACCTTGCACCACTTC | AGG | 2141 |
| 3018 | −1 | TTGAATTTGGTGACCTGAAG | TGG | 2142 |
| 3031 | −1 | GAGTGTTGAAAGCTTGAATT | TGG | 2143 |
| 3053 | −1 | AATAAAAGCTAAGTGTGTTT | TGG | 2144 |
| 3120 | 1 | CTTGTTCATATTATAATTAA | TGG | 2145 |
| 3136 | −1 | GCATCACAAATGCAAACTTG | AGG | 2146 |
| 3159 | 1 | ATTTGTGATGCTTGTGTGCA | TGG | 2147 |
| 3165 | 1 | GATGCTTGTGTGCATGGCCA | TGG | 2148 |
| 3168 | 1 | GCTTGTGTGCATGGCCATGG | TGG | 2149 |
| 3169 | 1 | CTTGTGTGCATGGCCATGGT | GGG | 2150 |
| 3171 | −1 | CACCATTGGTGCTCCCACCA | TGG | 2151 |
| 3180 | 1 | GGCCATGGTGGGAGCACCAA | TGG | 2152 |
| 3185 | −1 | CATGATATGGCATTCACCAT | TGG | 2153 |
| 3198 | −1 | GTTCACTTGGCCACATGATA | TGG | 2154 |
| 3199 | 1 | ATGGTGAATGCCATATCATG | TGG | 2155 |
| 3211 | −1 | GTGCTAGGGCACTGTTCACT | TGG | 2156 |
| 3225 | −1 | GAAAGGGATGCATGGTGCTA | GGG | 2157 |
| 3226 | −1 | GGAAAGGGATGCATGGTGCT | AGG | 2158 |
| 3233 | −1 | TTTGTGAGGAAAGGGATGCA | TGG | 2159 |
| 3241 | −1 | CTCCACCCTTTGTGAGGAAA | GGG | 2160 |
| 3242 | −1 | GCTCCACCCTTTGTGAGGAA | AGG | 2161 |
| 3246 | 1 | ATGCATCCCTTTCCTCACAA | AGG | 2162 |
| 3247 | 1 | TGCATCCCTTTCCTCACAAA | GGG | 2163 |
| 3247 | −1 | GAGGAGCTCCACCCTTTGTG | AGG | 2164 |
| 3250 | 1 | ATCCCTTTCCTCACAAAGGG | TGG | 2165 |
| 3264 | 1 | AAAGGGTGGAGCTCCTCCTC | CGG | 2166 |
| 3266 | −1 | CCGCTGCAACAAGCCGGAGG | AGG | 2167 |
| 3269 | −1 | ACTCCGCTGCAACAAGCCGG | AGG | 2168 |
| 3272 | −1 | CTAACTCCGCTGCAACAAGC | CGG | 2169 |
| 3277 | 1 | CCTCCTCCGGCTTGTTGCAG | CGG | 2170 |
| 3286 | 1 | GCTTGTTGCAGCGGAGTTAG | AGG | 2171 |
| 3295 | 1 | AGCGGAGTTAGAGGCCTTCT | CGG | 2172 |
| 3298 | −1 | TGGTTCTTAAAGCACCGAGA | AGG | 2173 |
| 3318 | −1 | GGCGGCCTGGCGGTCTGCGG | TGG | 2174 |
| 3321 | −1 | ACAGGCGGCCTGGCGGTCTG | CGG | 2175 |
| 3324 | 1 | AAGAACCACCGCAGACCGCC | AGG | 2176 |
| 3328 | −1 | GGCAGTTACAGGCGGCCTGG | CGG | 2177 |
| 3331 | −1 | TGAGGCAGTTACAGGCGGCC | TGG | 2178 |
| 3336 | −1 | GGCTTTGAGGCAGTTACAGG | CGG | 2179 |
| 3339 | −1 | AGCGGCTTTGAGGCAGTTAC | AGG | 2180 |
| 3349 | −1 | GGGAACCGGCAGCGGCTTTG | AGG | 2181 |
| 3355 | 1 | AACTGCCTCAAAGCCGCTGC | CGG | 2182 |
| 3357 | −1 | GCCACGAAGGGAACCGGCAG | CGG | 2183 |
| 3363 | −1 | GTTGAGGCCACGAAGGGAAC | CGG | 2184 |
| 3367 | 1 | GCCGCTGCCGGTTCCCTTCG | TGG | 2185 |
| 3369 | −1 | GCCTTGGTTGAGGCCACGAA | GGG | 2186 |
| 3370 | −1 | TGCCTTGGTTGAGGCCACGA | AGG | 2187 |
| 3379 | 1 | TCCCTTCGTGGCCTCAACCA | AGG | 2188 |
| 3379 | −1 | CGGCGGCGTTGCCTTGGTTG | AGG | 2189 |
| 3385 | −1 | GGAGGGCGGCGGCGTTGCCT | TGG | 2190 |
| 3396 | −1 | GCATCTTCCAGGGAGGGCGG | CGG | 2191 |
| 3399 | −1 | ACCGCATCTTCCAGGGAGGG | CGG | 2192 |
| 3400 | 1 | GGCAACGCCGCCGCCCTCCC | TGG | 2193 |
| 3402 | −1 | GACACCGCATCTTCCAGGGA | GGG | 2194 |
| 3403 | −1 | TGACACCGCATCTTCCAGGG | AGG | 2195 |
| 3406 | −1 | TGCTGACACCGCATCTTCCA | GGG | 2196 |
| 3407 | −1 | ATGCTGACACCGCATCTTCC | AGG | 2197 |
| 3409 | 1 | GCCGCCCTCCCTGGAAGATG | CGG | 2198 |
| 3431 | −1 | GTGGAGGTGCTGATCTTGTA | AGG | 2199 |
| 3447 | −1 | ATACGTAGCACAGTTGGTGG | AGG | 2200 |
| 3450 | −1 | AACATACGTAGCACAGTTGG | TGG | 2201 |
| 3453 | −1 | AGAAACATACGTAGCACAGT | TGG | 2202 |
| 3492 | −1 | AATTAAGAAATAAATAAAAA | AGG | 2203 |
| 3508 | 1 | TTTATTTATTTCTTAATTTT | TGG | 2204 |
| 3542 | −1 | ATAGTAATACTAATTATTGA | AGG | 2205 |
| 3581 | −1 | AAATTACTAAAAGTTGCTGA | CGG | 2206 |
| 3610 | 1 | AATTTTACTTAGCATTTAAT | CGG | 2207 |

TABLE 11
gRNA's targeting sequences of Ara h10.

| Position | | | | |
|---|---|---|---|---|
| on SEQ ID | | | | SEQ ID |
| NO: 2208 | Strand | Sequence | PAM | NO: |
| 68 | 1 | ACAAACTAACAATTGCAAGT | AGG | 2210 |
| 98 | −1 | ACTAAATAAACTTCTGTTTT | AGG | 2211 |
| 149 | 1 | AGCATTTGAAAGTAAATTTT | TGG | 2212 |
| 150 | 1 | GCATTTGAAAGTAAATTTTT | GGG | 2213 |
| 178 | 1 | TTTAGTAGAGTATTTATCAA | TGG | 2214 |
| 211 | −1 | CAAGGCAAATGATGCTTGAA | CGG | 2215 |
| 229 | −1 | CGTGGGTGACCCATTGGACA | AGG | 2216 |
| 230 | 1 | GCATCATTTGCCTTGTCCAA | TGG | 2217 |
| 231 | 1 | CATCATTTGCCTTGTCCAAT | GGG | 2218 |
| 235 | −1 | CTGCACCGTGGGTGACCCAT | TGG | 2219 |
| 241 | 1 | CTTGTCCAATGGGTCACCCA | CGG | 2220 |
| 246 | −1 | TGATGCTCCCCCTGCACCGT | GGG | 2221 |
| 247 | 1 | CAATGGGTCACCCACGGTGC | AGG | 2222 |
| 247 | −1 | CTGATGCTCCCCCTGCACCG | TGG | 2223 |
| 248 | 1 | AATGGGTCACCCACGGTGCA | GGG | 2224 |
| 249 | 1 | ATGGGTCACCCACGGTGCAG | GGG | 2225 |
| 250 | 1 | TGGGTCACCCACGGTGCAGG | GGG | 2226 |
| 259 | 1 | CACGGTGCAGGGGGAGCATC | AGG | 2227 |
| 268 | 1 | GGGGGAGCATCAGGTGAAAA | CGG | 2228 |
| 269 | 1 | GGGGAGCATCAGGTGAAAAC | GGG | 2229 |
| 273 | 1 | AGCATCAGGTGAAAACGGGC | AGG | 2230 |
| 274 | 1 | GCATCAGGTGAAAACGGGCA | GGG | 2231 |
| 275 | 1 | CATCAGGTGAAAACGGGCAG | GGG | 2232 |
| 290 | 1 | GGCAGGGGAACTTGATTTCG | TGG | 2233 |
| 331 | 1 | ACAAAAGCACAGCCATCAAC | AGG | 2234 |
| 332 | −1 | GGGAAGAAACAGCCTGTTGA | TGG | 2235 |
| 352 | −1 | AGTTGAAAACAAAAGAAAGG | GGG | 2236 |
| 353 | −1 | GAGTTGAAAACAAAAGAAAG | GGG | 2237 |
| 354 | −1 | GGAGTTGAAAACAAAAGAAA | GGG | 2238 |
| 355 | −1 | AGGAGTTGAAAACAAAAGAA | AGG | 2239 |
| 375 | −1 | GTGCTCAACTTCTTGATGAA | AGG | 2240 |
| 406 | −1 | CAACATTCGAGAATGGGATT | GGG | 2241 |
| 407 | −1 | ACAACATTCGAGAATGGGAT | TGG | 2242 |
| 412 | −1 | TGTGGACAACATTCGAGAAT | GGG | 2243 |
| 413 | −1 | ATGTGGACAACATTCGAGAA | TGG | 2244 |
| 430 | −1 | TAAGCCACCAATCTTTCATG | TGG | 2245 |
| 434 | 1 | ATGTTGTCCACATGAAAGAT | TGG | 2246 |
| 437 | 1 | TTGTCCACATGAAAGATTGG | TGG | 2247 |
| 444 | 1 | CATGAAAGATTGGTGGCTTA | TGG | 2248 |
| 449 | 1 | AAGATTGGTGGCTTATGGAT | TGG | 2249 |
| 450 | 1 | AGATTGGTGGCTTATGGATT | GGG | 2250 |
| 451 | 1 | GATTGGTGGCTTATGGATTG | GGG | 2251 |
| 454 | 1 | TGGTGGCTTATGGATTGGGG | AGG | 2252 |
| 466 | −1 | GCTCCCCACAACGGTAAGTG | TGG | 2253 |
| 472 | 1 | GGAGGCCACACTTACCGTTG | TGG | 2254 |
| 473 | 1 | GAGGCCACACTTACCGTTGT | GGG | 2255 |
| 474 | 1 | AGGCCACACTTACCGTTGTG | GGG | 2256 |
| 475 | −1 | GTGCTTCTTGCTCCCCACAA | CGG | 2257 |
| 528 | 1 | AAGTTCTCTTGAAATTTTTC | TGG | 2258 |
| 544 | −1 | TGATATGAGTGTAGAAGGGG | AGG | 2259 |
| 547 | −1 | GCTTGATATGAGTGTAGAAG | GGG | 2260 |
| 548 | −1 | TGCTTGATATGAGTGTAGAA | GGG | 2261 |
| 549 | −1 | GTGCTTGATATGAGTGTAGA | AGG | 2262 |
| 575 | −1 | CAATCTAAATCCAGCAGACA | AGG | 2263 |
| 576 | 1 | CAAGCACATTCCTTGTCTGC | TGG | 2264 |
| 606 | 1 | TTGTTGTAATCAACCTTTTC | AGG | 2265 |
| 608 | −1 | AGAAAATCGTTTTCCTGAAA | AGG | 2266 |
| 655 | 1 | AGTAAAAATCAGTAAAAAAA | TGG | 2267 |
| 724 | 1 | AGAAAATGTATTACCTCCGT | TGG | 2268 |
| 726 | −1 | GCCCTCGGCATAACCAACGG | AGG | 2269 |
| 729 | −1 | GCTGCCCTCGGCATAACCAA | CGG | 2270 |
| 735 | 1 | TACCTCCGTTGGTTATGCCG | AGG | 2271 |
| 736 | 1 | ACCTCCGTTGGTTATGCCGA | GGG | 2272 |
| 741 | −1 | CGGAAAGTAGCAGCTGCCCT | CGG | 2273 |
| 755 | 1 | AGGGCAGCTGCTACTTTCCG | AGG | 2274 |
| 761 | −1 | GAAAAATATACATAACTCCT | CGG | 2275 |
| 777 | 1 | GAGTTATGTATATTTTTCCC | TGG | 2276 |
| 783 | −1 | GGATGCCTGAAAACTCTCCA | GGG | 2277 |
| 784 | −1 | AGGATGCCTGAAAACTCTCC | AGG | 2278 |
| 789 | 1 | TTTTTCCCTGGAGAGTTTTC | AGG | 2279 |
| 804 | −1 | GATCGGTTTGATAAAGAGAA | AGG | 2280 |
| 821 | −1 | TGTTGAAAGAATTGCTTGAT | CGG | 2281 |
| 857 | 1 | GTATTAGAGACATTCATTTT | TGG | 2282 |
| 877 | −1 | AGAAATAGGATTTGGTGCTC | TGG | 2283 |
| 885 | −1 | ATTGTGGAAGAAATAGGATT | TGG | 2284 |
| 891 | −1 | AAAGATATTGTGGAAGAAAT | AGG | 2285 |
| 901 | −1 | TCAAGAAAAGAAAGATATTG | TGG | 2286 |
| 951 | 1 | CTGTTGATATTGCCTTTGTT | TGG | 2287 |
| 952 | −1 | CTATTGCAGATGCCAAACAA | AGG | 2288 |
| 998 | 1 | ACATTTTGAAATAGAATGTG | AGG | 2289 |
| 1041 | 1 | AGATATTTTTCGAATGAAAG | CGG | 2290 |
| 1070 | −1 | AGAAAGCACATTATAACGTA | AGG | 2291 |
| 1105 | −1 | CAAAAAATACAACAAGAAAA | TGG | 2292 |
| 1135 | 1 | TTTGCTGTAATGAAAAATTT | TGG | 2293 |
| 1212 | −1 | ATTTTCACTTCGGTGAGTTG | TGG | 2294 |
| 1222 | −1 | TGCATGAACAATTTTCACTT | CGG | 2295 |
| 1250 | −1 | TTCGGTACCCTTTGTGATTT | TGG | 2296 |
| 1253 | 1 | GCACAGAACCAAAATCACAA | AGG | 2297 |
| 1254 | 1 | CACAGAACCAAAATCACAAA | GGG | 2298 |
| 1268 | −1 | CACATGAACGTTTTTCGGTT | CGG | 2299 |
| 1273 | −1 | TTAACCACATGAACGTTTTT | CGG | 2300 |
| 1280 | 1 | CGAACCGAAAAACGTTCATG | TGG | 2301 |
| 1337 | −1 | TCTTCAGTACTTCTTTTGCA | TGG | 2302 |
| 1396 | −1 | TGTGCTTGTTCTTATTTGTG | TGG | 2303 |
| 1444 | −1 | AATTTGAAAAACTTTTCTTT | TGG | 2304 |
| 1467 | 1 | TTTTTCAAATTTCATCAATT | AGG | 2305 |
| 1497 | 1 | TGAAAATGACATTCGCAGAT | TGG | 2306 |
| 1548 | 1 | ATATCTTTCTTTAAGTATCA | AGG | 2307 |
| 1567 | −1 | TGGATAGATTTCAGGACGAA | TGG | 2308 |
| 1575 | −1 | CATTATATTGGATAGATTTC | AGG | 2309 |
| 1587 | −1 | GTAAAATTGTCTCATTATAT | TGG | 2310 |
| 1608 | 1 | GAGACAATTTTACAGTAGTA | TGG | 2311 |
| 1658 | −1 | GAATTGAATAAGTTTTGTTT | AGG | 2312 |
| 1727 | −1 | AGTTTTGGTTCGGTAGGTTG | TGG | 2313 |
| 1733 | −1 | TGAATGAGTTTTGGTTCGGT | AGG | 2314 |
| 1737 | −1 | CGCATGAATGAGTTTTGGTT | CGG | 2315 |
| 1742 | −1 | TTCTACGCATGAATGAGTTT | TGG | 2316 |
| 1765 | −1 | TTCGGTGGCTTTTGTGATTT | TGG | 2317 |
| 1780 | −1 | ATGAACGTTTTTCAGTTCGG | TGG | 2318 |
| 1783 | −1 | CACATGAACGTTTTTCAGTT | CGG | 2319 |
| 1795 | 1 | CGAACTGAAAAACGTTCATG | TGG | 2320 |
| 1900 | −1 | CGTGCTTGTTCTTATTGGTG | TGG | 2321 |
| 1905 | −1 | ATATACGTGCTTGTTCTTAT | TGG | 2322 |
| 1935 | −1 | CATTCAATTTAGTGTAACTA | GGG | 2323 |
| 1936 | −1 | TCATTCAATTTAGTGTAACT | AGG | 2324 |
| 1973 | 1 | TAACAACAAATTTCATTCCA | AGG | 2325 |
| 1979 | −1 | TGTACAATATAACTAGGCCT | TGG | 2326 |
| 1985 | −1 | ATGATTTGTACAATATAACT | AGG | 2327 |
| 2035 | −1 | TTAGTTTTACTGGTTTGCTT | TGG | 2328 |
| 2045 | −1 | TAGTTGCATTTTAGTTTTAC | TGG | 2329 |
| 2112 | 1 | TAGTAATTTTTTCATACCTT | TGG | 2330 |
| 2117 | −1 | ACAGCAACAAAGACTACCAA | AGG | 2331 |
| 2144 | 1 | TTGCTGTTTTCGTTTGTTTC | TGG | 2332 |
| 2184 | −1 | TGCAGAAACCACTACTAGAA | AGG | 2333 |
| 2187 | 1 | TTCTGATTCCTTTCTAGTAG | TGG | 2334 |
| 2245 | 1 | TTGAGAGAGATTCGAAAAGA | CGG | 2335 |
| 2325 | 1 | AAGTAGTCATTTCATTTGTA | TGG | 2336 |
| 2326 | 1 | AGTAGTCATTTCATTTGTAT | GGG | 2337 |
| 2327 | 1 | GTAGTCATTTCATTTGTATG | GGG | 2338 |
| 2360 | 1 | TCACGTTTATTTATTGAGAT | TGG | 2339 |
| 2361 | 1 | CACGTTTATTTATTGAGATT | GGG | 2340 |
| 2381 | 1 | GGGAAGCGCATTAGTTGTTT | TGG | 2341 |
| 2382 | 1 | GGAAGCGCATTAGTTGTTTT | GGG | 2342 |
| 2387 | 1 | CGCATTAGTTGTTTTGGGTT | TGG | 2343 |
| 2400 | −1 | ATACATCTATGTAATCAAGT | TGG | 2344 |
| 2429 | −1 | TCTATAATAATAATAAAAAC | GGG | 2345 |
| 2430 | −1 | GTCTATAATAATAATAAAAA | CGG | 2346 |
| 2520 | −1 | ATAATTGAGAGAGATTAATT | AGG | 2347 |
| 2607 | −1 | TATAATTATCTGTGTATGTA | AGG | 2348 |
| 2758 | 1 | TATACTTATATTAGTTGTAT | TGG | 2349 |
| 2776 | −1 | TTTGGTGTGCCATTTTCTCG | AGG | 2350 |
| 2778 | 1 | TGGCGTAAACCTCGAGAAAA | TGG | 2351 |
| 2794 | −1 | CTTGAGATCAGGCTTCGCTT | TGG | 2352 |
| 2805 | −1 | ATGGCTACATACTTGAGATC | AGG | 2353 |
| 2824 | −1 | GATTCATTCACATGAGTGCA | TGG | 2354 |
| 2883 | 1 | TACTCTTTTGCAATTGACTG | AGG | 2355 |
| 2895 | −1 | TTAACAGGTTTCAGTGTGAG | TGG | 2356 |
| 2910 | −1 | AAGGGCTTTGCATGGTTAAC | AGG | 2357 |
| 2918 | −1 | TGTTGAGTAAGGGCTTTGCA | TGG | 2358 |
| 2928 | −1 | AGGTGCGAGTTGTTGAGTAA | GGG | 2359 |
| 2929 | −1 | TAGGTGCGAGTTGTTGAGTA | AGG | 2360 |
| 2948 | −1 | TTGCAAGGTGGGTGACACGT | AGG | 2361 |
| 2959 | −1 | GAAAAGTTGGGTTGCAAGGT | GGG | 2362 |
| 2960 | −1 | AGAAAAGTTGGGTTGCAAGG | TGG | 2363 |
| 2963 | −1 | TATAGAAAAGTTGGGTTGCA | AGG | 2364 |
| 2971 | −1 | GTGTTTCTTATAGAAAAGTT | GGG | 2365 |
| 2972 | −1 | TGTGTTTCTTATAGAAAAGT | TGG | 2366 |
| 3009 | −1 | GTTTGTGTATGCATGTGACG | TGG | 2367 |
| 3031 | −1 | AGAAGGAATGATATTTAATT | TGG | 2368 |
| 3048 | −1 | AGGGAGAAGAGGTAAAGAGA | AGG | 2369 |
| 3059 | −1 | AAGCAGGTTTCAGGGAGAAG | AGG | 2370 |
| 3067 | −1 | TCTGAATGAAGCAGGTTTCA | GGG | 2371 |
| 3068 | −1 | CTCTGAATGAAGCAGGTTTC | AGG | 2372 |
| 3075 | −1 | TCTTTTACTCTGAATGAAGC | AGG | 2373 |
| 3095 | 1 | TTCAGAGTAAAAGAAACATA | AGG | 2374 |
| 3104 | 1 | AAAGAAACATAAGGAAGAAA | AGG | 2375 |
| 3105 | 1 | AAGAAACATAAGGAAGAAAA | GGG | 2376 |
| 3122 | −1 | CAGTCATGGCTGATGGTTGC | AGG | 2377 |
| 3129 | −1 | GTACGGTCAGTCATGGCTGA | TGG | 2378 |
| 3136 | −1 | TGGTTGGGTACGGTCAGTCA | TGG | 2379 |
| 3146 | −1 | GGACAGTGTGTGGTTGGGTA | CGG | 2380 |
| 3151 | −1 | GACTTGGACAGTGTGTGGTT | GGG | 2381 |
| 3152 | −1 | GGACTTGGACAGTGTGTGGT | TGG | 2382 |
| 3156 | −1 | GTGTGGACTTGGACAGTGTG | TGG | 2383 |
| 3167 | −1 | GGCCAGCTGTGGTGTGGACT | TGG | 2384 |
| 3173 | −1 | CGAAACGGCCAGCTGTGGTG | TGG | 2385 |
| 3176 | 1 | GTCCAAGTCCACACCACAGC | TGG | 2386 |
| 3178 | −1 | GTCGCCGAAACGGCCAGCTG | TGG | 2387 |
| 3185 | 1 | CACACCACAGCTGGCCGTTT | CGG | 2388 |
| 3188 | −1 | CGGCAGCGGTGTCGCCGAAA | CGG | 2389 |
| 3200 | 1 | CGTTTCGGCGACACCGCTGC | CGG | 2390 |
| 3202 | −1 | ATAGCGGTTAGTTCCGGCAG | CGG | 2391 |
| 3208 | −1 | GTCGGCATAGCGGTTAGTTC | CGG | 2392 |
| 3218 | −1 | ACGGGCCTCTGTCGGCATAG | CGG | 2393 |
| 3224 | 1 | ACTAACCGCTATGCCGACAG | AGG | 2394 |
| 3226 | −1 | AGATGTTGACGGGCCTCTGT | CGG | 2395 |
| 3236 | −1 | CGATAACCTTAGATGTTGAC | GGG | 2396 |
| 3237 | −1 | GCGATAACCTTAGATGTTGA | CGG | 2397 |
| 3241 | 1 | CAGAGGCCCGTCAACATCTA | AGG | 2398 |
| 3259 | −1 | GATAGGGAGTCCAGTGATGA | CGG | 2399 |
| 3260 | 1 | AAGGTTATCGCCGTCATCAC | TGG | 2400 |
| 3272 | 1 | GTCATCACTGGACTCCCTAT | CGG | 2401 |
| 3275 | 1 | ATCACTGGACTCCCTATCGG | CGG | 2402 |
| 3275 | −1 | ATAGCAACGTGCCGCCGATA | GGG | 2403 |
| 3276 | −1 | AATAGCAACGTGCCGCCGAT | AGG | 2404 |
| 3295 | 1 | CGGCACGTTGCTATTGTTCG | CGG | 2405 |
| 3296 | 1 | GGCACGTTGCTATTGTTCGC | GGG | 2406 |
| 3297 | 1 | GCACGTTGCTATTGTTCGCG | GGG | 2407 |
| 3311 | 1 | TTCGCGGGGCTTGCCCTTGC | CGG | 2408 |
| 3313 | −1 | CCCAAGCAGGGTTCCGGCAA | GGG | 2409 |
| 3314 | −1 | GCCCAAGCAGGGTTCCGGCA | AGG | 2410 |
| 3319 | −1 | CGCCAGCCCAAGCAGGGTTC | CGG | 2411 |
| 3323 | 1 | GCCCTTGCCGGAACCCTGCT | TGG | 2412 |
| 3324 | 1 | CCCTTGCCGGAACCCTGCTT | GGG | 2413 |
| 3325 | −1 | GGTCACCGCCAGCCCAAGCA | GGG | 2414 |
| 3326 | −1 | TGGTCACCGCCAGCCCAAGC | AGG | 2415 |
| 3328 | 1 | TGCCGGAACCCTGCTTGGGC | TGG | 2416 |
| 3331 | 1 | CGGAACCCTGCTTGGGCTGG | CGG | 2417 |
| 3346 | −1 | GAAGAGGATGAAAAGCGGGG | TGG | 2418 |
| 3349 | −1 | GCTGAAGAGGATGAAAAGCG | GGG | 2419 |
| 3350 | −1 | GGCTGAAGAGGATGAAAAGC | GGG | 2420 |
| 3351 | −1 | GGGCTGAAGAGGATGAAAAG | CGG | 2421 |
| 3362 | −1 | GAACTATGACAGGGCTGAAG | AGG | 2422 |
| 3371 | −1 | TGGTGGCTGGAACTATGACA | GGG | 2423 |
| 3372 | −1 | ATGGTGGCTGGAACTATGAC | AGG | 2424 |
| 3384 | −1 | AGCCCAACGACAATGGTGGC | TGG | 2425 |
| 3388 | −1 | CGAGAGCCCAACGACAATGG | TGG | 2426 |
| 3391 | −1 | CACCGAGAGCCCAACGACAA | TGG | 2427 |
| 3392 | 1 | GTTCCAGCCACCATTGTCGT | TGG | 2428 |
| 3393 | 1 | TTCCAGCCACCATTGTCGTT | GGG | 2429 |
| 3400 | 1 | CACCATTGTCGTTGGGCTCT | CGG | 2430 |
| 3403 | 1 | CATTGTCGTTGGGCTCTCGG | TGG | 2431 |
| 3406 | 1 | TGTCGTTGGGCTCTCGGTGG | CGG | 2432 |
| 3407 | 1 | GTCGTTGGGCTCTCGGTGGC | GGG | 2433 |
| 3408 | 1 | TCGTTGGGCTCTCGGTGGCG | GGG | 2434 |
| 3422 | 1 | GTGGCGGGGTTCTTGACGTC | AGG | 2435 |
| 3431 | 1 | TTCTTGACGTCAGGTGCATG | TGG | 2436 |
| 3432 | 1 | TCTTGACGTCAGGTGCATGT | GGG | 2437 |
| 3439 | 1 | GTCAGGTGCATGTGGGCTGA | CGG | 2438 |
| 3440 | 1 | TCAGGTGCATGTGGGCTGAC | GGG | 2439 |
| 3441 | 1 | CAGGTGCATGTGGGCTGACG | GGG | 2440 |
| 3459 | 1 | CGGGGCTGTCTTCGTTCTCG | TGG | 2441 |
| 3460 | 1 | GGGGCTGTCTTCGTTCTCGT | GGG | 2442 |
| 3477 | 1 | CGTGGGTCATGAATTACATC | CGG | 2443 |
| 3485 | −1 | GCACCGATCCGTGGGTCTGC | CGG | 2444 |
| 3488 | 1 | AATTACATCCGGCAGACCCA | CGG | 2445 |
| 3493 | 1 | CATCCGGCAGACCCACGGAT | CGG | 2446 |
| 3493 | −1 | CTGCTCCGGCACCGATCCGT | GGG | 2447 |
| 3494 | −1 | GCTGCTCCGGCACCGATCCG | TGG | 2448 |
| 3499 | 1 | GCAGACCCACGGATCGGTGC | CGG | 2449 |
| 3507 | −1 | TTTGCCATTTCCAGCTGCTC | CGG | 2450 |
| 3508 | 1 | CGGATCGGTGCCGGAGCAGC | TGG | 2451 |
| 3514 | 1 | GGTGCCGGAGCAGCTGGAAA | TGG | 2452 |
| 3529 | 1 | GGAAATGGCAAAGCACCGCA | TGG | 2453 |
| 3533 | −1 | AACCGGCCACGTCAGCCATG | CGG | 2454 |
| 3538 | 1 | AAAGCACCGCATGGCTGACG | TGG | 2455 |
| 3542 | 1 | CACCGCATGGCTGACGTGGC | CGG | 2456 |
| 3550 | −1 | CGTCTTCTGTCCAACGTAAC | CGG | 2457 |
| 3551 | 1 | GCTGACGTGGCCGGTTACGT | TGG | 2458 |
| 3565 | 1 | TTACGTTGGACAGAAGACGA | AGG | 2459 |
| 3572 | 1 | GGACAGAAGACGAAGGATGT | AGG | 2460 |
| 3586 | 1 | GGATGTAGGACAGAAGACCA | AGG | 2461 |
| 3592 | −1 | TATCTCTTGCCCAACTTCCT | TGG | 2462 |
| 3593 | 1 | GGACAGAAGACCAAGGAAGT | TGG | 2463 |
| 3594 | 1 | GACAGAAGACCAAGGAAGTT | GGG | 2464 |
| 3613 | 1 | TGGGCAAGAGATACAGACCA | AGG | 2465 |
| 3619 | 1 | AGAGATACAGACCAAGGCTC | AGG | 2466 |
| 3619 | −1 | TCTCTTTGAATCCTGAGCCT | TGG | 2467 |
| 3647 | 1 | AAGAGAACTTGATAGAATAG | TGG | 2468 |
| 3660 | 1 | AGAATAGTGGTTTAAGCTTA | AGG | 2469 |
| 3668 | 1 | GGTTTAAGCTTAAGGATGAA | AGG | 2470 |
| 3669 | 1 | GTTTAAGCTTAAGGATGAAA | GGG | 2471 |
| 3670 | 1 | TTTAAGCTTAAGGATGAAAG | GGG | 2472 |
| 3678 | 1 | TAAGGATGAAAGGGGTCTAA | TGG | 2473 |
| 3679 | 1 | AAGGATGAAAGGGGTCTAAT | GGG | 2474 |
| 3688 | 1 | AGGGGTCTAATGGGTTTTGA | TGG | 2475 |

TABLE 12
gRNA's targeting sequences of Ara h11.

| Position | | | | SEQ |
|---|---|---|---|---|
| on SEQ ID | | | | ID |
| NO: 2476 | Strand | Sequence | PAM | NO: |
| 120 | −1 | AAAAATGAATTCAAGTCGCT | TGG | 2478 |
| 170 | −1 | TTTACCTTTTTTATATAAAG | GGG | 2479 |
| 171 | −1 | ATTTACCTTTTTTATATAAA | GGG | 2480 |
| 172 | −1 | TATTTACCTTTTTTATATAA | AGG | 2481 |
| 177 | 1 | TTGACCCCTTTATATAAAAA | AGG | 2482 |
| 209 | 1 | TAGTGTCTAACTTATTAAAA | AGG | 2483 |
| 295 | 1 | AATTTAATCACAACAATAAA | AGG | 2484 |
| 309 | −1 | TTTTCTTTTGGTAGATTTTA | TGG | 2485 |
| 321 | −1 | CATAACTTTTTTTTTTCTTT | TGG | 2486 |
| 372 | 1 | GTTTTTGAAAAATATTTAAT | AGG | 2487 |
| 384 | −1 | AAATTAATTAAAAATTAAAA | TGG | 2488 |
| 426 | 1 | CAAATTGTTGTCCAAATAAG | AGG | 2489 |
| 426 | −1 | TTAGATAAGTCCCTCTTATT | TGG | 2490 |
| 427 | 1 | AAATTGTTGTCCAAATAAGA | GGG | 2491 |
| 457 | 1 | TAAGAATTTTAAAAATTATT | AGG | 2492 |
| 550 | 1 | GAAATTAATTTGTTTAAGAA | TGG | 2493 |
| 551 | 1 | AAATTAATTTGTTTAAGAAT | GGG | 2494 |
| 552 | 1 | AATTAATTTGTTTAAGAATG | GGG | 2495 |
| 553 | 1 | ATTAATTTGTTTAAGAATGG | GGG | 2496 |
| 559 | 1 | TTGTTTAAGAATGGGGGTAT | TGG | 2497 |
| 672 | −1 | TAAAATATTTAGAATGAATT | AGG | 2498 |
| 722 | 1 | TTTAGTAATGATAATAATAA | TGG | 2499 |
| 788 | 1 | TTAATTTAGTTATATAGCTA | AGG | 2500 |
| 848 | −1 | TCATATGATTAGTAATTTAT | AGG | 2501 |
| 885 | −1 | TATCACTAGCTAGTTATAAA | TGG | 2502 |
| 933 | −1 | TATCTTTCGCATTGAAAAAG | TGG | 2503 |
| 990 | 1 | CATTAGAAAACAAATTATGA | AGG | 2504 |
| 1039 | 1 | TATCACTTACTAAAACCGAA | AGG | 2505 |
| 1043 | −1 | ATGTATAGGAATATTCCTTT | CGG | 2506 |
| 1057 | −1 | ATCCCATAACATGCATGTAT | AGG | 2507 |
| 1065 | 1 | ATTCCTATACATGCATGTTA | TGG | 2508 |
| 1066 | 1 | TTCCTATACATGCATGTTAT | GGG | 2509 |
| 1084 | −1 | ATATATATATAAAACAACAT | TGG | 2510 |
| 1115 | −1 | TTTACTATTTTTCAAAAGGT | AGG | 2511 |
| 1119 | −1 | TTACTTTACTATTTTTCAAA | AGG | 2512 |
| 1151 | −1 | TTATATACGTAGTTTAATTT | TGG | 2513 |
| 1170 | 1 | AAACTACGTATATAAAAAGA | AGG | 2514 |
| 1186 | −1 | TCTATGTGCTTTTATTCTTT | TGG | 2515 |
| 1241 | 1 | TAAAAATACTGTTTATTAAA | TGG | 2516 |
| 1288 | −1 | AATATTATCTCAAAAGATTA | AGG | 2517 |
| 1372 | 1 | TATTTCGTTTACAGTGAGAA | TGG | 2518 |
| 1427 | 1 | CTCTTGTAGCTAATGACTAA | TGG | 2519 |
| 1432 | 1 | GTAGCTAATGACTAATGGAA | AGG | 2520 |
| 1439 | 1 | ATGACTAATGGAAAGGTTGT | TGG | 2521 |
| 1442 | 1 | ACTAATGGAAAGGTTGTTGG | TGG | 2522 |
| 1460 | −1 | TTTTTGAATTTGTCACAGCA | CGG | 2523 |
| 1514 | −1 | TAATAATAGCTTTAGGCGAT | GGG | 2524 |
| 1515 | −1 | TTAATAATAGCTTTAGGCGA | TGG | 2525 |
| 1521 | −1 | GCAGGTTTAATAATAGCTTT | AGG | 2526 |
| 1539 | −1 | TCATTCACAAATTCTTCAGC | AGG | 2527 |
| 1568 | 1 | AATGAAAAAACTCCAACACT | TGG | 2528 |
| 1569 | −1 | TTCATAAACTTACCAAGTGT | TGG | 2529 |
| 1604 | −1 | CGTTCTTAACGTAATAAGCA | TGG | 2530 |
| 1616 | 1 | CATGCTTATTACGTTAAGAA | CGG | 2531 |
| 1633 | 1 | GAACGGTATGAAAATTGAAG | AGG | 2532 |
| 1662 | −1 | TTAATTTGTGGAACATATTA | TGG | 2533 |
| 1674 | −1 | TACATAGATCATTTAATTTG | TGG | 2534 |
| 1689 | 1 | AAATTAAATGATCTATGTAG | TGG | 2535 |
| 1692 | 1 | TTAAATGATCTATGTAGTGG | TGG | 2536 |
| 1812 | −1 | CTACATGAAGGTGAACTTTT | AGG | 2537 |
| 1824 | −1 | ACCCAAGCACCTCTACATGA | AGG | 2538 |
| 1826 | 1 | AAAAGTTCACCTTCATGTAG | AGG | 2539 |
| 1833 | 1 | CACCTTCATGTAGAGGTGCT | TGG | 2540 |
| 1834 | 1 | ACCTTCATGTAGAGGTGCTT | GGG | 2541 |
| 1921 | −1 | CTTGCATGCAAATTCAGATT | TGG | 2542 |
| 1950 | −1 | TACCATTAGACAAATTAATT | TGG | 2543 |
| 1959 | 1 | TGCCAAATTAATTTGTCTAA | TGG | 2544 |
| 1975 | 1 | CTAATGGTATTGTAGCAATG | TGG | 2545 |
| 1993 | 1 | TGTGGTATTTTATTTTCAGT | AGG | 2546 |
| 2019 | 1 | GTTCATAAAAAAATATAAAA | TGG | 2547 |
| 2027 | 1 | AAAAATATAAAATGGTTAAT | TGG | 2548 |
| 2052 | −1 | AAATCAGTTATTTTAAAATT | TGG | 2549 |
| 2086 | −1 | TAAAATTAGTTATAGTTTAT | GGG | 2550 |
| 2087 | −1 | CTAAAATTAGTTATAGTTTA | TGG | 2551 |
| 2105 | 1 | CTATAACTAATTTTAGAAAG | TGG | 2552 |
| 2136 | −1 | TAATCTGTCGATTTACAAAC | TGG | 2553 |
| 2162 | 1 | ACAGATTAACAAAACAAAAT | CGG | 2554 |
| 2316 | 1 | AAATTAATTTTGTATAAAAT | TGG | 2555 |
| 2344 | −1 | TTTGTAAAAAAATTTACTTG | AGG | 2556 |
| 2391 | −1 | AATTATCAAATTTTTTATGA | TGG | 2557 |
| 2411 | 1 | AAAATTTGATAATTTTACAT | TGG | 2558 |
| 2482 | 1 | AAGTAATAAAGATCAATCTT | TGG | 2559 |
| 2602 | −1 | CCAAAAAATATATATTTTAG | TGG | 2560 |
| 2613 | 1 | CCACTAAAATATATATTTTT | TGG | 2561 |
| 2682 | −1 | TTCTAATAATTAATTTTAAT | AGG | 2562 |
| 2740 | 1 | AATTATATATATATATATGC | AGG | 2563 |
| 2822 | −1 | TTTTCTTTTCTTTTCTTTGT | TGG | 2564 |
| 2835 | 1 | AACAAAGAAAAGAAAAGAAA | AGG | 2565 |
| 2836 | 1 | ACAAAGAAAAGAAAAGAAAA | GGG | 2566 |
| 2842 | 1 | AAAAGAAAAGAAAAGGGAAA | AGG | 2567 |
| 2869 | 1 | CGTGTCATGAAACCCAGAAA | TGG | 2568 |
| 2870 | 1 | GTGTCATGAAACCCAGAAAT | GGG | 2569 |
| 2870 | −1 | TGCATGCAAAACCCATTTCT | GGG | 2570 |
| 2871 | −1 | CTGCATGCAAAACCCATTTC | TGG | 2571 |
| 2885 | 1 | GAAATGGGTTTTGCATGCAG | CGG | 2572 |
| 2911 | −1 | GGGTGTCGTGGCACGTGTTG | AGG | 2573 |
| 2923 | −1 | AATTCACAAGGTGGGTGTCG | TGG | 2574 |
| 2931 | −1 | ATCTTCACAATTCACAAGGT | GGG | 2575 |
| 2932 | −1 | AATCTTCACAATTCACAAGG | TGG | 2576 |
| 2935 | −1 | GGCAATCTTCACAATTCACA | AGG | 2577 |
| 2952 | 1 | GAATTGTGAAGATTGCCACC | CGG | 2578 |
| 2956 | −1 | AGGAGATAGGTGTCTCCGGG | TGG | 2579 |
| 2959 | −1 | AGGAGGAGATAGGTGTCTCC | GGG | 2580 |
| 2960 | −1 | GAGGAGGAGATAGGTGTCTC | CGG | 2581 |
| 2969 | −1 | AATTGGGGAGAGGAGGAGAT | AGG | 2582 |
| 2976 | −1 | GGTTTTAAATTGGGGAGAGG | AGG | 2583 |
| 2979 | −1 | GTGGGTTTTAAATTGGGGAG | AGG | 2584 |
| 2984 | −1 | AAAATGTGGGTTTTAAATTG | GGG | 2585 |
| 2985 | −1 | GAAAATGTGGGTTTTAAATT | GGG | 2586 |
| 2986 | −1 | GGAAAATGTGGGTTTTAAAT | TGG | 2587 |
| 2997 | −1 | GAATAGAGAGCGGAAAATGT | GGG | 2588 |
| 2998 | −1 | TGAATAGAGAGCGGAAAATG | TGG | 2589 |
| 3007 | −1 | TAATGCGAATGAATAGAGAG | CGG | 2590 |
| 3037 | −1 | AATTATGAAGTGAAATGAAA | GGG | 2591 |
| 3038 | −1 | TAATTATGAAGTGAAATGAA | AGG | 2592 |
| 3062 | 1 | TTCATAATTAATTAATAACA | TGG | 2593 |
| 3081 | 1 | ATGGCTGAAGCACTCTACTA | CGG | 2594 |
| 3084 | 1 | GCTGAAGCACTCTACTACGG | CGG | 2595 |
| 3096 | −1 | TTGGTTGCTCTTGGCGTTGG | CGG | 2596 |
| 3099 | −1 | ACCTTGGTTGCTCTTGGCGT | TGG | 2597 |
| 3105 | −1 | GGGTGGACCTTGGTTGCTCT | TGG | 2598 |
| 3109 | 1 | GCCAACGCCAAGAGCAACCA | AGG | 2599 |
| 3115 | −1 | TTGACAAGCTGGGTGGACCT | TGG | 2600 |
| 3122 | −1 | GGTGGCCTTGACAAGCTGGG | TGG | 2601 |
| 3125 | −1 | GGTGGTGGCCTTGACAAGCT | GGG | 2602 |
| 3126 | −1 | CGGTGGTGGCCTTGACAAGC | TGG | 2603 |
| 3128 | 1 | AAGGTCCACCCAGCTTGTCA | AGG | 2604 |
| 3140 | −1 | TCCGGCGACAACAGCGGTGG | TGG | 2605 |
| 3143 | −1 | GCCTCCGGCGACAACAGCGG | TGG | 2606 |
| 3146 | −1 | GGAGCCTCCGGCGACAACAG | CGG | 2607 |
| 3150 | 1 | GCCACCACCGCTGTTGTCGC | CGG | 2608 |
| 3153 | 1 | ACCACCGCTGTTGTCGCCGG | AGG | 2609 |
| 3158 | −1 | GAGGATCAAGAGGGAGCCTC | CGG | 2610 |
| 3167 | −1 | AAGGCCGGCGAGGATCAAGA | GGG | 2611 |
| 3168 | −1 | CAAGGCCGGCGAGGATCAAG | AGG | 2612 |
| 3174 | 1 | GGCTCCCTCTTGATCCTCGC | CGG | 2613 |
| 3177 | −1 | CGGCCAGCACAAGGCCGGCG | AGG | 2614 |
| 3182 | −1 | GGTGCCGGCCAGCACAAGGC | CGG | 2615 |
| 3185 | 1 | GATCCTCGCCGGCCTTGTGC | TGG | 2616 |
| 3186 | −1 | TGACGGTGCCGGCCAGCACA | AGG | 2617 |
| 3189 | 1 | CTCGCCGGCCTTGTGCTGGC | CGG | 2618 |
| 3197 | −1 | TGTGAGGCCAATGACGGTGC | CGG | 2619 |
| 3201 | 1 | GTGCTGGCCGGCACCGTCAT | TGG | 2620 |
| 3203 | −1 | GATCGTTGTGAGGCCAATGA | CGG | 2621 |
| 3213 | −1 | AGAGCGGTGTGATCGTTGTG | AGG | 2622 |
| 3229 | −1 | GGGCTGAAGATCACGAAGAG | CGG | 2623 |
| 3242 | 1 | GCTCTTCGTGATCTTCAGCC | CGG | 2624 |
| 3249 | −1 | TGACAGCTGGCACAAGCACC | GGG | 2625 |
| 3250 | −1 | ATGACAGCTGGCACAAGCAC | CGG | 2626 |
| 3262 | −1 | AGTGCCACAGTGATGACAGC | TGG | 2627 |
| 3269 | 1 | TGTGCCAGCTGTCATCACTG | TGG | 2628 |
| 3279 | 1 | GTCATCACTGTGGCACTCTT | AGG | 2629 |
| 3284 | 1 | CACTGTGGCACTCTTAGGCT | TGG | 2630 |
| 3285 | 1 | ACTGTGGCACTCTTAGGCTT | GGG | 2631 |
| 3286 | 1 | CTGTGGCACTCTTAGGCTTG | GGG | 2632 |
| 3293 | 1 | ACTCTTAGGCTTGGGGTTCT | TGG | 2633 |
| 3300 | 1 | GGCTTGGGGTTCTTGGCCTC | TGG | 2634 |
| 3303 | 1 | TTGGGGTTCTTGGCCTCTGG | AGG | 2635 |
| 3305 | −1 | CGCCACGCCGAAGCCTCCAG | AGG | 2636 |
| 3309 | 1 | TTCTTGGCCTCTGGAGGCTT | CGG | 2637 |
| 3314 | 1 | GGCCTCTGGAGGCTTCGGCG | TGG | 2638 |
| 3317 | 1 | CTCTGGAGGCTTCGGCGTGG | CGG | 2639 |
| 3337 | 1 | CGGCAATAACAGTGCTGACG | TGG | 2640 |
| 3346 | 1 | CAGTGCTGACGTGGATCTAT | AGG | 2641 |
| 3357 | 1 | TGGATCTATAGGTACGTAAC | AGG | 2642 |
| 3372 | 1 | GTAACAGGTAAGCATCCACC | TGG | 2643 |
| 3376 | −1 | TCCAATTGGTTGGCGCCAGG | TGG | 2644 |
| 3379 | −1 | GTGTCCAATTGGTTGGCGCC | AGG | 2645 |
| 3386 | 1 | TCCACCTGGCGCCAACCAAT | TGG | 2646 |
| 3386 | −1 | GCGGGCTGTGTCCAATTGGT | TGG | 2647 |
| 3390 | −1 | TGTGGCGGGCTGTGTCCAAT | TGG | 2648 |
| 3404 | −1 | CTTGCCCATCAGCTTGTGGC | GGG | 2649 |
| 3405 | −1 | CCTTGCCCATCAGCTTGTGG | CGG | 2650 |
| 3408 | −1 | GCGCCTTGCCCATCAGCTTG | TGG | 2651 |
| 3410 | 1 | CACAGCCCGCCACAAGCTGA | TGG | 2652 |
| 3411 | 1 | ACAGCCCGCCACAAGCTGAT | GGG | 2653 |
| 3416 | 1 | CCGCCACAAGCTGATGGGCA | AGG | 2654 |
| 3431 | 1 | GGGCAAGGCGCGTGAGATTA | AGG | 2655 |
| 3438 | 1 | GCGCGTGAGATTAAGGACTT | TGG | 2656 |
| 3456 | 1 | TTTGGTCAACAACAAACCAG | TGG | 2657 |
| 3457 | 1 | TTGGTCAACAACAAACCAGT | GGG | 2658 |
| 3458 | 1 | TGGTCAACAACAAACCAGTG | GGG | 2659 |
| 3461 | −1 | TCAAGAAGCCTGGGCCCCAC | TGG | 2660 |
| 3464 | 1 | ACAACAAACCAGTGGGGCCC | AGG | 2661 |
| 3470 | −1 | ATGGTATGCTCAAGAAGCCT | GGG | 2662 |
| 3471 | −1 | GATGGTATGCTCAAGAAGCC | TGG | 2663 |
| 3489 | −1 | AACAAAGATGCAAACGAAGA | TGG | 2664 |

TABLE 13
gRNA's targeting sequences of Ara h14.

| Position | | | | SEQ |
|---|---|---|---|---|
| on SEQ ID | | | | ID |
| NO: 2665 | Strand | Sequence | PAM | NO: |
| 41 | 1 | TAGTAGTAAATTATGAAATA | TGG | 2667 |
| 408 | 1 | ACAATTTTGTAAAAAAAAAA | AGG | 2668 |
| 412 | 1 | TTTTGTAAAAAAAAAAAGGT | TGG | 2669 |
| 459 | −1 | GAATTAGAGAAATGATCTAT | TGG | 2670 |
| 490 | −1 | ACCTGTATGTCTGAGCGTGG | AGG | 2671 |
| 493 | −1 | TACACCTGTATGTCTGAGCG | TGG | 2672 |
| 500 | 1 | GCCTCCACGCTCAGACATAC | AGG | 2673 |
| 612 | 1 | TAAAAGTTTTTTACAAACAG | TGG | 2674 |
| 739 | 1 | TTCTTGATAATTATGTTAAA | AGG | 2675 |
| 774 | 1 | CTAAAAAATCACATTCAATC | CGG | 2676 |
| 782 | −1 | AATACAAAAAAATAAAAAAC | CGG | 2677 |
| 800 | 1 | TTTATTTTTTTGTATTGATT | AGG | 2678 |
| 819 | −1 | AAAAAAGTTAATATTTTTTT | TGG | 2679 |
| 839 | 1 | ATATTAACTTTTTTAGTACA | AGG | 2680 |
| 926 | 1 | TATTTTTTGAAATAATTGTC | AGG | 2681 |
| 960 | 1 | ATTTTTTTAAAAAATAATTG | TGG | 2682 |
| 963 | 1 | TTTTTAAAAAATAATTGTGG | AGG | 2683 |
| 983 | −1 | TCTTTTATGTCTATAATAAA | TGG | 2684 |
| 1043 | −1 | AGATAACGTAAGGTTAATTA | TGG | 2685 |
| 1053 | −1 | TTGCTAGGTTAGATAACGTA | AGG | 2686 |
| 1068 | −1 | CTCCGAGTTCATCATTTGCT | AGG | 2687 |
| 1077 | 1 | AACCTAGCAAATGATGAACT | CGG | 2688 |
| 1094 | −1 | AATTATATATGGGACTCTTA | TGG | 2689 |
| 1104 | −1 | TAAAATTATAAATTATATAT | GGG | 2690 |
| 1105 | −1 | ATAAAATTATAAATTATATA | TGG | 2691 |
| 1165 | −1 | AACAGGTGCTCTTTTCTGCT | TGG | 2692 |
| 1182 | −1 | TGTTCGCATTACAAGGAAAC | AGG | 2693 |
| 1189 | −1 | GTTGTGTTGTTCGCATTACA | AGG | 2694 |
| 1260 | −1 | GGCCTTTTCTTGTTGAATGG | AGG | 2695 |
| 1263 | −1 | TGTGGCCTTTTCTTGTTGAA | TGG | 2696 |
| 1269 | 1 | CACCTCCATTCAACAAGAAA | AGG | 2697 |
| 1281 | −1 | TTGAAACTTGGGTTTGGCTG | TGG | 2698 |
| 1287 | −1 | CAAATTTTGAAACTTGGGTT | TGG | 2699 |
| 1292 | −1 | AAAATCAAATTTTGAAACTT | GGG | 2700 |
| 1293 | −1 | GAAAATCAAATTTTGAAACT | TGG | 2701 |
| 1315 | −1 | TTAATGATTATTAAATAGAT | AGG | 2702 |
| 1345 | 1 | TTAATTTAATTTCCCTTTGC | AGG | 2703 |
| 1346 | −1 | TCGGATGTATGTCCTGCAAA | GGG | 2704 |
| 1347 | −1 | ATCGGATGTATGTCCTGCAA | AGG | 2705 |
| 1365 | −1 | ATAACTTAATTATTTTTTAT | CGG | 2706 |
| 1458 | 1 | ATAAATAAATATCCAATTGT | TGG | 2707 |
| 1459 | −1 | ATATTAAGTTATCCAACAAT | TGG | 2708 |
| 1511 | −1 | AGTTATGGAGAGGTTAAACG | GGG | 2709 |
| 1512 | −1 | TAGTTATGGAGAGGTTAAAC | GGG | 2710 |
| 1513 | −1 | ATAGTTATGGAGAGGTTAAA | CGG | 2711 |
| 1521 | −1 | TGAAGATCATAGTTATGGAG | AGG | 2712 |
| 1526 | −1 | TAAAGTGAAGATCATAGTTA | TGG | 2713 |
| 1570 | −1 | TTGAAGAACTCAAATCTACA | AGG | 2714 |
| 1590 | 1 | TTTGAGTTCTTCAAGCTATT | TGG | 2715 |
| 1613 | −1 | CCCTTCTTTTTTAAAATTAC | AGG | 2716 |
| 1623 | 1 | TCCTGTAATTTTAAAAAAGA | AGG | 2717 |
| 1624 | 1 | CCTGTAATTTTAAAAAAGAA | GGG | 2718 |
| 1663 | −1 | CACCATAAAGTCAATTTATT | TGG | 2719 |
| 1672 | 1 | AACCAAATAAATTGACTTTA | TGG | 2720 |
| 1693 | 1 | GGTGACAAAGATGAAAACAT | AGG | 2721 |
| 1713 | −1 | ATATATGCATTGATAAATTG | TGG | 2722 |
| 1749 | −1 | TAAAAGTTTTGCTATTACAA | AGG | 2723 |
| 1851 | −1 | ATATTCATATTTAATGGATT | GGG | 2724 |
| 1852 | −1 | AATATTCATATTTAATGGAT | TGG | 2725 |
| 1857 | −1 | ACTCTAATATTCATATTTAA | TGG | 2726 |
| 1887 | −1 | TTGGAAGAAGTAAAAAGATG | CGG | 2727 |
| 1906 | −1 | TCATTTTTACATAGAGATGT | TGG | 2728 |
| 2204 | 1 | TTGTTAGCATGATACGATGT | TGG | 2729 |
| 2205 | 1 | TGTTAGCATGATACGATGTT | GGG | 2730 |
| 2240 | 1 | AATAACATAAAATAAAATAA | AGG | 2731 |
| 2422 | −1 | ATAAAATGCACAAAAATGGA | AGG | 2732 |
| 2426 | −1 | CTTCATAAAATGCACAAAAA | TGG | 2733 |
| 2438 | 1 | CATTTTTGTGCATTTTATGA | AGG | 2734 |
| 2439 | 1 | ATTTTTGTGCATTTTATGAA | GGG | 2735 |
| 2476 | 1 | AGTGTTACTTTCTTAAACAT | AGG | 2736 |
| 2499 | 1 | AACATAATGAAACGATTGAA | AGG | 2737 |
| 2518 | −1 | GATAAAGGATTTTCATGATG | AGG | 2738 |
| 2533 | −1 | TTCGTTTATTAGAAGGATAA | AGG | 2739 |
| 2540 | −1 | AACATCTTTCGTTTATTAGA | AGG | 2740 |
| 2839 | −1 | GTTGGATTTAATTTTAAGTG | AGG | 2741 |
| 2857 | −1 | ATATTTTTTAAGGTGAAAGT | TGG | 2742 |
| 2867 | −1 | GTGTAACTAGATATTTTTTA | AGG | 2743 |
| 2896 | 1 | TACACTGTTAATAAATTACT | AGG | 2744 |
| 2902 | 1 | GTTAATAAATTACTAGGCAC | CGG | 2745 |
| 2910 | −1 | CGTGCTTGTGACATGCATGC | CGG | 2746 |
| 2950 | −1 | GTGGTGGGTTTGGAGAACGG | TGG | 2747 |
| 2953 | −1 | GAGGTGGTGGGTTTGGAGAA | CGG | 2748 |
| 2960 | −1 | GTTTGATGAGGTGGTGGGTT | TGG | 2749 |
| 2965 | −1 | GATGAGTTTGATGAGGTGGT | GGG | 2750 |
| 2966 | −1 | GGATGAGTTTGATGAGGTGG | TGG | 2751 |
| 2969 | −1 | TGTGGATGAGTTTGATGAGG | TGG | 2752 |
| 2972 | −1 | GCTTGTGGATGAGTTTGATG | AGG | 2753 |
| 2987 | −1 | GACATTGATATCGGTGCTTG | TGG | 2754 |
| 2996 | −1 | GAGTGGGAAGACATTGATAT | CGG | 2755 |
| 3012 | −1 | GAAGATTAATAATGGAGAGT | GGG | 2756 |
| 3013 | −1 | TGAAGATTAATAATGGAGAG | TGG | 2757 |
| 3020 | −1 | AGGTTTTTGAAGATTAATAA | TGG | 2758 |
| 3035 | 1 | TATTAATCTTCAAAAACCTA | AGG | 2759 |
| 3040 | −1 | TTTGTTCTCTTTTCTTCCTT | AGG | 2760 |
| 3054 | 1 | AAGGAAGAAAAGAGAACAAA | AGG | 2761 |
| 3067 | 1 | GAACAAAAGGAATCCCAAAA | AGG | 2762 |
| 3068 | 1 | AACAAAAGGAATCCCAAAAA | GGG | 2763 |
| 3069 | −1 | GAAAAGAAAAGGCCCTTTTT | GGG | 2764 |
| 3070 | −1 | AGAAAAGAAAAGGCCCTTTT | TGG | 2765 |
| 3080 | −1 | GATTGTTAAGAGAAAAGAAA | AGG | 2766 |
| 3106 | 1 | TAACAATCAAACAACAGCAA | TGG | 2767 |
| 3136 | 1 | TACTGATCGTGCACCTCACC | AGG | 2768 |
| 3138 | −1 | GTGTGAACTTGAACCTGGTG | AGG | 2769 |
| 3143 | −1 | TGGGGGTGTGAACTTGAACC | TGG | 2770 |
| 3160 | −1 | GTCGACGCGTTGTGTGGTGG | GGG | 2771 |
| 3161 | −1 | CGTCGACGCGTTGTGTGGTG | GGG | 2772 |
| 3162 | −1 | ACGTCGACGCGTTGTGTGGT | GGG | 2773 |
| 3163 | −1 | AACGTCGACGCGTTGTGTGG | TGG | 2774 |
| 3166 | −1 | TGGAACGTCGACGCGTTGTG | TGG | 2775 |
| 3185 | 1 | CGCGTCGACGTTCCACGCCG | CGG | 2776 |
| 3186 | −1 | CTAACATCGTAGCCGCGGCG | TGG | 2777 |
| 3191 | −1 | CACCACTAACATCGTAGCCG | CGG | 2778 |
| 3200 | 1 | CGCCGCGGCTACGATGTTAG | TGG | 2779 |
| 3203 | 1 | CGCGGCTACGATGTTAGTGG | TGG | 2780 |
| 3206 | 1 | GGCTACGATGTTAGTGGTGG | TGG | 2781 |
| 3233 | 1 | AAGACTCTTCTCCCCGAGAG | AGG | 2782 |
| 3233 | −1 | AGGTGGACGGACCTCTCTCG | GGG | 2783 |
| 3234 | −1 | GAGGTGGACGGACCTCTCTC | GGG | 2784 |
| 3235 | −1 | AGAGGTGGACGGACCTCTCT | CGG | 2785 |
| 3246 | −1 | GCGATGATTTGAGAGGTGGA | CGG | 2786 |
| 3250 | −1 | GACGGCGATGATTTGAGAGG | TGG | 2787 |
| 3253 | −1 | GAGGACGGCGATGATTTGAG | AGG | 2788 |
| 3268 | −1 | AGTGGGGACGCCGACGAGGA | CGG | 2789 |
| 3269 | 1 | CAAATCATCGCCGTCCTCGT | CGG | 2790 |
| 3272 | −1 | CCCCAGTGGGGACGCCGACG | AGG | 2791 |
| 3281 | 1 | GTCCTCGTCGGCGTCCCCAC | TGG | 2792 |
| 3282 | 1 | TCCTCGTCGGCGTCCCCACT | GGG | 2793 |
| 3283 | 1 | CCTCGTCGGCGTCCCCACTG | GGG | 2794 |
| 3284 | 1 | CTCGTCGGCGTCCCCACTGG | GGG | 2795 |
| 3284 | −1 | GCAACAGAGTGCCCCCAGTG | GGG | 2796 |
| 3285 | −1 | AGCAACAGAGTGCCCCCAGT | GGG | 2797 |
| 3286 | −1 | GAGCAACAGAGTGCCCCCAG | TGG | 2798 |
| 3305 | 1 | GGCACTCTGTTGCTCCTCTC | CGG | 2799 |
| 3308 | −1 | CGAGAAGTGAAAGGCCGGAG | AGG | 2800 |
| 3313 | −1 | GGTTCCGAGAAGTGAAAGGC | CGG | 2801 |
| 3317 | −1 | TTATGGTTCCGAGAAGTGAA | AGG | 2802 |
| 3320 | 1 | CTCTCCGGCCTTTCACTTCT | CGG | 2803 |
| 3332 | 1 | TCACTTCTCGGAACCATAAT | CGG | 2804 |
| 3333 | 1 | CACTTCTCGGAACCATAATC | GGG | 2805 |
| 3334 | −1 | GGCAATTGCCAGCCCGATTA | TGG | 2806 |
| 3337 | 1 | TCTCGGAACCATAATCGGGC | TGG | 2807 |
| 3352 | 1 | CGGGCTGGCAATTGCCACCC | CGG | 2808 |
| 3355 | −1 | GAAGAAGATAAAAACCGGGG | TGG | 2809 |
| 3358 | −1 | GCTGAAGAAGATAAAAACCG | GGG | 2810 |
| 3359 | 7 | GGCTGAAGAAGATAAAAACC | GGG | 2811 |
| 3360 | −1 | GGGCTGAAGAAGATAAAAAC | CGG | 2812 |
| 3373 | 1 | GGTTTTTATCTTCTTCAGCC | CGG | 2813 |
| 3380 | −1 | CGACCGCGGGAACTATAACC | GGG | 2814 |
| 3381 | −1 | ACGACCGCGGGAACTATAAC | CGG | 2815 |
| 3388 | 1 | CAGCCCGGTTATAGTTCCCG | CGG | 2816 |
| 3393 | −1 | AGTCCAATGGTAACGACCGC | GGG | 2817 |
| 3394 | −1 | AAGTCCAATGGTAACGACCG | CGG | 2818 |
| 3401 | 1 | GTTCCCGCGGTCGTTACCAT | TGG | 2819 |
| 3406 | −1 | ACCAGTGACTGCAAGTCCAA | TGG | 2820 |
| 3416 | 1 | ACCATTGGACTTGCAGTCAC | TGG | 2821 |
| 3427 | 1 | TGCAGTCACTGGTATTCTCA | CGG | 2822 |
| 3430 | 1 | AGTCACTGGTATTCTCACGG | CGG | 2823 |
| 3431 | 1 | GTCACTGGTATTCTCACGGC | GGG | 2824 |
| 3440 | 1 | ATTCTCACGGCGGGAGCATG | TGG | 2825 |
| 3449 | 1 | GCGGGAGCATGTGGACTAAC | CGG | 2826 |
| 3450 | 1 | CGGGAGCATGTGGACTAACC | GGG | 2827 |
| 3457 | −1 | CCATGACAAAGACATCAGCC | CGG | 2828 |
| 3468 | 1 | CCGGGCTGATGTCTTTGTCA | TGG | 2829 |
| 3490 | 1 | GATGATTAACTTCATCCGAC | AGG | 2830 |
| 3494 | −1 | CCGTCGTCCCATGTACCTGT | CGG | 2831 |
| 3497 | 1 | AACTTCATCCGACAGGTACA | TGG | 2832 |
| 3498 | 1 | ACTTCATCCGACAGGTACAT | GGG | 2833 |
| 3505 | 1 | CCGACAGGTACATGGGACGA | CGG | 2834 |
| 3511 | 1 | GGTACATGGGACGACGGTGC | CGG | 2835 |
| 3519 | −1 | TTCACTGAGTCCAGCTGATC | CGG | 2836 |
| 3520 | 1 | GACGACGGTGCCGGATCAGC | TGG | 2837 |
| 3534 | 1 | ATCAGCTGGACTCAGTGAAG | CGG | 2838 |
| 3541 | 1 | GGACTCAGTGAAGCGGCGCA | TGG | 2839 |
| 3544 | 1 | CTCAGTGAAGCGGCGCATGG | CGG | 2840 |
| 3550 | 1 | GAAGCGGCGCATGGCGGACA | TGG | 2841 |
| 3553 | 1 | GCGGCGCATGGCGGACATGG | CGG | 2842 |
| 3562 | 1 | GGCGGACATGGCGGATTACG | TGG | 2843 |
| 3563 | 1 | GCGGACATGGCGGATTACGT | GGG | 2844 |
| 3564 | 1 | CGGACATGGCGGATTACGTG | GGG | 2845 |
| 3577 | 1 | TTACGTGGGGCAGAAGACAA | AGG | 2846 |
| 3584 | 1 | GGGCAGAAGACAAAGGATGC | TGG | 2847 |
| 3596 | −1 | GGGCCTTAGTCTGTATCTCT | TGG | 2848 |
| 3604 | 1 | TGGCCAAGAGATACAGACTA | AGG | 2849 |
| 3610 | 1 | AGAGATACAGACTAAGGCCC | AGG | 2850 |
| 3616 | −1 | TGATGACCTCTTAACATCCT | GGG | 2851 |
| 3617 | −1 | ATGATGACCTCTTAACATCC | TGG | 2852 |
| 3621 | 1 | CTAAGGCCCAGGATGTTAAG | AGG | 2853 |

TABLE 14
gRNA's targeting sequences of Ara h15.

| Position | | | | SEQ |
|---|---|---|---|---|
| on SEQ ID | | | | ID |
| NO: 2854 | Strand | Sequence | PAM | NO: |
| 61 | 1 | TGAAAATAACTTTTCTTTTG | TGG | 2856 |
| 73 | 1 | TTCTTTTGTGGAATTGTCTA | TGG | 2857 |
| 76 | 1 | TTTTGTGGAATTGTCTATGG | TGG | 2858 |
| 77 | 1 | TTTGTGGAATTGTCTATGGT | GGG | 2859 |
| 78 | 1 | TTGTGGAATTGTCTATGGTG | GGG | 2860 |
| 86 | 1 | TTGTCTATGGTGGGGAAAGT | TGG | 2861 |
| 92 | 1 | ATGGTGGGGAAAGTTGGTTC | TGG | 2862 |
| 105 | −1 | TATTTTAATCAGGTGCTTTA | AGG | 2863 |
| 115 | −1 | ATATTTCGAATATTTTAATC | AGG | 2864 |
| 130 | 1 | ATTAAAATATTCGAAATATG | TGG | 2865 |
| 154 | −1 | TAGAATTATTTTCAACGGTA | CGG | 2866 |
| 159 | −1 | CATAGTAGAATTATTTTCAA | CGG | 2867 |
| 175 | 1 | GAAAATAATTCTACTATGTT | TGG | 2868 |
| 187 | −1 | TTGCAATTATATAAATTCTG | GGG | 2869 |
| 188 | −1 | TTTGCAATTATATAAATTCT | GGG | 2870 |
| 189 | −1 | GTTTGCAATTATATAAATTC | TGG | 2871 |
| 203 | 1 | GAATTTATATAATTGCAAAC | CGG | 2872 |
| 204 | 1 | AATTTATATAATTGCAAACC | GGG | 2873 |
| 211 | 1 | ATAATTGCAAACCGGGTTAA | AGG | 2874 |
| 211 | −1 | TTTCCACATTCCCTTTAACC | CGG | 2875 |
| 212 | 1 | TAATTGCAAACCGGGTTAAA | GGG | 2876 |
| 219 | 1 | AAACCGGGTTAAAGGGAATG | TGG | 2877 |
| 232 | 1 | GGGAATGTGGAAAATGAGAA | CGG | 2878 |
| 265 | −1 | AATACGGCCCAATCACTCTC | TGG | 2879 |
| 268 | 1 | GAAGTTAGCCAGAGAGTGAT | TGG | 2880 |
| 269 | 1 | AAGTTAGCCAGAGAGTGATT | GGG | 2881 |
| 278 | 1 | AGAGAGTGATTGGGCCGTAT | TGG | 2882 |
| 281 | −1 | TTAGGTCACAAGGTCCAATA | CGG | 2883 |
| 291 | −1 | TATATCATGCTTAGGTCACA | AGG | 2884 |
| 299 | −1 | TTACCTCTTATATCATGCTT | AGG | 2885 |
| 307 | 1 | TGACCTAAGCATGATATAAG | AGG | 2886 |
| 330 | 1 | TAATGCTTTTTATATGTAGC | TGG | 2887 |
| 356 | 1 | ATTTGTAGATTATTATTTTT | TGG | 2888 |
| 419 | −1 | TACTTTTTTTTTGTTTACGG | TGG | 2889 |
| 422 | −1 | ACCTACTTTTTTTTTGTTTA | CGG | 2890 |
| 432 | 1 | ACCGTAAACAAAAAAAAAGT | AGG | 2891 |
| 509 | 1 | TAATAACAATTGTTATCCTG | AGG | 2892 |
| 514 | −1 | TTTATATTGTTAGTATCCTC | AGG | 2893 |
| 528 | 1 | GAGGATACTAACAATATAAA | TGG | 2894 |
| 657 | −1 | ACTATTATTAGTATTATATT | AGG | 2895 |
| 716 | 1 | GTTGAAGAATACTACTCCTT | AGG | 2896 |
| 721 | −1 | ACTTTTTTCTTCTTATCCTA | AGG | 2897 |
| 860 | 1 | AACAAAAAATAATTATGTGA | AGG | 2898 |
| 927 | 1 | TTTAAAGAATAAAAATTTTG | AGG | 2899 |
| 1013 | −1 | TTCTTTTAAATTGTTTATAG | TGG | 2900 |
| 1118 | −1 | CAAATTAGAAAAATATTATT | TGG | 2901 |
| 1178 | −1 | TTCAAACGGAGAAAAAAATT | CGG | 2902 |
| 1192 | −1 | CAAAATAATATTTGTTCAAA | CGG | 2903 |
| 1208 | 1 | TGAACAAATATTATTTTGAA | AGG | 2904 |
| 1262 | 1 | TAAAATAATTTTTTTTAGAA | AGG | 2905 |
| 1273 | 1 | TTTTTAGAAAGGAAAAAAAT | AGG | 2906 |
| 1283 | 1 | GGAAAAAAATAGGTGAATCA | AGG | 2907 |
| 1360 | 1 | AAACTAGAAAGTCAATTTTA | AGG | 2908 |
| 1427 | −1 | TTTCTGATAACTAAGTGTCT | TGG | 2909 |
| 1510 | 1 | CATTAATTATAAATGTTTCC | TGG | 2910 |
| 1517 | −1 | CTTCAGGACTTTTAAGGTCC | AGG | 2911 |
| 1523 | −1 | AAGTATCTTCAGGACTTTTA | AGG | 2912 |
| 1533 | −1 | TCCACGGTGAAAGTATCTTC | AGG | 2913 |
| 1543 | 1 | TCCTGAAGATACTTTCACCG | TGG | 2914 |
| 1549 | −1 | AAAGAAGGAGGCTTCTTCCA | CGG | 2915 |
| 1561 | −1 | AGGGGAGAGACAAAAGAAGG | AGG | 2916 |
| 1564 | −1 | ATAAGGGGAGAGACAAAAGA | AGG | 2917 |
| 1579 | −1 | TGCTTGAGAATTGAGATAAG | GGG | 2918 |
| 1580 | −1 | GTGCTTGAGAATTGAGATAA | GGG | 2919 |
| 1581 | −1 | TGTGCTTGAGAATTGAGATA | AGG | 2920 |
| 1623 | −1 | AAAAAGTTGCATGGGAATAT | TGG | 2921 |
| 1631 | −1 | AATTAAAAAAAAAGTTGCAT | GGG | 2922 |
| 1632 | −1 | TAATTAAAAAAAAAGTTGCA | TGG | 2923 |
| 1773 | −1 | ACTTTAACTATGTATTGAAT | TGG | 2924 |
| 1847 | −1 | CTTATAAAATTATAAAGCTT | GGG | 2925 |
| 1848 | −1 | TCTTATAAAATTATAAAGCT | TGG | 2926 |
| 1981 | −1 | GTAAACTCATTGGTATATTA | TGG | 2927 |
| 1991 | −1 | AAATTAAATAGTAAACTCAT | TGG | 2928 |
| 2014 | −1 | AAAACCACTTCGATATTTTT | GGG | 2929 |
| 2015 | −1 | AAAAACCACTTCGATATTTT | TGG | 2930 |
| 2021 | 1 | ATTTCCCAAAAATATCGAAG | TGG | 2931 |
| 2059 | −1 | AAAAAAAAATGTTTAAATGT | GGG | 2932 |
| 2060 | −1 | AAAAAAAAAATGTTTAAATG | TGG | 2933 |
| 2083 | 1 | ATTTTTTTTTTATTAAAAAT | AGG | 2934 |
| 2098 | 1 | AAAATAGGTAACTTTACTCA | AGG | 2935 |
| 2136 | 1 | ATAGATTTTATAATTTTTTT | TGG | 2936 |
| 2137 | 1 | TAGATTTTATAATTTTTTTT | GGG | 2937 |
| 2565 | 1 | ATAAAAATTTAATTACAAAT | TGG | 2938 |
| 2595 | −1 | ATTTTTTAAATTAAAAATTA | GGG | 2939 |
| 2596 | −1 | TATTTTTTAAATTAAAAATT | AGG | 2940 |
| 2712 | −1 | ACGAGATATTTAAATTTTTT | TGG | 2941 |
| 2779 | −1 | GTAAACCATAAATAGGTTCT | TGG | 2942 |
| 2785 | 1 | AAAAACCAAGAACCTATTTA | TGG | 2943 |
| 2786 | −1 | CTGATGAGTAAACCATAAAT | AGG | 2944 |
| 2819 | −1 | TCATAACATTAATTAATTAA | CGG | 2945 |
| 2850 | −1 | TTTGAAATAACTGTCTATTT | GGG | 2946 |
| 2851 | −1 | CTTTGAAATAACTGTCTATT | TGG | 2947 |
| 2868 | 1 | AGACAGTTATTTCAAAGAAA | AGG | 2948 |
| 2912 | −1 | CCATGGCATGCTTTCAATAA | TGG | 2949 |
| 2923 | 1 | CCATTATTGAAAGCATGCCA | TGG | 2950 |
| 2929 | −1 | CCTTTCTCTGCTGGGGACCA | TGG | 2951 |
| 2936 | −1 | ACAGGACCCTTTCTCTGCTG | GGG | 2952 |
| 2937 | −1 | TACAGGACCCTTTCTCTGCT | GGG | 2953 |
| 2938 | −1 | TTACAGGACCCTTTCTCTGC | TGG | 2954 |
| 2940 | 1 | CCATGGTCCCCAGCAGAGAA | AGG | 2955 |
| 2941 | 1 | CATGGTCCCCAGCAGAGAAA | GGG | 2956 |
| 2954 | −1 | TGATCACCATACATTGTTAC | AGG | 2957 |
| 2959 | 1 | AAGGGTCCTGTAACAATGTA | TGG | 2958 |
| 2990 | −1 | CACCTGTCCCTTTAACTGAC | TGG | 2959 |
| 2993 | 1 | AGAAGACACCAGTCAGTTAA | AGG | 2960 |
| 2994 | 1 | GAAGACACCAGTCAGTTAAA | GGG | 2961 |
| 2999 | 1 | CACCAGTCAGTTAAAGGGAC | AGG | 2962 |
| 3021 | −1 | TTTGCATGAAACTGAGGTGG | AGG | 2963 |
| 3024 | 1 | GGCTTTGCATGAAACTGAGG | TGG | 2964 |
| 3027 | −1 | GGCGGCTTTGCATGAAACTG | AGG | 2965 |
| 3045 | −1 | GGTGCATTGCTGTGTCGTGG | CGG | 2966 |
| 3048 | −1 | GAGGGTGCATTGCTGTGTCG | TGG | 2967 |
| 3066 | −1 | GTTAAGCTGGCAAAAGGGGA | GGG | 2968 |
| 3067 | −1 | GGTTAAGCTGGCAAAAGGGG | AGG | 2969 |
| 3070 | −1 | GGAGGTTAAGCTGGCAAAAG | GGG | 2970 |
| 3071 | −1 | GGGAGGTTAAGCTGGCAAAA | GGG | 2971 |
| 3072 | −1 | AGGGAGGTTAAGCTGGCAAA | AGG | 2972 |
| 3079 | −1 | GCAATGAAGGGAGGTTAAGC | TGG | 2973 |
| 3088 | −1 | ATGTATATAGCAATGAAGGG | AGG | 2974 |
| 3091 | −1 | GGAATGTATATAGCAATGAA | GGG | 2975 |
| 3092 | −1 | GGGAATGTATATAGCAATGA | AGG | 2976 |
| 3112 | −1 | AAGAGTGAGAGAGAAGTGAA | GGG | 2977 |
| 3113 | −1 | GAAGAGTGAGAGAGAAGTGA | AGG | 2978 |
| 3135 | −1 | ACAAGAAGTGATGGGGTTTG | AGG | 2979 |
| 3142 | −1 | TTTTTAGACAAGAAGTGATG | GGG | 2980 |
| 3143 | −1 | ATTTTTAGACAAGAAGTGAT | GGG | 2981 |
| 3144 | −1 | AATTTTTAGACAAGAAGTGA | TGG | 2982 |
| 3178 | −1 | GTAAATGGGTTTTTGGTGGC | TGG | 2983 |
| 3182 | −1 | AATGGTAAATGGGTTTTTGG | TGG | 2984 |
| 3185 | −1 | CATAATGGTAAATGGGTTTT | TGG | 2985 |
| 3192 | −1 | GATCAGACATAATGGTAAAT | GGG | 2986 |
| 3193 | −1 | TGATCAGACATAATGGTAAA | TGG | 2987 |
| 3200 | −1 | CCTTGTTTGATCAGACATAA | TGG | 2988 |
| 3211 | 1 | CCATTATGTCTGATCAAACA | AGG | 2989 |
| 3216 | 1 | ATGTCTGATCAAACAAGGAC | AGG | 2990 |
| 3222 | 1 | GATCAAACAAGGACAGGCTA | TGG | 2991 |
| 3225 | 1 | CAAACAAGGACAGGCTATGG | AGG | 2992 |
| 3228 | 1 | ACAAGGACAGGCTATGGAGG | AGG | 2993 |
| 3231 | 1 | AGGACAGGCTATGGAGGAGG | AGG | 2994 |
| 3232 | 1 | GGACAGGCTATGGAGGAGGA | GGG | 2995 |
| 3240 | 1 | TATGGAGGAGGAGGGTCCTA | TGG | 2996 |
| 3245 | −1 | TCCACCATAGGATGATCCAT | AGG | 2997 |
| 3252 | 1 | GGGTCCTATGGATCATCCTA | TGG | 2998 |
| 3255 | 1 | TCCTATGGATCATCCTATGG | TGG | 2999 |
| 3257 | −1 | ATAGGTGCCTCCTCCACCAT | AGG | 3000 |
| 3258 | 1 | TATGGATCATCCTATGGTGG | AGG | 3001 |
| 3261 | 1 | GGATCATCCTATGGTGGAGG | AGG | 3002 |
| 3270 | 1 | TATGGTGGAGGAGGCACCTA | TGG | 3003 |
| 3275 | −1 | GGTTCCATAAGATGAACCAT | AGG | 3004 |
| 3282 | 1 | GGCACCTATGGTTCATCTTA | TGG | 3005 |
| 3296 | −1 | GTTAGTACTGGGGTCATAGG | AGG | 3006 |
| 3299 | −1 | TTGGTTAGTACTGGGGTCAT | AGG | 3007 |
| 3306 | −1 | GTATAGGTTGGTTAGTACTG | GGG | 3008 |
| 3307 | −1 | CGTATAGGTTGGTTAGTACT | GGG | 3009 |
| 3308 | −1 | GCGTATAGGTTGGTTAGTAC | TGG | 3010 |
| 3318 | −1 | TGATGGCTTGGCGTATAGGT | TGG | 3011 |
| 3322 | −1 | AACTTGATGGCTTGGCGTAT | AGG | 3012 |
| 3330 | −1 | CTGTCATGAACTTGATGGCT | TGG | 3013 |
| 3335 | −1 | TGATGCTGTCATGAACTTGA | TGG | 3014 |
| 3354 | 1 | TTCATGACAGCATCAACCAT | TGG | 3015 |
| 3359 | −1 | GATCAAGAATGAGACACCAA | TGG | 3016 |
| 3378 | 1 | GTCTCATTCTTGATCCTGTC | TGG | 3017 |
| 3379 | 1 | TCTCATTCTTGATCCTGTCT | GGG | 3018 |
| 3381 | −1 | CAGTGAGGATCAACCCAGAC | AGG | 3019 |
| 3393 | 1 | CTGTCTGGGTTGATCCTCAC | TGG | 3020 |
| 3396 | −1 | AACCTATGACAGTTCCAGTG | AGG | 3021 |
| 3405 | 1 | ATCCTCACTGGAACTGTCAT | AGG | 3022 |
| 3433 | −1 | GGACTGAAGATAACAAGAAG | TGG | 3023 |
| 3454 | −1 | ATGGCAGCAGGGACAAGGAT | AGG | 3024 |
| 3459 | −1 | GGGTTATGGCAGCAGGGACA | AGG | 3025 |
| 3465 | −1 | GTGCAAGGGTTATGGCAGCA | GGG | 3026 |
| 3466 | −1 | AGTGCAAGGGTTATGGCAGC | AGG | 3027 |
| 3473 | −1 | AGCAGCCAGTGCAAGGGTTA | TGG | 3028 |
| 3479 | 1 | TGCTGCCATAACCCTTGCAC | TGG | 3029 |
| 3479 | −1 | TCCACCAGCAGCCAGTGCAA | GGG | 3030 |
| 3480 | −1 | ATCCACCAGCAGCCAGTGCA | AGG | 3031 |
| 3486 | 1 | ATAACCCTTGCACTGGCTGC | TGG | 3032 |
| 3489 | 1 | ACCCTTGCACTGGCTGCTGG | TGG | 3033 |
| 3504 | 1 | GCTGGTGGATTTTTGTTCTC | TGG | 3034 |
| 3507 | 1 | GGTGGATTTTTGTTCTCTGG | TGG | 3035 |
| 3513 | 1 | TTTTTGTTCTCTGGTGGCTG | TGG | 3036 |
| 3533 | −1 | CAACCATGACAATGCAGCAA | TGG | 3037 |
| 3541 | 1 | CTGCCATTGCTGCATTGTCA | TGG | 3038 |
| 3561 | 1 | TGGTTGTACAGCTATGTCAC | TGG | 3039 |
| 3562 | 1 | GGTTGTACAGCTATGTCACT | GGG | 3040 |
| 3576 | 1 | GTCACTGGGAAACACCCTGC | TGG | 3041 |
| 3579 | −1 | CAAGCCTATCAGAGCCAGCA | GGG | 3042 |
| 3580 | −1 | TCAAGCCTATCAGAGCCAGC | AGG | 3043 |
| 3586 | 1 | AACACCCTGCTGGCTCTGAT | AGG | 3044 |
| 3603 | 1 | GATAGGCTTGATTATGCTAA | AGG | 3045 |
| 3604 | 1 | ATAGGCTTGATTATGCTAAA | GGG | 3046 |
| 3605 | 1 | TAGGCTTGATTATGCTAAAG | GGG | 3047 |
| 3620 | 1 | TAAAGGGGTGATTGCTGATA | AGG | 3048 |
| 3625 | 1 | GGGTGATTGCTGATAAGGCT | AGG | 3049 |
| 3626 | 1 | GGTGATTGCTGATAAGGCTA | GGG | 3050 |
| 3635 | 1 | TGATAAGGCTAGGGATGTTA | AGG | 3051 |
| 3640 | 1 | AGGCTAGGGATGTTAAGGAC | AGG | 3052 |
| 3641 | 1 | GGCTAGGGATGTTAAGGACA | GGG | 3053 |
| 3647 | 1 | GGATGTTAAGGACAGGGCCA | AGG | 3054 |
| 3653 | −1 | ACCAGCACCAGCATAATCCT | TGG | 3055 |
| 3657 | 1 | GACAGGGCCAAGGATTATGC | TGG | 3056 |
| 3663 | 1 | GCCAAGGATTATGCTGGTGC | TGG | 3057 |
| 3667 | 1 | AGGATTATGCTGGTGCTGGT | AGG | 3058 |
| 3668 | 1 | GGATTATGCTGGTGCTGGTA | GGG | 3059 |
| 3674 | 1 | TGCTGGTGCTGGTAGGGCTC | AGG | 3060 |
| 3677 | 1 | TGGTGCTGGTAGGGCTCAGG | AGG | 3061 |
| 3678 | 1 | GGTGCTGGTAGGGCTCAGGA | GGG | 3062 |
| 3687 | 1 | AGGGCTCAGGAGGGCACACA | AGG | 3063 |
| 3688 | 1 | GGGCTCAGGAGGGCACACAA | GGG | 3064 |

Reference is now made to Table 15, listing the genomic sequences, the amino acid sequences and the gRNA's targeting sequences (regulatory sequences upstream of the first ATG and coding sequences) of each FAD2 gene FAD2-1, FAD2-2, FAD2-3 and FAD2-4.

TABLE 15
Genomic sequences, the amino acid sequences and
the gRNA's targeting sequences of each FAD2 gene.

| | Genomic | AA | gRNA |
|---|---|---|---|
| Gene name | SEQ ID NO: | SEQ ID NO: | SEQ ID NO: |
| FAD2-1 | 3065 | 3066 | 3067-3409 |
| FAD2-2 | 3410 | 3411 | 3412-3808 |
| FAD2-3 | 3809 | 3810 | 3811-4104 |
| FAD2-4 | 4105 | 4106 | 4107-4400 |

Reference is now made to Tables 16-19 presenting gRNA molecules targeted for silencing FAD2-1, FAD2-2, FAD2-3 and FAD2-4, respectively. The term ‘PAM’ refers hereinafter to Protospacer Adjacent Motif, which is a 2-6 base pair DNA sequence immediately following the DNA sequence targeted by the Cas9 nuclease in the CRISPR bacterial adaptive immune system.

The gRNAs are targeting coding sequences of FAD2 genes and regulatory sequences upstream of FAD2 genes first ATG.

TABLE 16
gRNA's targeting sequences of FAD2-1.

| Position | | | | SEQ |
|---|---|---|---|---|
| on SEQ ID | | | | ID |
| NO: 3065 | Strand | Sequence | PAM | NO: |
| 26 | −1 | AGGGGTATGGGGCGCAAAGA | AGG | 3067 |
| 37 | −1 | GAAGGGGGCAGAGGGGTATG | GGG | 3068 |
| 38 | −1 | AGAAGGGGGCAGAGGGGTAT | GGG | 3069 |
| 39 | −1 | GAGAAGGGGGCAGAGGGGTA | TGG | 3070 |
| 44 | −1 | AATGAGAGAAGGGGGCAGAG | GGG | 3071 |
| 45 | −1 | AAATGAGAGAAGGGGGCAGA | GGG | 3072 |
| 46 | −1 | GAAATGAGAGAAGGGGGCAG | AGG | 3073 |
| 52 | −1 | TGTGAGGAAATGAGAGAAGG | GGG | 3074 |
| 53 | −1 | GTGTGAGGAAATGAGAGAAG | GGG | 3075 |
| 54 | −1 | GGTGTGAGGAAATGAGAGAA | GGG | 3076 |
| 55 | −1 | AGGTGTGAGGAAATGAGAGA | AGG | 3077 |
| 68 | −1 | CGGCAAATGAAGAAGGTGTG | AGG | 3078 |
| 75 | −1 | TGTGTGGCGGCAAATGAAGA | AGG | 3079 |
| 88 | −1 | AGGAGGAGGAATGTGTGTGG | CGG | 3080 |
| 9 | −1 | CGGAGGAGGAGGAATGTGTG | TGG | 3081 |
| 102 | −1 | AGAAGGTGTGACGGAGGAGG | AGG | 3082 |
| 105 | −1 | AGGAGAAGGTGTGACGGAGG | AGG | 3083 |
| 108 | −1 | TGGAGGAGAAGGTGTGACGG | AGG | 3084 |
| 111 | −1 | GGATGGAGGAGAAGGTGTGA | CGG | 3085 |
| 119 | −1 | GTGTACCTGGATGGAGGAGA | AGG | 3086 |
| 125 | 1 | TCACACCTTCTCCTCCATCC | AGG | 3087 |
| 125 | −1 | AAGGAGGTGTACCTGGATGG | AGG | 3088 |
| 128 | −1 | GAGAAGGAGGTGTACCTGGA | TGG | 3089 |
| 132 | −1 | GAGGGAGAAGGAGGTGTACC | TGG | 3090 |
| 141 | −1 | GAGGAGAAAGAGGGAGAAGG | AGG | 3091 |
| 144 | −1 | ATTGAGGAGAAAGAGGGAGA | AGG | 3092 |
| 150 | −1 | AATTGAATTGAGGAGAAAGA | GGG | 3093 |
| 151 | −1 | GAATTGAATTGAGGAGAAAG | AGG | 3094 |
| 160 | −1 | CATGGCATTGAATTGAATTG | AGG | 3095 |
| 178 | −1 | TGATTAATAAGCAGGTAGCA | TGG | 3096 |
| 186 | −1 | AGTGCGATTGATTAATAAGC | AGG | 3097 |
| 223 | −1 | TAGTTAGATCTAGGCGAGTG | CGG | 3098 |
| 232 | −1 | TGAAAAGGTTAGTTAGATCT | AGG | 3099 |
| 247 | 1 | GATCTAACTAACCTTTTCAC | TGG | 3100 |
| 247 | −1 | ATCTATGGCATCCAGTGAAA | AGG | 3101 |
| 262 | −1 | TTAATGAACGAAGAGATCTA | TGG | 3102 |
| 287 | 1 | GTTCATTAATTCATTATATG | TGG | 3103 |
| 290 | 1 | CATTAATTCATTATATGTGG | TGG | 3104 |
| 309 | 1 | GTGGTAATTTCTGATTCAGT | AGG | 3105 |
| 310 | 1 | TGGTAATTTCTGATTCAGTA | GGG | 3106 |
| 323 | −1 | GCTTCAATTATGTAACACAA | TGG | 3107 |
| 355 | −1 | AAGCAAGGAGGGAGGTAAGG | GGG | 3108 |
| 356 | −1 | AAAGCAAGGAGGGAGGTAAG | GGG | 3109 |
| 357 | −1 | GAAAGCAAGGAGGGAGGTAA | GGG | 3110 |
| 358 | −1 | GGAAAGCAAGGAGGGAGGTA | AGG | 3111 |
| 363 | −1 | CATGAGGAAAGCAAGGAGGG | AGG | 3112 |
| 366 | −1 | AGACATGAGGAAAGCAAGGA | GGG | 3113 |
| 367 | −1 | AAGACATGAGGAAAGCAAGG | AGG | 3114 |
| 370 | −1 | AATAAGACATGAGGAAAGCA | AGG | 3115 |
| 379 | −1 | GAAATAAAAAATAAGACATG | AGG | 3116 |
| 401 | −1 | GAGATAACACATTCTTGATA | CGG | 3117 |
| 455 | −1 | TGCTATGATGAAAGGGAAAG | AGG | 3118 |
| 462 | −1 | TTTGTGATGCTATGATGAAA | GGG | 3119 |
| 463 | −1 | TTTTGTGATGCTATGATGAA | AGG | 3120 |
| 494 | −1 | CAAACAAGTGGAGACATAAT | TGG | 3121 |
| 506 | −1 | CTTGGAAAGAGACAAACAAG | TGG | 3122 |
| 524 | −1 | CGACGGAAAGAATCGTTGCT | TGG | 3123 |
| 541 | −1 | AAAATAATAGTAAAATACGA | CGG | 3124 |
| 565 | 1 | CTATTATTTTCTAACTTCTA | AGG | 3125 |
| 628 | 1 | TTTTTTTCTTTTTTAACGTG | CGG | 3126 |
| 635 | 1 | CTTTTTTAACGTGCGGCTAA | TGG | 3127 |
| 652 | 1 | TAATGGCTATGAGCATATTT | AGG | 3128 |
| 657 | 1 | GCTATGAGCATATTTAGGTA | AGG | 3129 |
| 666 | 1 | ATATTTAGGTAAGGTTCTCT | CGG | 3130 |
| 691 | −1 | ACAAAAAATCCGGAAATGTG | AGG | 3131 |
| 693 | 1 | AAGCAACAACCTCACATTTC | CGG | 3132 |
| 701 | −1 | GAGAAACCGTACAAAAAATC | CGG | 3133 |
| 706 | 1 | ACATTTCCGGATTTTTTGTA | CGG | 3134 |
| 727 | −1 | TGGGCTATTTTGTTGGAGAA | CGG | 3135 |
| 734 | −1 | AAGGGTCTGGGCTATTTTGT | TGG | 3136 |
| 746 | −1 | AATGAATAAGAAAAGGGTCT | GGG | 3137 |
| 747 | −1 | AAATGAATAAGAAAAGGGTC | TGG | 3138 |
| 752 | −1 | ATTGGAAATGAATAAGAAAA | GGG | 3139 |
| 753 | −1 | TATTGGAAATGAATAAGAAA | AGG | 3140 |
| 770 | −1 | TAATTACGATAAAATAATAT | TGG | 3141 |
| 803 | −1 | AAAATGCTGTTGGTAAGTAG | CGG | 3142 |
| 813 | −1 | GAAATTGATAAAAATGCTGT | TGG | 3143 |
| 1019 | 1 | ACTTTTACTTATTCTAATTT | AGG | 3144 |
| 1020 | 1 | CTTTTACTTATTCTAATTTA | GGG | 3145 |
| 1033 | 1 | TAATTTAGGGACTAATCTTT | TGG | 3146 |
| 1054 | 1 | GGCAAGCAAATTAAAAAATA | AGG | 3147 |
| 1057 | 1 | AAGCAAATTAAAAAATAAGG | TGG | 3148 |
| 1073 | 1 | AAGGTGGCACGCAGATCTCG | TGG | 3149 |
| 1156 | 1 | TATTATTTTTTTTTTTCTCT | AGG | 3150 |
| 1159 | 1 | TATTTTTTTTTTTCTCTAGG | TGG | 3151 |
| 1202 | 1 | GTAGTATTCTATGTGTAATT | TGG | 3152 |
| 1215 | 1 | TGTAATTTGGACTAATTGAT | TGG | 3153 |
| 1252 | 1 | TTTGATTCTATGATCCTAGT | TGG | 3154 |
| 1253 | 1 | TTGATTCTATGATCCTAGTT | GGG | 3155 |
| 1255 | −1 | TGTGAGTTGTAAACCCAACT | AGG | 3156 |
| 1282 | 1 | ACTCACAAATAGCCAATGCT | AGG | 3157 |
| 1283 | −1 | AATTGAGGCTTTCCTAGCAT | TGG | 3158 |
| 1298 | −1 | AAGGGTAATTTCGTTAATTG | AGG | 3159 |
| 1314 | 1 | ATTAACGAAATTACCCTTGT | TGG | 3160 |
| 1316 | −1 | TGATCTTTCTAAACCAACAA | GGG | 3161 |
| 1317 | −1 | ATGATCTTTCTAAACCAACA | AGG | 3162 |
| 1342 | −1 | GTGCGCAAGTTGACGTCGGT | TGG | 3163 |
| 1346 | −1 | AGAAGTGCGCAAGTTGACGT | CGG | 3164 |
| 1365 | 1 | AACTTGCGCACTTCTATTAT | AGG | 3165 |
| 1419 | 1 | TTGAGAGTAGACAGAATTAA | AGG | 3166 |
| 1429 | 1 | ACAGAATTAAAGGTCAAAAA | AGG | 3167 |
| 1467 | 1 | TCATCGTGTTATGCACAAGT | TGG | 3168 |
| 1491 | 1 | ACAAATTAAAAATGCAAAAC | AGG | 3169 |
| 1492 | 1 | CAAATTAAAAATGCAAAACA | GGG | 3170 |
| 1535 | −1 | TAATATCATATAGTATAAAC | AGG | 3171 |
| 1569 | −1 | GTAAATTCTTAACTATATGT | TGG | 3172 |
| 1616 | 1 | TCTTATTTTTGTTACTTTTA | CGG | 3173 |
| 1631 | −1 | TTAAAATTAGAAACATTATG | GGG | 3174 |
| 1632 | −1 | ATTAAAATTAGAAACATTAT | GGG | 3175 |
| 1633 | −1 | CATTAAAATTAGAAACATTA | TGG | 3176 |
| 1681 | 1 | TGAAAATCTTAAAATCGATT | TGG | 3177 |
| 1759 | −1 | CTAGCTAATAAATGTTGAGT | TGG | 3178 |
| 1784 | −1 | ATTTTTAAAAAGGTACTACT | AGG | 3179 |
| 1794 | −1 | GGTGAAATTAATTTTTAAAA | AGG | 3180 |
| 1815 | −1 | ATAAAGGTTGGAGCTTTTGT | TGG | 3181 |
| 1827 | −1 | CTAAATACCACTATAAAGGT | TGG | 3182 |
| 1831 | 1 | AAAAGCTCCAACCTTTATAG | TGG | 3183 |
| 1831 | −1 | ACTACTAAATACCACTATAA | AGG | 3184 |
| 1858 | 1 | TAGTAGTTAGTGTGTTTATG | AGG | 3185 |
| 1870 | 1 | TGTTTATGAGGTTACAAGAG | AGG | 3186 |
| 1871 | 1 | GTTTATGAGGTTACAAGAGA | GGG | 3187 |
| 1883 | −1 | ACCCAAAAGGAGCATTGGAG | TGG | 3188 |
| 1888 | −1 | TTCCTACCCAAAAGGAGCAT | TGG | 3189 |
| 1892 | 1 | GGCCACTCCAATGCTCCTTT | TGG | 3190 |
| 1893 | 1 | GCCACTCCAATGCTCCTTTT | GGG | 3191 |
| 1896 | −1 | CTTATCTCTTCCTACCCAAA | AGG | 3192 |
| 1897 | 1 | CTCCAATGCTCCTTTTGGGT | AGG | 3193 |
| 1924 | 1 | GATAAGAAAATTGAAGAGAA | TGG | 3194 |
| 1981 | 1 | ATTCATGTTTAGTAGTTTGT | TGG | 3195 |
| 1989 | 1 | TTAGTAGTTTGTTGGTGTCA | TGG | 3196 |
| 2002 | 1 | GGTGTCATGGTAAAAATCTA | TGG | 3197 |
| 2021 | 1 | ATGGCAATTTGTTATAATTG | AGG | 3198 |
| 2022 | 1 | TGGCAATTTGTTATAATTGA | GGG | 3199 |
| 2026 | 1 | AATTTGTTATAATTGAGGGA | CGG | 3200 |
| 2041 | 1 | AGGGACGGCATTTCCGAGAG | AGG | 3201 |
| 2043 | −1 | GTATATGTTATTACCTCTCT | CGG | 3202 |
| 2061 | 1 | AGGTAATAACATATACAGTG | AGG | 3203 |
| 2087 | 1 | CTCTGAGCGAGTACTTTATT | AGG | 3204 |
| 2088 | 1 | TCTGAGCGAGTACTTTATTA | GGG | 3205 |
| 2092 | 1 | AGCGAGTACTTTATTAGGGT | CGG | 3206 |
| 2110 | −1 | TGTGTTCTTTTGCAGTTTTG | GGG | 3207 |
| 2111 | −1 | ATGTGTTCTTTTGCAGTTTT | GGG | 3208 |
| 2112 | −1 | AATGTGTTCTTTTGCAGTTT | TGG | 3209 |
| 2147 | −1 | CACATGAAATGGGTAAAAAA | AGG | 3210 |
| 2157 | −1 | GTATGAGTGGCACATGAAAT | GGG | 3211 |
| 2158 | −1 | GGTATGAGTGGCACATGAAA | TGG | 3212 |
| 2170 | −1 | CTGTAGTTGACTGGTATGAG | TGG | 3213 |
| 2179 | −1 | TAACGCTTACTGTAGTTGAC | TGG | 3214 |
| 2193 | 1 | GTCAACTACAGTAAGCGTTA | AGG | 3215 |
| 2232 | −1 | AATTAAGAATTTCTTTAAAA | AGG | 3216 |
| 2355 | −1 | TACCAACCAGAAAAGAAAAC | AGG | 3217 |
| 2360 | 1 | ATGTATCCTGTTTTCTTTTC | TGG | 3218 |
| 2364 | 1 | ATCCTGTTTTCTTTTCTGGT | TGG | 3219 |
| 2376 | 1 | TTTCTGGTTGGTACCGTCTG | TGG | 3220 |
| 2378 | −1 | TATCATATCATAGCCACAGA | CGG | 3221 |
| 2392 | 1 | TCTGTGGCTATGATATGATA | TGG | 3222 |
| 2405 | 1 | TATGATATGGACCAATTATG | TGG | 3223 |
| 2405 | −1 | ATGCTACTCCTCCACATAAT | TGG | 3224 |
| 2408 | 1 | GATATGGACCAATTATGTGG | AGG | 3225 |
| 2434 | 1 | AGCATTTAGATTGCATAATT | AGG | 3226 |
| 2435 | 1 | GCATTTAGATTGCATAATTA | GGG | 3227 |
| 2514 | −1 | GTTCTTCAATTAAATAATTA | TGG | 3228 |
| 2536 | −1 | ATTGTGTCCTATATCTGACA | TGG | 3229 |
| 2540 | 1 | TGAAGAACCATGTCAGATAT | AGG | 3230 |
| 2595 | −1 | AAACCAATAGTGGAGCACAC | GGG | 3231 |
| 2596 | −1 | AAAACCAATAGTGGAGCACA | CGG | 3232 |
| 2603 | 1 | AATCCCGTGTGCTCCACTAT | TGG | 3233 |
| 2605 | −1 | TGTGTGTCCAAAACCAATAG | TGG | 3234 |
| 2609 | 1 | GTGTGCTCCACTATTGGTTT | TGG | 3235 |
| 2621 | 1 | ATTGGTTTTGGACACACACT | AGG | 3236 |
| 2638 | −1 | GGTCCCGTCCAAAGTAGATA | GGG | 3237 |
| 2639 | −1 | CGGTCCCGTCCAAAGTAGAT | AGG | 3238 |
| 2641 | 1 | AGGCAAGTCCCTATCTACTT | TGG | 3239 |
| 2645 | 1 | AAGTCCCTATCTACTTTGGA | CGG | 3240 |
| 2646 | 1 | AGTCCCTATCTACTTTGGAC | GGG | 3241 |
| 2651 | 1 | CTATCTACTTTGGACGGGAC | CGG | 3242 |
| 2659 | −1 | ATCAGAGCCAATGGGGTCTC | CGG | 3243 |
| 2663 | 1 | GACGGGACCGGAGACCCCAT | TGG | 3244 |
| 2666 | −1 | TTTGTGGATCAGAGCCAATG | GGG | 3245 |
| 2667 | −1 | TTTTGTGGATCAGAGCCAAT | GGG | 3246 |
| 2668 | −1 | TTTTTGTGGATCAGAGCCAA | TGG | 3247 |
| 2682 | −1 | AAATTTGGTGAATATTTTTG | TGG | 3248 |
| 2697 | −1 | CACTGCATGTGCAATAAATT | TGG | 3249 |
| 2709 | 1 | CAAATTTATTGCACATGCAG | TGG | 3250 |
| 2722 | −1 | ATATTAGCAATTCTCTACAA | TGG | 3251 |
| 2754 | −1 | CTTCCAAAAGACTACATGTT | AGG | 3252 |
| 2762 | 1 | TTGCCTAACATGTAGTCTTT | TGG | 3253 |
| 2766 | 1 | CTAACATGTAGTCTTTTGGA | AGG | 3254 |
| 2784 | 1 | GAAGGTTTATTTTGTATTTG | TGG | 3255 |
| 2831 | −1 | TATTTTTTAACCGAAAAATC | AGG | 3256 |
| 2832 | 1 | TGCATGCATGCCTGATTTTT | CGG | 3257 |
| 2865 | −1 | CTTTTTAGGTTATAATAATT | AGG | 3258 |
| 2879 | −1 | TCAGATTGACAAGTCTTTTT | AGG | 3259 |
| 2961 | −1 | CACACAAATTATTATGGTTA | AGG | 3260 |
| 2967 | −1 | ATATGTCACACAAATTATTA | TGG | 3261 |
| 2991 | T | GAGTCATGGCCTTTCAAAGC | AGG | 3262 |
| 2993 | 1 | TGACATATGCCTGCTTTGAA | AGG | 3263 |
| 3005 | T | AACTTTGCCTGCATGAGTCA | TGG | 3264 |
| 3009 | 1 | TGAAAGGCCATGACTCATGC | AGG | 3265 |
| 3020 | 1 | GACTCATGCAGGCAAAGTTA | TGG | 3266 |
| 3035 | −1 | AATTTCTCTTTGATATTACG | AGG | 3267 |
| 3066 | −1 | AATTTAGACAAAAATTTCAT | AGG | 3268 |
| 3101 | 1 | CTTAAAAGTAAAGATAGAGT | AGG | 3269 |
| 3104 | 1 | AAAAGTAAAGATAGAGTAGG | TGG | 3270 |
| 3105 | 1 | AAAGTAAAGATAGAGTAGGT | GGG | 3271 |
| 3122 | −1 | TACAAAATTCAAAAATAAAA | GGG | 3272 |
| 3123 | −1 | CTACAAAATTCAAAAATAAA | AGG | 3273 |
| 3146 | −1 | TATTTTTCTTATTAGATGAT | TGG | 3274 |
| 3192 | 1 | ACAAAAGCAGAGCGTATCCA | TGG | 3275 |
| 3198 | −1 | GAAATTGGTACAAGAAGCCA | TGG | 3276 |
| 3213 | −1 | AGTAATGTGCCAGGAGAAAT | TGG | 3277 |
| 3215 | 1 | CTTCTTGTACCAATTTCTCC | TGG | 3278 |
| 3222 | −1 | AAGCGAATTAGTAATGTGCC | AGG | 3279 |
| 3235 | 1 | TGGCACATTACTAATTCGCT | TGG | 3280 |
| 3289 | −1 | TTTATCGAAGAGTTATCTAT | CGG | 3281 |
| 3341 | −1 | CCCATTATTCAAATCGATGG | GGG | 3282 |
| 3342 | −1 | ACCCATTATTCAAATCGATG | GGG | 3283 |
| 3343 | −1 | GACCCATTATTCAAATCGAT | GGG | 3284 |
| 3344 | −1 | AGACCCATTATTCAAATCGA | TGG | 3285 |
| 3351 | 1 | TCCCCCATCGATTTGAATAA | TGG | 3286 |
| 3352 | 1 | CCCCCATCGATTTGAATAAT | GGG | 3287 |
| 3363 | 1 | TTGAATAATGGGTCTCAATT | CGG | 3288 |
| 3364 | 1 | TGAATAATGGGTCTCAATTC | GGG | 3289 |
| 3365 | 1 | GAATAATGGGTCTCAATTCG | GGG | 3290 |
| 3366 | 1 | AATAATGGGTCTCAATTCGG | GGG | 3291 |
| 3367 | 1 | ATAATGGGTCTCAATTCGGG | GGG | 3292 |
| 3376 | 1 | CTCAATTCGGGGGGAAGAAC | TGG | 3293 |
| 3393 | −1 | CTGGTAGCGCGAACGAGAAA | AGG | 3294 |
| 3407 | 1 | TTTCTCGTTCGCGCTACCAG | CGG | 3295 |
| 3412 | −1 | ATAATGGTCTCGTGCTCCGC | TGG | 3296 |
| 3428 | −1 | TAGTCAGAGTATTTTGATAA | TGG | 3297 |
| 3465 | −1 | AAATTATAGAATAAAAAACA | TGG | 3298 |
| 3538 | T | GCAGGAAATTTTATGATTTT | TGG | 3299 |
| 3556 | −1 | ATGGAAAGGCGCAAGATTGC | AGG | 3300 |
| 3570 | −1 | GAACACAGAATTATATGGAA | AGG | 3301 |
| 3575 | −1 | ATCATGAACACAGAATTATA | TGG | 3302 |
| 3622 | 1 | ATTTTTGTATTTGTAAATGC | TGG | 3303 |
| 3627 | 1 | TGTATTTGTAAATGCTGGAA | AGG | 3304 |
| 3647 | 1 | AGGTGTTTTGCTTTTATTTG | AGG | 3305 |
| 3674 | −1 | CCAATCAGTAAGTAAAAAAA | TGG | 3306 |
| 3685 | 1 | CCATTTTTTTACTTACTGAT | TGG | 3307 |
| 3686 | 1 | CATTTTTTTACTTACTGATT | GGG | 3308 |
| 3691 | 1 | TTTTACTTACTGATTGGGAA | AGG | 3309 |
| 3720 | −1 | TTATAGTCGTCACTGTTTAC | AGG | 3310 |
| 3746 | −1 | TACACAAGAACATTATTAAC | GGG | 3311 |
| 3747 | −1 | CTACACAAGAACATTATTAA | CGG | 3312 |
| 3800 | 1 | TTCTGCTTTAAAGACCACAC | AGG | 3313 |
| 3803 | −1 | GGACAATGTGTGTGCCTGTG | TGG | 3314 |
| 3824 | −1 | TTGTGCAGAAAGAATGAGAA | TGG | 3315 |
| 3891 | 1 | AGATAGTGCTAAATTTTATT | CGG | 3316 |
| 3892 | 1 | GATAGTGCTAAATTTTATTC | GGG | 3317 |
| 3952 | 1 | TGCGCTGTTATGTTATGTAC | AGG | 3318 |
| 3958 | 1 | GTTATGTTATGTACAGGTTG | CGG | 3319 |
| 3959 | 1 | TTATGTTATGTACAGGTTGC | GGG | 3320 |
| 3966 | 1 | ATGTACAGGTTGCGGGAAGA | TGG | 3321 |
| 3967 | 1 | TGTACAGGTTGCGGGAAGAT | GGG | 3322 |
| 3973 | 1 | GGTTGCGGGAAGATGGGAGC | TGG | 3323 |
| 3976 | 1 | TGCGGGAAGATGGGAGCTGG | CGG | 3324 |
| 3988 | −1 | TTGAAGGAGGAACAGAGGTT | CGG | 3325 |
| 3993 | −1 | CCTGTTTGAAGGAGGAACAG | AGG | 3326 |
| 4001 | −1 | TCGACCGTCCTGTTTGAAGG | AGG | 3327 |
| 4004 | 1 | CCTCTGTTCCTCCTTCAAAC | AGG | 3328 |
| 4004 | −1 | GCCTCGACCGTCCTGTTTGA | AGG | 3329 |
| 4008 | 1 | TGTTCCTCCTTCAAACAGGA | CGG | 3330 |
| 4014 | 1 | TCCTTCAAACAGGACGGTCG | AGG | 3331 |
| 4046 | −1 | CTGAAGGGAGGTTTTTCATG | CGG | 3332 |
| 4058 | −1 | ACTTGGCTGAGACTGAAGGG | AGG | 3333 |
| 4061 | −1 | TTGACTTGGCTGAGACTGAA | GGG | 3334 |
| 4062 | −1 | CTTGACTTGGCTGAGACTGA | AGG | 3335 |
| 4075 | −1 | GTGGGATGGCCTTCTTGACT | TGG | 3336 |
| 4077 | 1 | CAGTCTCAGCCAAGTCAAGA | AGG | 3337 |
| 4089 | −1 | CTGAAAACAGTGAGGTGGGA | TGG | 3338 |
| 4093 | −1 | AACGCTGAAAACAGTGAGGT | GGG | 3339 |
| 4094 | −1 | GAACGCTGAAAACAGTGAGG | TGG | 3340 |
| 4097 | −1 | ACAGAACGCTGAAAACAGTG | AGG | 3341 |
| 4123 | −1 | AGACAACATAGGAGAATGAG | CGG | 3342 |
| 4134 | −1 | GGTGAGATCATAGACAACAT | AGG | 3343 |
| 4155 | −1 | ATAGTAGAGGCAAAAGGCTA | TGG | 3344 |
| 4161 | −1 | GGCGGCATAGTAGAGGCAAA | AGG | 3345 |
| 4168 | −1 | AATTGGTGGCGGCATAGTAG | AGG | 3346 |
| 4179 | −1 | TTTGTGGAAGTAATTGGTGG | CGG | 3347 |
| 4182 | −1 | GAGTTTGTGGAAGTAATTGG | TGG | 3348 |
| 4185 | −1 | AGAGAGTTTGTGGAAGTAAT | TGG | 3349 |
| 4195 | −1 | ATAAAGGGGAAGAGAGTTTG | TGG | 3350 |
| 4209 | −1 | CCAGGCCAAGTAAAATAAAG | GGG | 3351 |
| 4210 | −1 | GCCAGGCCAAGTAAAATAAA | GGG | 3352 |
| 4211 | −1 | AGCCAGGCCAAGTAAAATAA | AGG | 3353 |
| 4215 | 1 | CTCTTCCCCTTTATTTTACT | TGG | 3354 |
| 4220 | 1 | CCCCTTTATTTTACTTGGCC | TGG | 3355 |
| 4227 | −1 | GACAGCCCCATAGAGAAGCC | AGG | 3356 |
| 4231 | 1 | TACTTGGCCTGGCTTCTCTA | TGG | 3357 |
| 4232 | 1 | ACTTGGCCTGGCTTCTCTAT | GGG | 3358 |
| 4233 | 1 | CTTGGCCTGGCTTCTCTATG | GGG | 3359 |
| 4243 | 1 | CTTCTCTATGGGGCTGTCCA | AGG | 3360 |
| 4249 | −1 | CACCGGTAAGGATGCAACCT | TGG | 3361 |
| 4258 | 1 | GTCCAAGGTTGCATCCTTAC | CGG | 3362 |
| 4261 | −1 | CGATGACCCAAACACCGGTA | AGG | 3363 |
| 4265 | 1 | GTTGCATCCTTACCGGTGTT | TGG | 3364 |
| 4266 | 1 | TTGCATCCTTACCGGTGTTT | GGG | 3365 |
| 4266 | −1 | GTGCGCGATGACCCAAACAC | CGG | 3366 |
| 4285 | 1 | TGGGTCATCGCGCACGAATG | TGG | 3367 |
| 4297 | −1 | GATAATCACTGAATGCATGG | TGG | 3368 |
| 4300 | −1 | ATTGATAATCACTGAATGCA | TGG | 3369 |
| 4313 | 1 | ATGCATTCAGTGATTATCAA | TGG | 3370 |
| 4330 | 1 | CAATGGCTTGATGATACAGT | TGG | 3371 |
| 4342 | −1 | AGAGGCCAGAGTGGAGAACA | AGG | 3372 |
| 4348 | 1 | GTTGGCCTTGTTCTCCACTC | TGG | 3373 |
| 4351 | −1 | AAGGGACCAAGAGGCCAGAG | TGG | 3374 |
| 4356 | 1 | TGTTCTCCACTCTGGCCTCT | TGG | 3375 |
| 4360 | −1 | ATGAAAAGTAAGGGACCAAG | AGG | 3376 |
| 4369 | −1 | TGTATTTCCATGAAAAGTAA | GGG | 3377 |
| 4370 | −1 | CTGTATTTCCATGAAAAGTA | AGG | 3378 |
| 4373 | 1 | TCTTGGTCCCTTACTTTTCA | TGG | 3379 |
| 4393 | −1 | TGTTGGAGTGGTGGCGACGG | TGG | 3380 |
| 4396 | −1 | CGGTGTTGGAGTGGTGGCGA | CGG | 3381 |
| 4402 | −1 | GGGAACCGGTGTTGGAGTGG | TGG | 3382 |
| 4405 | −1 | CAAGGGAACCGGTGTTGGAG | TGG | 3383 |
| 4408 | 1 | CGTCGCCACCACTCCAACAC | CGG | 3384 |
| 4410 | −1 | CCTGTCAAGGGAACCGGTGT | TGG | 3385 |
| 4416 | −1 | TTCATCCCTGTCAAGGGAAC | CGG | 3386 |
| 4421 | 1 | CCAACACCGGTTCCCTTGAC | AGG | 3387 |
| 4422 | 1 | CAACACCGGTTCCCTTGACA | GGG | 3388 |
| 4422 | −1 | AAAGACTTCATCCCTGTCAA | GGG | 3389 |
| 4423 | −1 | CAAAGACTTCATCCCTGTCA | AGG | 3390 |
| 4448 | −1 | GCAATACTAGACTTCTTCTT | TGG | 3391 |
| 4463 | 1 | AGAAGAAGTCTAGTATTGCA | TGG | 3392 |
| 4466 | 1 | AGAAGTCTAGTATTGCATGG | TGG | 3393 |
| 4486 | −1 | CTCTTCCTAGTGGGTTATTA | AGG | 3394 |
| 4492 | 1 | AAGTACCTTAATAACCCACT | AGG | 3395 |
| 4495 | −1 | GTGTGAGGACTCTTCCTAGT | GGG | 3396 |
| 4496 | −1 | AGTGTGAGGACTCTTCCTAG | TGG | 3397 |
| 4510 | −1 | TGAGGGTAATAGTGAGTGTG | AGG | 3398 |
| 4527 | −1 | AAGTGGCCAACCAAGGGTGA | GGG | 3399 |
| 4528 | 1 | CTCACTATTACCCTCACCCT | TGG | 3400 |
| 4528 | −1 | AAAGTGGCCAACCAAGGGTG | AGG | 3401 |
| 4532 | 1 | CTATTACCCTCACCCTTGGT | TGG | 3402 |
| 4533 | −1 | CAAGTAAAGTGGCCAACCAA | GGG | 3403 |
| 4534 | −1 | CCAAGTAAAGTGGCCAACCA | AGG | 3404 |
| 4544 | −1 | ACATTAAAAGCCAAGTAAAG | TGG | 3405 |
| 4545 | 1 | CCTTGGTTGGCCACTTTACT | TGG | 3406 |
| 4560 | 1 | TTACTTGGCTTTTAATGTCT | CGG | 3407 |
| 4561 | 1 | TACTTGGCTTTTAATGTCTC | GGG | 3408 |
| 4565 | 1 | TGGCTTTTAATGTCTCGGGA | AGG | 3409 |

TABLE 17
gRNA's targeting sequences of FAD2-2.

| Position | | | | SEQ |
|---|---|---|---|---|
| on | | | | |
| SEQ ID | | | | ID |
| NO: 3410 | Strand | Sequence | PAM | NO: |
| 3026 | −1 | AGGGGTATGGGGCGCAAAGA | AGG | 3412 |
| 3037 | −1 | GAAGGGGGCAGAGGGGTATG | GGG | 3413 |
| 3038 | −1 | AGAAGGGGGCAGAGGGGTAT | GGG | 3414 |
| 3039 | −1 | GAGAAGGGGGCAGAGGGGTA | TGG | 3415 |
| 3044 | −1 | AATGAGAGAAGGGGGCAGAG | GGG | 3416 |
| 3045 | −1 | AAATGAGAGAAGGGGGCAGA | GGG | 3417 |
| 3046 | −1 | GAAATGAGAGAAGGGGGCAG | AGG | 3418 |
| 3052 | −1 | TGTGAGGAAATGAGAGAAGG | GGG | 3419 |
| 3053 | −1 | GTGTGAGGAAATGAGAGAAG | GGG | 3420 |
| 3054 | −1 | GGTGTGAGGAAATGAGAGAA | GGG | 3421 |
| 3055 | −1 | AGGTGTGAGGAAATGAGAGA | AGG | 3422 |
| 3068 | −1 | CGGCAAATGAAGAAGGTGTG | AGG | 3423 |
| 3075 | −1 | TGTGTGGCGGCAAATGAAGA | AGG | 3424 |
| 3088 | −1 | AGGAGGAGGAATGTGTGTGG | CGG | 3425 |
| 3091 | −1 | CGGAGGAGGAGGAATGTGTG | TGG | 3426 |
| 3102 | −1 | AGAAGGTGTGACGGAGGAGG | AGG | 3427 |
| 3105 | −1 | AGGAGAAGGTGTGACGGAGG | AGG | 3428 |
| 3108 | −1 | TGGAGGAGAAGGTGTGACGG | AGG | 3429 |
| 3111 | −1 | GGATGGAGGAGAAGGTGTGA | CGG | 3430 |
| 3119 | −1 | GTGTACCTGGATGGAGGAGA | AGG | 3431 |
| 3125 | 1 | TCACACCTTCTCCTCCATCC | AGG | 3432 |
| 3125 | −1 | AAGGAGGTGTACCTGGATGG | AGG | 3433 |
| 3128 | −1 | AAGAAGGAGGTGTACCTGGA | TGG | 3434 |
| 3132 | −1 | GAGGAAGAAGGAGGTGTACC | TGG | 3435 |
| 3141 | −1 | GAGGAGAAAGAGGAAGAAGG | AGG | 3436 |
| 3144 | −1 | ATTGAGGAGAAAGAGGAAGA | AGG | 3437 |
| 3151 | −1 | GAATTGAATTGAGGAGAAAG | AGG | 3438 |
| 3160 | −1 | CATGGCATTGAATTGAATTG | AGG | 3439 |
| 3178 | −1 | TGATTAATAAGCAGGTAGCA | TGG | 3440 |
| 3186 | 1 | AGTGCGATTGATTAATAAGC | AGG | 3441 |
| 3223 | −1 | TAGTTAGATCTAGGCGAGTG | CGG | 3442 |
| 3232 | −1 | TGAAAAGGTTAGTTAGATCT | AGG | 3443 |
| 3247 | 1 | GATCTAACTAACCTTTTCAT | TGG | 3444 |
| 3247 | −1 | ATCTATGGCATCCAATGAAA | AGG | 3445 |
| 3262 | −1 | TTAATGAACGAAGAGATCTA | TGG | 3446 |
| 3287 | 1 | GTTCATTAATTCATTAGATG | TGG | 3447 |
| 3290 | 1 | CATTAATTCATTAGATGTGG | TGG | 3448 |
| 3309 | 1 | GTGGTAATTTCTGATTCAGT | AGG | 3449 |
| 3310 | 1 | TGGTAATTTCTGATTCAGTA | GGG | 3450 |
| 3323 | −1 | GCTTCAATTATGTAACACAA | TGG | 3451 |
| 3355 | −1 | AAGCAAGGAGGGAGGTAAGG | GGG | 3452 |
| 3356 | −1 | AAAGCAAGGAGGGAGGTAAG | GGG | 3453 |
| 3357 | −1 | GAAAGCAAGGAGGGAGGTAA | GGG | 3454 |
| 3358 | −1 | GGAAAGCAAGGAGGGAGGTA | AGG | 3455 |
| 3363 | −1 | CATGAGGAAAGCAAGGAGGG | AGG | 3456 |
| 3366 | −1 | AGACATGAGGAAAGCAAGGA | GGG | 3457 |
| 3367 | −1 | AAGACATGAGGAAAGCAAGG | AGG | 3458 |
| 3370 | −1 | AATAAGACATGAGGAAAGCA | AGG | 3459 |
| 3379 | −1 | GAAATAAAAAATAAGACATG | AGG | 3460 |
| 3401 | −1 | GAGATAACACATTCTTGTTA | CGG | 3461 |
| 3417 | 1 | ACAAGAATGTGTTATCTCTG | AGG | 3462 |
| 3456 | −1 | TGCTACGATGAAAGAGAAAG | AGG | 3463 |
| 3495 | −1 | CAAACAAGTGGAGACAGAAT | TGG | 3464 |
| 3507 | −1 | CTTGGAAAGAGACAAACAAG | TGG | 3465 |
| 3525 | −1 | CGACTGAAAGAATCGTTGCT | TGG | 3466 |
| 3559 | 1 | TATTTTACTATTTTTTTATA | AGG | 3467 |
| 3632 | 1 | TTTTTTTCTTTTTTAACGTG | CGG | 3468 |
| 3649 | 1 | GTGCGGCTATGAGCATATTT | AGG | 3469 |
| 3654 | 1 | GCTATGAGCATATTTAGGTA | AGG | 3470 |
| 3663 | 1 | ATATTTAGGTAAGGTTCTCT | CGG | 3471 |
| 3688 | −1 | ACAAAAAATCCGGAAAGGTC | AGG | 3472 |
| 3690 | 1 | AAGCAACAACCTGACCTTTC | CGG | 3473 |
| 3693 | −1 | ACCGTACAAAAAATCCGGAA | AGG | 3474 |
| 3698 | −1 | GAGAAACCGTACAAAAAATC | CGG | 3475 |
| 3703 | 1 | ACCTTTCCGGATTTTTTGTA | CGG | 3476 |
| 3724 | −1 | TGGGCTATTTTGTTGGAGAA | CGG | 3477 |
| 3731 | −1 | AAGGGTTTGGGCTATTTTGT | TGG | 3478 |
| 3743 | −1 | ATGAATAAAGAAAAGGGTTT | GGG | 3479 |
| 3744 | −1 | AATGAATAAAGAAAAGGGTT | TGG | 3480 |
| 3749 | −1 | ATGGAAATGAATAAAGAAAA | GGG | 3481 |
| 3750 | −1 | TATGGAAATGAATAAAGAAA | AGG | 3482 |
| 3768 | −1 | GAATTAGCCAAAATAATATA | TGG | 3483 |
| 3772 | 1 | TTCATTTCCATATATTATTT | TGG | 3484 |
| 3784 | 1 | TATTATTTTGGCTAATTCTA | TGG | 3485 |
| 3787 | 1 | TATTTTGGCTAATTCTATGG | TGG | 3486 |
| 3798 | 1 | ATTCTATGGTGGCTCTGAAC | CGG | 3487 |
| 3806 | −1 | GTTAGACAAAATTTAGGTAC | CGG | 3488 |
| 3812 | −1 | AAATAAGTTAGACAAAATTT | AGG | 3489 |
| 3951 | −1 | AATAATTAGGGTAAAATAAT | AGG | 3490 |
| 3963 | −1 | TCCCTAAATTAGAATAATTA | GGG | 3491 |
| 3964 | −1 | GTCCCTAAATTAGAATAATT | AGG | 3492 |
| 3972 | 1 | TACCCTAATTATTCTAATTT | AGG | 3493 |
| 3973 | 1 | ACCCTAATTATTCTAATTTA | GGG | 3494 |
| 3986 | 1 | TAATTTAGGGACTAATCTTT | TGG | 3495 |
| 4010 | 1 | AAGAAAATTAAAAAATAACG | TGG | 3496 |
| 4106 | 1 | TGTATTATTTTTTTTTCTCT | AGG | 3497 |
| 4109 | 1 | ATTATTTTTTTTTCTCTAGG | TGG | 3498 |
| 4151 | 1 | GTAGTATTCTATGTCCAATT | TGG | 3499 |
| 4154 | −1 | ACCAACCAATTAGTCCAAAT | TGG | 3500 |
| 4160 | 1 | TATGTCCAATTTGGACTAAT | TGG | 3501 |
| 4164 | 1 | TCCAATTTGGACTAATTGGT | TGG | 3502 |
| 4203 | 1 | TTTGATTCTATGATCCTAGT | TGG | 3503 |
| 4204 | 1 | TTGATTCTATGATCCTAGTT | GGG | 3504 |
| 4206 | −1 | TGTGAGTTGTAAACCCAACT | AGG | 3505 |
| 4233 | 1 | ACTCACAAATAGCCAATGCT | AGG | 3506 |
| 4234 | −1 | AATTAAAGCTTTCCTAGCAT | TGG | 3507 |
| 4265 | 1 | ATTAACGAAATTACCCTTAT | TGG | 3508 |
| 4267 | −1 | TGATCTTTCTAAACCAATAA | GGG | 3509 |
| 4268 | −1 | ATGATCTTTCTAAACCAATA | AGG | 3510 |
| 4293 | −1 | GTGCGCAAGTTGACGTCGGT | TGG | 3511 |
| 4297 | −1 | AGAAGTGCGCAAGTTGACGT | CGG | 3512 |
| 4316 | 1 | AACTTGCGCACTTCTATTAT | AGG | 3513 |
| 4370 | 1 | TTTAGAGTAGACAGAATTAA | AGG | 3514 |
| 4381 | 1 | CAGAATTAAAGGTCAAAAAA | AGG | 3515 |
| 4444 | 1 | ATAAATTAAAAATGCAAAAC | AGG | 3516 |
| 4445 | 1 | TAAATTAAAAATGCAAAACA | GGG | 3517 |
| 4488 | −1 | AATAATATCATAGTATAAAG | CGG | 3518 |
| 4567 | 1 | TCTTATTTTTGTTACTTTTA | CGG | 3519 |
| 4582 | −1 | AAAAATAGAAACATTAGATG | GGG | 3520 |
| 4583 | −1 | AAAAAATAGAAACATTAGAT | GGG | 3521 |
| 4584 | −1 | TAAAAAATAGAAACATTAGA | TGG | 3522 |
| 4631 | 1 | TGAAAATCTTAAAATCGATT | TGG | 3523 |
| 4718 | −1 | CTAGCTAATAAATGTTGAGT | TGG | 3524 |
| 4746 | −1 | TTAATTTTTAAAAACTAAAA | AGG | 3525 |
| 4774 | −1 | ATAAAGGTTGGAGCTTTTGT | TGG | 3526 |
| 4786 | −1 | CTAAATACCACTATAAAGGT | TGG | 3527 |
| 4790 | 1 | AAAAGCTCCAACCTTTATAG | TGG | 3528 |
| 4790 | −1 | ACTACTAAATACCACTATAA | AGG | 3529 |
| 4817 | 1 | TAGTAGTTAGTATGTTTATG | AGG | 3530 |
| 4829 | 1 | TGTTTATGAGGTTACAAGAG | AGG | 3531 |
| 4830 | 1 | GTTTATGAGGTTACAAGAGA | GGG | 3532 |
| 4842 | −1 | ACCCAAAAGGAGCATTGGAG | TGG | 3533 |
| 4847 | −1 | TTCCTACCCAAAAGGAGCAT | TGG | 3534 |
| 4851 | 1 | GGCCACTCCAATGCTCCTTT | TGG | 3535 |
| 4852 | 1 | GCCACTCCAATGCTCCTTTT | GGG | 3536 |
| 4855 | −1 | CTTCTCTCTTCCTACCCAAA | AGG | 3537 |
| 4856 | 1 | CTCCAATGCTCCTTTTGGGT | AGG | 3538 |
| 4883 | 1 | GAGAAGAAAATTGAAGAGAA | TGG | 3539 |
| 4940 | 1 | ATTCATGTTTAGTAGTTTGT | TGG | 3540 |
| 4948 | 1 | TTAGTAGTTTGTTGGTGTCA | TGG | 3541 |
| 4961 | 1 | GGTGTCATGGTAAAAATCTA | TGG | 3542 |
| 4980 | 1 | ATGGCAATTTATTATAATTG | AGG | 3543 |
| 4981 | 1 | TGGCAATTTATTATAATTGA | GGG | 3544 |
| 4985 | 1 | AATTTATTATAATTGAGGGA | CGG | 3545 |
| 5000 | 1 | AGGGACGGCATTTCCGAGAG | AGG | 3546 |
| 5002 | −1 | GTATATGTTATTACCTCTCT | CGG | 3547 |
| 5020 | 1 | AGGTAATAACATATACAGTG | AGG | 3548 |
| 5046 | 1 | CTCTGAGCGAGTACTTTATT | AGG | 3549 |
| 5047 | 1 | TCTGAGCGAGTACTTTATTA | GGG | 3550 |
| 5051 | 1 | AGCGAGTACTTTATTAGGGT | CGG | 3551 |
| 5069 | −1 | TGTGTTCTTTTGCAGTTTTG | GGG | 3552 |
| 5070 | −1 | ATGTGTTCTTTTGCAGTTTT | GGG | 3553 |
| 5071 | −1 | AATGTGTTCTTTTGCAGTTT | TGG | 3554 |
| 5106 | −1 | ACATGAAATGGGTAAAAAAA | AGG | 3555 |
| 5117 | −1 | GTAGGAGTGGCACATGAAAT | GGG | 3556 |
| 5118 | −1 | GGTAGGAGTGGCACATGAAA | TGG | 3557 |
| 5130 | −1 | CTGTAGTTGACTGGTAGGAG | TGG | 3558 |
| 5135 | −1 | GCTTACTGTAGTTGACTGGT | AGG | 3559 |
| 5139 | −1 | CAACGCTTACTGTAGTTGAC | TGG | 3560 |
| 5153 | 1 | GTCAACTACAGTAAGCGTTG | AGG | 3561 |
| 5181 | 1 | TTTATATGAGATAATTTTTA | TGG | 3562 |
| 5182 | 1 | TTATATGAGATAATTTTTAT | GGG | 3563 |
| 5257 | 1 | AAAAGTGTGATTTATTGATT | TGG | 3564 |
| 5269 | −1 | TGTTGTTTTTATTTGAAGTG | TGG | 3565 |
| 5316 | −1 | CCCTTAGATGATGGAGTGTA | GGG | 3566 |
| 5317 | −1 | ACCCTTAGATGATGGAGTGT | AGG | 3567 |
| 5325 | −1 | TCTTTTTGACCCTTAGATGA | TGG | 3568 |
| 5326 | 1 | ACCCTACACTCCATCATCTA | AGG | 3569 |
| 5327 | 1 | CCCTACACTCCATCATCTAA | GGG | 3570 |
| 5374 | 1 | AGACAATTATAATATCTTCA | TGG | 3571 |
| 5375 | 1 | GACAATTATAATATCTTCAT | GGG | 3572 |
| 5391 | −1 | AAGAGGTTATAAAAATTGGT | GGG | 3573 |
| 5392 | −1 | AAAGAGGTTATAAAAATTGG | TGG | 3574 |
| 5395 | −1 | TTAAAAGAGGTTATAAAAAT | TGG | 3575 |
| 5408 | −1 | AGAAATACTTTTCTTAAAAG | AGG | 3576 |
| 5535 | −1 | TACCAACCAGAAAAGAAAAC | AGG | 3577 |
| 5540 | 1 | ATGTATCCTGTTTTCTTTTC | TGG | 3578 |
| 5544 | 1 | ATCCTGTTTTCTTTTCTGGT | TGG | 3579 |
| 5556 | 1 | TTTCTGGTTGGTACCGTCTG | TGG | 3580 |
| 5558 | −1 | TATCATATCATAGCCACAGA | CGG | 3581 |
| 5572 | 1 | TCTGTGGCTATGATATGATA | TGG | 3582 |
| 5585 | 1 | TATGATATGGACCAATTATG | TGG | 3583 |
| 5585 | −1 | ATGCTACTCCTCCACATAAT | TGG | 3584 |
| 5588 | 1 | GATATGGACCAATTATGTGG | AGG | 3585 |
| 5614 | 1 | AGCATTTAGATTGCATAATT | AGG | 3586 |
| 5615 | 1 | GCATTTAGATTGCATAATTA | GGG | 3587 |
| 5661 | −1 | TATGCAAGTTGCAATCTTGC | AGG | 3588 |
| 5694 | −1 | GTTCTTCAATTAAATAATTA | TGG | 3589 |
| 5716 | −1 | ATTGTGTCCTATATCTGACA | TGG | 3590 |
| 5720 | 1 | TGAAGAACCATGTCAGATAT | AGG | 3591 |
| 5775 | −1 | AAACCAATAGTGGAGCACAC | GGG | 3592 |
| 5776 | −1 | AAAACCAATAGTGGAGCACA | CGG | 3593 |
| 5783 | 1 | AATCCCGTGTGCTCCACTAT | TGG | 3594 |
| 5785 | −1 | TGTGTGTCCAAAACCAATAG | TGG | 3595 |
| 5789 | 1 | GTGTGCTCCACTATTGGTTT | TGG | 3596 |
| 5801 | 1 | ATTGGTTTTGGACACACACT | AGG | 3597 |
| 5818 | −1 | GGTCCCGTCCAAAGTAGATA | GGG | 3598 |
| 5819 | −1 | CGGTCCCGTCCAAAGTAGAT | AGG | 3599 |
| 5821 | 1 | AGGCAAGTCCCTATCTACTT | TGG | 3600 |
| 5825 | 1 | AAGTCCCTATCTACTTTGGA | CGG | 3601 |
| 5826 | 1 | AGTCCCTATCTACTTTGGAC | GGG | 3602 |
| 5831 | 1 | CTATCTACTTTGGACGGGAC | CGG | 3603 |
| 5839 | −1 | ATCAGAGCCAATGGGGTCTC | CGG | 3604 |
| 5843 | 1 | GACGGGACCGGAGACCCCAT | TGG | 3605 |
| 5846 | −1 | TTTGTGGATCAGAGCCAATG | GGG | 3606 |
| 5847 | −1 | TTTTGTGGATCAGAGCCAAT | GGG | 3607 |
| 5848 | −1 | TTTTTGTGGATCAGAGCCAA | TGG | 3608 |
| 5862 | −1 | AAATTTGGTGAATATTTTTG | TGG | 3609 |
| 5877 | −1 | CACTGCATGTGCAATAAATT | TGG | 3610 |
| 5889 | 1 | CAAATTTATTGCACATGCAG | TGG | 3611 |
| 5902 | −1 | ATATTAGCAATTCTCTACAA | TGG | 3612 |
| 5942 | 1 | TTGCGTAACATGTAGTCTTT | TGG | 3613 |
| 5946 | 1 | GTAACATGTAGTCTTTTGGA | AGG | 3614 |
| 5962 | 1 | TGGAAGGTTTATTTTGTATG | TGG | 3615 |
| 6009 | −1 | TATTTTTTAACCGAAAAATC | AGG | 3616 |
| 6010 | 1 | TGCATGCATGCCTGATTTTT | CGG | 3617 |
| 6043 | −1 | TCTTTTAGGTTATAATAATT | AGG | 3618 |
| 6057 | −1 | ATCAGATTGACAAGTCTTTT | AGG | 3619 |
| 6138 | −1 | CACACAAATTGTTAAGGTTA | AGG | 3620 |
| 6144 | −1 | ATATGTCACACAAATTGTTA | AGG | 3621 |
| 6168 | −1 | GAGTCATGGCCTTTCAAAGC | AGG | 3622 |
| 6170 | 1 | TGACATATTCCTGCTTTGAA | AGG | 3623 |
| 6182 | −1 | AACTTTGCCTGCATGAGTCA | TGG | 3624 |
| 6186 | 1 | TGAAAGGCCATGACTCATGC | AGG | 3625 |
| 6197 | 1 | GACTCATGCAGGCAAAGTTA | TGG | 3626 |
| 6212 | −1 | AATTTCTCTTTGATATTACG | AGG | 3627 |
| 6229 | 1 | AATATCAAAGAGAAATTCTT | TGG | 3628 |
| 6243 | −1 | AATTTAGACAAAAATTTCAT | AGG | 3629 |
| 6279 | 1 | TTAAAAGTAAAGAATAGAGT | AGG | 3630 |
| 6282 | 1 | AAAGTAAAGAATAGAGTAGG | TGG | 3631 |
| 6283 | 1 | AAGTAAAGAATAGAGTAGGT | GGG | 3632 |
| 6300 | −1 | CGCAAGATTCAAAAATAAAA | GGG | 3633 |
| 6301 | −1 | TCGCAAGATTCAAAAATAAA | AGG | 3634 |
| 6324 | 1 | GAATCTTGCGAGTTAAATTT | AGG | 3635 |
| 6343 | −1 | TAGTCAGAGTATTTTGATAA | TGG | 3636 |
| 6380 | −1 | AAATTATCGAATAAAAAACA | TGG | 3637 |
| 6408 | −1 | GTCAGTTGAAAATATAGAAA | AGG | 3638 |
| 6458 | −1 | GCAGGAAATTTTATGATTTT | TGG | 3639 |
| 6476 | −1 | ATGGAAAGGGGCAAGGTTGC | AGG | 3640 |
| 6483 | −1 | GAATTATATGGAAAGGGGCA | AGG | 3641 |
| 6488 | −1 | ACACAGAATTATATGGAAAG | GGG | 3642 |
| 6489 | −1 | AACACAGAATTATATGGAAA | GGG | 3643 |
| 6490 | −1 | GAACACAGAATTATATGGAA | AGG | 3644 |
| 6495 | −1 | ATCATGAACACAGAATTATA | TGG | 3645 |
| 6542 | 1 | ATTTTTGTATTTGTAAATGC | TGG | 3646 |
| 6547 | 1 | TGTATTTGTAAATGCTGGAA | AGG | 3647 |
| 6586 | −1 | GAAAAGTTAAAGTAAAAAAA | TGG | 3648 |
| 6619 | −1 | ATAACATACATAGTACATAA | AGG | 3649 |
| 6642 | 1 | ATGTATGTTATGTATTATAG | CGG | 3650 |
| 6653 | 1 | GTATTATAGCGGTTATAGCA | CGG | 3651 |
| 6673 | −1 | TTCTCACTCAGTAAGGGGAA | AGG | 3652 |
| 6678 | −1 | TTCCTTTCTCACTCAGTAAG | GGG | 3653 |
| 6679 | −1 | TTTCCTTTCTCACTCAGTAA | GGG | 3654 |
| 6680 | −1 | ATTTCCTTTCTCACTCAGTA | AGG | 3655 |
| 6687 | 1 | TTCCCCTTACTGAGTGAGAA | AGG | 3656 |
| 6716 | −1 | TTATAGTCATCACTGTTTAC | AGG | 3657 |
| 6742 | −1 | TATACAAGAACATTATTAAC | AGG | 3658 |
| 6799 | −1 | ATGAGAATGGAAAATGTGTG | TGG | 3659 |
| 6812 | −1 | TTGTGCAGAAAGAATGAGAA | TGG | 3660 |
| 6879 | 1 | AGATAGTGCTAAATTTTATT | CGG | 3661 |
| 6880 | 1 | GATAGTGCTAAATTTTATTC | GGG | 3662 |
| 6940 | 1 | TGCACTGTTATGTTATGTAC | AGG | 3663 |
| 6946 | 1 | GTTATGTTATGTACAGGTTG | CGG | 3664 |
| 6947 | 1 | TTATGTTATGTACAGGTTGC | GGG | 3665 |
| 6954 | 1 | ATGTACAGGTTGCGGGAAGA | TGG | 3666 |
| 6955 | 1 | TGTACAGGTTGCGGGAAGAT | GGG | 3667 |
| 6961 | 1 | GGTTGCGGGAAGATGGGAGC | TGG | 3668 |
| 6964 | 1 | TGCGGGAAGATGGGAGCTGG | CGG | 3669 |
| 6976 | −1 | TTGAAGGAGGAACAGAGGTT | CGG | 3670 |
| 6981 | −1 | CCTGTTTGAAGGAGGAACAG | AGG | 3671 |
| 6989 | −1 | TCGACCGTCCTGTTTGAAGG | AGG | 3672 |
| 6992 | 1 | CCTCTGTTCCTCCTTCAAAC | AGG | 3673 |
| 6992 | −1 | GCCTCGACCGTCCTGTTTGA | AGG | 3674 |
| 6996 | 1 | TGTTCCTCCTTCAAACAGGA | CGG | 3675 |
| 7002 | 1 | TCCTTCAAACAGGACGGTCG | AGG | 3676 |
| 7034 | −1 | CTGAAGGGAGGTTTTTCATG | CGG | 3677 |
| 7046 | −1 | ACTTGGCTGAGACTGAAGGG | AGG | 3678 |
| 7049 | −1 | TTGACTTGGCTGAGACTGAA | GGG | 3679 |
| 7050 | −1 | CTTGACTTGGCTGAGACTGA | AGG | 3680 |
| 7063 | −1 | GTGGGATGGCCTTCTTGACT | TGG | 3681 |
| 7065 | 1 | CAGTCTCAGCCAAGTCAAGA | AGG | 3682 |
| 7077 | −1 | CTGAAAACAGTGAGGTGGGA | TGG | 3683 |
| 7081 | −1 | AACGCTGAAAACAGTGAGGT | GGG | 3684 |
| 7082 | −1 | GAACGCTGAAAACAGTGAGG | TGG | 3685 |
| 7085 | −1 | ACAGAACGCTGAAAACAGTG | AGG | 3686 |
| 7111 | −1 | AGACAACATAGGAGAATGAG | CGG | 3687 |
| 7122 | −1 | GGTGAGATCATAGACAACAT | AGG | 3688 |
| 7143 | −1 | ATAGTAGAGGCAAAAGGCTA | TGG | 3689 |
| 7149 | −1 | GGCGGCATAGTAGAGGCAAA | AGG | 3690 |
| 7156 | −1 | AATTGGTGGCGGCATAGTAG | AGG | 3691 |
| 7167 | −1 | TTTGTGGAAGTAATTGGTGG | CGG | 3692 |
| 7170 | −1 | GAGTTTGTGGAAGTAATTGG | TGG | 3693 |
| 7173 | −1 | AGAGAGTTTGTGGAAGTAAT | TGG | 3694 |
| 7183 | −1 | ATAAAGGGGAAGAGAGTTTG | TGG | 3695 |
| 7197 | −1 | CCAGGCCAAGTAAAATAAAG | GGG | 3696 |
| 7198 | −1 | GCCAGGCCAAGTAAAATAAA | GGG | 3697 |
| 7199 | −1 | AGCCAGGCCAAGTAAAATAA | AGG | 3698 |
| 7203 | 1 | CTCTTCCCCTTTATTTTACT | TGG | 3699 |
| 7208 | 1 | CCCCTTTATTTTACTTGGCC | TGG | 3700 |
| 7215 | −1 | GACAGCCCCGTAGAGAAGCC | AGG | 3701 |
| 7219 | 1 | TACTTGGCCTGGCTTCTCTA | CGG | 3702 |
| 7220 | 1 | ACTTGGCCTGGCTTCTCTAC | GGG | 3703 |
| 7221 | 1 | CTTGGCCTGGCTTCTCTACG | GGG | 3704 |
| 7231 | 1 | CTTCTCTACGGGGCTGTCCA | AGG | 3705 |
| 7237 | −1 | CACCGGTAAGGATGCAACCT | TGG | 3706 |
| 7246 | 1 | GTCCAAGGTTGCATCCTTAC | CGG | 3707 |
| 7249 | −1 | CGATGACCCAAACACCGGTA | AGG | 3708 |
| 7253 | 1 | GTTGCATCCTTACCGGTGTT | TGG | 3709 |
| 7254 | 1 | TTGCATCCTTACCGGTGTTT | GGG | 3710 |
| 7254 | −1 | GTGTGCGATGACCCAAACAC | CGG | 3711 |
| 7273 | 1 | TGGGTCATCGCACACGAATG | TGG | 3712 |
| 7285 | −1 | GATAATCACTGAATGCATGG | TGG | 3713 |
| 7288 | −1 | ATTGATAATCACTGAATGCA | TGG | 3714 |
| 7301 | 1 | ATGCATTCAGTGATTATCAA | TGG | 3715 |
| 7314 | 1 | TTATCAATGGCTTGATGATA | CGG | 3716 |
| 7318 | 1 | CAATGGCTTGATGATACGGT | TGG | 3717 |
| 7330 | −1 | AGAGGCCAGAGTGGAGAACA | AGG | 3718 |
| 7336 | 1 | GTTGGCCTTGTTCTCCACTC | TGG | 3719 |
| 7339 | −1 | AAGGGACCAAGAGGCCAGAG | TGG | 3720 |
| 7344 | 1 | TGTTCTCCACTCTGGCCTCT | TGG | 3721 |
| 7348 | −1 | ATGAAAAGTAAGGGACCAAG | AGG | 3722 |
| 7357 | −1 | TGTATTTCCATGAAAAGTAA | GGG | 3723 |
| 7358 | −1 | CTGTATTTCCATGAAAAGTA | AGG | 3724 |
| 7361 | 1 | TCTTGGTCCCTTACTTTTCA | TGG | 3725 |
| 7381 | −1 | TGTTGGAGTGGTGGCGACGG | TGG | 3726 |
| 7384 | −1 | CGGTGTTGGAGTGGTGGCGA | CGG | 3727 |
| 7390 | −1 | GGGAACCGGTGTTGGAGTGG | TGG | 3728 |
| 7393 | −1 | CAAGGGAACCGGTGTTGGAG | TGG | 3729 |
| 7396 | 1 | CGTCGCCACCACTCCAACAC | CGG | 3730 |
| 7398 | −1 | CCTGTCAAGGGAACCGGTGT | TGG | 3731 |
| 7404 | −1 | TTCATCCCTGTCAAGGGAAC | CGG | 3732 |
| 7409 | 1 | CCAACACCGGTTCCCTTGAC | AGG | 3733 |
| 7410 | 1 | CAACACCGGTTCCCTTGACA | GGG | 3734 |
| 7410 | −1 | AAAGACTTCATCCCTGTCAA | GGG | 3735 |
| 7411 | −1 | CAAAGACTTCATCCCTGTCA | AGG | 3736 |
| 7436 | 1 | AAGTCTTTGTGCCAAAGAAA | AGG | 3737 |
| 7436 | −1 | GCAATACTAGACCTTTTCTT | TGG | 3738 |
| 7451 | 1 | AGAAAAGGTCTAGTATTGCA | TGG | 3739 |
| 7454 | 1 | AAAGGTCTAGTATTGCATGG | TGG | 3740 |
| 7474 | −1 | CTCTTCCTAGTGGGTTATTA | AGG | 3741 |
| 7480 | 1 | AAGTACCTTAATAACCCACT | AGG | 3742 |
| 7483 | −1 | GTGTGAGCACTCTTCCTAGT | GGG | 3743 |
| 7484 | −1 | AGTGTGAGCACTCTTCCTAG | TGG | 3744 |
| 7515 | −1 | AAGTGGCCAACCAAGGGTGA | GGG | 3745 |
| 7516 | 1 | CTCACTATTACCCTCACCCT | TGG | 3746 |
| 7516 | −1 | AAAGTGGCCAACCAAGGGTG | AGG | 3747 |
| 7520 | 1 | CTATTACCCTCACCCTTGGT | TGG | 3748 |
| 7521 | −1 | CAAGTAAAGTGGCCAACCAA | GGG | 3749 |
| 7522 | −1 | CCAAGTAAAGTGGCCAACCA | AGG | 3750 |
| 7532 | −1 | ACATTAAAAGCCAAGTAAAG | TGG | 3751 |
| 7533 | 1 | CCTTGGTTGGCCACTTTACT | TGG | 3752 |
| 7548 | 1 | TTACTTGGCTTTTAATGTCT | CGG | 3753 |
| 7549 | 1 | TACTTGGCTTTTAATGTCTC | GGG | 3754 |
| 7553 | 1 | TGGCTTTTAATGTCTCGGGA | AGG | 3755 |
| 7565 | 1 | TCTCGGGAAGGCCTTATGAT | AGG | 3756 |
| 7565 | −1 | TGGCAAGCAAACCTATCATA | AGG | 3757 |
| 7585 | −1 | TTGGACCATATGGGTCATAG | TGG | 3758 |
| 7591 | 1 | GCTTGCCACTATGACCCATA | TGG | 3759 |
| 7594 | −1 | CGGAGTAGATTGGACCATAT | GGG | 3760 |
| 7595 | −1 | TCGGAGTAGATTGGACCATA | TGG | 3761 |
| 7604 | −1 | CGTTCGCGATCGGAGTAGAT | TGG | 3762 |
| 7614 | −1 | TATTTGAAGTCGTTCGCGAT | CGG | 3763 |
| 7642 | 1 | CAAATATACATATCAGATGC | TGG | 3764 |
| 7663 | 1 | GGAGTGCTTGCAGTTTCCTA | TGG | 3765 |
| 7668 | −1 | GACAAGACGAATAAGACCAT | AGG | 3766 |
| 7683 | 1 | TGGTCTTATTCGTCTTGTCC | TGG | 3767 |
| 7690 | 1 | ATTCGTCTTGTCCTGGCAAA | AGG | 3768 |
| 7690 | −1 | CCCAGGCAAGGCCTTTTGCC | AGG | 3769 |
| 7700 | 1 | TCCTGGCAAAAGGCCTTGCC | TGG | 3770 |
| 7701 | 1 | CCTGGCAAAAGGCCTTGCCT | GGG | 3771 |
| 7702 | −1 | AAACAGCCACAACCCAGGCA | AGG | 3772 |
| 7707 | 1 | AAAAGGCCTTGCCTGGGTTG | TGG | 3773 |
| 7707 | −1 | ACCATAAACAGCCACAACCC | AGG | 3774 |
| 7717 | 1 | GCCTGGGTTGTGGCTGTTTA | TGG | 3775 |
| 7733 | −1 | AATCCATTGACCACTAGCAA | TGG | 3776 |
| 7734 | 1 | TTATGGTGTTCCATTGCTAG | TGG | 3777 |
| 7741 | 1 | GTTCCATTGCTAGTGGTCAA | TGG | 3778 |
| 7749 | 1 | GCTAGTGGTCAATGGATTCT | TGG | 3779 |
| 7789 | −1 | AATCGTAATGTGGCAATGCA | GGG | 3780 |
| 7790 | −1 | GAATCGTAATGTGGCAATGC | AGG | 3781 |
| 7799 | −1 | CACTCGGATGAATCGTAATG | TGG | 3782 |
| 7811 | 1 | CACATTACGATTCATCCGAG | TGG | 3783 |
| 7812 | 1 | ACATTACGATTCATCCGAGT | GGG | 3784 |
| 7815 | −1 | TCCCCTCAACCAGTCCCACT | CGG | 3785 |
| 7817 | 1 | ACGATTCATCCGAGTGGGAC | TGG | 3786 |
| 7823 | 1 | CATCCGAGTGGGACTGGTTG | AGG | 3787 |
| 7824 | 1 | ATCCGAGTGGGACTGGTTGA | GGG | 3788 |
| 7825 | 1 | TCCGAGTGGGACTGGTTGAG | GGG | 3789 |
| 7833 | 1 | GGACTGGTTGAGGGGAGCAT | TGG | 3790 |
| 7842 | 1 | GAGGGGAGCATTGGCGACCG | TGG | 3791 |
| 7848 | −1 | GATTCCATAGTCTCTGTCCA | CGG | 3792 |
| 7855 | 1 | GCGACCGTGGACAGAGACTA | TGG | 3793 |
| 7869 | 1 | AGACTATGGAATCCTGAACA | AGG | 3794 |
| 7870 | −1 | TGTTATGGAAGACCTTGTTC | AGG | 3795 |
| 7885 | −1 | CATGTGTGTCAGTGATGTTA | TGG | 3796 |
| 7899 | 1 | TAACATCACTGACACACATG | TGG | 3797 |
| 7926 | −1 | TGCATGGTAATGCGGCATTG | TGG | 3798 |
| 7934 | −1 | GCCTCCATTGCATGGTAATG | CGG | 3799 |
| 7941 | 1 | AATGCCGCATTACCATGCAA | TGG | 3800 |
| 7942 | −1 | CATTTGTAGCCTCCATTGCA | TGG | 3801 |
| 7944 | 1 | GCCGCATTACCATGCAATGG | AGG | 3802 |
| 7971 | 1 | AAATGCAATAAAACCAATTT | TGG | 3803 |
| 7972 | 1 | AATGCAATAAAACCAATTTT | GGG | 3804 |
| 7973 | −1 | CGATAATACTCTCCCAAAAT | TGG | 3805 |
| 7985 | 1 | CAATTTTGGGAGAGTATTAT | CGG | 3806 |
| 7993 | 1 | GGAGAGTATTATCGGTTCGA | TGG | 3807 |
| 7994 | 1 | GAGAGTATTATCGGTTCGAT | GGG | 3808 |

TABLE 18
gRNA's targeting sequences of FAD2-3.

| Position | | | | SEQ |
|---|---|---|---|---|
| on SEQ ID | | | | ID |
| NO: 3809 | Strand | Sequence | PAM | NO: |
| 10 | −1 | TAGCTAGAGTGGCGAAGTGA | TGG | 3811 |
| 21 | −1 | ACCACAGGAAGTAGCTAGAG | TGG | 3812 |
| 31 | 1 | GCCACTCTAGCTACTTCCTG | TGG | 3813 |
| 34 | 1 | ACTCTAGCTACTTCCTGTGG | TGG | 3814 |
| 36 | −1 | TGATTCTGCTCAACCACCAC | AGG | 3815 |
| 67 | −1 | CAAAGAAGATCTTTGGAGGA | TGG | 3816 |
| 71 | −1 | CCAGCAAAGAAGATCTTTGG | AGG | 3817 |
| 74 | −1 | CGGCCAGCAAAGAAGATCTT | TGG | 3818 |
| 82 | 1 | CCTCCAAAGATCTTCTTTGC | TGG | 3819 |
| 94 | −1 | CAAAAAGAAAGATGAAGATT | CGG | 3820 |
| 115 | 1 | ATCTTTCTTTTTGAGTATGA | AGG | 3821 |
| 129 | −1 | AAGATTTAGTGACGTAATCT | CGG | 3822 |
| 152 | 1 | CACTAAATCTTGCCACTTTT | TGG | 3823 |
| 153 | −1 | CTGAGATAATCACCAAAAAG | TGG | 3824 |
| 176 | −1 | AGAAAACCAAAAGTATGTTG | TGG | 3825 |
| 181 | 1 | TCTCAGCCACAACATACTTT | TGG | 3826 |
| 202 | −1 | CTATCAAGTTAATGATGGCA | AGG | 3827 |
| 207 | −1 | CAAGTCTATCAAGTTAATGA | TGG | 3828 |
| 248 | 1 | CAGCTTCTTCTTTTTCTTTT | TGG | 3829 |
| 276 | −1 | GGTTATTACACAGGAAACTA | TGG | 3830 |
| 285 | −1 | ATCTTCTTCGGTTATTACAC | AGG | 3831 |
| 297 | −1 | TCATCTTTGCTTATCTTCTT | CGG | 3832 |
| 316 | 1 | GATAAGCAAAGATGAGAGAG | AGG | 3833 |
| 344 | 1 | GAAATTTGAACACTAGCTAA | TGG | 3834 |
| 345 | 1 | AAATTTGAACACTAGCTAAT | GGG | 3835 |
| 380 | −1 | CTACCTTATGCAAGGGAAGC | TGG | 3836 |
| 387 | −1 | CTGCACGCTACCTTATGCAA | GGG | 3837 |
| 388 | 1 | AGTCCAGCTTCCCTTGCATA | AGG | 3838 |
| 388 | −1 | GCTGCACGCTACCTTATGCA | AGG | 3839 |
| 406 | 1 | TAAGGTAGCGTGCAGCACAA | TGG | 3840 |
| 423 | 1 | CAATGGCAATCAATCAATTC | AGG | 3841 |
| 457 | −1 | TAAATTGCCATTTATTGCAT | GGG | 3842 |
| 458 | −1 | TTAAATTGCCATTTATTGCA | TGG | 3843 |
| 461 | 1 | CATGTTTCCCATGCAATAAA | TGG | 3844 |
| 479 | 1 | AATGGCAATTTAATGAAATT | TGG | 3845 |
| 493 | 1 | GAAATTTGGAATCCATCACA | TGG | 3846 |
| 494 | −1 | ACAAGCATTATACCATGTGA | TGG | 3847 |
| 514 | 1 | GGTATAATGCTTGTATCCGT | TGG | 3848 |
| 519 | −1 | CATTTGCCTCATGCTTCCAA | CGG | 3849 |
| 524 | 1 | TTGTATCCGTTGGAAGCATG | AGG | 3850 |
| 552 | 1 | GTTAACATTTGCTTTCCTGA | TGG | 3851 |
| 556 | −1 | TGCTTGATTTCAAATCCATC | AGG | 3852 |
| 571 | 1 | ATGGATTTGAAATCAAGCAT | TGG | 3853 |
| 595 | 1 | TCTTAAGCATATAACACAGT | TGG | 3854 |
| 596 | 1 | CTTAAGCATATAACACAGTT | GGG | 3855 |
| 601 | 1 | GCATATAACACAGTTGGGCT | TGG | 3856 |
| 618 | −1 | TACTTGGGCTAGAATGATTT | TGG | 3857 |
| 633 | −1 | GAATGGAAAATAAACTACTT | GGG | 3858 |
| 634 | −1 | TGAATGGAAAATAAACTACT | TGG | 3859 |
| 650 | −1 | GTTAGTTTCTTGTGGCTGAA | TGG | 3860 |
| 658 | −1 | TAATATGAGTTAGTTTCTTG | TGG | 3861 |
| 692 | 1 | ATGCTGAATATTTTAATCAT | TGG | 3862 |
| 693 | 1 | TGCTGAATATTTTAATCATT | GGG | 3863 |
| 718 | 1 | AGCAACATTTTCTTTTATTT | CGG | 3864 |
| 730 | −1 | ACAATGTAGTGTTTTAAATT | GGG | 3865 |
| 731 | −1 | GACAATGTAGTGTTTTAAAT | TGG | 3866 |
| 758 | −1 | TTAATTTATTGTGATCATTG | TGG | 3867 |
| 777 | 1 | ATCACAATAAATTAATATCA | AGG | 3868 |
| 800 | 1 | CTGAAAGTTCATAAGCATAT | TGG | 3869 |
| 801 | 1 | TGAAAGTTCATAAGCATATT | GGG | 3870 |
| 819 | −1 | TTGGGCCGAAAATGAAATTC | TGG | 3871 |
| 825 | 1 | TTGCACCAGAATTTCATTTT | CGG | 3872 |
| 837 | −1 | TGTTGTGCAGATTTTGAGTT | GGG | 3873 |
| 838 | −1 | TTGTTGTGCAGATTTTGAGT | TGG | 3874 |
| 937 | −1 | AAACTCTAATTTAATTTTAA | TGG | 3875 |
| 961 | −1 | TTGATACAATTTGATGAGTT | TGG | 3876 |
| 985 | 1 | ATTGTATCAAGAGAAGTCAG | AGG | 3877 |
| 992 | 1 | CAAGAGAAGTCAGAGGTAAA | AGG | 3878 |
| 1014 | −1 | GAAAATGATATGCATGTAGA | AGG | 3879 |
| 1055 | −1 | TAACCCAATTTGGAGTGACA | GGG | 3880 |
| 1056 | −1 | GTAACCCAATTTGGAGTGAC | AGG | 3881 |
| 1062 | 1 | AACTCCCTGTCACTCCAAAT | TGG | 3882 |
| 1063 | 1 | ACTCCCTGTCACTCCAAATT | GGG | 3883 |
| 1065 | −1 | CTTTCCTATGTAACCCAATT | TGG | 3884 |
| 1072 | 1 | CACTCCAAATTGGGTTACAT | AGG | 3885 |
| 1110 | 1 | TCTTCACTGCTGTGCATCTA | CGG | 3886 |
| 1126 | 1 | TCTACGGTCATAAGTGTGTC | TGG | 3887 |
| 1127 | 1 | CTACGGTCATAAGTGTGTCT | GGG | 3888 |
| 1128 | 1 | TACGGTCATAAGTGTGTCTG | GGG | 3889 |
| 1141 | −1 | TTAGCCATTGAAACACAACT | TGG | 3890 |
| 1148 | 1 | GGGACCAAGTTGTGTTTCAA | TGG | 3891 |
| 1159 | 1 | GTGTTTCAATGGCTAAGATA | TGG | 3892 |
| 1160 | 1 | TGTTTCAATGGCTAAGATAT | GGG | 3893 |
| 1176 | −1 | GCATAAACAAGTATTTTCAA | TGG | 3894 |
| 1201 | −1 | TTTGTTGACCGAAAGCTATA | GGG | 3895 |
| 1202 | −1 | CTTTGTTGACCGAAAGCTAT | AGG | 3896 |
| 1204 | 1 | TTATGCTGCCCTATAGCTTT | CGG | 3897 |
| 1236 | −1 | AATCCCCAAATTAGAAGTTG | TGG | 3898 |
| 1242 | 1 | CAGAACCACAACTTCTAATT | TGG | 3899 |
| 1243 | 1 | AGAACCACAACTTCTAATTT | GGG | 3900 |
| 1244 | 1 | GAACCACAACTTCTAATTTG | GGG | 3901 |
| 1265 | 1 | GGATTAAATGAAAAGTGACC | TGG | 3902 |
| 1272 | −1 | GTGTGCTTCACCAACTCACC | AGG | 3903 |
| 1273 | 1 | TGAAAAGTGACCTGGTGAGT | TGG | 3904 |
| 1292 | 1 | TTGGTGAAGCACACTGCTTT | AGG | 3905 |
| 1293 | 1 | TGGTGAAGCACACTGCTTTA | GGG | 3906 |
| 1303 | 1 | CACTGCTTTAGGGTTGACCT | AGG | 3907 |
| 1309 | −1 | TTGCTGAGTTCCTCTCTCCT | AGG | 3908 |
| 1310 | 1 | TTAGGGTTGACCTAGGAGAG | AGG | 3909 |
| 1328 | 1 | AGAGGAACTCAGCAACATGC | AGG | 3910 |
| 1329 | 1 | GAGGAACTCAGCAACATGCA | GGG | 3911 |
| 1368 | 1 | TTCTCATCATTCTGAAGCGA | AGG | 3912 |
| 1388 | −1 | GAACATAAACTTCAAAACAC | TGG | 3913 |
| 1455 | 1 | AGACAGTGCTACCTAGTCAA | AGG | 3914 |
| 1455 | −1 | GGCGGAATGTTCCTTTGACT | AGG | 3915 |
| 1473 | −1 | GATTTAGTTCCTCATTCTGG | CGG | 3916 |
| 1475 | 1 | AGGAACATTCCGCCAGAATG | AGG | 3917 |
| 1476 | −1 | TGTGATTTAGTTCCTCATTC | TGG | 3918 |
| 1489 | 1 | AGAATGAGGAACTAAATCAC | AGG | 3919 |
| 1502 | 1 | AAATCACAGGCAAGCAAATC | TGG | 3920 |
| 1522 | 1 | TGGTTCATCACATAGTAAAG | AGG | 3921 |
| 1552 | −1 | TTACAATCTGTAAAGCATGA | AGG | 3922 |
| 1600 | 1 | TATATTTCTGTAGTTTCTTG | AGG | 3923 |
| 1607 | 1 | CTGTAGTTTCTTGAGGTAAA | AGG | 3924 |
| 1643 | 1 | TTCAAGTAAAAGTCTAAGAG | TGG | 3925 |
| 1687 | −1 | AAGAAATCTGAAATCACTCA | AGG | 3926 |
| 1736 | 1 | TCTTGTATCTTGTACCTGTG | AGG | 3927 |
| 1739 | −1 | CTAAGAAAAGGGTACCTCAC | AGG | 3928 |
| 1750 | −1 | TGCTAACCCAACTAAGAAAA | GGG | 3929 |
| 1751 | −1 | GTGCTAACCCAACTAAGAAA | AGG | 3930 |
| 1754 | 1 | TGAGGTACCCTTTTCTTAGT | TGG | 3931 |
| 1755 | 1 | GAGGTACCCTTTTCTTAGTT | GGG | 3932 |
| 1780 | 1 | AGCACTAAGAGTGAAGAATT | AGG | 3933 |
| 1803 | 1 | AGTTTTAACCTAACTTGACT | TGG | 3934 |
| 1806 | 1 | TAGCATAACCAAGTCAAGTT | AGG | 3935 |
| 1845 | 1 | AAAAGATTGTGTCAATTCTG | TGG | 3936 |
| 1851 | 1 | TTGTGTCAATTCTGTGGAAT | TGG | 3937 |
| 1894 | −1 | AGTCTCCACCACAACAGTGG | TGG | 3938 |
| 1897 | 1 | TGAAAATTCCACCACTGTTG | TGG | 3939 |
| 1897 | −1 | TCCAGTCTCCACCACAACAG | TGG | 3940 |
| 1900 | 1 | AAATTCCACCACTGTTGTGG | TGG | 3941 |
| 1907 | 1 | ACCACTGTTGTGGTGGAGAC | TGG | 3942 |
| 1914 | 1 | TTGTGGTGGAGACTGGACGT | AGG | 3943 |
| 1929 | 1 | GACGTAGGTTGCATTGCACA | AGG | 3944 |
| 1942 | 1 | TTGCACAAGGCAACCGAACC | AGG | 3945 |
| 1944 | −1 | CACCGTCATGTATCCTGGTT | CGG | 3946 |
| 1949 | −1 | GCTGACACCGTCATGTATCC | TGG | 3947 |
| 1953 | 1 | AACCGAACCAGGATACATGA | CGG | 3948 |
| 1981 | −1 | TCAGAAAACAGAACAGAACA | AGG | 3949 |
| 2036 | 1 | TTGTCTCATAAATTTTTCGC | TGG | 3950 |
| 2076 | 1 | AGATTGCAAGTTTGTTTAAA | AGG | 3951 |
| 2077 | 1 | GATTGCAAGTTTGTTTAAAA | GGG | 3952 |
| 2100 | 1 | TTTTGCATTAAAGTAAAAGA | AGG | 3953 |
| 2124 | −1 | GTAGAAGGCTTAAAGAAAGG | GGG | 3954 |
| 2125 | −1 | TGTAGAAGGCTTAAAGAAAG | GGG | 3955 |
| 2126 | −1 | TTGTAGAAGGCTTAAAGAAA | GGG | 3956 |
| 2127 | −1 | GTTGTAGAAGGCTTAAAGAA | AGG | 3957 |
| 2139 | −1 | ATATTTCTGAAGGTTGTAGA | AGG | 3958 |
| 2149 | −1 | ATAAGTCATAATATTTCTGA | AGG | 3959 |
| 2191 | −1 | TATTTTGATTTTCAATATTT | GGG | 3960 |
| 2192 | −1 | TTATTTTGATTTTCAATATT | TGG | 3961 |
| 2222 | −1 | TATATATATATATATATAGA | GGG | 3962 |
| 2223 | −1 | ATATATATATATATATATAG | AGG | 3963 |
| 2403 | 1 | GAGACATGAATTTAACCCTT | CGG | 3964 |
| 2407 | −1 | CTTACAGATAAGTTGCCGAA | GGG | 3965 |
| 2408 | −1 | ACTTACAGATAAGTTGCCGA | AGG | 3966 |
| 2436 | −1 | AAACACTACATGAATGATTT | TGG | 3967 |
| 2459 | 1 | ATGTAGTGTTTATTTTTTTG | TGG | 3968 |
| 2499 | −1 | CTTAGAATGAGATAAGTTAA | AGG | 3969 |
| 2522 | −1 | CAAAAAACAAGAAACATTTA | TGG | 3970 |
| 2538 | 1 | AATGTTTCTTGTTTTTTGAT | TGG | 3971 |
| 2541 | 1 | GTTTCTTGTTTTTTGATTGG | TGG | 3972 |
| 2544 | 1 | TCTTGTTTTTTGATTGGTGG | AGG | 3973 |
| 2551 | 1 | TTTTGATTGGTGGAGGAATT | TGG | 3974 |
| 2564 | 1 | AGGAATTTGGTGTTACAAAT | TGG | 3975 |
| 2591 | −1 | AAATCAGTCGAGCTATTTGA | GGG | 3976 |
| 2592 | −1 | AAAATCAGTCGAGCTATTTG | AGG | 3977 |
| 2616 | 1 | GACTGATTTTGATTATAATT | TGG | 3978 |
| 2629 | 1 | TATAATTTGGAATATTTGAA | AGG | 3979 |
| 2669 | −1 | GCACTATTTTTAGAGACTCT | TGG | 3980 |
| 2706 | 1 | CATAATTGCTTAAGTTAAAC | TGG | 3981 |
| 2840 | 1 | ATTTTGTTTATCTTAACTTT | TGG | 3982 |
| 2907 | 1 | AAAAAAAAAAAAAACTTATT | AGG | 3983 |
| 2908 | 1 | AAAAAAAAAAAAACTTATTA | GGG | 3984 |
| 2923 | 1 | TATTAGGGTTGATGCTAAAT | TGG | 3985 |
| 2926 | 1 | TAGGGTTGATGCTAAATTGG | TGG | 3986 |
| 2927 | 1 | AGGGTTGATGCTAAATTGGT | GGG | 3987 |
| 2966 | −1 | ACAAGTTAAGGTATTTTAGT | TGG | 3988 |
| 2978 | −1 | TTTCGAGTTACAACAAGTTA | AGG | 3989 |
| 3017 | −1 | ACTCACATTGGGTTTTTATA | GGG | 3990 |
| 3018 | −1 | CACTCACATTGGGTTTTTAT | AGG | 3991 |
| 3028 | −1 | GTTGTTGTCTCACTCACATT | GGG | 3992 |
| 3029 | −1 | AGTTGTTGTCTCACTCACAT | TGG | 3993 |
| 3069 | 1 | ATTACATAAAACCTTAAACG | TGG | 3994 |
| 3069 | −1 | GAATCTCGCAGCCACGTTTA | AGG | 3995 |
| 3088 | 1 | GTGGCTGCGAGATTCATCAT | AGG | 3996 |
| 3118 | 1 | CTCACTTCTCTTCTCTCTGT | TGG | 3997 |
| 3132 | 1 | CTCTGTTGGAATTGCTTTCA | CGG | 3998 |
| 3157 | −1 | GCAGAATGTTTTATAATTAA | AGG | 3999 |
| 3236 | 1 | AAAAATTGAACTCGTGTTGC | TGG | 4000 |
| 3261 | −1 | GCATGTTGATGTTATAGAAA | TGG | 4001 |
| 3280 | 1 | ATAACATCAACATGCATGCT | TGG | 4002 |
| 3322 | −1 | TCAAAATTCAGAAAAATTCA | AGG | 4003 |
| 3341 | 1 | TTTTCTGAATTTTGAAGCAA | AGG | 4004 |
| 3342 | 1 | TTTCTGAATTTTGAAGCAAA | GGG | 4005 |
| 3343 | 1 | TTCTGAATTTTGAAGCAAAG | GGG | 4006 |
| 3348 | 1 | AATTTTGAAGCAAAGGGGTG | AGG | 4007 |
| 3367 | −1 | GAAATCCAGTAGAAATAACA | TGG | 4008 |
| 3373 | 1 | TTCTTCCATGTTATTTCTAC | TGG | 4009 |
| 3440 | −1 | ATAACAAAACTGTGTGTCTG | AGG | 4010 |
| 3460 | 1 | CACAGTTTTGTTATCCCAGC | TGG | 4011 |
| 3463 | −1 | GAACGGCTATGAAACCAGCT | GGG | 4012 |
| 3464 | −1 | CGAACGGCTATGAAACCAGC | TGG | 4013 |
| 3480 | −1 | AAAGAAAAATGAAAAACGAA | CGG | 4014 |
| 3513 | 1 | TTATTTATTTATTTATTCTT | TGG | 4015 |
| 3539 | −1 | GAAGGAGTTCAATGAAAAAA | GGG | 4016 |
| 3540 | −1 | AGAAGGAGTTCAATGAAAAA | AGG | 4017 |
| 3554 | 1 | TTTTTCATTGAACTCCTTCT | TGG | 4018 |
| 3557 | −1 | GATAGGTGTCATTTCCAAGA | AGG | 4019 |
| 3574 | −1 | TATGTAAAGATATAAATGAT | AGG | 4020 |
| 3616 | 1 | GAAAATTGTCATATAGAAAT | AGG | 4021 |
| 3641 | −1 | TTTGCAACAGAATAATCTTA | TGG | 4022 |
| 3676 | −1 | ATTAAAAAGAAAAAGTGATG | GGG | 4023 |
| 3677 | −1 | TATTAAAAAGAAAAAGTGAT | GGG | 4024 |
| 3678 | −1 | ATATTAAAAAGAAAAAGTGA | TGG | 4025 |
| 3702 | 1 | TTTTTAATATTTTTATTTTC | TGG | 4026 |
| 3716 | −1 | CATTAATTATTGCTTGGACT | TGG | 4027 |
| 3722 | −1 | GGGTCCCATTAATTATTGCT | TGG | 4028 |
| 3728 | 1 | CAAGTCCAAGCAATAATTAA | TGG | 4029 |
| 3729 | 1 | AAGTCCAAGCAATAATTAAT | GGG | 4030 |
| 3742 | −1 | TATCCATGAAAAAGTAAAAA | GGG | 4031 |
| 3743 | −1 | CTATCCATGAAAAAGTAAAA | AGG | 4032 |
| 3750 | 1 | GGACCCTTTTTACTTTTTCA | TGG | 4033 |
| 3771 | 1 | GGATAGTAAATATATAAATA | AGG | 4034 |
| 3812 | 1 | AACATTACAACTTCTCTTCT | TGG | 4035 |
| 3846 | −1 | TAACCTCAAGGAAAAGATTA | TGG | 4036 |
| 3854 | 1 | CATCCATAATCTTTTCCTTG | AGG | 4037 |
| 3858 | −1 | AGAAAAGAAGATTAACCTCA | AGG | 4038 |
| 3887 | −1 | ATATAATTCAGTCATTTTCA | AGG | 4039 |
| 3946 | −1 | AGGAAATGGCAATGGATTTC | TGG | 4040 |
| 3954 | −1 | ACTACTAAAGGAAATGGCAA | TGG | 4041 |
| 3960 | −1 | AATTATACTACTAAAGGAAA | TGG | 4042 |
| 3966 | −1 | AGAATGAATTATACTACTAA | AGG | 4043 |
| 3979 | 1 | TTTAGTAGTATAATTCATTC | TGG | 4044 |
| 4021 | 1 | GAAAATATTTGCATACATTA | AGG | 4045 |
| 4036 | 1 | CATTAAGGCATTAGTTTTGA | TGG | 4046 |
| 4075 | 1 | TGATGTATCATAATTGCTTC | TGG | 4047 |
| 4076 | 1 | GATGTATCATAATTGCTTCT | GGG | 4048 |
| 4088 | −1 | ACAAAATCATATATCAGTGA | GGG | 4049 |
| 4089 | −1 | GACAAAATCATATATCAGTG | AGG | 4050 |
| 4119 | 1 | TGTCATTGTGTATTTGTGTT | TGG | 4051 |
| 4161 | −1 | CTTGTATTAGTATACGTGTG | AGG | 4052 |
| 4188 | −1 | TTGTAAGGGGCATGATTCTG | AGG | 4053 |
| 4201 | −1 | CACTATAATATAGTTGTAAG | GGG | 4054 |
| 4202 | −1 | ACACTATAATATAGTTGTAA | GGG | 4055 |
| 4203 | −1 | CACACTATAATATAGTTGTA | AGG | 4056 |
| 4273 | −1 | CTTCTTTGGATTTTGGATGG | TGG | 4057 |
| 4276 | −1 | CAACTTCTTTGGATTTTGGA | TGG | 4058 |
| 4280 | −1 | CACACAACTTCTTTGGATTT | TGG | 4059 |
| 4287 | −1 | TACAACACACACAACTTCTT | TGG | 4060 |
| 4302 | 1 | AGAAGTTGTGTGTGTTGTAG | TGG | 4061 |
| 4318 | −1 | GAGCATGAAGAGTGTGTCAA | GGG | 4062 |
| 4319 | −1 | AGAGCATGAAGAGTGTGTCA | AGG | 4063 |
| 4352 | −1 | GCATGCATCTTCTATAATGA | AGG | 4064 |
| 4381 | 1 | CATGCTTTCTTGTCAATTGA | AGG | 4065 |
| 4382 | 1 | ATGCTTTCTTGTCAATTGAA | GGG | 4066 |
| 4415 | 1 | CATCACTCATACTAGTCACT | TGG | 4067 |
| 4457 | −1 | CACCAATTTCCAAACACATG | TGG | 4068 |
| 4459 | 1 | ATTTGAATGCCACATGTGTT | TGG | 4069 |
| 4466 | 1 | TGCCACATGTGTTTGGAAAT | TGG | 4070 |
| 4505 | −1 | AAATAGAGAAAAGAAAAGAA | AGG | 4071 |
| 4523 | 1 | TTTCTTTTCTCTATTTTTAT | AGG | 4072 |
| 4533 | 1 | CTATTTTTATAGGATTCCCA | AGG | 4073 |
| 4538 | −1 | TTGAAGTTCTATTATGCCTT | GGG | 4074 |
| 4539 | −1 | TTTGAAGTTCTATTATGCCT | TGG | 4075 |
| 4557 | 1 | ATAATAGAACTTCAAATAAC | TGG | 4076 |
| 4585 | −1 | ACCAGAAAAACAGCAAAGAA | AGG | 4077 |
| 4595 | 1 | TCCTTTCTTTGCTGTTTTTC | TGG | 4078 |
| 4601 | 1 | CTTTGCTGTTTTTCTGGTCA | TGG | 4079 |
| 4727 | 1 | CTAACTCTTTCTTTCTACAT | TGG | 4080 |
| 4734 | 1 | TTTCTTTCTACATTGGTAAC | AGG | 4081 |
| 4756 | 1 | GAGCTTTAACAACACAACAA | TGG | 4082 |
| 4757 | 1 | AGCTTTAACAACACAACAAT | GGG | 4083 |
| 4763 | 1 | AACAACACAACAATGGGAGC | TGG | 4084 |
| 4766 | 1 | AACACAACAATGGGAGCTGG | AGG | 4085 |
| 4767 | 1 | ACACAACAATGGGAGCTGGA | GGG | 4086 |
| 4809 | 1 | CTCAAAAGAAGCCTCTTTCA | AGG | 4087 |
| 4809 | −1 | GAATGTGGAACCCTTGAAAG | AGG | 4088 |
| 4810 | 1 | TCAAAAGAAGCCTCTTTCAA | GGG | 4089 |
| 4824 | −1 | CTGAATGGAGGGTTTGAATG | TGG | 4090 |
| 4835 | −1 | GTTGGCCAACACTGAATGGA | GGG | 4091 |
| 4836 | −1 | AGTTGGCCAACACTGAATGG | AGG | 4092 |
| 4839 | −1 | TTGAGTTGGCCAACACTGAA | TGG | 4093 |
| 4841 | 1 | TCAAACCCTCCATTCAGTGT | TGG | 4094 |
| 4853 | −1 | GTGGAATTGCTTTCTTGAGT | TGG | 4095 |
| 4872 | −1 | GAACGTTCAAAGCAATGTGG | TGG | 4096 |
| 4875 | −1 | AGAGAACGTTCAAAGCAATG | TGG | 4097 |
| 4912 | −1 | TAAGAGATCATAGACAACAT | AGG | 4098 |
| 4927 | 1 | TGTTGTCTATGATCTCTTAG | TGG | 4099 |
| 4939 | −1 | GGCAATGTAGAAGAGTAAGT | AGG | 4100 |
| 4960 | −1 | AAGCTTGTGGAAATAAGTGG | TGG | 4101 |
| 4963 | −1 | TGGAAGCTTGTGGAAATAAG | TGG | 4102 |
| 4973 | −1 | AAAATGGGTATGGAAGCTTG | TGG | 4103 |
| 4983 | −1 | GCAAGGAAGGAAAATGGGTA | TGG | 4104 |

TABLE 19
gRNA's targeting sequences of FAD2-4.

| Position | | | | |
|---|---|---|---|---|
| on | | | | SEQ |
| SEQ ID | | | | ID |
| NO: 4105 | Strand | Sequence | PAM | NO: |
| 10 | −1 | TAGCTAGAGTGGCGAAGTGA | TGG | 4107 |
| 21 | −1 | ACCACAGGAAGTAGCTAGAG | TGG | 4108 |
| 31 | 1 | GCCACTCTAGCTACTTCCTG | TGG | 4109 |
| 34 | 1 | ACTCTAGCTACTTCCTGTGG | TGG | 4110 |
| 36 | −1 | TGATTCTGCTCAACCACCAC | AGG | 4111 |
| 67 | −1 | CAAAGAAGATCTTTGGAGGA | TGG | 4112 |
| 71 | −1 | CCAGCAAAGAAGATCTTTGG | AGG | 4113 |
| 74 | −1 | CGGCCAGCAAAGAAGATCTT | TGG | 4114 |
| 82 | 1 | CCTCCAAAGATCTTCTTTGC | TGG | 4115 |
| 94 | −1 | CAAAAAGAAAGATGAAGATT | CGG | 4116 |
| 115 | 1 | ATCTTTCTTTTTGAGTATGA | AGG | 4117 |
| 129 | −1 | AAGATTTAGTGACGTAATCT | CGG | 4118 |
| 152 | 1 | CACTAAATCTTGCCACTTTT | TGG | 4119 |
| 153 | −1 | CTGAGATAATCACCAAAAAG | TGG | 4120 |
| 176 | −1 | AGAAAACCAAAAGTATGTTG | TGG | 4121 |
| 181 | 1 | TCTCAGCCACAACATACTTT | TGG | 4122 |
| 202 | −1 | CTATCAAGTTAATGATGGCA | AGG | 4123 |
| 207 | −1 | CAAGTCTATCAAGTTAATGA | TGG | 4124 |
| 248 | 1 | CAGCTTCTTCTTTTTCTTTT | TGG | 4125 |
| 276 | −1 | GGTTATTACACAGGAAACTA | TGG | 4126 |
| 285 | −1 | ATCTTCTTCGGTTATTACAC | AGG | 4127 |
| 297 | −1 | TCATCTTTGCTTATCTTCTT | CGG | 4128 |
| 316 | 1 | GATAAGCAAAGATGAGAGAG | AGG | 4129 |
| 344 | 1 | GAAATTTGAACACTAGCTAA | TGG | 4130 |
| 345 | 1 | AAATTTGAACACTAGCTAAT | GGG | 4131 |
| 380 | −1 | CTACCTTATGCAAGGGAAGC | TGG | 4132 |
| 387 | −1 | CTGCACGCTACCTTATGCAA | GGG | 4133 |
| 388 | 1 | AGTCCAGCTTCCCTTGCATA | AGG | 4134 |
| 388 | −1 | GCTGCACGCTACCTTATGCA | AGG | 4135 |
| 406 | 1 | TAAGGTAGCGTGCAGCACAA | TGG | 4136 |
| 423 | 1 | CAATGGCAATCAATCAATTC | AGG | 4137 |
| 457 | −1 | TAAATTGCCATTTATTGCAT | GGG | 4138 |
| 458 | −1 | TTAAATTGCCATTTATTGCA | TGG | 4139 |
| 461 | 1 | CATGTTTCCCATGCAATAAA | TGG | 4140 |
| 479 | 1 | AATGGCAATTTAATGAAATT | TGG | 4141 |
| 493 | 1 | GAAATTTGGAATCCATCACA | TGG | 4142 |
| 494 | −1 | ACAAGCATTATACCATGTGA | TGG | 4143 |
| 514 | 1 | GGTATAATGCTTGTATCCGT | TGG | 4144 |
| 519 | −1 | CATTTGCCTCATGCTTCCAA | CGG | 4145 |
| 524 | 1 | TTGTATCCGTTGGAAGCATG | AGG | 4146 |
| 552 | 1 | GTTAACATTTGCTTTCCTGA | TGG | 4147 |
| 556 | −1 | TGCTTGATTTCAAATCCATC | AGG | 4148 |
| 571 | 1 | ATGGATTTGAAATCAAGCAT | TGG | 4149 |
| 595 | 1 | TCTTAAGCATATAACACAGT | TGG | 4150 |
| 596 | 1 | CTTAAGCATATAACACAGTT | GGG | 4151 |
| 601 | 1 | GCATATAACACAGTTGGGCT | TGG | 4152 |
| 618 | −1 | TACTTGGGCTAGAATGATTT | TGG | 4153 |
| 633 | −1 | GAATGGAAAATAAACTACTT | GGG | 4154 |
| 634 | −1 | TGAATGGAAAATAAACTACT | TGG | 4155 |
| 650 | −1 | GTTAGTTTCTTGTGGCTGAA | TGG | 4156 |
| 658 | −1 | TAATATGAGTTAGTTTCTTG | TGG | 4157 |
| 692 | 1 | ATGCTGAATATTTTAATCAT | TGG | 4158 |
| 693 | 1 | TGCTGAATATTTTAATCATT | GGG | 4159 |
| 718 | 1 | AGCAACATTTTCTTTTATTT | CGG | 4160 |
| 730 | −1 | ACAATGTAGTGTTTTAAATT | GGG | 4161 |
| 731 | −1 | GACAATGTAGTGTTTTAAAT | TGG | 4162 |
| 758 | −1 | TTAATTTATTGTGATCATTG | TGG | 4163 |
| 777 | 1 | ATCACAATAAATTAATATCA | AGG | 4164 |
| 800 | 1 | CTGAAAGTTCATAAGCATAT | TGG | 4165 |
| 801 | 1 | TGAAAGTTCATAAGCATATT | GGG | 4166 |
| 819 | −1 | TTGGGCCGAAAATGAAATTC | TGG | 4167 |
| 825 | 1 | TTGCACCAGAATTTCATTTT | CGG | 4168 |
| 837 | −1 | TGTTGTGCAGATTTTGAGTT | GGG | 4169 |
| 838 | −1 | TTGTTGTGCAGATTTTGAGT | TGG | 4170 |
| 937 | −1 | AAACTCTAATTTAATTTTAA | TGG | 4171 |
| 961 | −1 | TTGATACAATTTGATGAGTT | TGG | 4172 |
| 985 | 1 | ATTGTATCAAGAGAAGTCAG | AGG | 4173 |
| 992 | 1 | CAAGAGAAGTCAGAGGTAAA | AGG | 4174 |
| 1014 | −1 | GAAAATGATATGCATGTAGA | AGG | 4175 |
| 1055 | −1 | TAACCCAATTTGGAGTGACA | GGG | 4176 |
| 1056 | −1 | GTAACCCAATTTGGAGTGAC | AGG | 4177 |
| 1062 | 1 | AACTCCCTGTCACTCCAAAT | TGG | 4178 |
| 1063 | 1 | ACTCCCTGTCACTCCAAATT | GGG | 4179 |
| 1065 | −1 | CTTTCCTATGTAACCCAATT | TGG | 4180 |
| 1072 | 1 | CACTCCAAATTGGGTTACAT | AGG | 4181 |
| 1110 | 1 | TCTTCACTGCTGTGCATCTA | CGG | 4182 |
| 1126 | 1 | TCTACGGTCATAAGTGTGTC | TGG | 4183 |
| 1127 | 1 | CTACGGTCATAAGTGTGTCT | GGG | 4184 |
| 1128 | 1 | TACGGTCATAAGTGTGTCTG | GGG | 4185 |
| 1141 | −1 | TTAGCCATTGAAACACAACT | TGG | 4186 |
| 1148 | 1 | GGGACCAAGTTGTGTTTCAA | TGG | 4187 |
| 1159 | 1 | GTGTTTCAATGGCTAAGATA | TGG | 4188 |
| 1160 | 1 | TGTTTCAATGGCTAAGATAT | GGG | 4189 |
| 1176 | −1 | GCATAAACAAGTATTTTCAA | TGG | 4190 |
| 1201 | −1 | TTTGTTGACCGAAAGCTATA | GGG | 4191 |
| 1202 | −1 | CTTTGTTGACCGAAAGCTAT | AGG | 4192 |
| 1204 | 1 | TTATGCTGCCCTATAGCTTT | CGG | 4193 |
| 1236 | −1 | AATCCCCAAATTAGAAGTTG | TGG | 4194 |
| 1242 | 1 | CAGAACCACAACTTCTAATT | TGG | 4195 |
| 1243 | 1 | AGAACCACAACTTCTAATTT | GGG | 4196 |
| 1244 | 1 | GAACCACAACTTCTAATTTG | GGG | 4197 |
| 1265 | 1 | GGATTAAATGAAAAGTGACC | TGG | 4198 |
| 1272 | −1 | GTGTGCTTCACCAACTCACC | AGG | 4199 |
| 1273 | 1 | TGAAAAGTGACCTGGTGAGT | TGG | 4200 |
| 1292 | 1 | TTGGTGAAGCACACTGCTTT | AGG | 4201 |
| 1293 | 1 | TGGTGAAGCACACTGCTTTA | GGG | 4202 |
| 1303 | 1 | CACTGCTTTAGGGTTGACCT | AGG | 4203 |
| 1309 | −1 | TTGCTGAGTTCCTCTCTCCT | AGG | 4204 |
| 1310 | 1 | TTAGGGTTGACCTAGGAGAG | AGG | 4205 |
| 1328 | 1 | AGAGGAACTCAGCAACATGC | AGG | 4206 |
| 1329 | 1 | GAGGAACTCAGCAACATGCA | GGG | 4207 |
| 1368 | 1 | TTCTCATCATTCTGAAGCGA | AGG | 4208 |
| 1388 | −1 | GAACATAAACTTCAAAACAC | TGG | 4209 |
| 1455 | 1 | AGACAGTGCTACCTAGTCAA | AGG | 4210 |
| 1455 | −1 | GGCGGAATGTTCCTTTGACT | AGG | 4211 |
| 1473 | −1 | GATTTAGTTCCTCATTCTGG | CGG | 4212 |
| 1475 | 1 | AGGAACATTCCGCCAGAATG | AGG | 4213 |
| 1476 | −1 | TGTGATTTAGTTCCTCATTC | TGG | 4214 |
| 1489 | 1 | AGAATGAGGAACTAAATCAC | AGG | 4215 |
| 1502 | 1 | AAATCACAGGCAAGCAAATC | TGG | 4216 |
| 1522 | 1 | TGGTTCATCACATAGTAAAG | AGG | 4217 |
| 1552 | −1 | TTACAATCTGTAAAGCATGA | AGG | 4218 |
| 1600 | 1 | TATATTTCTGTAGTTTCTTG | AGG | 4219 |
| 1607 | 1 | CTGTAGTTTCTTGAGGTAAA | AGG | 4220 |
| 1643 | 1 | TTCAAGTAAAAGTCTAAGAG | TGG | 4221 |
| 1687 | −1 | AAGAAATCTGAAATCACTCA | AGG | 4222 |
| 1736 | 1 | TCTTGTATCTTGTACCTGTG | AGG | 4223 |
| 1739 | −1 | CTAAGAAAAGGGTACCTCAC | AGG | 4224 |
| 1750 | −1 | TGCTAACCCAACTAAGAAAA | GGG | 4225 |
| 1751 | −1 | GTGCTAACCCAACTAAGAAA | AGG | 4226 |
| 1754 | 1 | TGAGGTACCCTTTTCTTAGT | TGG | 4227 |
| 1755 | 1 | GAGGTACCCTTTTCTTAGTT | GGG | 4228 |
| 1780 | 1 | AGCACTAAGAGTGAAGAATT | AGG | 4229 |
| 1803 | −1 | AGTTTTAACCTAACTTGACT | TGG | 4230 |
| 1806 | 1 | TAGCATAACCAAGTCAAGTT | AGG | 4231 |
| 1845 | 1 | AAAAGATTGTGTCAATTCTG | TGG | 4232 |
| 1851 | 1 | TTGTGTCAATTCTGTGGAAT | TGG | 4233 |
| 1894 | −1 | AGTCTCCACCACAACAGTGG | TGG | 4234 |
| 1897 | 1 | TGAAAATTCCACCACTGTTG | TGG | 4235 |
| 1897 | −1 | TCCAGTCTCCACCACAACAG | TGG | 4236 |
| 1900 | 1 | AAATTCCACCACTGTTGTGG | TGG | 4237 |
| 1907 | 1 | ACCACTGTTGTGGTGGAGAC | TGG | 4238 |
| 1914 | 1 | TTGTGGTGGAGACTGGACGT | AGG | 4239 |
| 1929 | 1 | GACGTAGGTTGCATTGCACA | AGG | 4240 |
| 1942 | 1 | TTGCACAAGGCAACCGAACC | AGG | 4241 |
| 1944 | −1 | CACCGTCATGTATCCTGGTT | CGG | 4242 |
| 1949 | −1 | GCTGACACCGTCATGTATCC | TGG | 4243 |
| 1953 | 1 | AACCGAACCAGGATACATGA | CGG | 4244 |
| 1981 | −1 | TCAGAAAACAGAACAGAACA | AGG | 4245 |
| 2036 | 1 | TTGTCTCATAAATTTTTCGC | TGG | 4246 |
| 2076 | 1 | AGATTGCAAGTTTGTTTAAA | AGG | 4247 |
| 2077 | 1 | GATTGCAAGTTTGTTTAAAA | GGG | 4248 |
| 2100 | 1 | TTTTGCATTAAAGTAAAAGA | AGG | 4249 |
| 2124 | −1 | GTAGAAGGCTTAAAGAAAGG | GGG | 4250 |
| 2125 | −1 | TGTAGAAGGCTTAAAGAAAG | GGG | 4251 |
| 2126 | −1 | TTGTAGAAGGCTTAAAGAAA | GGG | 4252 |
| 2127 | −1 | GTTGTAGAAGGCTTAAAGAA | AGG | 4253 |
| 2139 | −1 | ATATTTCTGAAGGTTGTAGA | AGG | 4254 |
| 2149 | −1 | ATAAGTCATAATATTTCTGA | AGG | 4255 |
| 2191 | −1 | TATTTTGATTTTCAATATTT | GGG | 4256 |
| 2192 | −1 | TTATTTTGATTTTCAATATT | TGG | 4257 |
| 2222 | −1 | TATATATATATATATATAGA | GGG | 4258 |
| 2223 | −1 | ATATATATATATATATATAG | AGG | 4259 |
| 2403 | 1 | GAGACATGAATTTAACCCTT | CGG | 4260 |
| 2407 | −1 | CTTACAGATAAGTTGCCGAA | GGG | 4261 |
| 2408 | −1 | ACTTACAGATAAGTTGCCGA | AGG | 4262 |
| 2436 | −1 | AAACACTACATGAATGATTT | TGG | 4263 |
| 2459 | 1 | ATGTAGTGTTTATTTTTTTG | TGG | 4264 |
| 2499 | −1 | CTTAGAATGAGATAAGTTAA | AGG | 4265 |
| 2522 | −1 | CAAAAAACAAGAAACATTTA | TGG | 4266 |
| 2538 | 1 | AATGTTTCTTGTTTTTTGAT | TGG | 4267 |
| 2541 | 1 | GTTTCTTGTTTTTTGATTGG | TGG | 4268 |
| 2544 | 1 | TCTTGTTTTTTGATTGGTGG | AGG | 4269 |
| 2551 | 1 | TTTTGATTGGTGGAGGAATT | TGG | 4270 |
| 2564 | 1 | AGGAATTTGGTGTTACAAAT | TGG | 4271 |
| 2591 | −1 | AAATCAGTCGAGCTATTTGA | GGG | 4272 |
| 2592 | −1 | AAAATCAGTCGAGCTATTTG | AGG | 4273 |
| 2616 | 1 | GACTGATTTTGATTATAATT | TGG | 4274 |
| 2629 | 1 | TATAATTTGGAATATTTGAA | AGG | 4275 |
| 2669 | −1 | GCACTATTTTTAGAGACTCT | TGG | 4276 |
| 2706 | 1 | CATAATTGCTTAAGTTAAAC | TGG | 4277 |
| 2840 | 1 | ATTTTGTTTATCTTAACTTT | TGG | 4278 |
| 2907 | 1 | AAAAAAAAAAAAAACTTATT | AGG | 4279 |
| 2908 | 1 | AAAAAAAAAAAAACTTATTA | GGG | 4280 |
| 2923 | 1 | TATTAGGGTTGATGCTAAAT | TGG | 4281 |
| 2926 | 1 | TAGGGTTGATGCTAAATTGG | TGG | 4282 |
| 2927 | 1 | AGGGTTGATGCTAAATTGGT | GGG | 4283 |
| 2966 | −1 | ACAAGTTAAGGTATTTTAGT | TGG | 4284 |
| 2978 | −1 | TTTCGAGTTACAACAAGTTA | AGG | 4285 |
| 3017 | −1 | ACTCACATTGGGTTTTTATA | GGG | 4286 |
| 3018 | −1 | CACTCACATTGGGTTTTTAT | AGG | 4287 |
| 3028 | −1 | GTTGTTGTCTCACTCACATT | GGG | 4288 |
| 3029 | −1 | AGTTGTTGTCTCACTCACAT | TGG | 4289 |
| 3069 | 1 | ATTACATAAAACCTTAAACG | TGG | 4290 |
| 3069 | −1 | GAATCTCGCAGCCACGTTTA | AGG | 4291 |
| 3088 | 1 | GTGGCTGCGAGATTCATCAT | AGG | 4292 |
| 3118 | 1 | CTCACTTCTCTTCTCTCTGT | TGG | 4293 |
| 3132 | 1 | CTCTGTTGGAATTGCTTTCA | CGG | 4294 |
| 3157 | −1 | GCAGAATGTTTTATAATTAA | AGG | 4295 |
| 3236 | 1 | AAAAATTGAACTCGTGTTGC | TGG | 4296 |
| 3261 | −1 | GCATGTTGATGTTATAGAAA | TGG | 4297 |
| 3280 | 1 | ATAACATCAACATGCATGCT | TGG | 4298 |
| 3322 | −1 | TCAAAATTCAGAAAAATTCA | AGG | 4299 |
| 3341 | 1 | TTTTCTGAATTTTGAAGCAA | AGG | 4300 |
| 3342 | 1 | TTTCTGAATTTTGAAGCAAA | GGG | 4301 |
| 3343 | 1 | TTCTGAATTTTGAAGCAAAG | GGG | 4302 |
| 3348 | 1 | AATTTTGAAGCAAAGGGGTG | AGG | 4303 |
| 3367 | −1 | GAAATCCAGTAGAAATAACA | TGG | 4304 |
| 3373 | 1 | TTCTTCCATGTTATTTCTAC | TGG | 4305 |
| 3440 | −1 | ATAACAAAACTGTGTGTCTG | AGG | 4306 |
| 3460 | 1 | CACAGTTTTGTTATCCCAGC | TGG | 4307 |
| 3463 | −1 | GAACGGCTATGAAACCAGCT | GGG | 4308 |
| 3464 | −1 | CGAACGGCTATGAAACCAGC | TGG | 4309 |
| 3480 | −1 | AAAGAAAAATGAAAAACGAA | CGG | 4310 |
| 3513 | 1 | TTATTTATTTATTTATTCTT | TGG | 4311 |
| 3539 | −1 | GAAGGAGTTCAATGAAAAAA | GGG | 4312 |
| 3540 | −1 | AGAAGGAGTTCAATGAAAAA | AGG | 4313 |
| 3554 | 1 | TTTTTCATTGAACTCCTTCT | TGG | 4314 |
| 3557 | −1 | GATAGGTGTCATTTCCAAGA | AGG | 4315 |
| 3574 | −1 | TATGTAAAGATATAAATGAT | AGG | 4316 |
| 3616 | 1 | GAAAATTGTCATATAGAAAT | AGG | 4317 |
| 3641 | −1 | TTTGCAACAGAATAATCTTA | TGG | 4318 |
| 3676 | −1 | ATTAAAAAGAAAAAGTGATG | GGG | 4319 |
| 3677 | −1 | TATTAAAAAGAAAAAGTGAT | GGG | 4320 |
| 3678 | −1 | ATATTAAAAAGAAAAAGTGA | TGG | 4321 |
| 3702 | 1 | TTTTTAATATTTTTATTTTC | TGG | 4322 |
| 3716 | −1 | CATTAATTATTGCTTGGACT | TGG | 4323 |
| 3722 | −1 | GGGTCCCATTAATTATTGCT | TGG | 4324 |
| 3728 | 1 | CAAGTCCAAGCAATAATTAA | TGG | 4325 |
| 3729 | 1 | AAGTCCAAGCAATAATTAAT | GGG | 4326 |
| 3742 | −1 | TATCCATGAAAAAGTAAAAA | GGG | 4327 |
| 3743 | −1 | CTATCCATGAAAAAGTAAAA | AGG | 4328 |
| 3750 | 1 | GGACCCTTTTTACTTTTTCA | TGG | 4329 |
| 3771 | 1 | GGATAGTAAATATATAAATA | AGG | 4330 |
| 3812 | 1 | AACATTACAACTTCTCTTCT | TGG | 4331 |
| 3846 | −1 | TAACCTCAAGGAAAAGATTA | TGG | 4332 |
| 3854 | 1 | CATCCATAATCTTTTCCTTG | AGG | 4333 |
| 3858 | −1 | AGAAAAGAAGATTAACCTCA | AGG | 4334 |
| 3887 | −1 | ATATAATTCAGTCATTTTCA | AGG | 4335 |
| 3946 | −1 | AGGAAATGGCAATGGATTTC | TGG | 4336 |
| 3954 | −1 | ACTACTAAAGGAAATGGCAA | TGG | 4337 |
| 3960 | −1 | AATTATACTACTAAAGGAAA | TGG | 4338 |
| 3966 | −1 | AGAATGAATTATACTACTAA | AGG | 4339 |
| 3979 | 1 | TTTAGTAGTATAATTCATTC | TGG | 4340 |
| 4021 | 1 | GAAAATATTTGCATACATTA | AGG | 4341 |
| 4036 | 1 | CATTAAGGCATTAGTTTTGA | TGG | 4342 |
| 4075 | 1 | TGATGTATCATAATTGCTTC | TGG | 4343 |
| 4076 | 1 | GATGTATCATAATTGCTTCT | GGG | 4344 |
| 4088 | −1 | ACAAAATCATATATCAGTGA | GGG | 4345 |
| 4089 | −1 | GACAAAATCATATATCAGTG | AGG | 4346 |
| 4119 | 1 | TGTCATTGTGTATTTGTGTT | TGG | 4347 |
| 4161 | −1 | CTTGTATTAGTATACGTGTG | AGG | 4348 |
| 4188 | −1 | TTGTAAGGGGCATGATTCTG | AGG | 4349 |
| 4201 | −1 | CACTATAATATAGTTGTAAG | GGG | 4350 |
| 4202 | −1 | ACACTATAATATAGTTGTAA | GGG | 4351 |
| 4203 | −1 | CACACTATAATATAGTTGTA | AGG | 4352 |
| 4273 | −1 | CTTCTTTGGATTTTGGATGG | TGG | 4353 |
| 4276 | −1 | CAACTTCTTTGGATTTTGGA | TGG | 4354 |
| 4280 | −1 | CACACAACTTCTTTGGATTT | TGG | 4355 |
| 4287 | −1 | TACAACACACACAACTTCTT | TGG | 4356 |
| 4302 | 1 | AGAAGTTGTGTGTGTTGTAG | TGG | 4357 |
| 4318 | −1 | GAGCATGAAGAGTGTGTCAA | GGG | 4358 |
| 4319 | −1 | AGAGCATGAAGAGTGTGTCA | AGG | 4359 |
| 4352 | −1 | GCATGCATCTTCTATAATGA | AGG | 4360 |
| 4381 | 1 | CATGCTTTCTTGTCAATTGA | AGG | 4361 |
| 4382 | 1 | ATGCTTTCTTGTCAATTGAA | GGG | 4362 |
| 4415 | 1 | CATCACTCATACTAGTCACT | TGG | 4363 |
| 4457 | −1 | CACCAATTTCCAAACACATG | TGG | 4364 |
| 4459 | 1 | ATTTGAATGCCACATGTGTT | TGG | 4365 |
| 4466 | 1 | TGCCACATGTGTTTGGAAAT | TGG | 4366 |
| 4505 | −1 | AAATAGAGAAAAGAAAAGAA | AGG | 4367 |
| 4523 | 1 | TTTCTTTTCTCTATTTTTAT | AGG | 4368 |
| 4533 | 1 | CTATTTTTATAGGATTCCCA | AGG | 4369 |
| 4538 | −1 | TTGAAGTTCTATTATGCCTT | GGG | 4370 |
| 4539 | −1 | TTTGAAGTTCTATTATGCCT | TGG | 4371 |
| 4557 | 1 | ATAATAGAACTTCAAATAAC | TGG | 4372 |
| 4585 | −1 | ACCAGAAAAACAGCAAAGAA | AGG | 4373 |
| 4595 | 1 | TCCTTTCTTTGCTGTTTTTC | TGG | 4374 |
| 4601 | 1 | CTTTGCTGTTTTTCTGGTCA | TGG | 4375 |
| 4727 | 1 | CTAACTCTTTCTTTCTACAT | TGG | 4376 |
| 4734 | 1 | TTTCTTTCTACATTGGTAAC | AGG | 4377 |
| 4756 | 1 | GAGCTTTAACAACACAACAA | TGG | 4378 |
| 4757 | 1 | AGCTTTAACAACACAACAAT | GGG | 4379 |
| 4763 | 1 | AACAACACAACAATGGGAGC | TGG | 4380 |
| 4766 | 1 | AACACAACAATGGGAGCTGG | AGG | 4381 |
| 4767 | 1 | ACACAACAATGGGAGCTGGA | GGG | 4382 |
| 4809 | 1 | CTCAAAAGAAGCCTCTTTCA | AGG | 4383 |
| 4809 | −1 | GAATGTGGAACCCTTGAAAG | AGG | 4384 |
| 4810 | 1 | TCAAAAGAAGCCTCTTTCAA | GGG | 4385 |
| 4824 | −1 | CTGAATGGAGGGTTTGAATG | TGG | 4386 |
| 4835 | −1 | GTTGGCCAACACTGAATGGA | GGG | 4387 |
| 4836 | −1 | AGTTGGCCAACACTGAATGG | AGG | 4388 |
| 4839 | −1 | TTGAGTTGGCCAACACTGAA | TGG | 4389 |
| 4841 | 1 | TCAAACCCTCCATTCAGTGT | TGG | 4390 |
| 4853 | −1 | GTGGAATTGCTTTCTTGAGT | TGG | 4391 |
| 4872 | −1 | GAACGTTCAAAGCAATGTGG | TGG | 4392 |
| 4875 | −1 | AGAGAACGTTCAAAGCAATG | TGG | 4393 |
| 4912 | −1 | TAAGAGATCATAGACAACAT | AGG | 4394 |
| 4927 | 1 | TGTTGTCTATGATCTCTTAG | TGG | 4395 |
| 4939 | −1 | GGCAATGTAGAAGAGTAAGT | AGG | 4396 |
| 4960 | −1 | AAGCTTGTGGAAATAAGTGG | TGG | 4397 |
| 4963 | −1 | TGGAAGCTTGTGGAAATAAG | TGG | 4398 |
| 4973 | −1 | AAAATGGGTATGGAAGCTTG | TGG | 4399 |
| 4983 | −1 | GCAAGGAAGGAAAATGGGTA | TGG | 4400 |

The above gRNA molecules have been cloned into suitable vectors and their sequence has been verified. In addition, different Cas9 versions have been analyzed for optimal compatibility between the Cas9 protein activity and the gRNA molecule in the peanut plant.

The efficiency of the designed gRNA molecules has been validated by transiently transforming peanut tissue culture. A plasmid carrying a gRNA sequence together with the Cas9 gene has been transformed into peanut protoplasts. The protoplast cells have been grown for a short period of time and then were analyzed for existence of genome editing events. The positive constructs have been subjected to the herein established stable transformation protocol into peanut plant tissue for producing genome edited peanut plants in Ara h and/or FAD2 genes.
Stage 3: Transforming peanut plants using Agrobacterium or biolistics (gene gun) methods.

For Agrobacterium and biolistics, a DNA plasmid carrying (Cas9+gene specific gRNA) can be used. A vector containing a selection marker, Cas9 gene and relevant gene specific gRNA's is constructed. For biolistics, Ribonucleoprotein (RNP) complexes carrying (Cas9 protein+gene specific gRNA) are used. RNP complexes are created by mixing the Cas9 protein with relevant gene specific gRNA's.

According to some embodiments of the present invention, transformation of various peanut tissues was performed using particle bombardment of:
DNA vectors
Ribonucleoprotein complex (RNP's)

According to further embodiments of the present invention, transformation of various peanut tissues was performed using Agrobacterium (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)
Stage 4: Selection of transformed peanut plants presenting Ara h and FAD2 related phenotypes as described above.

It should be appreciated that embodiments formed from combinations of features set forth in separate embodiments are also within the scope of the present invention.

While certain features of the invention have been illustrated and described herein, modifications, substitutions, and equivalents are included within the scope of the invention.

The terminology used herein is for the purpose of describing particular embodiments only and is not intended to be limiting of the invention. As used herein, the singular forms “a,” “an” and “the” are intended to include plural forms as well, unless the context clearly indicates otherwise.

It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

All publications, patent applications, patents, and other references mentioned in the disclosures of these publications in their entireties are hereby incorporated by reference into this application in order to more fully describe the state of the art to which this invention pertains.

In case of conflict, the patent specification, including definitions, will prevail. In addition, the materials, methods, and examples are illustrative only and not intended to be limiting.

Throughout this application various publications, published patent applications and published patents are referenced.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

### REFERENCES

Xie, Kabin, and Yinong Yang. “RNA-guided genome editing in plants using a CRISPR-Cas system.” Molecular plant 6.6 (2013): 1975-1983.

1. A method for producing a modified peanut plant with at least one improved phenotypic trait comprising characteristics of conferring reduced allergic response and/or having altered fatty acid content, wherein the method comprises steps of genetically modifying using targeted genome editing, at least one regulatory gene involved in allergen synthesis selected from Ara h (Arachis hypogaea) regulatory genes, for conferring the reduced allergic response, and/or at least one regulatory gene involved in fatty acid metabolism selected from FAD (Fatty Acid Desaturase) regulatory genes for conferring the altered fatty acid content.

2. (canceled)

3. (canceled)

4. The method according to claim 1, wherein the at least one regulatory gene involved in allergen synthesis is selected from the group consisting of Ara h1 having a genomic nucleotide sequence as set forth in SEQ ID NO:1 or a functional variant thereof, Ara h1.1 having a genomic nucleotide sequence as set forth in SEQ ID NO:341 or a functional variant thereof, Ara h2 having a genomic nucleotide sequence as set forth in SEQ ID NO:686 or a functional variant thereof, Ara h3 having a genomic nucleotide sequence as set forth in SEQ ID NO:908 or a functional variant thereof, Ara h5 having a genomic nucleotide sequence as set forth in SEQ ID NO:1161 or a functional variant thereof, Ara h6 having a genomic nucleotide sequence as set forth in SEQ ID NO:1348 or a functional variant thereof, Ara h7 having a genomic nucleotide sequence as set forth in SEQ ID NO:1514 or a functional variant thereof, Ara h8 having a genomic nucleotide sequence as set forth in SEQ ID NO:1690 or a functional variant thereof, Ara h9 having a genomic nucleotide sequence as set forth in SEQ ID NO: 1960 or a functional variant thereof, Ara h10 having a genomic nucleotide sequence as set forth in SEQ ID NO:2208 or a functional variant thereof, Ara h11 having a genomic nucleotide sequence as set forth in SEQ ID NO:2476 or a functional variant thereof, Ara h14 having a genomic nucleotide sequence as set forth in SEQ ID NO:2665 or a functional variant thereof, Ara h15 having a genomic nucleotide sequence as set forth in SEQ ID NO:2854 or a functional variant thereof, or any combination thereof, wherein the functional variant has at least 75% sequence identity to the corresponding Ara h nucleotide sequence.

5. The method according to claim 1, wherein the at least one regulatory gene involved in allergen synthesis encodes a polypeptide sequence selected from the group consisting of Ara h1 having a polypeptide sequence as set forth in SEQ ID NO:2 or a functional variant thereof, Ara h1.1 having a polypeptide sequence as set forth in SEQ ID NO:342 or a functional variant thereof, Ara h2 having a polypeptide sequence as set forth in SEQ ID NO:687 or a functional variant thereof, Ara h3 having a polypeptide sequence as set forth in SEQ ID NO:909 or a functional variant thereof, Ara h5 having a polypeptide sequence as set forth in SEQ ID NO:1162 or a functional variant thereof, Ara h6 having a polypeptide sequence as set forth in SEQ ID NO: 1349 or a functional variant thereof, Ara h7 having a polypeptide sequence as set forth in SEQ ID NO:1515 or a functional variant thereof, Ara h8 having a polypeptide sequence as set forth in SEQ ID NO: 1691 or a functional variant thereof, Ara h9 having a polypeptide sequence as set forth in SEQ ID NO: 1961 or a functional variant thereof, Ara h10 having a polypeptide sequence as set forth in SEQ ID NO:2209 or a functional variant thereof, Ara h11 having a polypeptide sequence as set forth in SEQ ID NO:2477 or a functional variant thereof, Ara h14 having a polypeptide sequence as set forth in SEQ ID NO:2666 or a functional variant thereof, Ara h15 having a polypeptide sequence as set forth in SEQ ID NO: 2855 or a functional variant thereof, or any combination thereof, wherein the functional variant has at least 75% sequence identity to the corresponding Ara h polypeptide sequence.

6. (canceled)

7. The method according to claim 1, wherein the at least one regulatory gene involved in fatty acid metabolism is selected from the group consisting of into FAD2-1 having a genomic nucleotide sequence as set forth in SEQ ID NO: 3065 or a functional variant thereof, FAD2-2 having a genomic nucleotide sequence as set forth in SEQ ID NO:3410 or a functional variant thereof, FAD2-3 having a genomic nucleotide sequence as set forth in SEQ ID NO:3809 or a functional variant thereof, FAD2-4 having a genomic nucleotide sequence as set forth in SEQ ID NO:4105 or a functional variant thereof, or any combination thereof, wherein the functional variant has at least 75% sequence identity to the corresponding FAD2 nucleotide sequence.

8. The method according to claim 1, wherein the at least one regulatory gene involved in fatty acid metabolism encodes a polypeptide selected from the group consisting of into FAD2-1 having a polypeptide sequence as set forth in SEQ ID NO:3066 or a functional variant thereof, FAD2-2 having a polypeptide sequence as set forth in SEQ ID NO:3411 or a functional variant thereof, FAD2-3 having a polypeptide sequence as set forth in SEQ ID NO:3810 or a functional variant thereof, FAD2-4 having a polypeptide sequence as set forth in SEQ ID NO:4106 or a functional variant thereof, or any combination thereof, wherein the functional variant has at least 75% sequence identity to the corresponding FAD2 polypeptide sequence.

9. (canceled)

10. The method according to claim 1, wherein the genetic modification in the Ara h (Arachis hypogaea) regulatory genes confers altered levels of allergens, different expression of allergens, expression of modified or mutated allergen molecules and/or lower, decreased, reduced and/or eliminated allergic response.

11. The method according to claim 10, wherein the altered levels of allergens are lower, decreased, reduced and/or eliminated.

12. The method according to claim 10, wherein the altered levels of allergens, different expression of allergens, expression of modified or mutated allergen molecules confer lower, decreased, reduced and/or eliminated allergic response.

13. The method according to claim 1, wherein the genetic modification in FAD (Fatty Acid Desaturase) regulatory genes confers altered levels or ratios of fatty acid content.

14. The method according to claim 13, wherein the altered levels or ratios of fatty acid content are (a) characterized by an altered fatty acid transport and/or an altered fatty acid conversion, and/or (b) selected from oleic:linoleic acid ratio of high oleic to linoleic acid ratio.

15. (canceled)

16. The method according to claim 1, wherein the at least one phenotypic trait is selected from the group consisting of lower allergic response, decreased allergic response, reduced allergic response, eliminated allergic response, longer shelf-life, high oleic acid to linoleic acid ratio, healthier fatty acid properties, and any combination thereof.

17. The method according to claim 1, wherein the peanut plant is an Ara h/FAD double mutant.

18. (canceled)

19. The method according to claim 1, wherein the targeted genome editing is selected from meganuclease, Clustered regularly interspaced short palindromic repeats (CRISPR) and CRISPR-associated (Cas) gene (CRISPR/Cas), Transcription activator-like effector nucleases (TALEN) or Zinc-finger nucleases (ZFNs) or any combination thereof.

20. (canceled)

21. (canceled)

22. The method according to claim 1, wherein the at least one regulatory gene involved in allergen synthesis and/or fatty acid metabolism is edited through deleting, silencing, downregulating, de-regulating, deactivating, knockout, loss of function, reducing expression, at least partially deleting, at least partially silencing, at least partially deactivating, removing, partially removing, duplicating, inverting, missense mutation, nonsense mutation, insertion, deletion, indel, substitution or duplication or any combination thereof of the regulatory region or coding region of the gene.

23. The method according to claim 1, wherein the at least one genetic modification in the regulatory gene involved in allergen synthesis is generated in planta via introduction of a construct comprising (a) Cas DNA and guide RNA (gRNA) sequence selected from the group consisting of SEQ ID NO:3-340, SEQ ID NO: 343-685, SEQ ID NO:688-907, SEQ ID NO:910-1160, SEQ ID NO: 1163-1347, SEQ ID NO: 1350-1513, SEQ ID NO: 1516-1689, SEQ ID NO: 1692-1959, SEQ ID NO: 1962-2207, SEQ ID NO:2210-2475, SEQ ID NO:2478-2664, SEQ ID NO:2667-2853, and SEQ ID NO: 2856-3064 for the Ara h 1, Ara h1.1, Ara h2, Ara h3, Ara h5, Ara h6, Ara h7, Ara h8, Ara h9, Ara h10, Ara h11, Ara h14, Ara h15, respectively or any combination thereof, or (b) a ribonucleoprotein (RNP) complex comprising Cas protein and gRNA sequence selected from the group consisting of SEQ ID NO:3-340, SEQ ID NO:343-685, SEQ ID NO: 688-907, SEQ ID NO:910-1160, SEQ ID NO:1163-1347, SEQ ID NO: 1350-1513, SEQ ID NO:1516-1689, SEQ ID NO:1692-1959, SEQ ID NO: 1962-2207, SEQ ID NO: 2210-2475, SEQ ID NO:2478-2664, SEQ ID NO:2667-2853, and SEQ ID NO:2856-3064 for the Ara h 1, Ara h1.1, Ara h2, Ara h3, Ara h5, Ara h6, Ara h7, Ara h8, Ara h9, Ara h10, Ara h11, Ara h14, Ara h15, respectively, or any combination thereof.

24. The method according to claim 1, wherein the at least one genetic modification in the regulatory gene involved in fatty acid metabolism is generated in planta via introduction of a construct comprising (a) Cas DNA and guide RNA (gRNA) sequence selected from the group consisting of SEQ ID NO:3067-3409, SEQ ID NO:3412-3808, SEQ ID NO:3811-4104, SEQ ID NO:4107-4400 for the FAD2-1-FAD2-4, respectively, or any combination thereof, or (b) a ribonucleoprotein (RNP) complex comprising Cas protein and gRNA sequence selected from the group consisting of SEQ ID NO:3067-3409, SEQ ID NO:3412-3808, SEQ ID NO:3811-4104, SEQ ID NO:4107-4400 for the FAD2-1-FAD2-4, respectively, or any combination thereof.

25. (canceled)

26. The method according to claim 1, wherein the method comprises the steps of:
a. selecting, from a peanut plant genome, at least one regulatory gene and/or a promotor of a regulatory gene involved in allergen synthesis and/or fatty acid metabolism of the peanuts plant;
b. synthesizing or designing at least one guide RNA (gRNA) comprising a nucleotide sequence complementary to the at least one selected peanut gene;
c. transforming peanut plant cells, cell nucleus, tissues, seeds, or progeny cell of said peanut plant with a construct comprising (a) Cas nucleotide sequence operably linked to the at least one gRNA, or (b) a ribonucleoprotein (RNP) complex comprising Cas protein and the at least one gRNA;
d. culturing the peanut plant cells, cell nucleus, tissues, seeds, or progeny cell thereof;
e. selecting the peanut cells, cell nucleus, tissues, seeds, or progeny cell thereof which express desired mutations/genomic modifications in the at least one selected peanut regulatory gene and/or a promotor of a regulatory gene, and
f. regenerating a peanut plant from the transformed plant cell, cell nucleus, or tissue, seed or plant progeny cell thereof carrying the desired mutations/genomic modifications; and
g. screening the regenerated peanut plants for a peanut plant with at least one improved phenotypic trait related to at least one regulatory gene and/or a promotor of a regulatory gene involved in allergen synthesis and/or at least one regulatory gene and/or a promotor of a regulatory gene involved in fatty acid metabolism.

27. (canceled)

28. (canceled)

29. (canceled)

30. A modified peanut plant, plant part, plant cell, plant progeny, plant tissue, plant seed, plant pod, a tissue culture, a tissue culture of regenerable cells, protoplasts or callus, produced by the method of claim 1.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. Harvestable parts, products or products thereof derived from a modified peanut plant according to claim 30, wherein said harvestable parts are preferably shoot biomass pods and/or seeds.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. Use of (a) a nucleotide sequence as set forth in SEQ ID NO:1, SEQ ID NO: 341, SEQ ID NO: 686, SEQ ID NO: 908, SEQ ID NO: 1161, SEQ ID NO: 1348, SEQ ID NO: 1514, SEQ ID NO: 1690, SEQ ID NO: 1960, SEQ ID NO:2208, SEQ ID NO:2476, SEQ ID NO:2665 and SEQ ID NO:2854 and/or (b) a nucleotide sequence as set forth in at least one of SEQ ID NO:3-340, SEQ ID NO:3-340, SEQ ID NO:343-685, SEQ ID NO:688-907, SEQ ID NO:910-1160, SEQ ID NO:1163-1347, SEQ ID NO: 1350-1513, SEQ ID NO: 1516-1689, SEQ ID NO: 1692-1959, SEQ ID NO: 1962-2207, SEQ ID NO:2210-2475, SEQ ID NO:2478-2664, SEQ ID NO:2667-2853, SEQ ID NO:2856-3064 and any combination thereof for targeted genome modification of peanut, for generating and/or producing a modified peanut plant with at least one genetically modified regulatory gene involved in allergen synthesis, and (b) a nucleotide sequence as set forth in SEQ ID NO: 3065, SEQ ID NO:3410, SEQ ID NO:3809 and SEQ ID NO:4105, and/or a nucleotide sequence as set forth in at least one of SEQ ID NO:3067-3409, SEQ ID NO:3412-3808, SEQ ID NO:3811-4104, SEQ ID NO:4107-4400 and any combination thereof, for targeted genome modification of peanut, for generating and/or producing a modified peanut plant with at least one genetically modified regulatory gene involved in fatty acid metabolism.

78. (canceled)
